SEQUENCE LISTING (187 sequences)

SEQ ID NO: 1 - protein, 20 aa, synthetic construct
MQSGTHWRVLGLCLLSVGVW

SEQ ID NO: 2 - protein, 104 aa, synthetic construct
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 3 - protein, 26 aa, synthetic construct
VMSVATIVIVDICITGGLLLLVYYWS

SEQ ID NO: 4 - protein, 55 aa, synthetic construct
KNRKAKAKPVTRGAGAGGRQRGQNKERPPPVPNPDYEPIRKGQRDLYSGLNQRRI

SEQ ID NO: 5 - RNA, 624 nt, synthetic construct
  source 1..624: mol_type = other RNA
atgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatctga

SEQ ID NO: 6 - DNA, 621 bp, synthetic construct
  source 1..621: mol_type = other DNA
atgcagagcggcacccactggagagtgctgggcctgtgcctgctgagcgtgggcgtgtggggccaggacggcaacgaggagatgggcggcatcacccagaccccctacaaggtgagcatcagcggcaccaccgtgatcctgacctgcccccagtaccccggcagcgagatcctgtggcagcacaacgacaagaacatcggcggcgacgaggacgacaagaacatcggcagcgacgaggaccacctgagcctgaaggagttcagcgagctggagcagagcggctactacgtgtgctaccccagaggcagcaagcccgaggacgccaacttctacctgtacctgagagccagagtgtgcgagaactgcatggagatggacgtgatgagcgtggccaccatcgtgatcgtggacatctgcatcaccggcggcctgctgctgctggtgtactactggagcaagaacagaaaggccaaggccaagcccgtgaccagaggcgccggcgccggcggcagacagagaggccagaacaaggagagacccccccccgtgcccaaccccgactacgagcccatcagaaagggccagagagacctgtacagcggcctgaaccagagaagaatc

SEQ ID NO: 7 - protein, 207 aa, synthetic construct
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 8 - protein, 20 aa, synthetic construct
MEHSTFLSGLVLATLLSQVS

SEQ ID NO: 9 - protein, 84 aa, synthetic construct [sequence omitted]

SEQ ID NO: 10 - protein, 21 aa, synthetic construct
GIIVTDVIATLLLALGVFCFA

SEQ ID NO: 11 - protein, 45 aa, synthetic construct
GHETGRLSGAADTQALLRNDQVYQPLRDRDDAQYSHLGGNWARNK

SEQ ID NO: 12 - RNA, 516 nt, synthetic construct
  source 1..516: mol_type = other RNA
atggaacatagcacgtttctctctggcctggtactggctacccttctctcgcaagtgagccccttcaagatacctatagaggaacttgaggacagagtgtttgtgaattgcaataccagcatcacatgggtagagggaacggtgggaacactgctctcagacattacaagactggacctgggaaaacgcatcctggacccacgaggaatatataggtgtaatgggacagatatatacaaggacaaagaatctaccgtgcaagttcattatcgaatgtgccagagctgtgtggagctggatccagccaccgtggctggcatcattgtcactgatgtcattgccactctgctccttgctttgggagtcttctgctttgctggacatgagactggaaggctgtctggggctgccgacacacaagctctgttgaggaatgaccaggtctatcagcccctccgagatcgagatgatgctcagtacagccaccttggaggaaactgggctcggaacaagtga

SEQ ID NO: 13 - RNA, 513 nt, synthetic construct
  source 1..513: mol_type = other RNA
atggagcacagcaccttcctgagcggcctggtgctggccaccctgctgagccaggtgagccccttcaagatccccatcgaggagctggaggacagagtgttcgtgaactgcaacaccagcatcacctgggtggagggcaccgtgggcaccctgctgagcgacatcaccagactggacctgggcaagagaatcctggaccccagaggcatctacagatgcaacggcaccgacatctacaaggacaaggagagcaccgtgcaggtgcactacagaatgtgccagagctgcgtggagctggaccccgccaccgtggccggcatcatcgtgaccgacgtgatcgccaccctgctgctggccctgggcgtgttctgcttcgccggccacgagaccggcagactgagcggcgccgccgacacccaggccctgctgagaaacgaccaggtgtaccagcccctgagagacagagacgacgcccagtacagccacctgggcggcaactgggccagaaacaag

SEQ ID NO: 14 - protein, 171 aa, synthetic construct
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 15 - protein, 22 aa, synthetic construct
MEQGKGLAVLILAIILLQGTLA

SEQ ID NO: 16 - protein, 94 aa, synthetic construct
QSIKGNHLVKVYDYQEDGSVLLTCDAEAKNITWFKDGKMIGFLTEDKKKWNLGSNAKDPRGMYQCKGSQNKSKPLQVYYRMCQNCIELNAATIS

SEQ ID NO: 17 - protein, 21 aa, synthetic construct
GFLFAEIVSIFVLAVGVYFIA

SEQ ID NO: 18 - protein, 45 aa, synthetic construct
GQDGVRQSRASDKQTLLPNDQLYQPLKDREDDQYSHLQGNQLRRN

SEQ ID NO: 19 - RNA, 549 nt, synthetic construct
  source 1..549: mol_type = other RNA
atggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgagggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaattga

SEQ ID NO: 20 - RNA, 546 nt, synthetic construct
  source 1..546: mol_type = other RNA
atggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgtgggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaat

SEQ ID NO: 21 - protein, 182 aa, synthetic construct
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 22 - protein, 21 aa, synthetic construct
MKWKALFTAAILQAQLPITEA

SEQ ID NO: 23 - protein, 9 aa, synthetic construct
QSFGLLDPK

SEQ ID NO: 24 - protein, 21 aa, synthetic construct
LCYLLDGILFIYGVILTALFL

SEQ ID NO: 25 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 26 - RNA, 492 nt, synthetic construct
  source 1..492: mol_type = other RNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 27 - protein, 164 aa, synthetic construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 28 - RNA, 495 nt, synthetic construct
  source 1..495: mol_type = other RNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 29 - protein, 18 aa, synthetic construct
EGRGSLLTCGDVEENPGP

SEQ ID NO: 30 - protein, 19 aa, synthetic construct
ATNFSLLKQAGDVEENPGP

SEQ ID NO: 31 - protein, 20 aa, synthetic construct
QCTNYALLKLAGDVESNPGP

SEQ ID NO: 32 - protein, 22 aa, synthetic construct
VKQTLNFDLLKLAGDVESNPGP

SEQ ID NO: 33 - DNA, 624 bp, synthetic construct
  source 1..624: mol_type = other DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgctttgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggcccc

SEQ ID NO: 34 - DNA, 675 bp, synthetic construct
  source 1..675: mol_type = other DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgaggagtaagaggagcaggctcctgcacagtgactacatgaacatgactccccgccgccccgggcccacccgcaagcattaccagccctatgccccaccacgcgacttcgcagcctatcgctcaagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggcccc

SEQ ID NO: 35 - DNA, 747 bp, synthetic construct
  source 1..747: mol_type = other DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgaggagtaagaggagcaggctcctgcacagtgactacatgaacatgactccccgccgccccgggcccacccgcaagcattaccagccctatgccccaccacgcgacttcgcagcctatcgctcactttgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggcccc

SEQ ID NO: 36 - protein, 208 aa, synthetic construct
MKWKALFTAAILQAQLPITEAQSFGLLDPKLCYLLDGILFIYGVILTALFLLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGP

SEQ ID NO: 37 - protein, 225 aa, synthetic construct
MKWKALFTAAILQAQLPITEAQSFGLLDPKLCYLLDGILFIYGVILTALFLRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGP

SEQ ID NO: 38 - protein, 249 aa, synthetic construct
MKWKALFTAAILQAQLPITEAQSFGLLDPKLCYLLDGILFIYGVILTALFLRSKRSRLLHSDYMNMTPRRPGPTRKHYQPYAPPRDFAAYRSLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGP

SEQ ID NO: 39 - DNA, 72 bp, synthetic construct
  source 1..72: mol_type = other DNA
ctttgcgcacgcccacgccgcagccccgcccaagaagatggcaaagtctacatcaacatgccaggcaggggc

SEQ ID NO: 40 - DNA, 123 bp, synthetic construct
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctca

SEQ ID NO: 41 - DNA, 195 bp, synthetic construct
  source 1..195: mol_type = other DNA
aggagtaagaggagcaggctcctgcacagtgactacatgaacatgactccccgccgccccgggcccacccgcaagcattaccagccctatgccccaccacgcgacttcgcagcctatcgctcactttgcgcacgcccacgccgcagccccgcccaagaagatggcaaagtctacatcaacatgccaggcaggggc

SEQ ID NO: 42 - protein, 24 aa, synthetic construct
LCARPRRSPAQEDGKVYINMPGRG

SEQ ID NO: 43 - protein, 41 aa, synthetic construct
RSKRSRLLHSDYMNMTPRRPGPTRKHYQPYAPPRDFAAYRS

SEQ ID NO: 44 - protein, 65 aa, synthetic construct
RSKRSRLLHSDYMNMTPRRPGPTRKHYQPYAPPRDFAAYRSLCARPRRSPAQEDGKVYINMPGRG

SEQ ID NO: 45 - DNA, 2976 bp, synthetic construct
  source 1..2976: mol_type = other DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgctttgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggccccatggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgtgggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaatgtgaagcagaccctgaacttcgacctgctgaagctggccggcgacgtggagagcaaccccggccccatggagcacagcaccttcctgagcggcctggtgctggccaccctgctgagccaggtgagccccttcaagatccccatcgaggagctggaggacagagtgttcgtgaactgcaacaccagcatcacctgggtggagggcaccgtgggcaccctgctgagcgacatcaccagactggacctgggcaagagaatcctggaccccagaggcatctacagatgcaacggcaccgacatctacaaggacaaggagagcaccgtgcaggtgcactacagaatgtgccagagctgcgtggagctggaccccgccaccgtggccggcatcatcgtgaccgacgtgatcgccaccctgctgctggccctgggcgtgttctgcttcgccggccacgagaccggcagactgagcggcgccgccgacacccaggccctgctgagaaacgaccaggtgtaccagcccctgagagacagagacgacgcccagtacagccacctgggcggcaactgggccagaaacaaggagggcagaggcagcctgctgacctgcggcgacgtggaggagaaccccggccccatgcagagcggcacccactggagagtgctgggcctgtgcctgctgagcgtgggcgtgtggggccaggacggcaacgaggagatgggcggcatcacccagaccccctacaaggtgagcatcagcggcaccaccgtgatcctgacctgcccccagtaccccggcagcgagatcctgtggcagcacaacgacaagaacatcggcggcgacgaggacgacaagaacatcggcagcgacgaggaccacctgagcctgaaggagttcagcgagctggagcagagcggctactacgtgtgctaccccagaggcagcaagcccgaggacgccaacttctacctgtacctgagagccagagtgtgcgagaactgcatggagatggacgtgatgagcgtggccaccatcgtgatcgtggacatctgcatcaccggcggcctgctgctgctggtgtactactggagcaagaacagaaaggccaaggccaagcccgtgaccagaggcgccggcgccggcggcagacagagaggccagaacaaggagagacccccccccgtgcccaaccccgactacgagcccatcagaaagggccagagagacctgtacagcggcctgaaccagagaagaatcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagc

SEQ ID NO: 46 - protein, 992 aa, synthetic construct
MKWKALFTAAILQAQLPITEAQSFGLLDPKLCYLLDGILFIYGVILTALFLLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNVKQTLNFDLLKLAGDVESNPGPMEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 47 - DNA, 3027 bp, synthetic construct
  source 1..3027: mol_type = other DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgaggagtaagaggagcaggctcctgcacagtgactacatgaacatgactccccgccgccccgggcccacccgcaagcattaccagccctatgccccaccacgcgacttcgcagcctatcgctcaagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggccccatggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgtgggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaatgtgaagcagaccctgaacttcgacctgctgaagctggccggcgacgtggagagcaaccccggccccatggagcacagcaccttcctgagcggcctggtgctggccaccctgctgagccaggtgagccccttcaagatccccatcgaggagctggaggacagagtgttcgtgaactgcaacaccagcatcacctgggtggagggcaccgtgggcaccctgctgagcgacatcaccagactggacctgggcaagagaatcctggaccccagaggcatctacagatgcaacggcaccgacatctacaaggacaaggagagcaccgtgcaggtgcactacagaatgtgccagagctgcgtggagctggaccccgccaccgtggccggcatcatcgtgaccgacgtgatcgccaccctgctgctggccctgggcgtgttctgcttcgccggccacgagaccggcagactgagcggcgccgccgacacccaggccctgctgagaaacgaccaggtgtaccagcccctgagagacagagacgacgcccagtacagccacctgggcggcaactgggccagaaacaaggagggcagaggcagcctgctgacctgcggcgacgtggaggagaaccccggccccatgcagagcggcacccactggagagtgctgggcctgtgcctgctgagcgtgggcgtgtggggccaggacggcaacgaggagatgggcggcatcacccagaccccctacaaggtgagcatcagcggcaccaccgtgatcctgacctgcccccagtaccccggcagcgagatcctgtggcagcacaacgacaagaacatcggcggcgacgaggacgacaagaacatcggcagcgacgaggaccacctgagcctgaaggagttcagcgagctggagcagagcggctactacgtgtgctaccccagaggcagcaagcccgaggacgccaacttctacctgtacctgagagccagagtgtgcgagaactgcatggagatggacgtgatgagcgtggccaccatcgtgatcgtggacatctgcatcaccggcggcctgctgctgctggtgtactactggagcaagaacagaaaggccaaggccaagcccgtgaccagaggcgccggcgccggcggcagacagagaggccagaacaaggagagacccccccccgtgcccaaccccgactacgagcccatcagaaagggccagagagacctgtacagcggcctgaaccagagaagaatcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagc

SEQ ID NO: 48 - protein, 1009 aa, synthetic construct
MKWKALFTAAILQAQLPITEAQSFGLLDPKLCYLLDGILFIYGVILTALFLRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNVKQTLNFDLLKLAGDVESNPGPMEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 49 - DNA, 3099 bp, synthetic construct
  source 1..3099: mol_type = other DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgaggagtaagaggagcaggctcctgcacagtgactacatgaacatgactccccgccgccccgggcccacccgcaagcattaccagccctatgccccaccacgcgacttcgcagcctatcgctcactttgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggccccatggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgtgggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaatgtgaagcagaccctgaacttcgacctgctgaagctggccggcgacgtggagagcaaccccggccccatggagcacagcaccttcctgagcggcctggtgctggccaccctgctgagccaggtgagccccttcaagatccccatcgaggagctggaggacagagtgttcgtgaactgcaacaccagcatcacctgggtggagggcaccgtgggcaccctgctgagcgacatcaccagactggacctgggcaagagaatcctggaccccagaggcatctacagatgcaacggcaccgacatctacaaggacaaggagagcaccgtgcaggtgcactacagaatgtgccagagctgcgtggagctggaccccgccaccgtggccggcatcatcgtgaccgacgtgatcgccaccctgctgctggccctgggcgtgttctgcttcgccggccacgagaccggcagactgagcggcgccgccgacacccaggccctgctgagaaacgaccaggtgtaccagcccctgagagacagagacgacgcccagtacagccacctgggcggcaactgggccagaaacaaggagggcagaggcagcctgctgacctgcggcgacgtggaggagaaccccggccccatgcagagcggcacccactggagagtgctgggcctgtgcctgctgagcgtgggcgtgtggggccaggacggcaacgaggagatgggcggcatcacccagaccccctacaaggtgagcatcagcggcaccaccgtgatcctgacctgcccccagtaccccggcagcgagatcctgtggcagcacaacgacaagaacatcggcggcgacgaggacgacaagaacatcggcagcgacgaggaccacctgagcctgaaggagttcagcgagctggagcagagcggctactacgtgtgctaccccagaggcagcaagcccgaggacgccaacttctacctgtacctgagagccagagtgtgcgagaactgcatggagatggacgtgatgagcgtggccaccatcgtgatcgtggacatctgcatcaccggcggcctgctgctgctggtgtactactggagcaagaacagaaaggccaaggccaagcccgtgaccagaggcgccggcgccggcggcagacagagaggccagaacaaggagagacccccccccgtgcccaaccccgactacgagcccatcagaaagggccagagagacctgtacagcggcctgaaccagagaagaatcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagc

SEQ ID NO: 50 - protein, 1033 aa, synthetic construct
MKWKALFTAAILQAQLPITEAQSFGLLDPKLCYLLDGILFIYGVILTALFLRSKRSRLLHSDYMNMTPRRPGPTRKHYQPYAPPRDFAAYRSLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNVKQTLNFDLLKLAGDVESNPGPMEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 51 - DNA, 828 bp, synthetic construct
  source 1..828: mol_type = other DNA
atgaagaagcacctgaccacctttctggtcatcctgtggctgtacttctacagaggcaacggcaagaaccaggtggaacagagccctcagagcctgatcatcctggaaggcaagaactgcaccctgcagtgcaactacaccgtgtctcccttcagcaacctgcggtggtacaagcaggatacaggcagaggccctgtgtctctgaccatcatgaccttcagcgagaacaccaagagcaacggccggtacaccgccacactggatgccgatacaaagcagagcagcctgcacatcacagccagccagctgagcgatagcgccagctacatctgcgtggtgtccgatagaggcagcaccctgggcagactgtactttggcagaggcacccagctgaccgtgtggcccgatattcagaaccctgatcctgccgtgtaccagctgagagacagcaagtccagcgacaagagcgtgtgcctgttcaccgacttcgacagccagaccaacgtgtcccagagcaaggacagcgacgtgtacatcaccgacaagtgcgtgctggacatgcggagcatggacttcaagagcaatagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctgctgaaagtggccggcttcaacctgctgatgaccctgagactgtggtctagc

SEQ ID NO: 52 - protein, 276 aa, synthetic construct
MKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVSDRGSTLGRLYFGRGTQLTVWPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 53 - DNA, 828 bp, synthetic construct; identical to SEQ ID NO: 51
  source 1..828: mol_type = other DNA

SEQ ID NO: 54 - protein, 314 aa, synthetic construct
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSELRALGPSSYNSPLHFGNGTRLTVTDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 55 - DNA, 936 bp, synthetic construct
  source 1..936: mol_type = other DNA
atgaccatccggctgctgtgctatatgggcttctacttcctcggagccggcctgatggaagccgacatctaccagacacctagatacctggtcatcggcaccggcaaaaagatcaccctggaatgctcccagaccatgggccacgacaagatgtactggtatcagcaggaccccggcatggaactgcatctgatccactacagctacggcgtgaacagcaccgagaagggcgatctgtctagcgagagcaccgtgtccagaatccggaccgagcacttcccactgacactggaaagcgccagacctagccacaccagccagtacctgtgtgcctctagtgaaggcggaggactcaagttggcgaaaaacatccaatactttggtgctggcacccggctttctgtcctggacctgaacaaggtgttccctccagaggtggccgtgttcgagccttctgaggccgagatcagccacacacagaaagccacactcgtgtgtctggccaccggctttttccccgatcacgtggaactgtcttggtgggtcaacggcaaagaggtgcacagcggcgtctgcacagatccccagcctctgaaagaacagcccgctctgaacgacagccggtactgtctgagcagcagactgagagtgtccgccaccttctggcagaaccccagaaaccacttcagatgccaggtgcagttttacggcctgagcgagaacgacgagtggacccaggatagagccaagcctgtgacacagatcgtgtctgccgaagcctggggcagagccgattgtggctttaccagcgtgtcataccagcagggcgtgctgtctgccaccatcctgtatgagattctgctgggcaaagccactctgtacgccgtgctggtgtctgcccttgtgctgatggccatggtcaagagaaaggacttc

SEQ ID NO: 56 - protein, 312 aa, synthetic construct
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSEGGGLKLAKNIQYFGAGTRLSVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 57 - DNA, 933 bp, synthetic construct
  source 1..933: mol_type = other DNA
atgaccatccggctgctgtgctatatgggcttctacttcctcggagccggcctgatggaagccgacatctaccagacacctagatacctggtcatcggcaccggcaaaaagatcaccctggaatgctcccagaccatgggccacgacaagatgtactggtatcagcaggaccccggcatggaactgcatctgatccactacagctacggcgtgaacagcaccgagaagggcgatctgtctagcgagagcaccgtgtccagaatccggaccgagcacttcccactgacactggaaagcgccagacctagccacaccagccagtacctgtgtgcctctagtgaattcgccagctccgtgcgagggaatactatctactttggtgagggatcttggcttacggtagtagacctgaacaaggtgttccctccagaggtggccgtgttcgagccttctgaggccgagatcagccacacacagaaagccacactcgtgtgtctggccaccggctttttccccgatcacgtggaactgtcttggtgggtcaacggcaaagaggtgcacagcggcgtctgcacagatccccagcctctgaaagaacagcccgctctgaacgacagccggtactgtctgagcagcagactgagagtgtccgccaccttctggcagaaccccagaaaccacttcagatgccaggtgcagttttacggcctgagcgagaacgacgagtggacccaggatagagccaagcctgtgacacagatcgtgtctgccgaagcctggggcagagccgattgtggctttaccagcgtgtcataccagcagggcgtgctgtctgccaccatcctgtatgagattctgctgggcaaagccactctgtacgccgtgctggtgtctgcccttgtgctgatggccatggtcaagagaaaggacttc

SEQ ID NO: 58 - protein, 311 aa, synthetic construct
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSEFASSVRGNTIYFGEGSWLTVVDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 59 - DNA, 930 bp, synthetic construct
  source 1..930: mol_type = other DNA
atgaccatccggctgctgtgctatatgggcttctacttcctcggagccggcctgatggaagccgacatctaccagacacctagatacctggtcatcggcaccggcaaaaagatcaccctggaatgctcccagaccatgggccacgacaagatgtactggtatcagcaggaccccggcatggaactgcatctgatccactacagctacggcgtgaacagcaccgagaagggcgatctgtctagcgagagcaccgtgtccagaatccggaccgagcacttcccactgacactggaaagcgccagacctagccacaccagccagtacctgtgtgccactggacagggggcgcaagatacgcagtattttggcccaggcacccggctgacagtgctcgaggacctcaagaatgtgtttccgcccgaagtcgcggtttttgaaccatcagaagccgagatctctcatacacaaaaggcgacgctcgtatgcctcgcgacgggattttatccggaccacgtcgagctttcctggtgggttaacggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagaatcataccaacaaggagtattgagcgcgacaattctttacgaaattctgcttgggaaagcgactctgtacgcggtgctcgtgtccgctttggttcttatggcaatggttaaacgaaaggatagtaggggc

SEQ ID NO: 60 - protein, 310 aa, synthetic construct
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCATGQGAQDTQYFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 61 - DNA, 930 bp, synthetic construct
  source 1..930: mol_type = other DNA
atgaccatccggctgctgtgctatatgggcttctacttcctcggagccggcctgatggaagccgacatctaccagacacctagatacctggtcatcggcaccggcaaaaagatcaccctggaatgctcccagaccatgggccacgacaagatgtactggtatcagcaggaccccggcatggaactgcatctgatccactacagctacggcgtgaacagcaccgagaagggcgatctgtctagcgagagcaccgtgtccagaatccggaccgagcacttcccactgacactggaaagcgccagacctagccacaccagccagtacctgtgtgccagcagtgatggggtggggagcaatcagccccagcattttggtgatgggactcgactctccatcctagaggacctcaataaggtgtttccgcccgaagtcgcggtttttgaaccatcagaagccgagatctctcatacacaaaaggcgacgctcgtatgcctcgcgacgggatttttcccggaccacgtcgagctttcctggtgggttaacggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagtgtcataccaacaaggagtattgagcgcgacaattctttacgaaattctgcttgggaaagcgactctgtacgcggtgctcgtgtccgctttggttcttatggcaatggttaaacgaaaggacttc

SEQ ID NO: 62 - protein, 310 aa, synthetic construct
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSDGVGSNQPQHFGDGTRLSILEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 63 - DNA, 924 bp, synthetic construct
  source 1..924: mol_type = other DNA
atgaccatccggctgctgtgctatatgggcttctacttcctcggagccggcctgatggaagccgacatctaccagacacctagatacctggtcatcggcaccggcaaaaagatcaccctggaatgctcccagaccatgggccacgacaagatgtactggtatcagcaggaccccggcatggaactgcatctgatccactacagctacggcgtgaacagcaccgagaagggcgatctgtctagcgagagcaccgtgtccagaatccggaccgagcacttcccactgacactggaaagcgccagacctagccacaccagccagtacctgtgtgccagcagtgagggggctggaaacaccatatattttggagagggaagttggctcactgttgtagaggacctcaataaggtgtttccgcccgaagtcgcggtttttgaaccatcagaagccgagatctctcatacacaaaaggcgacgctcgtatgcctcgcgacgggatttttcccggaccacgtcgagctttcctggtgggttaacggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagtgtcataccaacaaggagtattgagcgcgacaattctttacgaaattctgcttgggaaagcgactctgtacgcggtgctcgtgtccgctttggttcttatggcaatggttaaacgaaaggacttc

SEQ ID NO: 64 - protein, 308 aa, synthetic construct
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSEGAGNTIYFGEGSWLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 65 - DNA, 927 bp, synthetic construct
  source 1..927: mol_type = other DNA
atgaccatccggctgctgtgctatatgggcttctacttcctcggagccggcctgatggaagccgacatctaccagacacctagatacctggtcatcggcaccggcaaaaagatcaccctggaatgctcccagaccatgggccacgacaagatgtactggtatcagcaggaccccggcatggaactgcatctgatccactacagctacggcgtgaacagcaccgagaagggcgatctgtctagcgagagcaccgtgtccagaatccggaccgagcacttcccactgacactggaaagcgccagacctagccacaccagccagtacctgtgtgccagcagtgacagggatagcaatcagccccagcattttggtgatgggactcgactctccatcctagaggacctcaataaggtgtttccgcccgaagtcgcggtttttgaaccatcagaagccgagatctctcatacacaaaaggcgacgctcgtatgcctcgcgacgggatttttcccggaccacgtcgagctttcctggtgggttaacggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagtgtcataccaacaaggagtattgagcgcgacaattctttacgaaattctgcttgggaaagcgactctgtacgcggtgctcgtgtccgctttggttcttatggcaatggttaaacgaaaggacttc

SEQ ID NO: 66 - protein, 309 aa, synthetic construct
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSDRDSNQPQHFGDGTRLSILEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 67 - DNA, 936 bp, synthetic construct
  source 1..936: mol_type = other DNA
atgaccatccggctgctgtgctatatgggcttctacttcctcggagccggcctgatggaagccgacatctaccagacacctagatacctggtcatcggcaccggcaaaaagatcaccctggaatgctcccagaccatgggccacgacaagatgtactggtatcagcaggaccccggcatggaactgcatctgatccactacagctacggcgtgaacagcaccgagaagggcgatctgtctagcgagagcaccgtgtccagaatccggaccgagcacttcccactgacactggaaagcgccagacctagccacaccagccagtacctgtgtgccagcgtgggtccggtaccctcctacaatgagcagttcttcgggccagggacacggctcaccgtgctagaggacctcaagaatgtgtttccgcccgaagtcgcggtttttgaaccatcagaagccgagatctctcatacacaaaaggcgacgctcgtatgcctcgcgacgggattttatccggaccacgtcgagctttcctggtgggttaacggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagaatcataccaacaaggagtattgagcgcgacaattctttacgaaattctgcttgggaaagcgactctgtacgcggtgctcgtgtccgctttggttcttatggcaatggttaaacgaaaggatagtaggggc

SEQ ID NO: 68 - protein, 312 aa, synthetic construct
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASVGPVPSYNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 69 - DNA, 939 bp, synthetic construct
  source 1..939: mol_type = other DNA
atgaccatccggctgctgtgctatatgggcttctacttcctcggagccggcctgatggaagccgacatctaccagacacctagatacctggtcatcggcaccggcaaaaagatcaccctggaatgctcccagaccatgggccacgacaagatgtactggtatcagcaggaccccggcatggaactgcatctgatccactacagctacggcgtgaacagcaccgagaagggcgatctgtctagcgagagcaccgtgtccagaatccggaccgagcacttcccactgacactggaaagcgccagacctagccacaccagccagtacctgtgtgccagcagtggggtgactagcgcctcctacaatgagcagttcttcgggccagggacacggctcaccgtgctagaggacctcaagaatgtgtttccgcccgaagtcgcggtttttgaaccatcagaagccgagatctctcatacacaaaaggcgacgctcgtatgcctcgcgacgggattttatccggaccacgtcgagctttcctggtgggttaacggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagaatcataccaacaaggagtattgagcgcgacaattctttacgaaattctgcttgggaaagcgactctgtacgcggtgctcgtgtccgctttggttcttatggcaatggttaaacgaaaggatagtaggggc

SEQ ID NO: 70 - protein, 313 aa, synthetic construct
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSGVTSASYNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 71 - DNA, 930 bp, synthetic construct
  source 1..930: mol_type = other DNA
atgaccatccggctgctgtgctatatgggcttctacttcctcggagccggcctgatggaagccgacatctaccagacacctagatacctggtcatcggcaccggcaaaaagatcaccctggaatgctcccagaccatgggccacgacaagatgtactggtatcagcaggaccccggcatggaactgcatctgatccactacagctacggcgtgaacagcaccgagaagggcgatctgtctagcgagagcaccgtgtccagaatccggaccgagcacttcccactgacactggaaagcgccagacctagccacaccagccagtacctgtgtgccagcagtggaggaggggaggagacccagtacttcgggccaggcacgcggctcctggtgctcgaggacctcaagaatgtgtttccgcccgaagtcgcggtttttgaaccatcagaagccgagatctctcatacacaaaaggcgacgctcgtatgcctcgcgacgggattttatccggaccacgtcgagctttcctggtgggttaacggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagaatcataccaacaaggagtattgagcgcgacaattctttacgaaattctgcttgggaaagcgactctgtacgcggtgctcgtgtccgctttggttcttatggcaatggttaaacgaaaggatagtaggggc

SEQ ID NO: 72 - protein, 310 aa, synthetic construct
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSGGGEETQYFGPGTRLLVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 73 - DNA, 927 bp, synthetic construct
  source 1..927: mol_type = other DNA
atgaccatccggctgctgtgctatatgggcttctacttcctcggagccggcctgatggaagccgacatctaccagacacctagatacctggtcatcggcaccggcaaaaagatcaccctggaatgctcccagaccatgggccacgacaagatgtactggtatcagcaggaccccggcatggaactgcatctgatccactacagctacggcgtgaacagcaccgagaagggcgatctgtctagcgagagcaccgtgtccagaatccggaccgagcacttcccactgacactggaaagcgccagacctagccacaccagccagtacctgtgtgccagcagtgcgcagggggtcagcgaaaaactgttttttggcagtggaacccagctctctgtcttggaggacctcaataaggtgtttccgcccgaagtcgcggtttttgaaccatcagaagccgagatctctcatacacaaaaggcgacgctcgtatgcctcgcgacgggatttttcccggaccacgtcgagctttcctggtgggttaacggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagtgtcataccaacaaggagtattgagcgcgacaattctttacgaaattctgcttgggaaagcgactctgtacgcggtgctcgtgtccgctttggttcttatggcaatggttaaacgaaaggacttc

SEQ ID NO: 74 - protein, 309 aa, synthetic construct
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSAQGVSEKLFFGSGTQLSVLEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 75 - DNA, 63 bp, synthetic construct
  source 1..63: mol_type = other DNA
actggacagggggcgcaagatacgcagtattttggcccaggcacccggctgacagtgctcgag

SEQ ID NO: 76 - DNA, 63 bp, synthetic construct
  source 1..63: mol_type = other DNA
agcagtgcccctggagggtctgaagctttctttggacaaggcaccagactcacagttgtagag

SEQ ID NO: 77 - DNA, 69 bp, synthetic construct
  source 1..69: mol_type = other DNA
agcagtgaactcgacagggaaggaaacaccatatattttggagagggaagttggctcactgttgtagag

SEQ ID NO: 78 - DNA, 69 bp, synthetic construct
  source 1..69: mol_type = other DNA
agcagtgatatgggacccgtctcctacgagcagtacttcgggccgggcaccaggctcacggtcacagag

SEQ ID NO: 79 - DNA, 69 bp, synthetic construct
  source 1..69: mol_type = other DNA
agcagtgaggaagagcggggcccctacgagcagtacttcgggccgggcaccaggctcacggtcacagag

SEQ ID NO: 80 - DNA, 69 bp, synthetic construct
  source 1..69: mol_type = other DNA
agcagtgatggggtggggagcaatcagccccagcattttggtgatgggactcgactctccatcctagag

SEQ ID NO: 81 - DNA, 57 bp, synthetic construct
  source 1..57: mol_type = other DNA
agcagtgacccctacgagcagtacttcgggccgggcaccaggctcacggtcacagag

SEQ ID NO: 82 - DNA, 75 bp, synthetic construct
  source 1..75: mol_type = other DNA
agcagtgaggccccaacaggaaccggggccaacgtcctgactttcggggccggcagcaggctgaccgtgctggag

SEQ ID NO: 83 - DNA, 72 bp, synthetic construct
  source 1..72: mol_type = other DNA
agcagtgatcctcgactagcggggggggtcgagcagtacttcgggccgggcaccaggctcacggtcacagag

SEQ ID NO: 84 - DNA, 63 bp, synthetic construct
  source 1..63: mol_type = other DNA
agcagtgagggggctggaaacaccatatattttggagagggaagttggctcactgttgtagag

SEQ ID NO: 85 - DNA, 66 bp, synthetic construct
  source 1..66: mol_type = other DNA
agcagttatgctacaggctacaatgagcagttcttcgggccagggacacggctcaccgtgctagag

SEQ ID NO: 86 - DNA, 69 bp, synthetic construct
  source 1..69: mol_type = other DNA
agcagtgaacggcagggctccacagatacgcagtattttggcccaggcacccggctgacagtgctcgag

SEQ ID NO: 87 - DNA, 72 bp, synthetic construct
  source 1..72: mol_type = other DNA
agcagtgctccgactagcgggagggacaatgagcagttcttcgggccagggacacggctcaccgtgctagag

SEQ ID NO: 88 - DNA, 78 bp, synthetic construct
  source 1..78: mol_type = other DNA
agcagtgaatggactagcggggggcccaacaccggggagctgttttttggagaaggctctaggctgaccgtactggag

SEQ ID NO: 89 - DNA, 69 bp, synthetic construct
  source 1..69: mol_type = other DNA
agcagtgaacggggggctagcacagatacgcagtattttggcccaggcacccggctgacagtgctcgag

SEQ ID NO: 90 - DNA, 81 bp, synthetic construct
  source 1..81: mol_type = other DNA
agcagtgaagggctagcgggagaacctctcttaggcaatgagcagttcttcgggccagggacacggctcaccgtgctagag

SEQ ID NO: 91 - DNA, 69 bp, synthetic construct
  source 1..69: mol_type = other DNA
agcagtgaggcaggcggccacacagatacgcagtattttggcccaggcacccggctgacagtgctcgag

SEQ ID NO: 92 - DNA, 60 bp, synthetic construct
  source 1..60: mol_type = other DNA
agcagtgaataccaagagacccagtacttcgggccaggcacgcggctcctggtgctcgag

SEQ ID NO: 93 - DNA, 66 bp, synthetic construct
  source 1..66: mol_type = other DNA
agcaccgacaggggatctttcggggagctgttttttggagaaggctctaggctgaccgtactggag

SEQ ID NO: 94 - DNA, 63 bp, synthetic construct
  source 1..63: mol_type = other DNA
ggaggagggacatctcaagagacccagtacttcgggccaggcacgcggctcctggtgctcgag

SEQ ID NO: 95 - DNA, 69 bp, synthetic construct
  source 1..69: mol_type = other DNA
agcagtccgactagcgggatgggggagacccagtacttcgggccaggcacgcggctcctggtgctcgag

SEQ ID NO: 96 - DNA, 63 bp, synthetic construct
  source 1..63: mol_type = other DNA
agcagtgagttcggggccaacgtcctgactttcggggccggcagcaggctgaccgtgctggag

SEQ ID NO: 97 - DNA, 72 bp, synthetic construct
  source 1..72: mol_type = other DNA
agcagtgtccgtagcgggagaggggacaatgagcagttcttcgggccagggacacggctcaccgtgctagag

SEQ ID NO: 98 - DNA, 63 bp, synthetic construct
  source 1..63: mol_type = other DNA
agcagtgtccaggaggaagatacgcagtattttggcccaggcacccggctgacagtgctcgag

SEQ ID NO: 99 - DNA, 60 bp, synthetic construct
  source 1..60: mol_type = other DNA
agcagtgatagtagctacgagcagtacttcgggccgggcaccaggctcacggtcacagag

SEQ ID NO: 100 - DNA, 72 bp, synthetic construct
  source 1..72: mol_type = other DNA
agcagtggtactacgggacaggaatcctacgagcagtacttcgggccgggcaccaggctcacggtcacagag

SEQ ID NO: 101 - DNA, 66 bp, synthetic construct
  source 1..66: mol_type = other DNA
agcagtgtaagggggaaccacaatgagcagttcttcgggccagggacacggctcaccgtgctagag

SEQ ID NO: 102 - DNA, 75 bp, synthetic construct
  source 1..75: mol_type = other DNA
agcagtgaacttcagcgggagggttctccagatacgcagtattttggcccaggcacccggctgacagtgctcgag

SEQ ID NO: 103 - DNA, 66 bp, synthetic construct
  source 1..66: mol_type = other DNA
agcagtgtccgggacagggatgaaaaactgttttttggcagtggaacccagctctctgtcttggag

SEQ ID NO: 104 - DNA, 66 bp, synthetic construct
  source 1..66: mol_type = other DNA
agcagtgagggtcagggaggttacgagcagtacttcgggccgggcaccaggctcacggtcacagag

SEQ ID NO: 105 - DNA, 66 bp, synthetic construct
  source 1..66: mol_type = other DNA
agcagtgacagggatagcaatcagccccagcattttggtgatgggactcgactctccatcctagag

SEQ ID NO: 106 - DNA, 72 bp, synthetic construct
  source 1..72: mol_type = other DNA
agcagtgatcggtctagcggagccaaaaacattcagtacttcggcgccgggacccggctctcagtgctggag

SEQ ID NO: 107 - DNA, 72 bp, synthetic construct
  source 1..72: mol_type = other DNA
agcagtgccacgactagcgggaggacagatacgcagtattttggcccaggcacccggctgacagtgctcgag

SEQ ID NO: 108 - DNA, 72 bp, synthetic construct
  source 1..72: mol_type = other DNA
agcagtgaatttcggcagcgggagtcctacgagcagtacttcgggccgggcaccaggctcacggtcacagag

SEQ ID NO: 109 - DNA, 69 bp, synthetic construct
  source 1..69: mol_type = other DNA
agcagtgaaatagcgggagtggcctacgagcagtacttcgggccgggcaccaggctcacggtcacagag

SEQ ID NO: 110 - DNA, 69 bp, synthetic construct
  source 1..69: mol_type = other DNA
agcgtgggtccggtaccctcctacaatgagcagttcttcgggccagggacacggctcaccgtgctagag

SEQ ID NO: 111 - DNA, 69 bp, synthetic construct
  source 1..69: mol_type = other DNA
agcagtgaacggcgcgggagacgggatacgcagtattttggcccaggcacccggctgacagtgctcgag

SEQ ID NO: 112 - DNA, 60 bp, synthetic construct
  source 1..60: mol_type = other DNA
agcagtgggacagggtacgagcagtacttcgggccgggcaccaggctcacggtcacagag

SEQ ID NO: 113 - DNA, 69 bp, synthetic construct
  source 1..69: mol_type = other DNA
agcagtgaccgtagcgggagctcctacgagcagtacttcgggccgggcaccaggctcacggtcacagag

SEQ ID NO: 114 - DNA, 60 bp, synthetic construct
  source 1..60: mol_type = other DNA
agcagtgacagcaccggggagctgttttttggagaaggctctaggctgaccgtactggag

SEQ ID NO: 115 - DNA, 63 bp, synthetic construct
  source 1..63: mol_type = other DNA
agcagtgctagcgggagcaatgagcagttcttcgggccagggacacggctcaccgtgctagag

SEQ ID NO: 116 - DNA, 66 bp, synthetic construct
  source 1..66: mol_type = other DNA
agcagtgacgggactagcggctacgagcagtacttcgggccgggcaccaggctcacggtcacagag

SEQ ID NO: 117 - DNA, 57 bp, synthetic construct
  source 1..57: mol_type = other DNA
agcagtgaatatgaaaaactgttttttggcagtggaacccagctctctgtcttggag

SEQ ID NO: 118 - DNA, 66 bp, synthetic construct
  source 1..66: mol_type = other DNA
agcagtgagtccggcccccgcaatgagcagttcttcgggccagggacacggctcaccgtgctagag

SEQ ID NO: 119 - DNA, 72 bp, synthetic construct
  source 1..72: mol_type = other DNA
agcagtggccgactagcgggagaggaagagacccagtacttcgggccaggcacgcggctcctggtgctcgag

SEQ ID NO: 120 - DNA, 66 bp, synthetic construct
  source 1..66: mol_type = other DNA
agcagtgagggtggcagggtcgatgagcagttcttcgggccagggacacggctcaccgtgctagag

SEQ ID NO: 121 - DNA, 60 bp, synthetic construct
  source 1..60: mol_type = other DNA
agcagtgaggctaactatggctacaccttcggttcggggaccaggttaaccgttgtagag

SEQ ID NO: 122 - DNA, 66 bp, synthetic construct
  source 1..66: mol_type = other DNA
agcagtcaggacggattgggatatggctacaccttcggttcggggaccaggttaaccgttgtagag

SEQ ID NO: 123 - DNA, 60 bp, synthetic construct
  source 1..60: mol_type = other DNA
agcagtgggcgcctccacgagcagtacttcgggccgggcaccaggctcacggtcacagag

SEQ ID NO: 124 - DNA, 66 bp, synthetic construct
  source 1..66: mol_type = other DNA
agcagtgaatataacagcacagatacgcagtattttggcccaggcacccggctgacagtgctcgag

SEQ ID NO: 125 - DNA, 63 bp, synthetic construct
  source 1..63: mol_type = other DNA
agcagtgaacccggattgggggagctgttttttggagaaggctctaggctgaccgtactggag

SEQ ID NO: 126 - DNA, 66 bp, synthetic construct
  source 1..66: mol_type = other DNA
agcatcctgggagaggggcggaatgagcagttcttcgggccagggacacggctcaccgtgctagag

SEQ ID NO: 127 - DNA, 66 bp, synthetic construct
  source 1..66: mol_type = other DNA
agcagtgccccgggacagatctatggctacaccttcggttcggggaccaggttaaccgttgtagag

SEQ ID NO: 128 - DNA, 60 bp, synthetic construct
  source 1..60: mol_type = other DNA
agcagtgacaaccaagagacccagtacttcgggccaggcacgcggctcctggtgctcgag

SEQ ID NO: 129 - DNA, 72 bp, synthetic construct
  source 1..72: mol_type = other DNA
agcagtggggtgactagcgcctcctacaatgagcagttcttcgggccagggacacggctcaccgtgctagag

SEQ ID NO: 130 - DNA, 78 bp, synthetic construct
  source 1..78: mol_type = other DNA
agcagtcctgagcccaccaccctagcgggagtccacgagcagtacttcgggccgggcaccaggctcacggtcacagag

SEQ ID NO: 131 - DNA, 66 bp, synthetic construct
  source 1..66: mol_type = other DNA
agcagtgggacacagagggctgaaaaactgttttttggcagtggaacccagctctctgtcttggag

SEQ ID NO: 132 - DNA, 69 bp, synthetic construct
  source 1..69: mol_type = other DNA
agcagtgggactagcgggagcacagatacgcagtattttggcccaggcacccggctgacagtgctcgag

SEQ ID NO: 133 - DNA, 69 bp, synthetic construct
  source 1..69: mol_type = other DNA
agcagtgaggcgggacagggttcctacgagcagtacttcgggccgggcaccaggctcacggtcacagag

SEQ ID NO: 134 - DNA, 63 bp, synthetic construct
  source 1..63: mol_type = other DNA
agcacctctagccgcaccggggagctgttttttggagaaggctctaggctgaccgtactggag

SEQ ID NO: 135 - DNA, 66 bp, synthetic construct
  source 1..66: mol_type = other DNA
agcagtgaaccgggggagcggaatgagcagttcttcgggccagggacacggctcaccgtgctagag

SEQ ID NO: 136 - DNA, 66 bp, synthetic construct
  source 1..66: mol_type = other DNA
agcagtgaaggtcgggttaactatggctacaccttcggttcggggaccaggttaaccgttgtagag

SEQ ID NO: 137 - DNA, 69 bp, synthetic construct
  source 1..69: mol_type = other DNA
agcagtgaatcagaagggggcacagatacgcagtattttggcccaggcacccggctgacagtgctcgag

SEQ ID NO: 138 - DNA, 81 bp, synthetic construct
  source 1..81: mol_type = other DNA
agcagtcccggggggactagcgggagggcacgtccctacgagcagtacttcgggccgggcaccaggctcacggtcacagag

SEQ ID NO: 139 - DNA, 69 bp, synthetic construct
  source 1..69: mol_type = other DNA
agcagtgggagggagggggacccctacgagcagtacttcgggccgggcaccaggctcacggtcacagag

SEQ ID NO: 140 - DNA, 66 bp, synthetic construct
  source 1..66: mol_type = other DNA
agcagtggactagcgaacaccggggagctgttttttggagaaggctctaggctgaccgtactggag

SEQ ID NO: 141 - DNA, 75 bp, synthetic construct
  source 1..75: mol_type = other DNA
agcagtgggacgacaggggatacacgcacagatacgcagtattttggcccaggcacccggctgacagtgctcgag

SEQ ID NO: 142 - DNA, 69 bp, synthetic construct
  source 1..69: mol_type = other DNA
agcagtgaagaccgggacaggggtcacgagcagtacttcgggccgggcaccaggctcacggtcacagag

SEQ ID NO: 143 - DNA, 60 bp, synthetic construct
  source 1..60: mol_type = other DNA
agcagtgaactagcgaatgagcagttcttcgggccagggacacggctcaccgtgctagag

SEQ ID NO: 144 - DNA, 63 bp, synthetic construct
  source 1..63: mol_type = other DNA
agcagtggaggaggggaggagacccagtacttcgggccaggcacgcggctcctggtgctcgag

SEQ ID NO: 145 - DNA, 75 bp, synthetic construct
  source 1..75: mol_type = other DNA
agcagtgaatatgcagggtggggcggcaatcagccccagcattttggtgatgggactcgactctccatcctagag

SEQ ID NO: 146 - DNA, 72 bp, synthetic construct
  source 1..72: mol_type = other DNA
agcagtgaattggacgggactagcgcctacgagcagtacttcgggccgggcaccaggctcacggtcacagag

SEQ ID NO: 147 - DNA, 66 bp, synthetic construct
  source 1..66: mol_type = other DNA
agcagtgcgcagggggtcagcgaaaaactgttttttggcagtggaacccagctctctgtcttggag

SEQ ID NO: 148 - DNA, 72 bp, synthetic construct
  source 1..72: mol_type = other DNA
agcagtgaagtggcgggagcggacaccggggagctgttttttggagaaggctctaggctgaccgtactggag

SEQ ID NO: 149 - DNA, 75 bp, synthetic construct
  source 1..75: mol_type = other DNA
agcagcggcagggggccaggggaaagtgcagatacgcagtattttggcccaggcacccggctgacagtgctcgag

SEQ ID NO: 150 - DNA, 765 bp, synthetic construct
  source 1..765: mol_type = other DNA
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaataa

SEQ ID NO: 151 - DNA, 765 bp, synthetic construct
  source 1..765: mol_type = other DNA
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaataa

SEQ ID NO: 152 - DNA, 747 bp, synthetic construct
  source 1..747: mol_type = other DNA
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccgtccaagtgcccagcatgggcagctcttcaccaatgggggtctctttctgcttggtgatggtactcctttttgcagtggacacaggactatatttctctgtgaagacaaacattcgaagctcaacaagagactggaaggaccataaatttaaatggagaaaggaccctcaagacaaataa

SEQ ID NO: 153 - DNA, 1011 bp, synthetic construct
  source 1..1011: mol_type = other DNA
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccgtccaagtgcccagcatgggcagctcttcaccaatggggctctgttaccttcttgacggtattctttttatttacggcgtcatcctcactgccctctttttgagggtcaagttttcaagatccgccgacgcacctgcttaccagcaaggacaaaatcaactttataacgaactcaaccttggtcggcgggaagaatatgatgtactcgacaaaagaagaggccgcgacccggagatgggtggcaaaccacagcggaggaaaaacccccaggaaggtctctataacgaattgcagaaagataaaatggccgaggcttattcagagatcggcatgaaaggagagcgacgacgaggaaaggggcatgatggtctgtatcaagggctcagcacggcaacaaaggacacatatgatgccttgcatatgcaggcgcttccgccgcgctaa

SEQ ID NO: 154 - DNA, 678 bp, synthetic construct
  source 1..678: mol_type = other DNA
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccgtccaagtgcccagcatgggcagctcttcaccaatgggggtctctttctgcttggtgatggtactcctttttgcagtggacacaggactatatttctctgtgaagacataa

SEQ ID NO: 155 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 156 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 157 - protein, 248 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITVQVPSMGSSSPMGVSFCLVMVLLFAVDTGLYFSVKTNIRSSTRDWKDHKFKWRKDPQDK

SEQ ID NO: 158 - protein, 336 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITVQVPSMGSSSPMGLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 159 - protein, 225 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITVQVPSMGSSSPMGVSFCLVMVLLFAVDTGLYFSVKT

SEQ ID NO: 160 - DNA, 39 bp, synthetic construct
  source 1..39: mol_type = other DNA
gtccaagtgcccagcatgggcagctcttcaccaatgggg

SEQ ID NO: 161 - protein, 13 aa, synthetic construct
VQVPSMGSSSPMG

SEQ ID NO: 162 - DNA, 63 bp, synthetic construct
  source 1..63: mol_type = other DNA
ctctgttaccttcttgacggtattctttttatttacggcgtcatcctcactgccctctttttg

SEQ ID NO: 163 - protein, 21 aa, synthetic construct
LCYLLDGILFIYGVILTALFL

SEQ ID NO: 164 - DNA, 342 bp, synthetic construct
  source 1..342: mol_type = other DNA
agggtcaagttttcaagatccgccgacgcacctgcttaccagcaaggacaaaatcaactttataacgaactcaaccttggtcggcgggaagaatatgatgtactcgacaaaagaagaggccgcgacccggagatgggtggcaaaccacagcggaggaaaaacccccaggaaggtctctataacgaattgcagaaagataaaatggccgaggcttattcagagatcggcatgaaaggagagcgacgacgaggaaaggggcatgatggtctgtatcaagggctcagcacggcaacaaaggacacatatgatgccttgcatatgcaggcgcttccgccgcgctaa

SEQ ID NO: 165 - protein, 113 aa, synthetic construct; identical to SEQ ID NO: 25

SEQ ID NO: 166 - DNA, 63 bp, synthetic construct
  source 1..63: mol_type = other DNA
gtctctttctgcttggtgatggtactcctttttgcagtggacacaggactatatttctctgtg

SEQ ID NO: 167 - protein, 21 aa, synthetic construct
VSFCLVMVLLFAVDTGLYFSV

SEQ ID NO: 168 - DNA, 78 bp, synthetic construct
  source 1..78: mol_type = other DNA
aagacaaacattcgaagctcaacaagagactggaaggaccataaatttaaatggagaaaggaccctcaagacaaataa

SEQ ID NO: 169 - protein, 25 aa, synthetic construct
KTNIRSSTRDWKDHKFKWRKDPQDK

SEQ ID NO: 170 - DNA, 1827 bp, synthetic construct
  source 1..1827: mol_type = other DNA
atgaagaagcacctgaccacctttctggtcatcctgtggctgtacttctacagaggcaacggcaagaaccaggtggaacagagccctcagagcctgatcatcctggaaggcaagaactgcaccctgcagtgcaactacaccgtgtctcccttcagcaacctgcggtggtacaagcaggatacaggcagaggccctgtgtctctgaccatcatgaccttcagcgagaacaccaagagcaacggccggtacaccgccacactggatgccgatacaaagcagagcagcctgcacatcacagccagccagctgagcgatagcgccagctacatctgcgtggtgtccgatagaggcagcaccctgggcagactgtactttggcagaggcacccagctgaccgtgtggcccgatattcagaaccctgatcctgccgtgtaccagctgagagacagcaagtccagcgacaagagcgtgtgcctgttcaccgacttcgacagccagaccaacgtgtcccagagcaaggacagcgacgtgtacatcaccgacaagtgcgtgctggacatgcggagcatggacttcaagagcaatagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctgctgaaagtggccggcttcaacctgctgatgaccctgagactgtggtctagcggaagcggcgccaccaatttcagcctgctgaaacaggctggcgacgtggaagagaaccccggacctatgaccatccggctgctgtgctatatgggcttctacttcctcggagccggcctgatggaagccgacatctaccagacacctagatacctggtcatcggcaccggcaaaaagatcaccctggaatgctcccagaccatgggccacgacaagatgtactggtatcagcaggaccccggcatggaactgcatctgatccactacagctacggcgtgaacagcaccgagaagggcgatctgtctagcgagagcaccgtgtccagaatccggaccgagcacttcccactgacactggaaagcgccagacctagccacaccagccagtacctgtgtgccactggacagggggcgcaagatacgcagtattttggcccaggcacccggctgacagtgctcgaggacctcaagaatgtgtttccgcccgaagtcgcggtttttgaaccatcagaagccgagatctctcatacacaaaaggcgacgctcgtatgcctcgcgacgggattttatccggaccacgtcgagctttcctggtgggttaacggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagaatcataccaacaaggagtattgagcgcgacaattctttacgaaattctgcttgggaaagcgactctgtacgcggtgctcgtgtccgctttggttcttatggcaatggttaaacgaaaggatagtaggggctaa

SEQ ID NO: 171 - DNA, 2658 bp, synthetic construct
  source 1..2658: mol_type = other DNA
atgaagaagcacctgaccacctttctggtcatcctgtggctgtacttctacagaggcaacggcaagaaccaggtggaacagagccctcagagcctgatcatcctggaaggcaagaactgcaccctgcagtgcaactacaccgtgtctcccttcagcaacctgcggtggtacaagcaggatacaggcagaggccctgtgtctctgaccatcatgaccttcagcgagaacaccaagagcaacggccggtacaccgccacactggatgccgatacaaagcagagcagcctgcacatcacagccagccagctgagcgatagcgccagctacatctgcgtggtgtccgatagaggcagcaccctgggcagactgtactttggcagaggcacccagctgaccgtgtggcccgatattcagaaccctgatcctgccgtgtaccagctgagagacagcaagtccagcgacaagagcgtgtgcctgttcaccgacttcgacagccagaccaacgtgtcccagagcaaggacagcgacgtgtacatcaccgacaagtgcgtgctggacatgcggagcatggacttcaagagcaatagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctgctgaaagtggccggcttcaacctgctgatgaccctgagactgtggtctagcggaagcggcgccaccaatttcagcctgctgaaacaggctggcgacgtggaagagaaccccggacctatgaccatccggctgctgtgctatatgggcttctacttcctcggagccggcctgatggaagccgacatctaccagacacctagatacctggtcatcggcaccggcaaaaagatcaccctggaatgctcccagaccatgggccacgacaagatgtactggtatcagcaggaccccggcatggaactgcatctgatccactacagctacggcgtgaacagcaccgagaagggcgatctgtctagcgagagcaccgtgtccagaatccggaccgagcacttcccactgacactggaaagcgccagacctagccacaccagccagtacctgtgtgccactggacagggggcgcaagatacgcagtattttggcccaggcacccggctgacagtgctcgaggacctcaagaatgtgtttccgcccgaagtcgcggtttttgaaccatcagaagccgagatctctcatacacaaaaggcgacgctcgtatgcctcgcgacgggattttatccggaccacgtcgagctttcctggtgggttaacggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagaatcataccaacaaggagtattgagcgcgacaattctttacgaaattctgcttgggaaagcgactctgtacgcggtgctcgtgtccgctttggttcttatggcaatggttaaacgaaaggatagtaggggcggtagtggacagtgtactaattatgctctcttgaaattggctggagatgttgagagcaatcccgggcccatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaataa

SEQ ID NO: 172 - DNA, 2658 bp, synthetic construct
  source 1..2658: mol_type = other DNA
atgaagaagcacctgaccacctttctggtcatcctgtggctgtacttctacagaggcaacggcaagaaccaggtggaacagagccctcagagcctgatcatcctggaaggcaagaactgcaccctgcagtgcaactacaccgtgtctcccttcagcaacctgcggtggtacaagcaggatacaggcagaggccctgtgtctctgaccatcatgaccttcagcgagaacaccaagagcaacggccggtacaccgccacactggatgccgatacaaagcagagcagcctgcacatcacagccagccagctgagcgatagcgccagctacatctgcgtggtgtccgatagaggcagcaccctgggcagactgtactttggcagaggcacccagctgaccgtgtggcccgatattcagaaccctgatcctgccgtgtaccagctgagagacagcaagtccagcgacaagagcgtgtgcctgttcaccgacttcgacagccagaccaacgtgtcccagagcaaggacagcgacgtgtacatcaccgacaagtgcgtgctggacatgcggagcatggacttcaagagcaatagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctgctgaaagtggccggcttcaacctgctgatgaccctgagactgtggtctagcggaagcggcgccaccaatttcagcctgctgaaacaggctggcgacgtggaagagaaccccggacctatgaccatccggctgctgtgctatatgggcttctacttcctcggagccggcctgatggaagccgacatctaccagacacctagatacctggtcatcggcaccggcaaaaagatcaccctggaatgctcccagaccatgggccacgacaagatgtactggtatcagcaggaccccggcatggaactgcatctgatccactacagctacggcgtgaacagcaccgagaagggcgatctgtctagcgagagcaccgtgtccagaatccggaccgagcacttcccactgacactggaaagcgccagacctagccacaccagccagtacctgtgtgccactggacagggggcgcaagatacgcagtattttggcccaggcacccggctgacagtgctcgaggacctcaagaatgtgtttccgcccgaagtcgcggtttttgaaccatcagaagccgagatctctcatacacaaaaggcgacgctcgtatgcctcgcgacgggattttatccggaccacgtcgagctttcctggtgggttaacggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagaatcataccaacaaggagtattgagcgcgacaattctttacgaaattctgcttgggaaagcgactctgtacgcggtgctcgtgtccgctttggttcttatggcaatggttaaacgaaaggatagtaggggcggtagtggacagtgtactaattatgctctcttgaaattggctggagatgttgagagcaatcccgggcccatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaataa

SEQ ID NO: 173 - DNA, 2640 bp, synthetic construct
  source 1..2640: mol_type = other DNA
atgaagaagcacctgaccacctttctggtcatcctgtggctgtacttctacagaggcaacggcaagaaccaggtggaacagagccctcagagcctgatcatcctggaaggcaagaactgcaccctgcagtgcaactacaccgtgtctcccttcagcaacctgcggtggtacaagcaggatacaggcagaggccctgtgtctctgaccatcatgaccttcagcgagaacaccaagagcaacggccggtacaccgccacactggatgccgatacaaagcagagcagcctgcacatcacagccagccagctgagcgatagcgccagctacatctgcgtggtgtccgatagaggcagcaccctgggcagactgtactttggcagaggcacccagctgaccgtgtggcccgatattcagaaccctgatcctgccgtgtaccagctgagagacagcaagtccagcgacaagagcgtgtgcctgttcaccgacttcgacagccagaccaacgtgtcccagagcaaggacagcgacgtgtacatcaccgacaagtgcgtgctggacatgcggagcatggacttcaagagcaatagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctgctgaaagtggccggcttcaacctgctgatgaccctgagactgtggtctagcggaagcggcgccaccaatttcagcctgctgaaacaggctggcgacgtggaagagaaccccggacctatgaccatccggctgctgtgctatatgggcttctacttcctcggagccggcctgatggaagccgacatctaccagacacctagatacctggtcatcggcaccggcaaaaagatcaccctggaatgctcccagaccatgggccacgacaagatgtactggtatcagcaggaccccggcatggaactgcatctgatccactacagctacggcgtgaacagcaccgagaagggcgatctgtctagcgagagcaccgtgtccagaatccggaccgagcacttcccactgacactggaaagcgccagacctagccacaccagccagtacctgtgtgccactggacagggggcgcaagatacgcagtattttggcccaggcacccggctgacagtgctcgaggacctcaagaatgtgtttccgcccgaagtcgcggtttttgaaccatcagaagccgagatctctcatacacaaaaggcgacgctcgtatgcctcgcgacgggattttatccggaccacgtcgagctttcctggtgggttaacggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagaatcataccaacaaggagtattgagcgcgacaattctttacgaaattctgcttgggaaagcgactctgtacgcggtgctcgtgtccgctttggttcttatggcaatggttaaacgaaaggatagtaggggcggtagtggacagtgtactaattatgctctcttgaaattggctggagatgttgagagcaatcccgggcccatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccgtccaagtgcccagcatgggcagctcttcaccaatgggggtctctttctgcttggtgatggtactcctttttgcagtggacacaggactatatttctctgtgaagacaaacattcgaagctcaacaagagactggaaggaccataaatttaaatggagaaaggaccctcaagacaaataa

SEQ ID NO: 174 - DNA, 2904 bp, synthetic construct
  source 1..2904: mol_type = other DNA
atgaagaagcacctgaccacctttctggtcatcctgtggctgtacttctacagaggcaacggcaagaaccaggtggaacagagccctcagagcctgatcatcctggaaggcaagaactgcaccctgcagtgcaactacaccgtgtctcccttcagcaacctgcggtggtacaagcaggatacaggcagaggccctgtgtctctgaccatcatgaccttcagcgagaacaccaagagcaacggccggtacaccgccacactggatgccgatacaaagcagagcagcctgcacatcacagccagccagctgagcgatagcgccagctacatctgcgtggtgtccgatagaggcagcaccctgggcagactgtactttggcagaggcacccagctgaccgtgtggcccgatattcagaaccctgatcctgccgtgtaccagctgagagacagcaagtccagcgacaagagcgtgtgcctgttcaccgacttcgacagccagaccaacgtgtcccagagcaaggacagcgacgtgtacatcaccgacaagtgcgtgctggacatgcggagcatggacttcaagagcaatagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctgctgaaagtggccggcttcaacctgctgatgaccctgagactgtggtctagcggaagcggcgccaccaatttcagcctgctgaaacaggctggcgacgtggaagagaaccccggacctatgaccatccggctgctgtgctatatgggcttctacttcctcggagccggcctgatggaagccgacatctaccagacacctagatacctggtcatcggcaccggcaaaaagatcaccctggaatgctcccagaccatgggccacgacaagatgtactggtatcagcaggaccccggcatggaactgcatctgatccactacagctacggcgtgaacagcaccgagaagggcgatctgtctagcgagagcaccgtgtccagaatccggaccgagcacttcccactgacactggaaagcgccagacctagccacaccagccagtacctgtgtgccactggacagggggcgcaagatacgcagtattttggcccaggcacccggctgacagtgctcgaggacctcaagaatgtgtttccgcccgaagtcgcggtttttgaaccatcagaagccgagatctctcatacacaaaaggcgacgctcgtatgcctcgcgacgggattttatccggaccacgtcgagctttcctggtgggttaacggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagaatcataccaacaaggagtattgagcgcgacaattctttacgaaattctgcttgggaaagcgactctgtacgcggtgctcgtgtccgctttggttcttatggcaatggttaaacgaaaggatagtaggggcggtagtggacagtgtactaattatgctctcttgaaattggctggagatgttgagagcaatcccgggcccatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccgtccaagtgcccagcatgggcagctcttcaccaatggggctctgttaccttcttgacggtattctttttatttacggcgtcatcctcactgccctctttttgagggtcaagttttcaagatccgccgacgcacctgcttaccagcaaggacaaaatcaactttataacgaactcaaccttggtcggcgggaagaatatgatgtactcgacaaaagaagaggccgcgacccggagatgggtggcaaaccacagcggaggaaaaacccccaggaaggtctctataacgaattgcagaaagataaaatggccgaggcttattcagagatcggcatgaaaggagagcgacgacgaggaaaggggcatgatggtctgtatcaagggctcagcacggcaacaaaggacacatatgatgccttgcatatgcaggcgcttccgccgcgctaa

SEQ ID NO: 175 - DNA, 2571 bp, synthetic construct
  source 1..2571: mol_type = other DNA
atgaagaagcacctgaccacctttctggtcatcctgtggctgtacttctacagaggcaacggcaagaaccaggtggaacagagccctcagagcctgatcatcctggaaggcaagaactgcaccctgcagtgcaactacaccgtgtctcccttcagcaacctgcggtggtacaagcaggatacaggcagaggccctgtgtctctgaccatcatgaccttcagcgagaacaccaagagcaacggccggtacaccgccacactggatgccgatacaaagcagagcagcctgcacatcacagccagccagctgagcgatagcgccagctacatctgcgtggtgtccgatagaggcagcaccctgggcagactgtactttggcagaggcacccagctgaccgtgtggcccgatattcagaaccctgatcctgccgtgtaccagctgagagacagcaagtccagcgacaagagcgtgtgcctgttcaccgacttcgacagccagaccaacgtgtcccagagcaaggacagcgacgtgtacatcaccgacaagtgcgtgctggacatgcggagcatggacttcaagagcaatagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctgctgaaagtggccggcttcaacctgctgatgaccctgagactgtggtctagcggaagcggcgccaccaatttcagcctgctgaaacaggctggcgacgtggaagagaaccccggacctatgaccatccggctgctgtgctatatgggcttctacttcctcggagccggcctgatggaagccgacatctaccagacacctagatacctggtcatcggcaccggcaaaaagatcaccctggaatgctcccagaccatgggccacgacaagatgtactggtatcagcaggaccccggcatggaactgcatctgatccactacagctacggcgtgaacagcaccgagaagggcgatctgtctagcgagagcaccgtgtccagaatccggaccgagcacttcccactgacactggaaagcgccagacctagccacaccagccagtacctgtgtgccactggacagggggcgcaagatacgcagtattttggcccaggcacccggctgacagtgctcgaggacctcaagaatgtgtttccgcccgaagtcgcggtttttgaaccatcagaagccgagatctctcatacacaaaaggcgacgctcgtatgcctcgcgacgggattttatccggaccacgtcgagctttcctggtgggttaacggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagaatcataccaacaaggagtattgagcgcgacaattctttacgaaattctgcttgggaaagcgactctgtacgcggtgctcgtgtccgctttggttcttatggcaatggttaaacgaaaggatagtaggggcggtagtggacagtgtactaattatgctctcttgaaattggctggagatgttgagagcaatcccgggcccatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccgtccaagtgcccagcatgggcagctcttcaccaatgggggtctctttctgcttggtgatggtactcctttttgcagtggacacaggactatatttctctgtgaagacataa

SEQ ID NO: 176 - DNA, 4463 bp, synthetic construct
  source 1..4463: mol_type = other DNA
aatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgaagaagcacctgaccacctttctggtcatcctgtggctgtacttctacagaggcaacggcaagaaccaggtggaacagagccctcagagcctgatcatcctggaaggcaagaactgcaccctgcagtgcaactacaccgtgtctcccttcagcaacctgcggtggtacaagcaggatacaggcagaggccctgtgtctctgaccatcatgaccttcagcgagaacaccaagagcaacggccggtacaccgccacactggatgccgatacaaagcagagcagcctgcacatcacagccagccagctgagcgatagcgccagctacatctgcgtggtgtccgatagaggcagcaccctgggcagactgtactttggcagaggcacccagctgaccgtgtggcccgatattcagaaccctgatcctgccgtgtaccagctgagagacagcaagtccagcgacaagagcgtgtgcctgttcaccgacttcgacagccagaccaacgtgtcccagagcaaggacagcgacgtgtacatcaccgacaagtgcgtgctggacatgcggagcatggacttcaagagcaatagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctgctgaaagtggccggcttcaacctgctgatgaccctgagactgtggtctagcggaagcggcgccaccaatttcagcctgctgaaacaggctggcgacgtggaagagaaccccggacctatgaccatccggctgctgtgctatatgggcttctacttcctcggagccggcctgatggaagccgacatctaccagacacctagatacctggtcatcggcaccggcaaaaagatcaccctggaatgctcccagaccatgggccacgacaagatgtactggtatcagcaggaccccggcatggaactgcatctgatccactacagctacggcgtgaacagcaccgagaagggcgatctgtctagcgagagcaccgtgtccagaatccggaccgagcacttcccactgacactggaaagcgccagacctagccacaccagccagtacctgtgtgccactggacagggggcgcaagatacgcagtattttggcccaggcacccggctgacagtgctcgaggacctcaagaatgtgtttccgcccgaagtcgcggtttttgaaccatcagaagccgagatctctcatacacaaaaggcgacgctcgtatgcctcgcgacgggattttatccggaccacgtcgagctttcctggtgggttaacggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagaatcataccaacaaggagtattgagcgcgacaattctttacgaaattctgcttgggaaagcgactctgtacgcggtgctcgtgtccgctttggttcttatggcaatggttaaacgaaaggatagtaggggctaacaattgcgcgtcatcatcgatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttca

SEQ ID NO: 177 - DNA, 5294 bp, synthetic construct
  source 1..5294: mol_type = other DNA
aatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgaagaagcacctgaccacctttctggtcatcctgtggctgtacttctacagaggcaacggcaagaaccaggtggaacagagccctcagagcctgatcatcctggaaggcaagaactgcaccctgcagtgcaactacaccgtgtctcccttcagcaacctgcggtggtacaagcaggatacaggcagaggccctgtgtctctgaccatcatgaccttcagcgagaacaccaagagcaacggccggtacaccgccacactggatgccgatacaaagcagagcagcctgcacatcacagccagccagctgagcgatagcgccagctacatctgcgtggtgtccgatagaggcagcaccctgggcagactgtactttggcagaggcacccagctgaccgtgtggcccgatattcagaaccctgatcctgccgtgtaccagctgagagacagcaagtccagcgacaagagcgtgtgcctgttcaccgacttcgacagccagaccaacgtgtcccagagcaaggacagcgacgtgtacatcaccgacaagtgcgtgctggacatgcggagcatggacttcaagagcaatagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctgctgaaagtggccggcttcaacctgctgatgaccctgagactgtggtctagcggaagcggcgccaccaatttcagcctgctgaaacaggctggcgacgtggaagagaaccccggacctatgaccatccggctgctgtgctatatgggcttctacttcctcggagccggcctgatggaagccgacatctaccagacacctagatacctggtcatcggcaccggcaaaaagatcaccctggaatgctcccagaccatgggccacgacaagatgtactggtatcagcaggaccccggcatggaactgcatctgatccactacagctacggcgtgaacagcaccgagaagggcgatctgtctagcgagagcaccgtgtccagaatccggaccgagcacttcccactgacactggaaagcgccagacctagccacaccagccagtacctgtgtgccactggacagggggcgcaagatacgcagtattttggcccaggcacccggctgacagtgctcgaggacctcaagaatgtgtttccgcccgaagtcgcggtttttgaaccatcagaagccgagatctctcatacacaaaaggcgacgctcgtatgcctcgcgacgggattttatccggaccacgtcgagctttcctggtgggttaacggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagaatcataccaacaaggagtattgagcgcgacaattctttacgaaattctgcttgggaaagcgactctgtacgcggtgctcgtgtccgctttggttcttatggcaatggttaaacgaaaggatagtaggggcggtagtggacagtgtactaattatgctctcttgaaattggctggagatgttgagagcaatcccgggcccatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaataacaattgcgcgtcatcatcgatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttca

SEQ ID NO: 178 - DNA, 5294 bp, synthetic construct
  source 1..5294: mol_type = other DNA
aatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgaagaagcacctgaccacctttctggtcatcctgtggctgtacttctacagaggcaacggcaagaaccaggtggaacagagccctcagagcctgatcatcctggaaggcaagaactgcaccctgcagtgcaactacaccgtgtctcccttcagcaacctgcggtggtacaagcaggatacaggcagaggccctgtgtctctgaccatcatgaccttcagcgagaacaccaagagcaacggccggtacaccgccacactggatgccgatacaaagcagagcagcctgcacatcacagccagccagctgagcgatagcgccagctacatctgcgtggtgtccgatagaggcagcaccctgggcagactgtactttggcagaggcacccagctgaccgtgtggcccgatattcagaaccctgatcctgccgtgtaccagctgagagacagcaagtccagcgacaagagcgtgtgcctgttcaccgacttcgacagccagaccaacgtgtcccagagcaaggacagcgacgtgtacatcaccgacaagtgcgtgctggacatgcggagcatggacttcaagagcaatagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctgctgaaagtggccggcttcaacctgctgatgaccctgagactgtggtctagcggaagcggcgccaccaatttcagcctgctgaaacaggctggcgacgtggaagagaaccccggacctatgaccatccggctgctgtgctatatgggcttctacttcctcggagccggcctgatggaagccgacatctaccagacacctagatacctggtcatcggcaccggcaaaaagatcaccctggaatgctcccagaccatgggccacgacaagatgtactggtatcagcaggaccccggcatggaactgcatctgatccactacagctacggcgtgaacagcaccgagaagggcgatctgtctagcgagagcaccgtgtccagaatccggaccgagcacttcccactgacactggaaagcgccagacctagccacaccagccagtacctgtgtgccactggacagggggcgcaagatacgcagtattttggcccaggcacccggctgacagtgctcgaggacctcaagaatgtgtttccgcccgaagtcgcggtttttgaaccatcagaagccgagatctctcatacacaaaaggcgacgctcgtatgcctcgcgacgggattttatccggaccacgtcgagctttcctggtgggttaacggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagaatcataccaacaaggagtattgagcgcgacaattctttacgaaattctgcttgggaaagcgactctgtacgcggtgctcgtgtccgctttggttcttatggcaatggttaaacgaaaggatagtaggggcggtagtggacagtgtactaattatgctctcttgaaattggctggagatgttgagagcaatcccgggcccatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaataacaattgcgcgtcatcatcgatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttca

SEQ ID NO: 179 - DNA, 5276 bp, synthetic construct
  source 1..5276: mol_type = other DNA
aatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgaagaagcacctgaccacctttctggtcatcctgtggctgtacttctacagaggcaacggcaagaaccaggtggaacagagccctcagagcctgatcatcctggaaggcaagaactgcaccctgcagtgcaactacaccgtgtctcccttcagcaacctgcggtggtacaagcaggatacaggcagaggccctgtgtctctgaccatcatgaccttcagcgagaacaccaagagcaacggccggtacaccgccacactggatgccgatacaaagcagagcagcctgcacatcacagccagccagctgagcgatagcgccagctacatctgcgtggtgtccgatagaggcagcaccctgggcagactgtactttggcagaggcacccagctgaccgtgtggcccgatattcagaaccctgatcctgccgtgtaccagctgagagacagcaagtccagcgacaagagcgtgtgcctgttcaccgacttcgacagccagaccaacgtgtcccagagcaaggacagcgacgtgtacatcaccgacaagtgcgtgctggacatgcggagcatggacttcaagagcaatagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctgctgaaagtggccggcttcaacctgctgatgaccctgagactgtggtctagcggaagcggcgccaccaatttcagcctgctgaaacaggctggcgacgtggaagagaaccccggacctatgaccatccggctgctgtgctatatgggcttctacttcctcggagccggcctgatggaagccgacatctaccagacacctagatacctggtcatcggcaccggcaaaaagatcaccctggaatgctcccagaccatgggccacgacaagatgtactggtatcagcaggaccccggcatggaactgcatctgatccactacagctacggcgtgaacagcaccgagaagggcgatctgtctagcgagagcaccgtgtccagaatccggaccgagcacttcccactgacactggaaagcgccagacctagccacaccagccagtacctgtgtgccactggacagggggcgcaagatacgcagtattttggcccaggcacccggctgacagtgctcgaggacctcaagaatgtgtttccgcccgaagtcgcggtttttgaaccatcagaagccgagatctctcatacacaaaaggcgacgctcgtatgcctcgcgacgggattttatccggaccacgtcgagctttcctggtgggttaacggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagaatcataccaacaaggagtattgagcgcgacaattctttacgaaattctgcttgggaaagcgactctgtacgcggtgctcgtgtccgctttggttcttatggcaatggttaaacgaaaggatagtaggggcggtagtggacagtgtactaattatgctctcttgaaattggctggagatgttgagagcaatcccgggcccatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccgtccaagtgcccagcatgggcagctcttcaccaatgggggtctctttctgcttggtgatggtactcctttttgcagtggacacaggactatatttctctgtgaagacaaacattcgaagctcaacaagagactggaaggaccataaatttaaatggagaaaggaccctcaagacaaataacaattgcgcgtcatcatcgatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttca

SEQ ID NO: 180 - DNA, 5543 bp, synthetic construct
  source 1..5543: mol_type = other DNA
aatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgaagaagcacctgaccacctttctggtcatcctgtggctgtacttctacagaggcaacggcaagaaccaggtggaacagagccctcagagcctgatcatcctggaaggcaagaactgcaccctgcagtgcaactacaccgtgtctcccttcagcaacctgcggtggtacaagcaggatacaggcagaggccctgtgtctctgaccatcatgaccttcagcgagaacaccaagagcaacggccggtacaccgccacactggatgccgatacaaagcagagcagcctgcacatcacagccagccagctgagcgatagcgccagctacatctgcgtggtgtccgatagaggcagcaccctgggcagactgtactttggcagaggcacccagctgaccgtgtggcccgatattcagaaccctgatcctgccgtgtaccagctgagagacagcaagtccagcgacaagagcgtgtgcctgttcaccgacttcgacagccagaccaacgtgtcccagagcaaggacagcgacgtgtacatcaccgacaagtgcgtgctggacatgcggagcatggacttcaagagcaatagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctgctgaaagtggccggcttcaacctgctgatgaccctgagactgtggtctagcggaagcggcgccaccaatttcagcctgctgaaacaggctggcgacgtggaagagaaccccggacctatgaccatccggctgctgtgctatatgggcttctacttcctcggagccggcctgatggaagccgacatctaccagacacctagatacctggtcatcggcaccggcaaaaagatcaccctggaatgctcccagaccatgggccacgacaagatgtactggtatcagcaggaccccggcatggaactgcatctgatccactacagctacggcgtgaacagcaccgagaagggcgatctgtctagcgagagcaccgtgtccagaatccggaccgagcacttcccactgacactggaaagcgccagacctagccacaccagccagtacctgtgtgccactggacagggggcgcaagatacgcagtattttggcccaggcacccggctgacagtgctcgaggacctcaagaatgtgtttccgcccgaagtcgcggtttttgaaccatcagaagccgagatctctcatacacaaaaggcgacgctcgtatgcctcgcgacgggattttatccggaccacgtcgagctttcctggtgggttaacggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagaatcataccaacaaggagtattgagcgcgacaattctttacgaaattctgcttgggaaagcgactctgtacgcggtgctcgtgtccgctttggttcttatggcaatggttaaacgaaaggatagtaggggcggtagtggacagtgtactaattatgctctcttgaaattggctggagatgttgagagcaatcccgggcccatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccgtccaagtgcccagcatgggcagctcttcaccaatggggctctgttaccttcttgacggtattctttttatttacggcgtcatcctcactgccctctttttgagggtcaagttttcaagatccgccgacgcacctgcttaccagcaaggacaaaatcaactttataacgaactcaaccttggtcggcgggaagaatatgatgtactcgacaaaagaagaggccgcgacccggagatgggtggcaaaccacagcggaggaaaaacccccaggaaggtctctataacgaattgcagaaagataaaatggccgaggcttattcagagatcggcatgaaaggagagcgacgacgaggaaaggggcatgatggtctgtatcaagggctcagcacggcaacaaaggacacatatgatgccttgcatatgcaggcgcttccgccgcgctaataacaattgcgcgtcatcatcgatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttca

SEQ ID NO: 181 - DNA, 5207 bp, synthetic construct
  source 1..5207: mol_type = other DNA
aatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgaagaagcacctgaccacctttctggtcatcctgtggctgtacttctacagaggcaacggcaagaaccaggtggaacagagccctcagagcctgatcatcctggaaggcaagaactgcaccctgcagtgcaactacaccgtgtctcccttcagcaacctgcggtggtacaagcaggatacaggcagaggccctgtgtctctgaccatcatgaccttcagcgagaacaccaagagcaacggccggtacaccgccacactggatgccgatacaaagcagagcagcctgcacatcacagccagccagctgagcgatagcgccagctacatctgcgtggtgtccgatagaggcagcaccctgggcagactgtactttggcagaggcacccagctgaccgtgtggcccgatattcagaaccctgatcctgccgtgtaccagctgagagacagcaagtccagcgacaagagcgtgtgcctgttcaccgacttcgacagccagaccaacgtgtcccagagcaaggacagcgacgtgtacatcaccgacaagtgcgtgctggacatgcggagcatggacttcaagagcaatagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctgctgaaagtggccggcttcaacctgctgatgaccctgagactgtggtctagcggaagcggcgccaccaatttcagcctgctgaaacaggctggcgacgtggaagagaaccccggacctatgaccatccggctgctgtgctatatgggcttctacttcctcggagccggcctgatggaagccgacatctaccagacacctagatacctggtcatcggcaccggcaaaaagatcaccctggaatgctcccagaccatgggccacgacaagatgtactggtatcagcaggaccccggcatggaactgcatctgatccactacagctacggcgtgaacagcaccgagaagggcgatctgtctagcgagagcaccgtgtccagaatccggaccgagcacttcccactgacactggaaagcgccagacctagccacaccagccagtacctgtgtgccactggacagggggcgcaagatacgcagtattttggcccaggcacccggctgacagtgctcgaggacctcaagaatgtgtttccgcccgaagtcgcggtttttgaaccatcagaagccgagatctctcatacacaaaaggcgacgctcgtatgcctcgcgacgggattttatccggaccacgtcgagctttcctggtgggttaacggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagaatcataccaacaaggagtattgagcgcgacaattctttacgaaattctgcttgggaaagcgactctgtacgcggtgctcgtgtccgctttggttcttatggcaatggttaaacgaaaggatagtaggggcggtagtggacagtgtactaattatgctctcttgaaattggctggagatgttgagagcaatcccgggcccatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccgtccaagtgcccagcatgggcagctcttcaccaatgggggtctctttctgcttggtgatggtactcctttttgcagtggacacaggactatatttctctgtgaagacataacaattgcgcgtcatcatcgatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttca

SEQ ID NO: 182 - DNA, 486 bp, synthetic construct
  source 1..486: mol_type = other DNA
atgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagc

SEQ ID NO: 183 - protein, 162 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 184 - DNA, 486 bp, synthetic construct
  source 1..486: mol_type = other DNA
atgaggagcagtccaggcaatatggagcggatagtaatttgtctcatggtaatattcctcggtactctggtacataaatcttcctctcaaggtcaggaccgccatatgattcgaatgcggcagctgattgacatagtcgatcaactgaagaactatgtgaatgatcttgtgcccgagtttttgccagcccctgaagacgtagaaactaattgtgagtggagtgccttttcctgctttcaaaaggcacagctgaaatccgccaacacgggcaataacgaacggataattaacgtatccattaagaagctgaagcggaagccgccctcaaccaatgcgggacggcggcaaaagcatcgcttgacctgtccgtcatgcgacagctacgagaaaaagcccccgaaggagttcttggaacgcttcaagagtctccttcagaaaatgattcaccagcacctgtcctcacggacgcacggaagcgaggacagt

SEQ ID NO: 185 - protein, 162 aa, synthetic construct
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 186 - DNA, 396 bp, synthetic construct
  source 1..396: mol_type = other DNA
ggccaggaccggcacatgatccggatgagacagctgatcgacatcgtggaccagctgaagaactacgtgaacgacctggtgcccgagttcctgcctgcccccgaggacgtggaaacaaactgcgagtggagcgccttcagctgcttccagaaggcccagctgaaaagcgccaacaccggcaacaacgagcggatcatcaacgtgtccatcaagaagctgaagcggaagccccccagcaccaacgccggaagaaggcagaagcacagactgacctgccccagctgcgacagctacgagaagaagccccctaaagagttcctggaacggttcaagagcctgctgcagaagatgatccaccagcacctgagcagccggacccacggctctgaggacagc

SEQ ID NO: 187 - protein, 132 aa, synthetic construct
GQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

(19) United States
(12) Patent Application Publication
(10) Pub. No.: US 2026/0308225 A1
(43) Pub. Date: Oct. 8, 2026

(54) MULTI-RECEPTOR NATURAL KILLER CELLS

(71) Applicant: BOARD OF REGENTS, THE UNIVERSITY OF TEXAS SYSTEM, Austin, TX (US)

(72) Inventors: Katy REZVANI, Houston, TX (US); Rafet BASAR, Houston, TX (US); Nadima UPRETY, Houston, TX (US); Bin LIU, Houston, TX (US)

(73) Assignee: BOARD OF REGENTS, THE UNIVERSITY OF TEXAS SYSTEM, Austin, TX (US)

(21) Appl. No.: 19/479,874

(22) PCT Filed: Dec. 20, 2023
(86) PCT No.: PCT/US2023/085140

§ 371 Date: Oct. 29, 2025

Related U.S. Application Data
(60) Provisional application No. 63/500,423, filed on May 5, 2023.
(60) Provisional application No. 63/601,150, filed on Nov. 20, 2023.

(51) Int. Cl. C07K 14/705; A61K 39/00; A61K 40/15; A61K 40/32; A61K 40/35; A61K 40/42; A61P 35/00; C07K 14/54; C07K 16/28; C07K 16/32; C12N 5/0783

(52) U.S. Cl. CPC C07K 14/7051; A61K 40/15; A61K 40/32; A61K 40/35; A61K 40/421; A61P 35/00; C07K 14/54; C07K 14/5443; C07K 16/28; C07K 16/2803; C07K 16/2809; C07K 16/2863; C07K 16/2878; C07K 16/2887; C07K 16/32; C12N 5/0646; A61K 2039/505; C07K 2317/24; C07K 2317/31; C12N 2510/00

(57) ABSTRACT

Embodiments of the disclosure include methods and compostions in which NK cells are modified by the hand of man to express a heterologous a) polypeptide comprising a CD16 Fc binding domain, b) a T Cell Receptor (TCR, e.g., an invariant T Cell Receptor (iTCR), and c) a CD3 co-receptor on NK cells that do not naturally express them. Such modified NK cells work effectively (with monospecific, bispecific, or multi-specific antibodies, that may have modified or non-modified Fc domains.

### CROSS REFERENCE TO RELATED APPLICATIONS

This application claims the benefit of priority of U.S. Provisional Patent Application 63/500,423 filed on May 5, 2023, and U.S. Provisional Patent Application 63/601,150 filed on Nov. 20, 2023, the contents of each of which are hereby incorporated by reference in their entirety.

### SEQUENCE LISTING

The instant application contains a Sequence Listing which has been submitted in ST26 format and is hereby incorporated by reference in its entirety. Said ST26 copy, created on May 2, 2023, is named MDAC_1358_Sequence_Listing.xml and is 256,797 bytes in size.

### TECHNICAL FIELD AND BACKGROUND

### I. Technical Field

This disclosure relates at least to the fields of immunology, cell biology, molecular biology, and medicine, including at least cancer medicine.

### II. Background

Natural killer (NK) cells have been studied as potential anti-tumor effectors, yet a number of barriers limit their therapeutic exploitation, mainly related to their lack of antigen specificity. One approach to overcome this is to transduce NK cells with a chimeric antigen receptor (CAR) or an engineered T-cell receptor (TCR) to target a desired antigen. In T cells, one can utilize a bispecific or multi-specific antibody, such as a bispecific T cell engager (BiTE) that binds CD3 on the surface of T cells and that also binds an antigen on the surface of target cells (e.g., cancer cells). CD3 is composed of four distinct chains, and in mammals, the complex contains a CD3γ chain, a CD3δ chain, and two CD3ε chains. These chains associate with the T-cell receptor (TCR) and the ζ-chain (zeta-chain) to generate an activation signal in T lymphocytes. However, NK cells do not naturally express the CD3 receptor complex or TCRs, and thus are not effectively utilized in conjunction with BiTEs. While NK cells naturally express FcγR proteins such as FcγRIII (CD16), polymorphisms in the extracellular Fc recognition domain can result in lack of (or poor) binding of non-Fc domain modified (e.g., glycoengineered) antibodies to NK cell surfaces.

The present disclosure satisfies a long-felt need in the art to improve upon the effectiveness, safety profile, & targeting capacity of immunotherapies, including those that utilize NK cells.

### SUMMARY

Autologous anti-CD19 chimeric antigen receptor (CAR) T-cells have been shown to induce remissions in 57-71% of patients with chronic lymphocytic leukemia (CLL), 52-82% of patients with diffuse large B-cell lymphoma (DLBCL), and 78-92% of patients with low grade non-Hodgkin lymphoma (LG-NHL). Indeed, there are currently multiple FDA-approved autologous anti-CD19 CAR T-cell products available for clinical use. However, CAR T-cells have recognized limitations including the cost of therapy and the time required to collect the T-cells and manufacture the product. Moreover, a proportion of patients treated with CAR T-cells develop toxicities such as cytokine release syndrome (CRS), neurotoxicity, or hemophagocytic lymphohistiocytosis (HLH), each of which carry significant morbidity. These limitations require CAR-T cells to be administered by specialized teams, further limiting access to these life-saving-therapies. Therefore, there is great interest in developing off-the-shelf cell therapies that are cost effective, safe and potent. Natural killer (NK) cells target cancer cells that downregulate HLA class-I molecules or express stress markers in a non-antigen specific manner, thus playing a critical role in cancer immunevigilance. These cells can be engineered to express various transgenes, and can be safely administered without the need for HLA-matching, thus, eliminating the need to produce an immunotherapy product on an individual patient basis. This property makes NK cells especially attractive for off-the-shelf therapy, thus, reducing the cost of manufacturing and expanding access to these potentially life-saving therapies to many more patients.

Embodiments of the disclosure include methods and compositions for treatment of an individual with a disorder, such as but not limited to an autoimmune disorder, cancer, and/or an infection, using adoptive cell therapy. In specific embodiments, the individual is provided a therapeutically effective amount of a bipartite therapy that includes a modified immune cell (e.g., a modified NK cell) and antibodies that are capable of being able to bind the NK cells to initiate signaling, activation, and/or killing of target cells. The disclosure concerns NK cells that have been modified to express multiple proteins that are not naturally expressed in NK cells and that work in conjunction together, including heterologous proteins on the surface of the NK cells that are naturally not present in NK cells. The disclosure also concerns NK cells that have been modified to overexpress proteins that do naturally occur in NK cells.

Provided herein are polynucleotides, which can comprise a sequence at least about 80%, 85%, 90%, 95%, 98%, or 100% identical to transcriptional reading frames comprising SEQ ID NO: 171-175. A polynucleotide encoding a sequence of interested can be comprised in a vector. A vector can comprise at least about 80%, 85%, 90%, 95%, 98%, or 100% sequence identity to SEQ ID NOs: 177-181.

Provided herein are polynucleotides comprising a sequence encoding a T cell receptor (TCR) alpha and TCR beta polypeptide, and/or TCR gamma and TCR delta polypeptide, and a polypeptide comprising a CD16 derived Fc binding domain. TCR polypeptides can be invariant TCR (iTCR) polypeptides. iTCRα and/or iTCRβ polypeptides and/or polynucleotides encoding the same can comprise a sequence at least about 80%, 85%, 90%, 95%, 98%, or 100% identical to any one or more of SEQ ID NOs: 51-149. An iTCRβ polypeptide can comprise a polynucleotide encoding a VP-DJ region that is at least about 80%, 85%, 90%, 95%, 98%, or 100% identical to any one or more of SEQ ID NOs: 75-149. An iTCRβ polypeptide and/or polynucleotide encoding the same can comprise a sequence at least about 80%, 85%, 90%, 95%, 98%, or 100% identical to any one or more of SEQ ID NO: NOs: 59-74. An encoded iTCRβ polypeptide can be at least about 80%, 85%, 90%, 95%, 98%, or 100% identical to SEQ ID NO: 60. A polynucleotide encoding an iTCRβ polypeptide can be at least about 80%, 85%, 90%, 95%, 98%, or 100% identical to SEQ ID NO: 59. An iTCRα polypeptide and/or polynucleotide encoding the same can comprise a sequence at least about 80%, 85%, 90%, 95%, 98%, or 100% identical to SEQ ID NOs: 51-52.

A polynucleotide comprising a sequence encoding a CD16 derived Fc binding domain can comprise a coding sequence for a human CD16A Fc binding domain. A polynucleotide encoding a polypeptide comprising a CD16 derived Fc binding domain can comprise the Fc binding domain being fused in N to C terminus order to an optional hinge domain, a transmembrane domain (TMD), and one or more optional intracellular signaling domains (ICD). A TMD can be derived from CD16, or CD3ζ. A TMD can comprise or consist of a sequence with at least about 90% identity SEQ ID NOs: 163 or 167. A hinge domain can be derived from CD32. In certain cases, a hinge domain can be a hinge domain as previously described in the art. A hinge domain can comprise or consist of a sequence with at least about 90% identity to SEQ ID NO: 161. An ICD can be derived from CD16 and/or CD3ζ. A polypeptide can comprise a CD16 derived Fc binding domain that does not comprise a mutation that renders the CD16 derived Fc binding domain resistant to cleavage. A coding sequence for a polypeptide comprising a CD16 derived Fc domain can be at least about 80%, 85%, 90%, 95%, 98%, or 100% identical to any one or more of SEQ ID NOs: 150-154. A polynucleotide provided herein can comprise a sequence encoding one or more cytokine sequences. A cytokine can comprise TL-15 and/or IL-21.

Also provided herein are engineered NK cells comprising polynucleotides disclosed herein. Additionally, provided herein are methods of treating a disease in an individual, said method comprising administering to an individual in need thereof an engineered NK cell comprising one or more polynucleotides disclosed herein.

Provided herein are engineered immune cells comprising one or more transgenic polynucleotides, where the transgenic polynucleotides encode: a) a CD3 protein complex comprising part or all of a single chain or any combination of CD3δ, CD3ε, CD3γ, or CD3ζ, b) optionally at least one cytokine, c) at least one TCRα and TCRβ chain and/or a TCRγ and TCRδ chain, and d) a polypeptide comprising a CD16 Fc binding domain. One or more transgenic polynucleotides can comprise multicistronic transcriptional open reading frames. The NK cells can be modified to express part or all of CD3δ, two of CD3ε, CD3γ, and/or CD3ζ. One or more of CD3δ, CD3ε, CD3γ, and/or CD3ζ can be linked to one or more heterologous intracellular signaling domains. A heterologous intracellular signaling domain can be selected from the group consisting of CD16, NKG2D, DAP10, DAP12, 2B4, 4-1BB, CD2, CD28, and a combination thereof. A heterologous intracellular signaling domain can comprise a DAP10 intracellular signaling domain. A heterologous intracellular signaling domain can comprise an amino acid sequence at least about 85% identical to SEQ ID NO: 42. A heterologous intracellular signaling domain can comprise a CD28 intracellular signaling domain. A heterologous intracellular signaling domain comprises an amino acid sequence at least about 85% identical to SEQ ID NO: 43. A heterologous intracellular signaling domain can comprise a DAP10 and CD28 intracellular signaling domain. A heterologous intracellular signaling domain can comprise an amino acid sequence at least about 85% identical to SEQ ID NO: 44.

An engineered immune cell provided herein can comprise coding sequences for a CD3 protein complex and at least one cytokine that can be comprised in a first multicistronic construct, wherein coding sequences for at least one TCRα and TCRβ chain and/or a TCRγ and TCRδ chain, and the polypeptide comprising a CD16 Fc binding domain can also be encoded by a second multicistronic construct. An engineered immune cell can include a coding sequence for a cytokine, wherein the cytokine comprises IL-2, IL-7, IL-12, IL-15, IL-18, IL-21, IL-23, and/or GMCSF. An engineered immune cell can be modified to express a polynucleotide sequence at least 85% identical to UT-NK15-DAP10 (SEQ ID NO: 45), UT-NK15-28 (SEQ ID NO: 47), or UTNK15-28-DAP10 (SEQ ID NO: 49). A cytokine can comprise IL-15 and/or IL-21. A cytokine can comprise IL-15 and can comprise a polypeptide sequence and/or polynucleotide sequence encoding the same that is at least 85% identical to any one or more of SEQ ID NOs: 182-183. A cytokine can comprise IL-21 and can comprise a polypeptide sequence and/or polynucleotide sequence encoding the same that is at least 85% identical to any one or more of SEQ ID NOs: 184-187.

An engineered immune cell provided herein can comprise a TCR, where the TCR polypeptides are invariant TCR (iTCR) polypeptides. An iTCRα and iTCRβ polypeptides and/or polynucleotides encoding the same can comprise a sequence at least about 80%, 85%, 90%, 95%, 98%, or 100% identical to any one or more of SEQ ID NOs: 51-149. An iTCRβ polypeptide can comprise a polynucleotide encoding a VP-DJ region that is at least about 80%, 85%, 90%, 95%, 98%, or 100% identical to any one or more of SEQ ID NOs: 75-149. An iTCRβ polypeptide and/or polynucleotide encoding the same can comprise a sequence at least about 80%, 85%, 90%, 95%, 98%, or 100% identical to any one or more of SEQ ID NO: NOs: 59-74. An encoded iTCRβ polypeptide can be at least about 80%, 85%, 90%, 95%, 98%, or 100% identical to SEQ ID NO: 60. A polynucleotide encoding the iTCRβ polypeptide can be at least about 80%, 85%, 90%, 95%, 98%, or 100% identical to SEQ ID NO: 59. An iTCRα polypeptide and/or polynucleotide encoding the same can comprise a sequence at least about 80%, 85%, 90%, 95%, 98%, or 100% identical to SEQ ID NO: 51-52.

An engineered immune cell provided herein can comprise a polypeptide comprising a CD16 Fc binding domain (e.g., a CD16 extracellular domain), wherein the CD16 Fc binding domain can comprise a human CD16 Fc binding domain. A polypeptide comprising a CD16 derived Fc binding domain can comprise a human CD16A Fc binding domain. A polypeptide comprising a CD16 Fc binding domain can be fused (in N to C terminus order) to an optional hinge domain, a transmembrane domain (TMD), and one or more optional intracellular signaling domains (ICD). A TMD can be derived from CD16, or CD3ζ. A TMD can comprise or consist of a sequence with at least about 90% identity to SEQ ID NOs: 163 or 167. A hinge domain can be derived from CD32. A hinge domain can comprise or consist of a sequence with at least about 90% identity to SEQ ID NO: 161. An ICD can be derived from CD16 and/or CD3ζ. An engineered immune cell provided herein can comprise a coding sequence that is least about 80%, 85%, 90%, 95%, 98%, or 100% identical to any one or more of SEQ ID NOs: 171-175.

An engineered immune cell provided herein can be a Natural Killer (NK) cell. An NK cell can be derived from cord blood (CB), peripheral blood (PB), bone marrow, stem cells, or a combination thereof. An NK cell can be a primary NK cell, and can not be derived from stem cells and/or induced pluripotent stem cells (iPSCs).

Also provided herein are compositions comprising engineered NK cells loaded with (e.g., complexed with) one or more antibodies. One or more antibodies can be one or more monospecific, bispecific, or multi-specific antibodies. At least one or more antibodies can comprise a glycoengineered Fc domain that has a high affinity to wild type CD16. At least one or more antibodies can comprise a non-glycoengineered Fc domain that has a low affinity to wild type CD16 Fc binding domains. A non-glycoengineered Fc domain can be loaded on (complexed to) the transgenic polypeptide comprising a CD16 Fc binding domain. One or more antibodies can comprise an IgG1 and/or IgG4 Fc domain.

One or more antibodies target antigens can be CD3, CD16, CD28, EGFR, c-MET, CD30, PSMA, MUC17, CD33, FLT3, STEAP1, BCMA, CLDN18.2, CD123, CD19, CD20, EpCAM, CEA, GPC3, CD38, CD33, CD22, HER2, GPA33, GD2, MUC16, GPRC5D, DLL-3, CLEC12A, and/or SSTR. One or more antibodies targets can be CD3, CD16, CD28, CD19, CD20, CD30, HER2, GPRC5D, EGFR, c-MET, FcRH5, and/or BCMA. One or more antibodies can comprise [fam]-trastuzumab deruxtecan, Abciximab, Adalimumab, Ado-trastuzumab emtansine, Aducanumab, Alemtuzumab, Alirocumab, Amivantamab, Anifrolumab, Ansuvimab, Atezolizumab, Atoltivimab with Maftivimab and Odesivimab-ebgn (aka Inmazeb), Avelumab, Basiliximab, Belantamab mafodotin, Belimumab, Benralizumab, Bevacizumab, Bezlotoxumab, Bimekizumab, Blinatumomab, Brentuximab vedotin, Brodalumab, Brolucizumab, Burosumab, Canakinumab, Caplacizumab, Casirivimab+imdevimab, Catumaxomab, Cemiplimab, Certolizumab pegol, Cetuximab, Cevostamab, Crizanlizumab, Daclizumab, Daratumumab, Denosumab, Dinutuximab, Donanemab, Dostarlimab, Dupilumab, Durvalumab, Eculizumab, Edrecolomab, Efalizumab, Elotuzumab, Emapalumab, Emicizumab, Enfortumab vedotin, Eptinezumab, Erenumab, Evinacumab, Evolocumab, Faricimab, Fremanezumab, Galcanezumab, Gemtuzumab, Gemtuzumab-Ozogamicin, Golimumab, Guselkumab, Ibalizumab, Ibritumomab tiuxetan, Idarucizumab, Imgatuzumab, Inebilizumab, Infliximab, Inolimomab, Inotuzumab, Inotuzumab-Ozogamicin, IPH61, Ipilimumab, Isatuximab, Ixekizumab, Lanadelumab, Lecanemab, Loncastuximab tesirine, Margetuximab, Mepolizumab, Mirvetuximab soravtansine, Mogamulizumab, Mosunetuzumab, Moxetumomab pasudotox, Muromonab-CD3, Narsoplimab, Natalizumab, Naxitamab, Nebacumab, Necitumumab, Nirsevimab, Nivolumab, Obiltoxaximab, Obinutuzumab, Ocrelizumab, Ofatumumab, Olaratumab, Omalizumab, Omburtamab, Oportuzumab monatox, Palivizumab, Panitumumab, Pembrolizumab, Penpulimab, Pertuzumab, Polatuzumab vedotin, Ramucirumab, Ranibizumab, Ravulizumab, Raxibacumab, Regdanvimab, Relatlimab, Reslizumab, Retifanlimab, Risankizumab, Rituximab, Romosozumab, Sacituzumab govitecan, Sarilumab, Satralizumab, Secukinumab, Siltuximab, Sintilimab, Sotrovimab, Spesolimab, Sutimlimab, Tafasitamab, Tebentafusp, Teclistamab, Teplizumab, Teprotumumab, Tezepelumab, Tildrakizumab, Tislelizumab, Tisotumab vedotin, Tixagevimab, cilgavimab, Tocilizumab, Toripalimab, Tositumomab-I131, Tralokinumab, Trastuzumab, Tremelimumab, Ublituximab, Ustekinumab, Vedolizumab, AMG 160/Acapatamab, AMG 199/TNB 585, AMG 330, AMG 427/EMIRODATAMAB, AMG 509, AMG 701, AMG 910, APVO414/ES414/MOR209, APV0436, Catumaxomab/Removab, CC-1, CC-93269/EM801, Cibisatamab/RG7802/RO6958688, CLN-049, Elranatamab/PF-06863135, EMB-06, Epcoritamab/GEN3013, ERY974, Flotetuzumab/MGD006, Glofitamab/RG6026/RO7082859, ISB 1342/GBR 1342, JNJ-63709178, JNJ-63898081, JNJ-67571244, JNJ-75348780, Linvoseltamab/REGN 5458, M701, M802, MGD007, Mosunetuzumab/RG7828, Nivatrotamab/Hu3F8-BsAb, Odronextamab/REGN1979, REGN4018, REGN5459, REGN7075, REGN5678, Talquetamab/JNJ-64407564, Tarlatamab/AMG 757, Tepoditamab/MCLA-117, TNB-383B, TNB-486, TNB-585, XmAb13676/Plamotamab, XmAb14045/Vibecotamab, XmAb18087/Tidutamab, and/or AFM13. One or more antibodies can comprise Elranatamab, Imgatuzumab, Margetuximab, Amivantamab, Blinatumomab, Obinutuzumab, IPH61 (also known as IPH6101 or SAR443579), Teclistamab, Cetuximab, Talquetamab, Pertuzumab, Trastuzumab, Tafasitamab, Brentuximab, and/or Rituximab. One or more antibodies can comprise or consist of Elranatamab. One or more antibodies can comprise or consist of Imgatuzumab. One or more antibodies can comprise or consist of Margetuximab. One or more antibodies can comprise or consist of Amivantamab. One or more antibodies can comprise or consist of Blinatumomab. One or more antibodies can comprise or consist of Obinutuzumab. One or more antibodies can comprise or consist of IPH61. One or more antibodies can comprise or consist of Teclistamab. One or more antibodies can comprise or consist of Cetuximab. One or more antibodies can comprise or consist of Rituximab. An NK cell can be engineered to express the one or more antibodies. One or more antibodies can comprise or consist of Talquetamab. One or more antibodies can comprise or consist of Pertuzumab. One or more antibodies can comprise or consist of Trastuzumab. One or more antibodies can comprise or consist of Tafasitamab. One or more antibodies can comprise or consist of Brentuximab.

An engineered NK cell can be modified to express one or more additional heterologous proteins selected from the group consisting of an antigen receptor, a cytokine, a homing receptor, a chemokine receptor, and a combination thereof. An engineered NK cell can be pre-activated with one or more cytokines. A pre-activation cytokine can be IL-2, IL-7, IL-12, IL-15, IL-18, IL-21, or any combination thereof. An engineered NK cell can comprise one or more engineered mutations in an endogenous gene. An engineered mutation in an endogenous gene can be a mutation in GR, TGFBR2, CISH, and/or CD38. An engineered NK cell provided herein can be comprised in a composition, optionally the composition can include a pharmaceutically acceptable excipient and/or be comprised in a delivery device.

Also provided herein are methods of treating a disease in an individual. Methods of treating an individual can comprise the step of administering to the individual a therapeutically effective amount of any one or more of the cells or compositions described herein. A disease can be an autoimmune disease, infection, and/or cancer. A disease can be cancer. A cancer can express tumor associated antigens (TAAs). A cancer can express CD19, CD20, CD30, HER2, GPRC5D, EGFR, c-MET, and/or BCMA. A cancer can be pancreatic cancer, colorectal cancer, ovarian cancer, kidney cancer, glioblastoma, breast cancer, renal cancer, myeloma, and/or leukemia. A method of treating an individual can further comprise administering to the individual at the same time or at different time, one or more monospecific, bispecific, and/or multispecific antibodies. One or more antibodies can comprise Elranatamab, Imgatuzumab, Margetuximab, Amivantamab, Blinatumomab, Obinutuzumab, IPH61 (also known as IPH6101 or SAR443579), Teclistamab, Cetuximab, and/or Rituximab. One or more antibodies can be administered at the same time as an engineered NK cell, and/or the one or more antibodies and the engineered NK cells can be complexed prior to administration to the individual. One or more antibodies can be administered prior to, after, and/or at the same time as an engineered NK cell. One or more antibodies can be administered more than one time prior to, after, and/or at the same time as an engineered NK cell.

Certain embodiments of the present invention are characterized through the following enumerated aspects.

Aspect 1, is a polynucleotide comprising a sequence at least about 80%, 85%, 90%, 95%, 98%, or 100% identical to transcriptional reading frames comprising SEQ ID NO: 171-175.

Aspect 2, is the polynucleotide of aspect 1, wherein the polynucleotide is comprised in a vector comprising at least about 80%, 85%, 90%, 95%, 98%, or 100% sequence identity to SEQ ID NOs: 177-181.

Aspect 3, is a polynucleotide comprising a sequence encoding a T cell receptor (TCR) alpha and TCR beta polypeptide, and/or TCR gamma and TCR delta polypeptide, and a polypeptide comprising a CD16 derived Fc binding domain.

Aspect 4, is the polynucleotide of aspect 3, wherein the TCR polypeptides are invariant TCR (iTCR) polypeptides.

Aspect 5, is the polynucleotide of aspect 4, wherein the iTCRα and iTCRβ polypeptides and/or polynucleotides encoding the same comprise a sequence at least about 80%, 85%, 90%, 95%, 98%, or 100% identical to any one or more of SEQ ID NOs: 51-149.

Aspect 6, is the polynucleotide of any one of aspects 3 to 5, wherein the iTCRβ polypeptide comprises a polynucleotide encoding a VP-DJ region that is at least about 80%, 85%, 90%, 95%, 98%, or 100% identical to any one or more of SEQ ID NOs: 75-149.

Aspect 7, is the polynucleotide of any one of aspects 3 to 6, wherein the iTCRβ polypeptide and/or polynucleotide encoding the same comprises a sequence at least about 80%, 85%, 90%, 95%, 98%, or 100% identical to any one or more of SEQ ID NO: NOs: 59-74.

Aspect 8, is the polynucleotide of any one of aspects 3 to 7, wherein the encoded iTCRβ polypeptide is at least about 80%, 85%, 90%, 95%, 98%, or 100% identical to SEQ ID NO: 60.

Aspect 9, is the polynucleotide of aspect 8, wherein the polynucleotide encoding the iTCRβ polypeptide is at least about 80%, 85%, 90%, 95%, 98%, or 100% identical to SEQ ID NO: 59.

Aspect 10, is the polynucleotide of any one of aspects 3 to 9, wherein the iTCRα polypeptide and/or polynucleotide encoding the same comprises a sequence at least about 80%, 85%, 90%, 95%, 98%, or 100% identical to SEQ ID NOs: 51-52.

Aspect 11, is the polynucleotide of any one of aspects 3 to 10, wherein the polypeptide comprising a CD16 derived Fc binding domain comprises a human CD16A Fc binding domain.

Aspect 12, is the polynucleotide of any one of aspects 3 to 11, further comprising the Fc binding domain being fused in N to C terminus order to an optional hinge domain, a transmembrane domain (TMD), and one or more optional intracellular signaling domains (ICD).

Aspect 13, is the polynucleotide of aspect 12, wherein the TMD is derived from CD16, or CD3ζ.

Aspect 14, is the polynucleotide of aspect 12 or 13, wherein the TMD comprises or consists of a sequence with at least about 90% identity SEQ ID NOs: 163 or 167.

Aspect 15, is the polynucleotide of any one of aspects 12 to 14, wherein the hinge domain is derived from CD32.

Aspect 16, is the polynucleotide of any one of aspects 12 to 15, comprising a hinge domain that comprises or consists of a sequence with at least about 90% identity to SEQ ID NO: 161.

Aspect 17, is the polynucleotide of any one of aspects 12 to 16, comprising an ICD derived from CD16 and/or CD3ζ.

Aspect 18, is the polynucleotide of any one of aspects 3 to 17, wherein the polypeptide comprising a CD16 derived Fc binding domain does not comprise a mutation that renders the CD16 derived Fc binding domain resistant to cleavage.

Aspect 19, is the polynucleotide of any one of aspects 3 to 18, comprising a coding sequence that is least about 80%, 85%, 90%, 95%, 98%, or 100% identical to any one or more of SEQ ID NOs: 150-154.

Aspect 20, is the polynucleotide of any one of aspects 3 to 19, wherein the polynucleotide further encodes one or more cytokine sequences.

Aspect 21, is the polynucleotide of aspect 20, wherein the cytokine comprises IL-15 and/or IL-21.

Aspect 22, is an engineered NK cell comprising the polynucleotide of any one of aspects 1-22.

Aspect 23, is a method of treating a disease in an individual, the method comprising administration of the engineered NK cells of aspect 22 to an individual in need thereof.

Aspect 24, is an engineered immune cell comprising, one or more transgenic polynucleotides encoding: a) a CD3 protein complex comprising part or all of a single chain or any combination of CD3δ, CD3ε, CD3γ, or CD3ζ, b) optionally at least one cytokine, c) at least one TCRα and TCRβ chain and/or a TCRγ and TCRδ chain, and d) a polypeptide comprising a CD16 Fc binding domain.

Aspect 25, is the engineered immune cell of aspect 24, wherein the one or more transgenic polynucleotides comprise multicistronic transcriptional open reading frames.

Aspect 26, is the engineered immune cell of aspect 24 or 25, wherein the cells are modified to express part or all of CD3δ, two of CD3ε, CD3γ, and/or CD3ζ.

Aspect 27, is the engineered immune cell of any one of aspects 24 to 26, wherein any one or more of CD3δ, CD3ε, CD3γ, and/or CD3ζ are linked to one or more heterologous intracellular signaling domains.

Aspect 28, is the engineered immune cell of aspect 27, wherein the heterologous intracellular signaling domain is selected from the group consisting of CD16, NKG2D, DAP10, DAP12, 2B4, 4-1BB, CD2, CD28, and a combination thereof.

Aspect 29, is the engineered immune cell of any one of aspects 27 to 28, wherein the heterologous intracellular signaling domain comprises a DAP10 intracellular signaling domain.

Aspect 30, is the engineered immune cell of aspect 29, wherein the heterologous intracellular signaling domain comprises an amino acid sequence at least about 85% identical to SEQ ID NO: 42.

Aspect 31, is the engineered immune cell of any one of aspects 27 to 30, wherein the heterologous intracellular signaling domain comprises a CD28 intracellular signaling domain.

Aspect 32, is the engineered immune cell of aspect 31, wherein the heterologous intracellular signaling domain comprises an amino acid sequence at least about 85% identical to SEQ ID NO: 43.

Aspect 33, is the engineered immune cell of any one of aspects 27 to 32, wherein the heterologous intracellular signaling domain comprises a DAP10 and CD28 intracellular signaling domain.

Aspect 34, is the engineered immune cell of aspect 33, wherein the heterologous intracellular signaling domain comprises an amino acid sequence at least about 85% identical to SEQ ID NO: 44.

Aspect 35, is the engineered immune cell of any one of aspects 24 to 34, wherein coding sequences for a CD3 protein complex and at least one cytokine are comprised in a first multicistronic construct, and wherein coding sequences for at least one TCRα and TCRβ chain and/or a TCRγ and TCRδ chain, and the polypeptide comprising a CD16 Fc binding domain are encoded by a second multicistronic construct.

Aspect 36, is the engineered immune cell of any one of aspects 24 to 35, comprising a coding sequence for a cytokine, wherein the cytokine comprises IL-2, IL-7, IL-12, IL-15, IL-18, IL-21, IL-23, and/or GMCSF.

Aspect 37, is the engineered immune cell of aspect 36, where the cell is modified to express a polynucleotide sequence at least 85% identical to UT-NK15-DAP10 (SEQ ID NO: 45), UT-NK15-28 (SEQ ID NO: 47), or UTNK15-28-DAP10 (SEQ ID NO: 49).

Aspect 38, is the engineered immune cell of aspect 37, wherein the cytokine comprises IL-15 and/or IL-21.

Aspect 39, is the engineered immune cell of aspect 38, wherein the cytokine comprises IL-15 and comprises a polypeptide sequence and/or polynucleotide sequence encoding the same that is at least 85% identical to any one or more of SEQ ID NOs: 182-183.

Aspect 40, is the engineered immune cell of aspect 38, wherein the cytokine comprises IL-21 and comprises a polypeptide sequence and/or polynucleotide sequence encoding the same that is at least 85% identical to any one or more of SEQ ID NOs: 184-187.

Aspect 41, is the engineered immune cell of any one of aspects 24 to 40, wherein the TCR polypeptides are invariant TCR (iTCR) polypeptides.

Aspect 42, is the engineered immune cell of aspect 41, wherein the iTCRα and iTCRβ polypeptides and/or polynucleotides encoding the same comprise a sequence at least about 80%, 85%, 90%, 95%, 98%, or 100% identical to any one or more of SEQ ID NOs: 51-149.

Aspect 43, is the engineered immune cell of aspect 41 or 42, wherein the iTCRβ polypeptide comprises a polynucleotide encoding a VP-DJ region that is at least about 80%, 85%, 90%, 95%, 98%, or 100% identical to any one or more of SEQ ID NOs: 75-149.

Aspect 44, is the engineered immune cell of any one of aspects 41 to 43, wherein the iTCRβ polypeptide and/or polynucleotide encoding the same comprises a sequence at least about 80%, 85%, 90%, 95%, 98%, or 100% identical to any one or more of SEQ ID NO: NOs: 59-74.

Aspect 45, is the engineered immune cell of any one of aspects 41 to 44, wherein the encoded iTCRβ polypeptide is at least about 80%, 85%, 90%, 95%, 98%, or 100% identical to SEQ ID NO: 60.

Aspect 46, is the engineered immune cell of aspect 45, wherein the polynucleotide encoding the iTCRβ polypeptide is at least about 80%, 85%, 90%, 95%, 98%, or 100% identical to SEQ ID NO: 59.

Aspect 47, is the engineered immune cell of any one of aspects 41 to 46, wherein the iTCRα polypeptide and/or polynucleotide encoding the same comprises a sequence at least about 80%, 85%, 90%, 95%, 98%, or 100% identical to SEQ ID NO: 51-52.

Aspect 48, is the engineered immune cell of any one of aspects 24 to 47, wherein the CD16 Fc binding domain comprising polypeptide comprises a human CD16 derived Fc binding domain.

Aspect 49, is the engineered immune cell of any one of aspects 24 to 48, wherein the polypeptide comprising a CD16 derived Fc binding domain comprises a human CD16A Fc binding domain.

Aspect 50, is the engineered immune cell of any one of aspects 24 to 49, wherein the Fc binding domain is fused in N to C terminus order to an optional hinge domain, a transmembrane domain (TMD), and one or more optional intracellular signaling domains (ICD).

Aspect 51, is the engineered immune cell of aspect 50, wherein the TMD is derived from CD16, or CD3ζ.

Aspect 52, is the engineered immune cell of aspect 50 or 51, wherein the TMD comprises or consists of a sequence with at least about 90% identity to SEQ ID NOs: 163 or 167.

Aspect 53, is the engineered immune cell of any one of aspects 50 to 52, wherein the hinge domain is derived from CD32.

Aspect 54, is the engineered immune cell of any one of aspects 50 to 53, wherein the hinge domain comprises or consists of a sequence with at least about 90% identity to SEQ ID NO: 161.

Aspect 55, is the engineered immune cell of any one of aspects 50 to 54, comprising an ICD derived from CD16 and/or CD3ζ.

Aspect 56, is the engineered immune cell of any one of aspects 24 to 55 comprising a coding sequence that is least about 80%, 85%, 90%, 95%, 98%, or 100% identical to any one or more of SEQ ID NOs: 171-175.

Aspect 57, is the engineered immune cell of any one of aspects 24 to 56, wherein the cell is a Natural Killer (NK) cell.

Aspect 58, is the engineered NK cell of aspect 57, wherein the NK cells are derived from cord blood (CB), peripheral blood (PB), bone marrow, stem cells, or a combination thereof.

Aspect 59, is the engineered NK cell of any one of aspects 57 to 58, wherein the NK cells are primary NK cells, and are not derived from stem cells and/or induced pluripotent stem cells (iPSCs).

Aspect 60, is the engineered NK cell of any one of aspects 57 to 59, wherein the NK cells are loaded with (complexed with) one or more antibodies.

Aspect 61, is the engineered NK cell of aspect 60, wherein the one or more antibodies are one or more monospecific, bispecific, or multi-specific antibodies.

Aspect 62, is the engineered NK cell of aspect 60 or 61, wherein at least one or more antibodies comprises a glycoengineered Fc domain that has a high affinity to wild type CD16.

Aspect 63, is the engineered NK cell of aspect 60 or 61, wherein the at least one or more antibodies comprise a non-glycoengineered Fc domain that has a low affinity to wild type CD16 Fc binding domains.

Aspect 64, is the engineered NK cell of aspect 63, wherein the non-glycoengineered Fc domain is loaded on (complexed to) the transgenic polypeptide comprising a CD16 Fc binding domain.

Aspect 65, is the engineered NK cell of any one of aspects 60 to 64, wherein the one or more antibodies comprise an IgG1 and/or IgG4 Fc domain.

Aspect 66, is the engineered NK cell of any one of aspects 60 to 65, wherein the one or more antibodies target antigens CD3, CD16, CD28, EGFR, c-MET, CD30, PSMA, MUC17, CD33, FLT3, STEAP1, BCMA, CLDN18.2, CD123, CD19, CD20, EpCAM, CEA, GPC3, CD38, CD33, CD22, HER2, GPA33, GD2, MUC16, GPRC5D, DLL-3, CLEC12A, FcRH5, and/or SSTR.

Aspect 67, is the engineered NK cell of any one of aspects 60 to 66, wherein the one or more antibodies target CD3, CD16, CD28, CD19, CD20, CD30, HER2, GPRC5D, EGFR, c-MET, and/or BCMA.

Aspect 68, is the engineered NK cell of any one of aspects 60 to 67, wherein the one or more antibodies comprise [fam]-trastuzumab deruxtecan, Abciximab, Adalimumab, Ado-trastuzumab emtansine, Aducanumab, Alemtuzumab, Alirocumab, Amivantamab, Anifrolumab, Ansuvimab, Atezolizumab, Atoltivimab with Maftivimab and Odesivimab-ebgn (aka Inmazeb), Avelumab, Basiliximab, Belantamab mafodotin, Belimumab, Benralizumab, Bevacizumab, Bezlotoxumab, Bimekizumab, Blinatumomab, Brentuximab vedotin, Brodalumab, Brolucizumab, Burosumab, Canakinumab, Caplacizumab, Casirivimab+imdevimab, Catumaxomab, Cemiplimab, Certolizumab pegol, Cetuximab, Cevostamab, Crizanlizumab, Daclizumab, Daratumumab, Denosumab, Dinutuximab, Donanemab, Dostarlimab, Dupilumab, Durvalumab, Eculizumab, Edrecolomab, Efalizumab, Elotuzumab, Emapalumab, Emicizumab, Enfortumab vedotin, Eptinezumab, Erenumab, Evinacumab, Evolocumab, Faricimab, Fremanezumab, Galcanezumab, Gemtuzumab, Gemtuzumab-Ozogamicin, Golimumab, Guselkumab, Ibalizumab, Ibritumomab tiuxetan, Idarucizumab, Imgatuzumab, Inebilizumab, Infliximab, Inolimomab, Inotuzumab, Inotuzumab-Ozogamicin, IPH61, Ipilimumab, Isatuximab, Ixekizumab, Lanadelumab, Lecanemab, Loncastuximab tesirine, Margetuximab, Mepolizumab, Mirvetuximab soravtansine, Mogamulizumab, Mosunetuzumab, Moxetumomab pasudotox, Muromonab-CD3, Narsoplimab, Natalizumab, Naxitamab, Nebacumab, Necitumumab, Nirsevimab, Nivolumab, Obiltoxaximab, Obinutuzumab, Ocrelizumab, Ofatumumab, Olaratumab, Omalizumab, Omburtamab, Oportuzumab monatox, Palivizumab, Panitumumab, Pembrolizumab, Penpulimab, Pertuzumab, Polatuzumab vedotin, Ramucirumab, Ranibizumab, Ravulizumab, Raxibacumab, Regdanvimab, Relatlimab, Reslizumab, Retifanlimab, Risankizumab, Rituximab, Romosozumab, Sacituzumab govitecan, Sarilumab, Satralizumab, Secukinumab, Siltuximab, Sintilimab, Sotrovimab, Spesolimab, Sutimlimab, Tafasitamab, Tebentafusp, Teclistamab, Teplizumab, Teprotumumab, Tezepelumab, Tildrakizumab, Tislelizumab, Tisotumab vedotin, Tixagevimab, cilgavimab, Tocilizumab, Toripalimab, Tositumomab-I131, Tralokinumab, Trastuzumab, Tremelimumab, Ublituximab, Ustekinumab, Vedolizumab, AMG 160/Acapatamab, AMG 199/TNB 585, AMG 330, AMG 427/EMIRODATAMAB, AMG 509, AMG 701, AMG 910, APVO414/ES414/MOR209, APV0436, Catumaxomab/Removab, CC-1, CC-93269/EM801, Cibisatamab/RG7802/RO6958688, CLN-049, Elranatamab/PF-06863135, EMB-06, Epcoritamab/GEN3013, ERY974, Flotetuzumab/MGD006, Glofitamab/RG6026/RO7082859, ISB 1342/GBR 1342, JNJ-63709178, JNJ-63898081, JNJ-67571244, JNJ-75348780, Linvoseltamab/REGN 5458, M701, M802, MGD007, Mosunetuzumab/RG7828, Nivatrotamab/Hu3F8-BsAb, Odronextamab/REGN1979, REGN4018, REGN5459, REGN7075, REGN5678, Talquetamab/JNJ-64407564, Tarlatamab/AMG 757, Tepoditamab/MCLA-117, TNB-383B, TNB-486, TNB-585, XmAb13676/Plamotamab, XmAbl4045/Vibecotamab, XmAb18087/Tidutamab, and/or AFM13.

Aspect 69, is the engineered NK cell of any one of aspects 60 to 68, wherein the one or more antibodies comprise Elranatamab, Imgatuzumab, Margetuximab, Amivantamab, Blinatumomab, Obinutuzumab, IPH61 (also known as IPH6101 or SAR443579), Teclistamab, Cetuximab, Talquetamab, Pertuzumab, Trastuzumab, Tafasitamab, Brentuximab, and/or Rituximab.

Aspect 70, is the engineered NK cell of aspect 69, wherein the one or more antibodies comprises or consists of Elranatamab.

Aspect 71, is the engineered NK cell of aspect 69, wherein the one or more antibodies comprises or consists of Imgatuzumab.

Aspect 72, is the engineered NK cell of aspect 69, wherein the one or more antibodies comprises or consists of Margetuximab.

Aspect 73, is the engineered NK cell of aspect 69, wherein the one or more antibodies comprises or consists of Amivantamab.

Aspect 74, is the engineered NK cell of aspect 69, wherein the one or more antibodies comprises or consists of Blinatumomab.

Aspect 75, is the engineered NK cell of aspect 69, wherein the one or more antibodies comprises or consists of Obinutuzumab.

Aspect 76, is the engineered NK cell of aspect 69, wherein the one or more antibodies comprises or consists of IPH61.

Aspect 77, is the engineered NK cell of aspect 69, wherein the one or more antibodies comprises or consists of Teclistamab.

Aspect 78, is the engineered NK cell of aspect 69, wherein the one or more antibodies comprises or consists of Cetuximab.

Aspect 79, is the engineered NK cell of aspect 69, wherein the one or more antibodies comprises or consists of Rituximab.

Aspect 80, is the engineered NK cell of aspect 69, wherein the one or more antibodies comprises or consists of Talquetamab.

Aspect 81, is the engineered NK cell of aspect 69, wherein the one or more antibodies comprises or consists of Pertuzumab.

Aspect 82, is the engineered NK cell of aspect 69, wherein the one or more antibodies comprises or consists of Trastuzumab.

Aspect 83, is the engineered NK cell of aspect 69, wherein the one or more antibodies comprises or consists of Tafasitamab.

Aspect 84, is the engineered NK cell of aspect 69, wherein the one or more antibodies comprises or consists of Brentuximab.

Aspect 85, is the engineered NK cell of any one of aspects 60 to 84, wherein the NK cell expresses the one or more antibodies.

Aspect 86, is the engineered NK cell of any one of aspects 57 to 85, wherein the NK cell is further modified to express one or more additional heterologous proteins selected from the group consisting of an antigen receptor, a cytokine, a homing receptor, a chemokine receptor, and a combination thereof.

Aspect 87, is the engineered NK cell of any one of aspects 57 to 86, wherein the NK cells are pre-activated with one or more cytokines.

Aspect 88, is the engineered NK cell of aspect 87, wherein the cytokines are IL-2, IL-7, IL-12, IL-15, IL-18, IL-21, or a combination thereof.

Aspect 89, is the engineered NK cell any one of aspects 57 to 88, wherein the NK cell further comprises one or more engineered mutations in an endogenous gene.

Aspect 90, is the engineered NK cell of aspect 89, wherein the endogenous gene is GR, TGFBR2, CISH, and/or CD38.

Aspect 91, is a composition comprising the engineered NK cell of any one of aspects 57 to 90.

Aspect 92, is the composition aspect 91, further comprising a pharmaceutically acceptable excipient.

Aspect 93, is the composition aspect 91 or 92, wherein the composition is comprised in a delivery device.

Aspect 94, is a method of treating a disease in an individual, the method comprising the step of administering to the individual a therapeutically effective amount of any one of the cells or compositions of aspects 24 to 91.

Aspect 95, is the method of aspect 94, wherein the disease is an autoimmune disease, infection, and/or cancer.

Aspect 96, is the method of aspect 95, wherein the disease is an autoimmune disease.

Aspect 97, is the method of aspect 95.1, wherein the autoimmune disease comprises a B cell related autoimmunity, systemic lupus erythematosus (SLE), Systemic scleroderma (SSc), multiple sclerosis (MS), Grave's disease, rheumatoid arthritis (RA), myositis, dermatomyositis. myasthenia gravis, Sjogren's syndrome, diffuse scleroderma, inflammatory myopathy, inflammatory myopathy, ANCA-associated systemic vasculitis, antiphospholipid syndrome, immune nephritis, ITP, refractory POEMS syndrome, amyloidosis, autoimmune hemolytic anemia, and/or vasculitis.

Aspect 98, is the method of aspect 95, wherein the disease is cancer.

Aspect 99, is the method of aspect 98, wherein the cancer expresses CD19, CD20, CD30, HER2, GPRC5D, EGFR, c-MET, and/or BCMA.

Aspect 100, is the method of aspect 98 or 99, wherein the cancer is pancreatic cancer, colorectal cancer, ovarian cancer, kidney cancer, glioblastoma, breast cancer, renal cancer, myeloma, and/or leukemia.

Aspect 101, is the method of aspect any one of aspects 95-100, further comprising administering to the individual at the same time or at different time, one or more monospecific, bispecific, and/or multispecific antibodies.

Aspect 102, is the method of aspect 101, wherein the one or more antibodies comprise Elranatamab, Imgatuzumab, Margetuximab, Amivantamab, Blinatumomab, Obinutuzumab, IPH61 (also known as IPH6101 or SAR443579), Teclistamab, Cetuximab, Talquetamab, Pertuzumab, Trastuzumab, Tafasitamab, Brentuximab, and/or Rituximab.

Aspect 103, is the method of aspect 101 or 102, wherein the one or more antibodies are administered at the same time, and/or the one or more antibodies and the engineered NK cells are complexed prior to administration to the individual.

Aspect 104, is the method of any one of aspects 101 to 103, wherein the one or more antibodies are administered more than once, including at least once at a time point after administration of the engineered NK cells.

Aspect 105, is the method of any one of aspects 101 to 104, wherein the one or more antibodies are administered more than once, including at least once at a time point before administration of the engineered NK cells.

Aspect 106, is the method of any one of aspects 101 to 105, wherein the one or more antibodies are administered more than once, including at least once at a time point before administration of the engineered NK cells and at least once at a time point after administration of the engineered NK cells.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein. Unless otherwise noted, two-way analysis of variance (ANOVA) was employed for statistical analysis, and p-value of less than 0.05 was considered statistically significant.

FIGS. 1A-1D shows in vitro expression of, and the cytotoxic function of, invariant Natural Killer T-cell Receptors (iTCRs)-transduced NK cells. Additional experimental details are provided in Example 6 below. FIG. 1A, depicts exemplary flow cytometry plots showing expression of three different iTCR pairs, each comprising iTCRα (SEQ ID NO: 52), and either iTCRβ chain 1 (SEQ ID NO: 54), iTCRβ chain 2 (SEQ ID NO: 56), or iTCRβ chain 3 (SEQ ID NO: 58), and CD3 on NK cells, using antibodies specific for iTCR target Vα24 and Vβ11 regions (y and x axis respectively). FIG. 1B, depicts exemplary flow cytometry plots showing binding of Blinatumomab (y axis) to three different CD3/iTCR complexes as described in (A), e.g., iTCR1, iTCR2, and iTCR3, respectively, expressing NK cells. NK cells were derived from three donors, cell donor 4 (CD4), cell donor 8 (CD8), or cell donor N (CDN), respectively. The binding of Blinatumomab on CD3 was confirmed by flow cytometry using the CAR19 detection kit from Miltenyi Biotech™. FIG. 1C, depicts results of IncuCyte® live cell imaging assays that were used to measure the cytotoxicity of NK cells pre-loaded with Blinatumomab and co-transduced with iTCR and UT-NK15, against GFP-expressing Raji tumor cells at a 3:1 effector to target ratio. T cells and iNKT cells were used as positive and negative controls, respectively. A reduction in GFP expression indicated cell death. Three NK donor lines were utilized, CB152, CB153, and CB154, respectively. NK cells were transduced with three different CD3/iTCR complexes as described in (FIG. 1A), e.g., iTCR1, iTCR2, or iTCR3, or left non-transduced (NT) (as negative control). FIG. 1D, depicts a bar chart showing residual tumor % (Y axis) for the data presented in FIG. 1C. Unpaired t test was utilized to compare iNKT with each of the different iTCRs individually. CD3/iTCR NK cells were significantly (P=<0.01) more cytotoxic than iNKT cells (N=3 replicates).

FIGS. 2A-2D shows the cloning of iTCR sequences from NK T Cells isolated from human cord blood, and the transgenic expression and efficacy of a subset of iTCR clones in transduced cord blood derived NK cells. Additional experimental details are provided in Example 6 below. FIG. 2A, depicts the isolation of iTCR clones from iNKT cells purified from five cord blood donors, and exemplary flow cytometry results showing expression of iTCRs on said donor iNKT cells. iNKT cells were isolated from 5 cord blood donors using iNKT isolation kit from Miltenyi Biotech™. Isolated cells were stimulated and expanded with irradiated cord blood PBMC(40 Gry) in the presence of 100 ng/ml of alpha-Galactosylceramide and 200 U/ml of IL-2. On day 7 after iNKT expansion, the purity of iNKT cultures was confirmed by iNKT specific antibodies against Va24 and VP 11. Total mRNA was extracted from iNKT cells, and cDNAs of the VP-DJ regions were cloned and sequenced (100 individual clones). FIG. 2B, depicts sequences of iTCRβ clone VP-DJ sequences. FIG. 2C, depicts the transgenic expression of Va24 and CD3 in transgenic CD3/iTCR expressing NK cell populations from three cord blood donors. The NK cells were co-transduced with iTCRs (eight randomly selected iTCRβ clones with coding sequences represented by SEQ ID NOs: 59, 61, 63, 65, 67, 69, 71, or 73, representing clones 3, 18, 24, 51, 56, 76, 93, and 96 respectively, and iTCRα coding sequence represented by SEQ ID NO: 51) and UT-NK15 on day 5. iTCR expression was confirmed by flow cytometry using iTCR specific antibodies against Va24 and antibodies against CD3. FIG. 2D, depicts a graph describing the results of IncuCyte® live cell imaging mediated measurements of cytotoxicity of NK cells co-transduced with iTCRs (as described in FIG. 2B) and UT-NK15, and pre-loaded with Blinatumomab (1 hour at 37° C.), against GFP expressing Raji cells at 3:1 target effector ratios. T cells were used as positive control and non-transduced (NT) NK cells were utilized as negative controls. A reduction in GFP expression indicated tumor cell death.

FIGS. 3A-3C shows polynucleotide construct layouts and graphical schematics for exemplary uTNK15 and T Cell Receptor (TCR)/Fc Receptor (FcR) (TCR/FcR) vectors (e.g., vectors comprising a coding sequence for one or more TCR chains and one or more polypeptides comprising an Fc binding domain derived from an Fc Receptor). FIG. 3A, shows a vector map for multicistronic construct uTNK15-28 (SEQ ID NO: 47) comprising an open reading frame (ORF) coding region for CD3 complex proteins and IL-15. FIG. 3B, shows a vector map for multicistronic TCR/FcR constructs described herein comprising open reading frames for an alpha (α; SEQ ID NO: 51) and beta (0, clone 3; SEQ ID NO: 59) invariant T Cell Receptor (iTCR) chains and a CD16 Fc binding domain comprising polypeptide. FIG. 3C, shows a graphical schematic for uTNK15-28, and iTCR/CD16 Fc binding domain polypeptide bearing constructs: TCR/FcR #1 (open reading frame (ORF) SEQ ID NO: 171, in vector SEQ ID NO: 177), TCR/FcR #2 (ORF SEQ ID NO: 172, in vector SEQ ID NO: 178), TCR/FcR #3 (ORF SEQ ID NO: 173, in vector SEQ ID NO: 179), TCR/FcR #4 (ORF SEQ ID NO: 174, in vector SEQ ID NO: 180), and TCR/FcR #5 (ORF SEQ ID NO: 175, in vector SEQ ID NO: 181).

FIG. 4 shows CB-NK and T cells transduced with various TCR/FcR constructs. The top row depicts T cells expanded with anti-CD3/28 beads in the presence of IL-2 (50 Iu/ML) that were transduced with the different TCR/FcR constructs at day 3. Transduction efficiency was determined 48 hours later by measuring the surface expression of iTCRvb11 and CD16. The middle row depicts cord blood derived NK cells expanded with irradiated (100 Gy) UAPC feeder cells (2:1 feeder cell: NK ratio) and recombinant human IL-2 (200 U/ml) in complete NK cell growth medium. Seven days after expansion, NK cells were transduced with the noted iTCR3 or one of TCR/FcR constructs #1-#5, comprising CD16 Fc binding domain polypeptide variant sequences SEQ ID NOs: 155-159 (encoded by SEQ ID NOs: 150-154, respectively) respectively. Transduction efficiency was determined 48 hours later by measuring surface expression of iTCRvb11, CD3 complex, and as depicted in the bottom row, heterologous CD16 Fc binding domain comprising polypeptides (measured as anti-flag antibody against flag tagged CD16 to avoid endogenous CD16 background signal).

FIGS. 5A-5F shows TCR/FcR construct transduced NK cells loaded with Blinatumomab (Blina) displaying enhanced killing of CD19+(Raji cell line) tumor cells. Cord blood derived NK cells were transduced with uTNK15, and iTCR3 (FIG. 5A) or one of TCR/FcR constructs #1-#5 (FIGS. 5B-5F). Non-transduced (NT) cord blood derived NK cells (NT NK cell) served as negative controls, while non-transduced T cells were used as positive controls. Cells were or were not loaded with Blinatumomab (100 μg/ml final concentration) for one hour at room temperature in PBS, cells were then washed prior to co-culturing with Raji cells. The Raji cells were labelled with chromium-51 and co-cultured with the immune cells at various effector to target (E:T) ratios (X axis). Cells were co-cultured for four hours and chromium release (corresponding to NK cell cytotoxicity against the cancer cells) was measured (y-axis). Compared to non-loaded TCR/FcR-NK cells, or loaded non-transduced (NT) NK Cells, Blinatumomab-loaded TCR/FcR transduced NK cells showed increased cytotoxicity against CD19+Raji cells.

FIG. 6 shows TCR/FcR transduced NK cells loaded with Blinatumomab and/or Obinutuzumab displaying enhanced killing of CD19+++/CD20+(Nalm6) tumor cells. Cord blood derived NK cells were transduced with uTNK15, and iTCR3 or one of TCR/FcR constructs #1-#5. Non-transduced (NT) cord blood derived NK cells (NT NK cell) served as negative controls. NK cells were left unloaded (PBS), loaded with Blinatumomab (100 μg/ml final concentration), loaded with Obinutuzumab (500 μg/ml final concentration), or loaded with Blinatumomab (100 μg/ml final concentration) and Obinutuzumab (500 μg/ml final concentration) for one hour at room temperature in PBS and then washed prior to co-culturing with tumor cells. Immune cells and Nalm6 cells were co-cultured at a 1:1 E:T ratio and real-time cytotoxicity of effector cells against the Nalm6 cells was measured every 2 hours over a 40 hour period. Compared to non-loaded TCR/FcR-NK cells or loaded NT NK Cells, Blinatumomab and/or Obinutuzumab loaded TCR/FcR transduced NK cells showed increased cytotoxicity against CD19+NALM6 cells.

FIG. 7 shows how the T cell engager, Teclistamab, can bind to TCR/FcR transduced NK cells and T cells, but not to non-transduced NK cells. NK cells were isolated from cord blood and expanded in complete media in the presence of irradiated (100 Gy) uAPC feeder cells (2:1 feeder cell:NK ratio) and recombinant human IL-2 (200 U/ml). Seven days following expansion, NK cells were transduced with uTNK15, and iTCR3 or one of TCR/FcR constructs #1-#5, or left non-transduced (NT). T cells were used as a positive control and loaded with Teclistamab (200 μg/ml final concentration) for one hour at room temperature in PBS and then washed prior to validation of Teclistamab binding. Flow cytometric analysis against anti-human IgG stained cells showed Teclistamab was bound to NK cells transduced with uTNK15, and iTCR3 or TCR/FcR #1-#5, and to T cells, but not NT NK cells.

FIGS. 8A-8F shows TCR/FcR transduced NK cells loaded with Teclistamab (Tecli) displaying enhanced killing of BCMA+(MM.1S, myeloma) tumor cells. Cord blood derived NK cells were transduced with uTNK15, and iTCR3 (FIG. 8A) or one of TCR/FcR constructs #1-#5 (FIGS. 8B-8F). Non-transduced (NT) cord blood derived NK cells (NT NK cell) served as negative controls, while non-transduced T cells were used as positive controls. Cells were or were not loaded with Teclistamab (200 μg/ml final concentration) for one hour at room temperature in PBS, cells were then washed prior to co-culturing with MM.1S cells. The MM.1 S cells were labelled with chromium-51 (cr51) and co-cultured with the immune cells at various effector to target (E:T) ratios (X axis). Cells were co-cultured for four hours and chromium release (corresponding to NK cell cytotoxicity against the cancer cells) was measured (y-axis). Compared to non-loaded TCR/FcR-NK cells, or loaded non-transduced (NT) NK Cells, Teclistamab-loaded TCR/FcR transduced NK cells showed increased cytotoxicity against BCMA+MM.1 S cells.

FIGS. 9A-9F shows TCR/FcR transduced NK cells loaded with Teclistamab (Tecli) displaying enhanced killing of BCMA+(H929, myeloma) tumor cells. Cord blood derived NK cells were transduced with uTNK15, and iTCR3 (FIG. 9A) or one of TCR/FcR constructs #1-#5 (FIGS. 9B-9F). Non-transduced (NT) cord blood derived NK cells (NT NK cell) served as negative controls, while non-transduced T cells were used as positive controls. Cells were or were not loaded with Teclistamab (200 μg/ml final concentration) for one hour at room temperature in PBS, cells were then washed prior to co-culturing with MM.1 S cells. The H929 cells were labelled with chromium-51 and co-cultured with the immune cells at various effector to target (E:T) ratios (X axis). Cells were co-cultured for four hours and chromium release (corresponding to NK cell cytotoxicity against the cancer cells) was measured (y-axis). Compared to non-loaded TCR/FcR-NK cells, or loaded non-transduced (NT) NK Cells, Teclistamab-loaded TCR/FcR transduced NK cells showed increased cytotoxicity against BCMA+H929 cells.

FIG. 10, shows binding of low Fcγ receptor affinity antibody (e.g., Cetuximab) to TCR/FcR transduced NK cells. NK cells were derived from cord blood and expanded with irradiated (100 Gy) UAPC feeder cells (2:1 feeder cell:NK ratio) and recombinant human IL-2 (200 U/ml) in complete NK cell growth medium (Click's/RPMI). NK cells were loaded with low Fc receptor affinity antibody (e.g., Cetuximab at 100 μg/ml final concentration) for one hour at 37° C. in Click's/RPMI media followed by washing before validating the binding of the low Fc receptor affinity antibody with flow cytometry. NK cells were stained with Alexa-Fluor647 affinity-purified F(ab′)₂ fragment goat anti-human IgG (H+L) antibody and analyzed by flow cytometry. The results showed that antibodies with low Fcγ receptor affinity Fc domains (e.g., Cetuximab) bound to high affinity CD16 (CD16 ha, (F158V)) TCR/FcR transduced NK cells at greater levels when compared to ITCR3 transduced NK cells, NT NK cells, or T cells.

FIG. 11, shows TCR/FcR transduced NK cells loaded with Cetuximab displaying enhanced killing of WiDR (colorectal cancer cells; CRC) compared to non-loaded TCR/FcR NK cells or loaded/non-loaded non-transduced (NT) NK Cells. NK cells were transduced with uTNK15, and iTCR3 or one of TCR/FcR constructs #1-#5. WiDR cells alone, Non-transduced (NT) cord blood derived NK cells (NT NK cell), and T cells served as served as controls. Cells were or were not loaded with Cetuximab (100 μg/ml final concentration) for one hour at 37° C. in Click's/RPMI media and then washed before co-culturing with tumor cells at 1:1 E:T ratio. Compared to Cetuximab alone, or to loaded/non-loaded NT NK cells or to non-loaded TCR/FcR NK cells, Cetuximab loaded TCR/FcR NK cells showed the highest cytotoxic activities against the EGFR+ CRC (WiDR) cell line. The lower the normalized cell index (Y axis), the higher the degree of cytotoxicity. SDS was used a positive control for cytotoxicity. NK cells transduced with TCR/FcR #4 or TCR/FcR #2 and loaded with cetuximab exerted the greatest levels of cytotoxicity against WiDR cells.

FIGS. 12A-12B, shows TCR/FcR transduced NK cells loaded with Cetuximab displaying enhanced killing of PATC148 cells (pancreatic ductal adenocarcinoma, PDAC) compared to non-loaded TCR/FcR NK cells or loaded/non-loaded non-transduced (NT) NK Cells. NK cells were transduced with uTNK15, and iTCR3 or one of TCR/FcR constructs #1-#5. PATC148 cells alone, Non-transduced (NT) cord blood derived NK cells (NT NK cell), and T cells served as controls. Cells were or were not loaded with Cetuximab (100 μg/ml final concentration) for one hour at 37° C. in Click's/RPMI media and then washed before co-culturing with tumor cells at 4:1 E:T ratio (FIG. 12A), or at 2:1 ET ratio (FIG. 12B). Compared to Cetuximab alone, or with loaded/non-loaded non-transduced (NT) NK cells or with non-loaded TCR/FcR NK cells, Cetuximab loaded TCR/FcR transduced NK cells showed the greatest cytotoxic activity against EGFR+ PATC148 (PDAC) cell lines. The lower the normalized cell index (Y axis), the higher the degree of cytotoxicity. SDS was used a positive control for cytotoxicity. NK cells transduced with TCR/FcR #4 or TCR/FcR #2 and loaded with cetuximab exerted the greatest levels of cytotoxicity against PATC148 (PDAC) cells.

FIGS. 13A-13B show TCR/FcR transduced NK cells loaded with Imgatuzumab displaying enhanced killing of PDAC or CRC cells when compared to non-loaded TCR/FcR NK cells or loaded/un-loaded non-transduced (NT) NK Cells. NT NK cells or NK cells transduced with uTNK15 and TCR/FcR construct #2 were loaded with Imgatuzumab (10 μg/ml final concentration) for one hour at room temperature in PBS and then washed before co-culture with EGFR+ PDAC (PATC-148, FIG. 13A) or EGFR+ colorectal cancer (WiDR, FIG. 13B) tumor cells at a 1:1 E:T ratio. Compared to Imgatuzumab alone, loaded/un-loaded NT-NK cells, or non-loaded TCR/FcR NK cells, Imgatuzumab loaded TCR/FcR transduced NK cells showed increased cytotoxic activity against EGFR+ PDAC (PATC 148) and colorectal (WiDR) cell lines.

FIGS. 14A-14C show TCR/FcR transduced NK cells loaded with Imgatuzumab displaying enhanced killing of 3D PDAC (PATC-148) tumor spheroids. FIG. 14A, displays representative images of PATC148 spheroids (PDAC tumor cell line transduced with GFP) either left alone, treated with NT NK Cells, or treated with NK cells transduced with uTNK15 and TCR/FcR construct #2, the NK cells were either non-loaded or loaded with Imgatuzumab (10 μg/ml final concentration) for one hour at room temperature in PBS and then washed before co-culture. The data showed significant decreases in spheroid size in the wells treated with Imgatuzumab loaded TCR/FcR transduced NK cells. FIG. 14B, depicts quantification of the total integrated green intensity over time observed in 14A, the data demonstrated a significant decrease in total integrated green intensity (FIG. 14C) when spheroids were treated with TCR/FcR transduced NK cells loaded with Imgatuzumab.

FIGS. 15A-15C, shows binding of Cetuximab, Amivantamab, or Imgatuzumab to TCR/FcR #2 transduced NK cells. NK cells were derived from cord blood and were expanded with irradiated (100 Gy) UAPC feeder cells (2:1 feeder cell:NK ratio) and recombinant human IL-2 (200 U/ml) in complete NK cell growth medium (Click's/RPMI). Non-transduced NK cells or NK cells transduced with TCR/FcR #2 were loaded with Cetuximab (250 μg/ml final concentration) (FIG. 15A), Amivantamab (100 μg/ml final concentration) (FIG. 15B), or Imgatuzumab (100 μg/ml final concentration) (FIG. 15C), for one hour at 37° C. in Click's/RPMI media before the cells were washed. NK cells were stained with Alexa-Fluor647 affinity-purified F(ab′)₂ fragment goat anti-human IgG (H+L) antibody and analyzed by flow cytometry to confirm the binding of Cetuximab, Amivantamab, or Imgatuzumab. The results showed that antibodies were bound to TCR/FcR transduced NK cells with higher affinity when compared to NT NK cells. Of note, unlike Amivantamab or Imgatuzumab, Cetuximab (which comprises an Fc domain with low binding affinity to WT CD16) was found to bind only to the CD16 ha TCR/FcR transduced NK cells.

FIGS. 16A-16C, depicts uTNK15 and TCR/FcR #2 transduced NK cells demonstrating enhanced antitumor activity against PATC-148 (PDAC) cell spheroids when loaded with Cetuximab (Cetux), Imgatuzumab (Imga), or Amivantamab (Ami) relative to NT NK cells. Antibody loaded TCR/FcR transduced NK cells showed enhanced killing of PDAC cells when compared to non-loaded TCR/FcR NK cells or loaded/non-loaded non-transduced (NT) NK Cells. FIG. 16A shows representative images of GFP transduced PDAC (PATC-148) spheroids that were either left alone, treated with non-loaded or loaded NT NK, or treated with non-loaded or loaded TCR/FcR #2 transduced NK cells. Loaded cells were prepared with Imgatuzumab (100 μg/ml final concentration), Amivantamab (100 μg/ml final concentration), or Cetuximab (250 μg/ml final concentration). A readily observable and significant decrease in spheroid size was observed in the wells treated with antibody-loaded TCR/FcR transduced NK cells. FIG. 16B is a graphical quantification of the total integrated green intensity over time from experiments as performed in FIG. 16A, the data shows a significant decrease (FIG. 16C) in total integrated green intensity when spheroids were treated with TCR/FcR transduced NK cells loaded with antibodies.

FIGS. 17A-17C depicts uTNK15 and TCR/FcR #2 transduced NK cells demonstrating enhanced antitumor activity against WiDR (CRC) cell spheroids when loaded with Cetuximab (Cetux), Imgatuzumab (Imga), or Amivantamab (Ami) relative to NT NK cells. Antibody loaded TCR/FcR transduced NK cells showed enhanced killing of CRC cells when compared to non-loaded TCR/FcR NK cells or loaded/non-loaded non-transduced (NT) NK Cells. FIG. 17A shows representative images of GFP transduced WiDR spheroids that were either left alone, treated with non-loaded or loaded NT NK, or treated with non-loaded or loaded TCR/FcR #2 transduced NK cells. Loaded cells were prepared with Imgatuzumab (100 μg/ml final concentration), Amivantamab (100 μg/ml final concentration), or Cetuximab (250 μg/ml final concentration). The data show significant decreases in spheroid size in the wells treated with antibody-loaded TCR/FcR transduced NK cells. FIG. 17B is a graphical quantification of the total integrated green intensity over time from experiments as performed in FIG. 16A, the data shows a significant decrease (FIG. 17C) in total integrated green intensity when spheroids were treated with TCR/FcR transduced NK cells loaded with antibodies.

FIGS. 18A-18F, depicts uTNK15 and TCR/FcR #2 transduced NK cells demonstrating enhanced antitumor activity against various solid tumor cell lines when loaded with anti-EGFR antibodies Cetuximab, Imgatuzumab, or Amivantamab. Antibody loaded TCR/FcR transduced NK cells showed enhanced killing when compared to non-loaded TCR/FcR NK cells or loaded/non-loaded non-transduced (NT) NK Cells. NK cells were either non-transduced (NT) or transduced with TCR/FcR #2 and were left unloaded or loaded with Cetuximab (250 μg/ml final concentration), Amivantamab (100 μg/ml final concentration), or Imgatuzumab (100 μg/ml final concentration) for one hour at 37° C. in Click's/RPMI media followed by washing. NK cells were co-cultured with tumor cells SKOV3 (FIG. 18A), PATC-148 (FIG. 18C), or WiDR (FIG. 18E) at 2:1 E:T ratios. Compared to antibodies alone, loaded/non-loaded non-transduced (NT) NK cells, or non-loaded TCR/FcR #2 NK cells, the antibody-loaded TCR/FcR #2 transduced NK cells showed significantly increased cytotoxic activity against EGFR+ SKOV3 (FIG. 18B), PATC-148 (FIG. 18D), or WiDR (FIG. 18F) cell lines.

FIGS. 19A-19B, shows binding of low Fcγ receptor affinity antibody (e.g., Rituximab) or high Fcγ receptor affinity antibody (Obinutuzumab) to TCR/FcR #2 transduced NK Cells. NK cells were derived from cord blood and were expanded with irradiated (100 Gy) UAPC feeder cells (2:1 feeder cell:NK ratio) and recombinant human IL-2 (200 U/ml) in complete NK cell growth medium (Click's/RPMI). Non-transduced NK cells or NK cells transduced with TCR/FcR #2 were loaded with Rituximab (100 μg/ml final concentration) (FIG. 19A), or Obinutuzumab (100 μg/ml final concentration) (FIG. 19B) for one hour at 37° C. in Click's/RPMI media followed by washing. NK cells were stained with Alexa-Fluor647 affinity-purified F(ab′)₂ fragment goat anti-human IgG (H+L) antibody and analyzed by flow cytometry. The results showed that low and high Fcγ receptor affinity antibodies were bound to TCR/FcR transduced NK cells at greater rates when compared to NT NK cells. Of note, unlike Obinutuzumab, Rituximab (which comprises an Fc domain with low binding affinity to WT CD16) was found to bind only to the CD16 ha TCR/FcR transduced NK cells.

FIGS. 20A-20B, depicts TCR/FcR #2 transduced NK cells demonstrating enhanced antitumor activity against Raji (B cell lymphoma) cells when loaded with anti-CD20 antibodies Rituximab or Obinutuzumab when compared to NT NK at various E:T ratios. NK cells were either non-transduced (NT) or transduced with TCR/FcR #2, and left unloaded or loaded with Rituximab (100 μg/ml final concentration) (FIG. 20A), or Obinutuzumab (100 μg/ml) for one hour at 37° C. in Click/RPMI media followed by washing. NK cells were co-cultured with chromium-51 labelled Raji cells at various E:T ratios (5:1, 2:1, 1:1, or 1:2), for four hours and chromium release (corresponding to the cytotoxicity of the cancer cells) was measured. Compared to non-loaded TCR/FcR NK cells or loaded NT NK cells, antibody-loaded TCR/FcR #2 transduced NK cells showed increased cytotoxicity against CD20+Raji cells.

FIG. 21, depicts an exemplary combination of therapeutic strategies as described herein (e.g., uTNK15 and TCR/FcR transduced NK cells loaded with antibodies).

FIGS. 22A-22B, shows uTNK15 and TCR/FcR transduced NK cells loaded with Elranatamab demonstrated enhanced antitumor activity against BCMA+multiple myeloma cells (e.g., MM.1S cells), and outlines a clinical trial comprising combination TCR/FcR transduced NK cells and Elranatamab. FIG. 22A shows the results of a Bioluminescence imaging (BLI) experiment, where mice were irradiated, inoculated with 5×10⁵ MM.1.S cells and sham injected or injected (IV) with a single infusion of 1×10⁷ NK cells that were either non-transduced or transduced with uTNK15 and TCR/FcR #2. Animals were imaged weekly. The results showed that mice receiving TCR/FcR transduced NK cells loaded with Elranatamab demonstrated enhanced antitumor activity in vivo relative to controls. FIG. 22B schematically outlines an exemplary clinical trial of engineered uTNK15 and TCR/FcR transduced NK cells (““TCR/FcR #2 NK cell” shorthand) coupled with Elranatamab.

FIG. 23, depicts binding of T cell engagers (e.g., Teclistamab (anti-BCMA), Elranatamab (anti-BCMA), and Blinatumumab (anti-CD19)) to uTNK15 and TCR/FcR #2 transduced NK cells but not to NT NK cells. Natural killer (NK) cells were extracted from cord blood and cultured in complete medium supplemented with irradiated (100 Gy) uAPC feeder cells at a ratio of 2:1 (feeder cell:NK). Additionally, recombinant human IL-2 was added at a concentration of 200 U/ml to facilitate expansion of the NK cells. Five days after expansion, NK cells were either not transduced, or transduced with the uTNK15 and TCR/FcR #2. These cells were then treated with Teclistamab (20 g/ml), Elranatamab (10 g/ml), or Blinatumumab (10 g/ml) for one hour at 37° C. in a complete medium. After incubation, the cells were washed and subjected to flow cytometric analysis to validate the binding. Before running on flow cytometry, cells that had been incubated with BCMA or CD19 antigen with His tag were stained with APC anti-His tag antibody (cat no: 362605, Biolegend). Flow cytometric analysis revealed that all T cell engagers could bind efficiently to engineered NK cells, but no binding was observed with NT NK cells.

FIGS. 24A-24B, depict uTNK15 and TCR/FcR #2 transduced NK cells loaded with bispecific anti-CD19/CD3 antibody blinatumomab (10 g/ml for 1 hour at 37° C. in click/RPMI media & washed prior to co-culture) demonstrating enhanced antitumor activity against CD19+tumor cells (e.g., Raji cells or B-LCL cells) at various E:T ratios in short term Cr51 assays when compared to blinatumomab loaded NT NK cells. FIG. 24A shows the results against B-LCL cells, where there was significantly more killing by engineered NK cells at 5:1, 2.5:1, and 1.25:1 E:T ratios. FIG. 24B shows the results against Raji cells, where there was significantly more killing by engineered NK cells at 5:1 and 2.5:1 E:T ratios. Compared to blinatumomab-loaded NT NK cells, blinatumomab-loaded engineered NK cells showed enhanced cytotoxicity against CD19+tumor cells.

FIG. 25, Blinatumomab loaded uTNK15 and TCR/FcR #2 transduced NK cells showed enhanced killing compared to unloaded uTNK15 and TCR/FcR #2 transduced NK cells or loaded/unloaded non-transduced (NT) NK Cells. NK cells were either NT (NT or NT NK shorthand for stats) or transduced with uTNK15 and TCR/FcR #2 (“TCR/FcR #2” shorthand for stats). NK cells were either left unloaded or loaded with blinatumomab (10 g/ml for 1 hour at 37° C. in click/RPMI media & washed prior to co-culture) before co-culturing with tumor cells at 1:1 effector to target (E:T) ratio. Compared to loaded or unloaded NT NK Cells, unloaded engineered NK cells, or Blinatumumab alone, Blinatumumab-loaded engineered NK cells showed significantly increased cytotoxic activity against CD19 transduced SKOV3 cancer cells.

FIGS. 26A-26B, depict uTNK15 and TCR/FcR #2 transduced NK cells loaded with bispecific anti-BCMA/CD3 antibody Teclistamab (20 g/ml for 1 hour at 37° C. in click/RPMI media & washed prior to co-culture) demonstrating enhanced antitumor activity against BCMA+tumor cells (e.g., MM1s cells, H929 cells) at various E:T ratios in short term Cr51 assays when compared to Teclistamab loaded non-transduced (NT) NK cells. FIG. 26A shows the results against MM1s cells, where there was significantly more killing by engineered NK cells at 5:1, 2.5:1, and 1.25:1 E:T ratios. FIG. 26B shows the results against H929 cells, where there was significantly more killing by engineered NK cells at 5:1, 2.5:1, 1.25:1, and 1:2 E:T ratios. Compared to Teclistamab-loaded NT NK cells, Teclistamab-loaded engineered NK cells showed enhanced cytotoxicity against BCMA+tumor cells.

FIGS. 27A-27B, depict uTNK15 and TCR/FcR #2 transduced NK cells loaded with bispecific anti-GPRC5D/CD3 antibody Talquetamab (20 g/ml for 1 hour at 37° C. in click/RPMI media & washed prior to co-culture) demonstrating enhanced antitumor activity against GPRC5D+ tumor cells (e.g., MM1s cells, H929 cells) at various E:T ratios in a short term Cr51 assays when compared to Talquetamab loaded non-transduced (NT) NK cells. FIG. 27A shows the results against MM1s cells. FIG. 27B shows the results against H929 cells. Compared to Talquetamab-loaded NT NK cells, Talquetamab-loaded engineered NK cells showed enhanced cytotoxicity against GPRC5D+ tumor cells.

FIGS. 28A-28B, depict uTNK15 and TCR/FcR #2 transduced NK cells loaded with bispecific anti-BCMA/CD3 antibody Elranatamab (10 g/ml for 1 hour at 37° C. in click/RPMI media & washed prior to co-culture) demonstrating enhanced antitumor activity against BCMA+tumor cells (e.g., MM1s cells, H929 cells) at various E:T ratios in short term Cr51 assays when compared to Elranatamab loaded non-transduced (NT) NK cells. FIG. 28A shows the results against MM1s cells, where there was significantly more killing by engineered NK cells at 5:1, 2.5:1, 1.25:1, and 1:2 E:T ratios. FIG. 28B shows the results against H929 cells, where there was significantly more killing by engineered NK cells at 5:1, 2.5:1, 1.25:1, and 1:2 E:T ratios. Compared to Elranatamab-loaded NT NK cells, Elranatamab-loaded engineered NK cells showed enhanced cytotoxicity against BCMA+tumor cells.

FIG. 29A-29B, Pertuzumab (anti-HER2 Ab with low affinity binding to wild type CD16) bound to uTNK15 and TCR/FcR #2 transduced NK cells, but not to non-transduced (NT) NK cells, and Pertuzumab loaded engineered NK cells displayed superior antitumor activity against HER2+SKOV3 ovarian cancer cells in a long term xCELLigence killing assay. FIG. 29A, shows Pertuzumab binding to engineered NK cells. NK cells were extracted from cord blood and cultured in complete medium supplemented with irradiated (100 Gy) uAPC feeder cells at a ratio of 2:1 (feeder cell:NK). Additionally, recombinant human IL-2 was added at a concentration of 200 U/ml to facilitate expansion of the NK cells. Five days after expansion, natural killer (NK) cells were either transduced with the uTNK15 and TCR/FcR #2 constructs, or left non-transduced (NT). These cells were then loaded with Pertuzumab (200 g/ml for one hour at 37° C. in a complete medium). After incubation, the cells were washed and subjected to flow cytometric analysis to validate the binding. Flow cytometric analysis revealed that Pertuzumab could bind engineered NK cells, but no binding was observed with NT NK cells. FIG. 29B, Pertuzumab loaded uTNK15 and TCR/FcR #2 transduced NK cells showed enhanced killing compared to unloaded uTNK15 and TCR/FcR #2 transduced NK cells or loaded/unloaded non-transduced (NT) NK Cells. NK cells were either NT (NT or NT NK shorthand for stats) or transduced with uTNK15 and TCR/FcR #2 (“TCR/FcR #2” shorthand for stats). NK cells were either left unloaded or loaded with Pertuzumab (200 g/ml for 1 hour at 37° C. in click/RPMI media & washed prior to co-culture) before co-culturing with tumor cells at 1:1 effector to target (E:T) ratio. Compared to loaded or unloaded NT NK Cells, unloaded engineered NK cells, or Pertuzumab alone, Pertuzumab-loaded engineered NK cells showed significantly increased cytotoxic activity against HER2+SKOV3 ovarian cancer cells.

FIGS. 30A-30B, Trastuzumab (anti-HER2) bound uTNK15 and TCR/FcR #2 transduced NK cells with higher affinity than non-transduced (NT) NK cells, and Trastuzumab loaded engineered NK cells displayed superior antitumor activity against HER2+SKOV3 ovarian cancer cells in a long term xCELLigence killing assay. FIG. 30A, shows Trastuzumab binding to engineered NK cells. NK cells were extracted from cord blood and cultured in complete medium supplemented with irradiated (100 Gy) uAPC feeder cells at a ratio of 2:1 (feeder cell:NK). Additionally, recombinant human IL-2 was added at a concentration of 200 U/ml to facilitate expansion of the NK cells. Five days after expansion, natural killer (NK) cells were either transduced with the uTNK15 and TCR/FcR #2 constructs, or left non-transduced (NT). These cells were then loaded with Trastuzumab (200 g/ml for one hour at 37° C. in a complete medium). After incubation, the cells were washed and subjected to flow cytometric analysis to validate the binding. Flow cytometric analysis revealed that Trastuzumab could bind engineered NK cells with higher affinity than was observed with NT NK cells. FIG. 30B, Trastuzumab loaded uTNK15 and TCR/FcR #2 transduced NK cells showed enhanced killing compared to unloaded uTNK15 and TCR/FcR #2 transduced NK cells or loaded/unloaded NT NK Cells. NK cells were either NT (“NT” or “NT NK” shorthand for stats) or transduced with uTNK15 and TCR/FcR #2 (“TCR/FcR #2” shorthand for stats). NK cells were either left unloaded or loaded with Trastuzumab (200 g/ml for 1 hour at 37° C. in click/RPMI media & washed prior to co-culture) before co-culturing with tumor cells at 1:1 effector to target (E:T) ratio. Compared to loaded or unloaded NT NK Cells, unloaded engineered NK cells, or Trastuzumab alone, Trastuzumab-loaded engineered NK cells showed significantly increased cytotoxic activity against HER2+SKOV3 ovarian cancer cells.

FIGS. 31A-31B, Tafasitamab (anti-CD19) bound uTNK15 and TCR/FcR #2 transduced NK cells with higher affinity than non-transduced (NT) NK cells, and Tafasitamab loaded engineered NK cells displayed superior antitumor activity against transduced CD19+SKOV3 ovarian cancer cells in a long term xCELLigence killing assay. FIG. 31A, NK cells were extracted from cord blood and cultured in complete medium supplemented with irradiated (100 Gy) uAPC feeder cells at a ratio of 2:1 (feeder cell:NK). Additionally, recombinant human IL-2 was added at a concentration of 200 U/ml to facilitate expansion of the NK cells. Five days after expansion, natural killer (NK) cells were either left non-transduced, or transduced with the uTNK15 and TCR/FcR #2 constructs. These cells were then treated with Tafasitamab (10 g/ml for 1 hour at 37° C. in complete media). After incubation, the cells were washed and subjected to flow cytometric analysis to validate the binding. Flow cytometric analysis revealed that Tafasitamab exhibited higher and stronger binding affinity towards engineered NK cells compared to NT NK cells. FIG. 31B, Tafasitamab loaded uTNK15 and TCR/FcR #2 transduced NK cells showed enhanced killing compared to unloaded uTNK15 and TCR/FcR #2 transduced NK cells or loaded/unloaded NT NK Cells. NK cells were either NT (“NT” or “NT NK” shorthand for stats) or transduced with uTNK15 and TCR/FcR #2 (“TCR/FcR #2” shorthand for stats). NK cells were either left unloaded or loaded with Tafasitamab (200 g/ml for 1 hour at 37° C. in click/RPMI media & washed prior to co-culture) before co-culturing with tumor cells at 1:1 effector to target (E:T) ratio. Compared to loaded or unloaded NT NK Cells, unloaded engineered NK cells, or Tafasitamab alone, Tafasitamab-loaded engineered NK cells showed significantly increased cytotoxic activity against transduced CD19+SKOV3 ovarian cancer cells.

FIGS. 32A-32B, Brentuximab (anti-CD30 antibody-drug conjugate) bound to uTNK15 and TCR/FcR #2 transduced NK cells with higher affinity than non-transduced (NT) NK cells, and Brentuximab loaded engineered NK cells displayed superior antitumor activity against CD30+Karpas tumor cells in IncuCyte cytotoxicity assays. FIG. 32A, NK cells were isolated from cord blood and expand in complete media in the presence of irradiated (100 Gy) uAPC feeder cells (2:1 feeder cell:NK ratio) and recombinant human IL-2 (200 U/ml). Seven days following expansion, NK cells were either left non-transduced, or transduced with the uTNK15 and TCR/FcR #2 constructs. These cells were loaded with Brentuximab (100 g/ml for one hour at 37° C. in complete medium) and washed prior to flow cytometric validation of the Brentuximab binding. Brentuximab bound to engineered NK cells with higher affinity compared to NT NK cells, as determined by staining with F(ab′)₂ Anti-Human antibody and flow cytometric analysis of NK cells. FIG. 32B, Brentuximab-loaded uTNK15 and TCR/FcR #2 transduced NK cells showed enhanced killing of CD30+ (Karpas) tumor cells compared to non-loaded engineered NK cells or brentuximab-loaded NT NK Cells in IncuCyte cytotoxicity assays. NK cells were derived from cord blood expanded as described in 31A, and either not transduced or transduced with uTNK15 and TCR/FcR #2 constructs. NT NK cells or engineered NK cells were loaded with Brentuximab (100 g/ml for one hour at 37° C. in complete medium and washed prior to co-culturing) and co-cultured with CD30+Karpas tumor cell line at 1:1 effector to target ratios. Real-time cytotoxicity of effector cells against Karpas cells was measured every 2 hour over 24-hour period. Compared to non-loaded engineered NK cells or NT NK cells loaded with Brentuximab, engineered NK cells loaded with Brentuximab showed increased cytotoxicity of CD30+Karpas cells.

### DETAILED DESCRIPTION

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

As used herein, the term “CD3 receptor complex” or “CD3 co-receptor complex” refers to the protein complex that in nature acts as a T cell co-receptor and is comprised of CD3ζ chain, CD3γ chain, a CD3δ chain, and two CD3ε chains (although in alternatives only one CD38 chain is used).

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure. In specific embodiments, a vector is engineered through recombinant nucleic acid technologies, and a cell is engineered through transfection or transduction of an engineered vector. Cells may be engineered to express heterologous proteins that are not naturally expressed by the cells, either because the heterologous proteins are recombinant or synthetic or because the cells do not naturally express the proteins.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “subject,” as used herein, generally refers to an individual having or is suspected of having cancer. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as benign or malignant neoplasias, or cancer. The subject may be undergoing or having undergone treatment. The subject may be asymptomatic. The subject may be healthy individuals but that are desirous of prevention of cancer. The term “individual” may be used interchangeably, in at least some cases. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants and includes in utero individuals. It is not intended that the term connote a need for medical treatment, therefore, an individual may voluntarily or involuntarily be part of experimentation whether clinical or in support of basic science studies.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of one or more symptoms of the disease or condition, or the delaying of the progression of the disease or condition. “Treatment” does not necessarily indicate complete eradication or cure of the disease or condition, or associated symptoms thereof. Treating may mean alleviation of at least one symptom of the disease or condition.

As used herein “TCR/CD3 complex” refers to a protein complex naturally found on the surface of T cells and that comprises T-cell receptor (TCR) α and β chains, invariant Natural Killer T-cell receptor (iTCR) α and β chains, and/or a T-cell receptor γ and δ chains, in addition to CD3ζ, CD3γ, CD3δ, and CD3ε chains.

### I. EMBODIMENTS OF THE DISCLOSURE

Natural killer (NK) cells are an emerging cellular immunotherapy for patients with malignant hematologic disease, as well as solid tumors. The present disclosure specifically relates to NK cells that have been modified to render the NK cells to have enhanced function as an immunotherapy compared to NK cells not so modified. The modifications allow for the NK cells to have greater versatility when used with other therapeutic agents and at least in some embodiments to have T cell-like activity by utilizing the CD3/TCR receptor complex. In specific embodiments, the NK cells are modified to express (i) either a single CD3 chain (CD3zeta, CD3 epsilon, CD3 delta, or CD3 gamma) or part or all of the human CD3 receptor complex (including any combination of CD3 delta, epsilon (one or two copies of epsilon), gamma, and zeta); or (ii) either a single CD3 chain or the human CD3 receptor complex (including any combination of CD3 delta, epsilon (one or two molecules), gamma, and zeta) as a full length protein or as a partial protein heterologously linked to one or more intracellular signaling domains); and (iii) the CD3 complex may or may not include the T-cell receptor (αβ or γδ) and/or iTCR receptor (a p). The disclosure concerns the use of CD3-expressing NK cells in the diagnosis and treatment of disease, including use of the cells in combination with bispecific or multi-specific antibodies in which one epitope of the antibody binds CD3 on the CD3-expressing NK cells. The CD3-expressing NK cells can either be pre-complexed ex vivo with the bi/multi-specific antibody to redirect their specificity toward the target antigen and/or combined in vivo. In diagnostic embodiments, labeled NK cells may be loaded with bispecific or multi-specific antibodies of any kind, including that comprise at least an anti-CD3 antibody, and the loaded, labeled NK cells may be monitored for trafficking to the site of the target antigen for which another antibody on the bispecific or multi-specific antibody binds.

In certain embodiments of the disclosure, a TCR recognizes antigens and/or epitopes presented by a major-histocompatibility complex (MHC). In certain embodiments, an antigen and/or epitope is a peptide, lipid, and/or glycolipid. In certain embodiments, a MHC is a class I MHC. In certain embodiments, a MHC is a class II MHC. In certain embodiments, an MHC is a non-classical MHC. In certain embodiments, an MHC is a class I-like MHC. In certain embodiments, an MHC is CD1d.

In certain embodiments, a TCR target antigen is not primarily what provides a transduced effector cell with target antigen specificity. In certain embodiments, a TCR acts primarily as a stabilizer for a CD3 co-receptor complex, while an antibody provides the primary target antigen specificity for a transduced effector cell.

### II. COMPOSITIONS OF THE DISCLOSURE

The disclosure concerns compositions that at least include modified NK cells that express at least parts of the TCR/CD3 complex. In some cases, the compositions also include monospecific, bispecific, and/or multi-specific antibodies, including in the same formulation, although in alternative embodiments the NK cells and antibodies are utilized as physically separate compositions.

### A. NK Cell TCR/CD3 Modifications

In particular embodiments, provided herein are NK cells that have been modified by the hand of man to express part or all of a CD3/TCR receptor complex. In specific embodiments, the NK cells are modified to include all components of the CD3 complex, including CD3ζ, CD3ε, CD3γ and CD3δ. In particular cases the full lengths of CD3ζ, CD3ε, CD3γ and CD3δ are utilized, including their extracellular domain, transmembrane domain, and intracellular domain, however in alternative embodiments only part of one or more of CD3ζ, CD3ε, CD3γ and CD3δ are utilized each of which that may or may not be combined with one or more intracellular signaling domains such as CD16, NKG2D, DAP10, DAP12, CD28, 41BB, 2B4, CD27, OX40, or any combination thereof.

In certain embodiments, an amino acid sequence (e.g., a polypeptide) may comprise an amino acid represented by a single letter “X” or a three letter code “Xaa”. In some embodiments, the amino acid represented by “X” or “Xaa” is any naturally occurring amino acid, such as but not limited to, Arginine (Arg, R), Histidine (His, H), Lysine (Lys, K), Aspartic Acid (Asp, D), Glutamic Acid (Glu, E), Serine (Ser, S), Threonine (Thr, T), Asparagine (Asn, N), Glutamine (Gln, Q), Glycine (Gly, G), Proline (Pro, P), Cysteine (Cys, C), Alanine (Ala, A), Valine (Val, V), Isoleucine (Ile, I), Leucine (Leu, L), Methionine (Met, M), Phenylalanine (Phe, F), Tyrosine (Tyr, Y), or Tryptophan (Trp, W).

In certain embodiments, particular sequences for any of the CD3 receptor components are utilized, including wildtype or mutants of the components so long as the CD3 receptor having the mutant is able to allow signaling through the CD3 complex leading to activation and killing of targets. In some cases, CD3/TCR complex associated polypeptides, polynucleotides encoding the same, and/or constructs comprising said polynucleotides are described in the Inventors international patent application publication WO2023004425A2 (PCT/US2022/074062), published on Jan. 26, 2023, which is incorporated herein by reference in its entirety for the purposes described herein.

In some cases, the following examples of sequences for CD3ε, CD3δ, CD3γ, and CD3ζ and are utilized for modification of the NK cells.

### CD3 Epsilon (CD3ε, CD3e) (UniProtKB-P07766 (CD3E_HUMAN))

Signal Peptide
(SEQ ID NO: 1)
MQSGTHWRVLGLCLLSVGVW
Extracellular Domain
sp|P07766|23-126
(SEQ ID NO: 2)
DGNEEMGGITQTPYKVSISGTTVILTCPQYPGSEILWQHNDKNIGGDEDD
KNIGSDEDHLSLKEFSELEQSGYYVCYPRGSKPEDANFYLYLRARVCENC
MEMD
Transmembrane Domain
sp|P07766|127-152
(SEQ ID NO: 3)
VMSVATIVIVDICITGGLLLLVYYWS
Intracellular Domain
sp|P07766|153-207
(SEQ ID NO: 4)
KNRKAKAKPVTRGAGAGGRQRGQNKERPPPVPNPDYEPIRKGQRDLYSGL
NQRRI

An example of a Homo sapiens CD3e molecule (CD3E), mRNA is at NCBI Reference Sequence: GENBANK® Accession No. NM_000733.4

(SEQ ID NO: 5)
ATGCAGTCGGGCACTCACTGGAGAGTTCTGGGCCTCTGCCTCTTATCAGTTGGCGTTTGGGG
GCAAGATGGTAATGAAGAAATGGGTGGTATTACACAGACACCATATAAAGTCTCCATCTCTG
GAACCACAGTAATATTGACATGCCCTCAGTATCCTGGATCTGAAATACTATGGCAACACAAT
GATAAAAACATAGGCGGTGATGAGGATGATAAAAACATAGGCAGTGATGAGGATCACCTGTC
ACTGAAGGAATTTTCAGAATTGGAGCAAAGTGGTTATTATGTCTGCTACCCCAGAGGAAGCA
AACCAGAAGATGCGAACTTTTATCTCTACCTGAGGGCAAGAGTGTGTGAGAACTGCATGGAG
ATGGATGTGATGTCGGTGGCCACAATTGTCATAGTGGACATCTGCATCACTGGGGGCTTGCT
GCTGCTGGTTTACTACTGGAGCAAGAATAGAAAGGCCAAGGCCAAGCCTGTGACACGAGGAG
CGGGTGCTGGCGGCAGGCAAAGGGGACAAAACAAGGAGAGGCCACCACCTGTTCCCAACCCA
GACTATGAGCCCATCCGGAAAGGCCAGCGGGACCTGTATTCTGGCCTGAATCAGAGACGCAT
CTGA

Examples of respective nucleic acid and amino acid CD3 epsilon sequences in their entirety are as follows (underlining refers to signal peptide sequence, which in some embodiments may be modified, omitted, and/or replaced with an alternative signal peptide):

(SEQ ID NO: 6)
ATGCAGAGCGGCACCCACTGGAGAGTGCTGGGCCTGTGCCTGCTGAGCGTGGGCGTGTGGGG
CCAGGACGGCAACGAGGAGATGGGCGGCATCACCCAGACCCCCTACAAGGTGAGCATCAGCG
GCACCACCGTGATCCTGACCTGCCCCCAGTACCCCGGCAGCGAGATCCTGTGGCAGCACAAC
GACAAGAACATCGGCGGCGACGAGGACGACAAGAACATCGGCAGCGACGAGGACCACCTGAG
CCTGAAGGAGTTCAGCGAGCTGGAGCAGAGCGGCTACTACGTGTGCTACCCCAGAGGCAGCA
AGCCCGAGGACGCCAACTTCTACCTGTACCTGAGAGCCAGAGTGTGCGAGAACTGCATGGAG
ATGGACGTGATGAGCGTGGCCACCATCGTGATCGTGGACATCTGCATCACCGGCGGCCTGCT
GCTGCTGGTGTACTACTGGAGCAAGAACAGAAAGGCCAAGGCCAAGCCCGTGACCAGAGGCG
CCGGCGCCGGCGGCAGACAGAGAGGCCAGAACAAGGAGAGACCCCCCCCCGTGCCCAACCCC
GACTACGAGCCCATCAGAAAGGGCCAGAGAGACCTGTACAGCGGCCTGAACCAGAGAAGAAT
C
(SEQ ID NO: 7)
MQSGTHWRVLGLCLLSVGVWGQDGNEEMGGITQTPYKVSISGTTVILTCPQYPGSEILWQHN
DKNIGGDEDDKNIGSDEDHLSLKEFSELEQSGYYVCYPRGSKPEDANFYLYLRARVCENCME
MDVMSVATIVIVDICITGGLLLLVYYWSKNRKAKAKPVTRGAGAGGRQRGQNKERPPPVPNP
DYEPIRKGQRDLYSGLNQRRI

### CD3 Delta (CD36, CD3d) (UniProtKB-P04234 (CD3D_HUMAN))

Signal Peptide
(SEQ ID NO: 8)
MEHSTFLSGLVLATLLSQVS
Extracellular Domain
sp|P04234|22-105
(SEQ ID NO: 9)
FKIPIEELEDRVFVNCNTSITWVEGTVGTLLSDITRLDLGKRILDPRGIY
RCNGTDIYKDKESTVQVHYRMCQSCVELDPATVA
Transmembrane Domain
sp|P04234|106-126
(SEQ ID NO: 10)
GIIVIDVIATLLLALGVFCFA
Intracellular Domain
sp|P04234|127-171
(SEQ ID NO: 11)
GHETGRLSGAADTQALLRNDQVYQPLRDRDDAQYSHLGGNWARNK

Homo sapiens CD3d molecule, delta (CD3-TCR complex), mRNA (cDNA clone MGC:88324 IMAGE:30412345), complete cds GENBANK®: BC070321.1

(SEQ ID NO: 12)
ATGGAACATAGCACGTTTCTCTCTGGCCTGGTACTGGCTACCCTTCTCTCGCAAGTGAGCCC
CTTCAAGATACCTATAGAGGAACTTGAGGACAGAGTGTTTGTGAATTGCAATACCAGCATCA
CATGGGTAGAGGGAACGGTGGGAACACTGCTCTCAGACATTACAAGACTGGACCTGGGAAAA
CGCATCCTGGACCCACGAGGAATATATAGGTGTAATGGGACAGATATATACAAGGACAAAGA
ATCTACCGTGCAAGTTCATTATCGAATGTGCCAGAGCTGTGTGGAGCTGGATCCAGCCACCG
TGGCTGGCATCATTGTCACTGATGTCATTGCCACTCTGCTCCTTGCTTTGGGAGTCTTCTGC
TTTGCTGGACATGAGACTGGAAGGCTGTCTGGGGCTGCCGACACACAAGCTCTGTTGAGGAA
TGACCAGGTCTATCAGCCCCTCCGAGATCGAGATGATGCTCAGTACAGCCACCTTGGAGGAA
ACTGGGCTCGGAACAAGTGA

Examples of respective nucleic acid and amino acid CD3 delta sequences in their entirety are as follows (underlining refers to signal peptide sequence, which in some embodiments may be modified, omitted, and/or replaced with an alternative signal peptide):

(SEQ ID NO: 13)
ATGGAGCACAGCACCTTCCTGAGCGGCCTGGTGCTGGCCACCCTGCTGAGCCAGGTGAGCCC
CTTCAAGATCCCCATCGAGGAGCTGGAGGACAGAGTGTTCGTGAACTGCAACACCAGCATCA
CCTGGGTGGAGGGCACCGTGGGCACCCTGCTGAGCGACATCACCAGACTGGACCTGGGCAAG
AGAATCCTGGACCCCAGAGGCATCTACAGATGCAACGGCACCGACATCTACAAGGACAAGGA
GAGCACCGTGCAGGTGCACTACAGAATGTGCCAGAGCTGCGTGGAGCTGGACCCCGCCACCG
TGGCCGGCATCATCGTGACCGACGTGATCGCCACCCTGCTGCTGGCCCTGGGCGTGTTCTGC
TTCGCCGGCCACGAGACCGGCAGACTGAGCGGCGCCGCCGACACCCAGGCCCTGCTGAGAAA
CGACCAGGTGTACCAGCCCCTGAGAGACAGAGACGACGCCCAGTACAGCCACCTGGGCGGCA
ACTGGGCCAGAAACAAG
(SEQ ID NO: 14)
MEHSTFLSGLVLATLLSQVSPFKIPIEELEDRVFVNCNTSITWVEGTVGTLLSDITRLDLGK
RILDPRGIYRCNGTDIYKDKESTVQVHYRMCQSCVELDPATVAGIIVTDVIATLLLALGVFC
FAGHETGRLSGAADTQALLRNDQVYQPLRDRDDAQYSHLGGNWARNK

### CD3 Gamma (CD3γ, CD3g) (T-Cell Surface Glycoprotein CD3 Gamma Chain Gene CD3G P09693)

Signal Peptide
(SEQ ID NO: 15)
MEQGKGLAVLILAIILLQGTLA
Extracellular Domain
sp|P09693|23-116
(SEQ ID NO: 16)
QSIKGNHLVKVYDYQEDGSVLLTCDAEAKNITWFKDGKMIGFLTEDKKKW
NLGSNAKDPRGMYQCKGSQNKSKPLQVYYRMCQNCIELNAATIS
Transmembrane Domain
sp|P09693|117-137
(SEQ ID NO: 17)
GFLFAEIVSIFVLAVGVYFIA
Intracellular Domain
sp|P09693|138-182
(SEQ ID NO: 18)
GQDGVRQSRASDKQTLLPNDQLYQPLKDREDDQYSHLQGNQLRRN

Homo sapiens CD3g molecule (CD3G), mRNA; NM_000073.3:81-629 Homo sapiens CD3g molecule (CD3G), mRNA

(SEQ ID NO: 19)
ATGGAACAGGGGAAGGGCCTGGCTGTCCTCATCCTGGCTATCATT
CTTCTTCAAGGTACTTTGGCCCAGTCAATCAAAGGAAACCACTTG
GTTAAGGTGTATGACTATCAAGAAGATGGTTCGGTACTTCTGACT
TGTGATGCAGAAGCCAAAAATATCACATGGTTTAAAGATGGGAAG
ATGATCGGCTTCCTAACTGAAGATAAAAAAAAATGGAATCTGGGA
AGTAATGCCAAGGACCCTCGAGGGATGTATCAGTGTAAAGGATCA
CAGAACAAGTCAAAACCACTCCAAGTGTATTACAGAATGTGTCAG
AACTGCATTGAACTAAATGCAGCCACCATATCTGGCTTTCTCTTT
GCTGAAATCGTCAGCATTTTCGTCCTTGCTGTTGGGGTCTACTTC
ATTGCTGGACAGGATGGAGTTCGCCAGTCGAGAGCTTCAGACAAG
CAGACTCTGTTGCCCAATGACCAGCTCTACCAGCCCCTCAAGGAT
CGAGAAGATGACCAGTACAGCCACCTTCAAGGAAACCAGTTGAGG
AGGAATTGA

Examples of respective nucleic acid and amino acid CD3 gamma sequences in their entirety are as follows (underlining refers to signal peptide sequence, which in some embodiments may be modified, omitted, and/or replaced with an alternative signal peptide):

(SEQ ID NO: 20)
ATGGAACAGGGGAAGGGCCTGGCTGTCCTCATCCTGGCTATCATT
CTTCTTCAAGGTACTTTGGCCCAGTCAATCAAAGGAAACCACTTG
GTTAAGGTGTATGACTATCAAGAAGATGGTTCGGTACTTCTGACT
TGTGATGCAGAAGCCAAAAATATCACATGGTTTAAAGATGGGAAG
ATGATCGGCTTCCTAACTGAAGATAAAAAAAAATGGAATCTGGGA
AGTAATGCCAAGGACCCTCGTGGGATGTATCAGTGTAAAGGATCA
CAGAACAAGTCAAAACCACTCCAAGTGTATTACAGAATGTGTCAG
AACTGCATTGAACTAAATGCAGCCACCATATCTGGCTTTCTCTTT
GCTGAAATCGTCAGCATTTTCGTCCTTGCTGTTGGGGTCTACTTC
ATTGCTGGACAGGATGGAGTTCGCCAGTCGAGAGCTTCAGACAAG
CAGACTCTGTTGCCCAATGACCAGCTCTACCAGCCCCTCAAGGAT
CGAGAAGATGACCAGTACAGCCACCTTCAAGGAAACCAGTTGAGG
AGGAAT
(SEQ ID NO: 21)
MEQGKGLAVLILAIILLQGTLAQSIKGNHLVKVYDYQEDGSVLLT
CDAEAKNITWFKDGKMIGFLTEDKKKWNLGSNAKDPRGMYQCKGS
QNKSKPLQVYYRMCQNCIELNAATISGELFAEIVSIFVLAVGVYF
IAGQDGVRQSRASDKQTLLPNDQLYQPLKDREDDQYSHLQGNQLR
RN

### CD3 Zeta (CD3ζ, CD3z)

Signal Peptide
sp|P20963|SP
(SEQ ID NO: 22)
MKWKALFTAAILQAQLPITEA
Extracellular Domain
sp|P20963|22-30 ECD
(SEQ ID NO: 23)
QSFGLLDPK
Transmembrane Domain
sp|P20963|31-51 tmd
(SEQ ID NO: 24)
LCYLLDGILFIYGVILTALFL
Intracellular Domain
sp|P20963|52-164 ICD
(SEQ ID NO: 25)
RVKFSRSADAPAYQQGQNQLYNELNLGRREEYDVLDKRRGRDPEM
GGKPQRRKNPQEGLYNELQKDKMAEAYSEIGMKGERRRGKGHDGL
YQGLSTATKDTYDALHMQALPPR

Examples of respective nucleic acid and amino acid CD3 zeta sequences in their entirety are as follows (underlining refers to signal peptide sequence, which in some embodiments may be modified, omitted, and/or replaced with an alternative signal peptide):

(SEQ ID NO: 26)
ATGAAGTGGAAGGCGCTTTTCACCGCGGCCATCCTGCAGGCACAG
TTGCCGATTACAGAGGCACAGAGCTTTGGCCTGCTGGATCCCAAA
CTCTGCTACCTGCTGGATGGAATCCTCTTCATCTATGGTGTCATT
CTCACTGCCTTGTTCCTGAGAGTGAAGTTCAGCAGGAGCGCAGAC
GCCCCCGCGTACCAGCAGGGCCAGAACCAGCTCTATAACGAGCTC
AATCTAGGACGAAGAGAGGAGTACGATGTTTTGGACAAGAGACGT
GGCCGGGACCCTGAGATGGGGGGAAAGCCGCAGAGAAGGAAGAAC
CCTCAGGAAGGCCTGTACAATGAACTGCAGAAAGATAAGATGGCG
GAGGCCTACAGTGAGATTGGGATGAAAGGCGAGCGCCGGAGGGGC
AAGGGGCACGATGGCCTTTACCAGGGTCTCAGTACAGCCACCAAG
GACACCTACGACGCCCTTCACATGCAGGCCCTGCCCCCTCGC
(SEQ ID NO: 27)
MKWKALFTAAILQAQLPITEAQSFGLLDPKLCYLLDGILFIYGVI
LTALFLRVKFSRSADAPAYQQGQNQLYNELNLGRREEYDVLDKRR
GRDPEMGGKPQRRKNPQEGLYNELQKDKMAEAYSEIGMKGERRRG
KGHDGLYQGLSTATKDTYDALHMQALPPR

Homo sapiens CD247 molecule (CD247; also referred to as CD3 Zeta), transcript variant 1, mRNA

### NCBI Reference Sequence: NM_198053.3

NM_198053.3:65-559 Homo sapiens CD247 molecule (CD247), transcript variant 1, mRNA

(SEQ ID NO: 28)
ATGAAGTGGAAGGCGCTTTTCACCGCGGCCATCCTGCAGGCACAG
TTGCCGATTACAGAGGCACAGAGCTTTGGCCTGCTGGATCCCAAA
CTCTGCTACCTGCTGGATGGAATCCTCTTCATCTATGGTGTCATT
CTCACTGCCTTGTTCCTGAGAGTGAAGTTCAGCAGGAGCGCAGAC
GCCCCCGCGTACCAGCAGGGCCAGAACCAGCTCTATAACGAGCTC
AATCTAGGACGAAGAGAGGAGTACGATGTTTTGGACAAGAGACGT
GGCCGGGACCCTGAGATGGGGGGAAAGCCGCAGAGAAGGAAGAAC
CCTCAGGAAGGCCTGTACAATGAACTGCAGAAAGATAAGATGGCG
GAGGCCTACAGTGAGATTGGGATGAAAGGCGAGCGCCGGAGGGGC
AAGGGGCACGATGGCCTTTACCAGGGTCTCAGTACAGCCACCAAG
GACACCTACGACGCCCTTCACATGCAGGCCCTGCCCCCTCGCTAA

In specific embodiments, the NK cells are modified to express one of more of the TCRα chain, the TCRβ chain, the TCRγ chain, and the TCRδ chain, and any combination P thereof may be utilized. In certain embodiments, the TCR may be an invariant Natural Killer cell TCR (iTCR). In a specific case, the NK cells are modified to express the T-cell receptor (TCR) αβ chains, iTCR ap chains, or the TCR γδ chains. In certain cases, the NK cells are modified to express part or all of only the constant region of one of more of the TCRα chain, iTCRα chain, the TCRβ chain, iTCRβ chain, the TCRγ chain, and the TCRδ chain. The NK cells may be modified to express part or all of only the constant region of the T-cell receptor (TCR) αβ chains, or the TCR γδ chains, or the iTCR ap chains. In cases wherein part of the constant region is utilized, the part of the constant region may be at least 50, 75, 100, 125, 150, 175, 200, 225, 250, 275, 300, 325, 350, 375, or 400 amino acids, including contiguous amino acids of any constant region. The part of the constant region may comprise at least 50, 55, 60, 65, 70, 75, 80, 85, 90, 91, 92, 93, 94, 95, 96, 97, 98, or 99% of the amino acids of a constant region, including contiguous amino acids of a constant region.

In specific cases, any sequences encompassed herein are utilized to modify the NK cells, although in other cases sequences that are related to these in identity are utilized. For example, related sequences that are at least 80, 85, 90, 95, 96, 97, 98, 99% identical to any sequence encompassed herein may be utilized in the disclosure.

Particular constructs for the expression of various TCR/CD3 proteins in the NK cells may be utilized, and in a variety of configurations. In specific cases, the NK cells may be transduced or transfected with one or more vectors to express any of the various proteins encompassed herein, including at least any one or more components of the TCR/CD3 complex. In specific cases, the one or more vectors themselves may or may not be multicistronic by being able ultimately to produce more than one separate polypeptide. In cases wherein one or more multicistronic vectors are employed, they may utilize one or more internal ribosome entry sites (IRES) and/or one or more 2A self-cleaving peptide sites. In some embodiments, a 2A self-cleaving peptide site is encoded by a codon optimized polynucleotide. In cases wherein one or more 2A sequences are utilized, the following may be used, where GSG is an optional linker:

T2A
(SEQ ID NO: 29)
(GSG) EGRGSLLTCGDVEENPGP
P2A
(SEQ ID NO: 30)
(GSG) ATNFSLLKQAGDVEENPGP
E2A
(SEQ ID NO: 31)
(GSG) QCTNYALLKLAGDVESNPGP
F2A
(SEQ ID NO: 32)
(GSG) VKQTLNFDLLKLAGDVESNPGP

In situations wherein multiple protein components are expressed from a multicistronic vector, the order in a 5′ to 3′ direction on the polynucleotide vector may be of any order, although in alternative cases they are present on the vector in a particular order. A multicistronic vector may express multiple components of the TCR/CD3 receptor complex and no other heterologous protein, or the multicistronic vector may express multiple components of the TCR/CD3 receptor complex and one or more other heterologous proteins. In specific embodiments, two or more multicistronic vectors are provided, each encoding one or more components of the TCR/CD3 receptor complex and one or more other heterologous proteins, such as a cytokine and/or an engineered receptor (e.g., a receptor comprising an extracellular portion of an Fc binding protein).

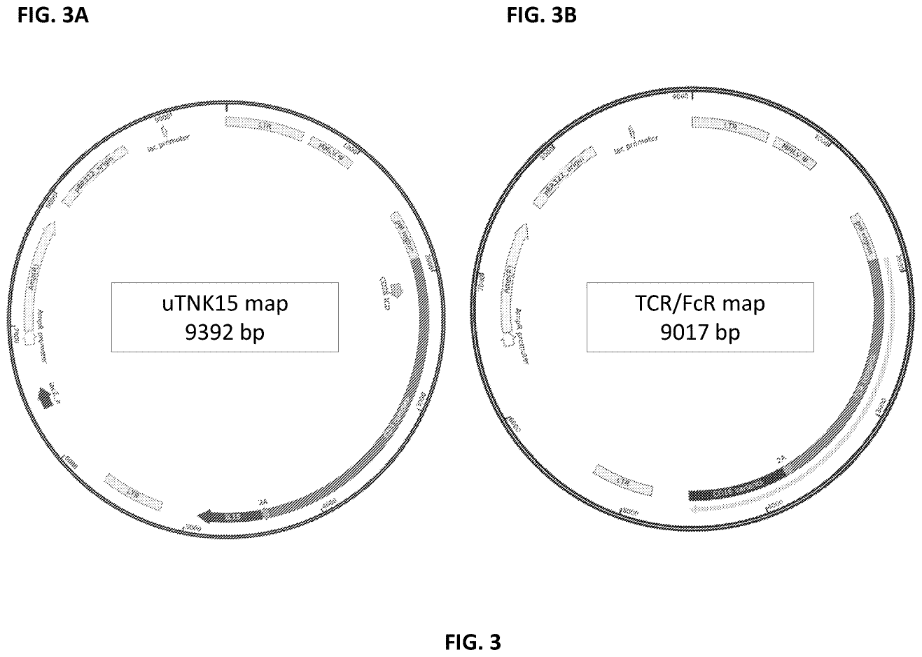

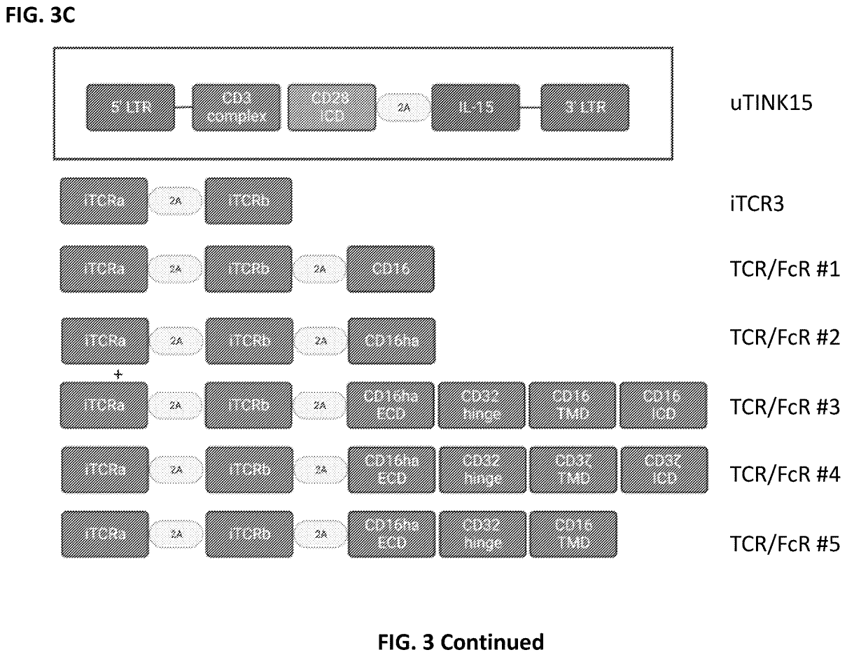

There is an example in FIGS. 3A and 3C of a multicistronic vector in which full lengths of CD3ε, CD3δ, CD3γ, and CD3ζ are present and separated by the same or different 2A self-cleaving peptide sites (“CD3 complex”). A multicistronic vector may include the signal peptide, extracellular domain, transmembrane domain, and intracellular domain of each of CD3ε, CD3δ, CD3γ, and CD3ζ.

FIG. 3C provides examples of various iTCR and/or CD16 expression constructs for engineering of NK cells. In particular embodiments of the disclosure, TCR/CD3 receptor complex components are expressed from different vectors in the NK cells. In any case, the vector(s) may express a TCR directed against, or not directed against, a particular antigen of interest, such as a cancer antigen or a viral antigen. The TCR may or may not comprise at least part of CD3ζ, including the intracellular domain of CD3ζ, in addition to the NK cells also expressing CD3ζ as a separate molecule from the TCR and as part of the CD3 receptor complex.

In specific embodiments, a TCR, such as an iTCR of the modified NK cells is utilized not necessarily as a therapeutic and/or targeting moiety aspect for the NK cells, but as a structural support or scaffold to facilitate function or enhanced function of the CD3 receptor complex. That is, the TCR may be any TCR and may not necessarily be utilized for its ability to target a particular antigen. In such cases, as non-limiting examples, a TCR that targets a viral antigen, a glycolipid, a bacterial antigen, etc. may be employed for NK cells that will be used for cancers that are not necessarily related to that particular virus, bacteria, and/or glycolipid. In other cases, the TCR is selected for the ability to target a particular antigen associated with a cancer of interest (e.g., a tumor associated antigen).

In certain embodiments, provided herein are CD3 constructs comprising a fusion with an intracellular co-stimulatory domain derived from CD16, NKG2D, DAP10, DAP12, 2B4, 4-1BB, CD2, CD28, DNAM, or any combination thereof. In certain embodiments, an intracellular co-stimulatory domain is fused to CD3δ, CD3ε, CD3γ, and/or CD3ζ. In certain embodiments, such a CD3 fusion construct comprises a CD3C fused to a DAP10 intracellular co-stimulatory domain. In certain embodiments, such a CD3 fusion construct comprises a CD3ζ fused to a CD28 intracellular co-stimulatory domain. In certain embodiments, such a CD3 fusion construct comprises a CD3ζ fused to a DAP10 intracellular co-stimulatory domain and a CD28 intracellular co-stimulatory domain. In certain embodiments, a CD3C fused to a DAP10 intracellular co-stimulatory domain is represented by a polynucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 33. In certain embodiments, a CD3C fused to a CD28 intracellular co-stimulatory domain is represented by a polynucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 34. In certain embodiments, a CD3C fused to a DAP10 intracellular co-stimulatory domain and a CD28 intracellular co-stimulatory domain is represented by a polynucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 35. In certain embodiments, a CD3C fused to a DAP10 intracellular co-stimulatory domain is represented by an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 36. In certain embodiments, a CD3C fused to a CD28 intracellular co-stimulatory domain is represented by an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 37. In certain embodiments, a CD3C fused to a DAP10 intracellular co-stimulatory domain and a CD28 intracellular co-stimulatory domain is represented by an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 38. In certain embodiments, a CD3C fused to an intracellular domain may not comprise a C terminal 2A domain. In certain embodiments, a CD3ζ fused to an intracellular domain may not comprise an N terminal signal peptide domain.

(SEQ ID NO: 33)
ATGAAGTGGAAGGCGCTTTTCACCGCGGCCATCCTGCAGGCACAGTTGCCGATTACAGAGGC
ACAGAGCTTTGGCCTGCTGGATCCCAAACTCTGCTACCTGCTGGATGGAATCCTCTTCATCT
ATGGTGTCATTCTCACTGCCTTGTTCCTGCTTTGCGCACGCCCACGCCGCAGCCCCGCCCAA
GAAGATGGCAAAGTCTACATCAACATGCCAGGCAGGGGCAGAGTGAAGTTCAGCAGGAGCGC
AGACGCCCCCGCGTACCAGCAGGGCCAGAACCAGCTCTATAACGAGCTCAATCTAGGACGAA
GAGAGGAGTACGATGTTTTGGACAAGAGACGTGGCCGGGACCCTGAGATGGGGGGAAAGCCG
CAGAGAAGGAAGAACCCTCAGGAAGGCCTGTACAATGAACTGCAGAAAGATAAGATGGCGGA
GGCCTACAGTGAGATTGGGATGAAAGGCGAGCGCCGGAGGGGCAAGGGGCACGATGGCCTTT
ACCAGGGTCTCAGTACAGCCACCAAGGACACCTACGACGCCCTTCACATGCAGGCCCTGCCC
CCTCGCCAGTGCACCAACTACGCCCTGCTGAAGCTGGCCGGCGACGTGGAGAGCAACCCCGG
CCCC
(SEQ ID NO: 34)
ATGAAGTGGAAGGCGCTTTTCACCGCGGCCATCCTGCAGGCACAGTTGCCGATTACAGAGGC
ACAGAGCTTTGGCCTGCTGGATCCCAAACTCTGCTACCTGCTGGATGGAATCCTCTTCATCT
ATGGTGTCATTCTCACTGCCTTGTTCCTGAGGAGTAAGAGGAGCAGGCTCCTGCACAGTGAC
TACATGAACATGACTCCCCGCCGCCCCGGGCCCACCCGCAAGCATTACCAGCCCTATGCCCC
ACCACGCGACTTCGCAGCCTATCGCTCAAGAGTGAAGTTCAGCAGGAGCGCAGACGCCCCCG
CGTACCAGCAGGGCCAGAACCAGCTCTATAACGAGCTCAATCTAGGACGAAGAGAGGAGTAC
GATGTTTTGGACAAGAGACGTGGCCGGGACCCTGAGATGGGGGGAAAGCCGCAGAGAAGGAA
GAACCCTCAGGAAGGCCTGTACAATGAACTGCAGAAAGATAAGATGGCGGAGGCCTACAGTG
AGATTGGGATGAAAGGCGAGCGCCGGAGGGGCAAGGGGCACGATGGCCTTTACCAGGGTCTC
AGTACAGCCACCAAGGACACCIACGACGCCCTTCACATGCAGGCCCTGCCCCCTCGCCAGTG
CACCAACTACGCCCTGCTGAAGCTGGCCGGCGACGTGGAGAGCAACCCCGGCCCC
(SEQ ID NO: 35)
ATGAAGTGGAAGGCGCTTTTCACCGCGGCCATCCTGCAGGCACAGTTGCCGATTACAGAGGC
ACAGAGCTTTGGCCTGCTGGATCCCAAACTCTGCTACCTGCTGGATGGAATCCTCTTCATCT
ATGGTGTCATTCTCACTGCCTTGTTCCTGAGGAGTAAGAGGAGCAGGCTCCTGCACAGTGAC
TACATGAACATGACTCCCCGCCGCCCCGGGCCCACCCGCAAGCATTACCAGCCCTATGCCCC
ACCACGCGACTTCGCAGCCTATCGCTCACTTTGCGCACGCCCACGCCGCAGCCCCGCCCAAG
AAGATGGCAAAGTCTACATCAACATGCCAGGCAGGGGCAGAGTGAAGTTCAGCAGGAGCGCA
GACGCCCCCGCGTACCAGCAGGGCCAGAACCAGCTCTATAACGAGCTCAATCTAGGACGAAG
AGAGGAGTACGATGTTTTGGACAAGAGACGTGGCCGGGACCCTGAGATGGGGGGAAAGCCGC
AGAGAAGGAAGAACCCTCAGGAAGGCCTGTACAATGAACTGCAGAAAGATAAGATGGCGGAG
GCCTACAGTGAGATTGGGATGAAAGGCGAGCGCCGGAGGGGCAAGGGGCACGATGGCCTTTA
CCAGGGTCTCAGTACAGCCACCAAGGACACCTACGACGCCCTTCACATGCAGGCCCTGCCCC
CTCGCCAGTGCACCAACTACGCCCTGCTGAAGCTGGCCGGCGACGTGGAGAGCAACCCCGGC
CCC
(SEQ ID NO: 36)
MKWKALFTAAILQAQLPITEAQSFGLLDPKLCYLLDGILFIYGVILTALFLLCARPRRSPAQ
EDGKVYINMPGRGRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATKDTYDALHMQALP
PRQCTNYALLKLAGDVESNPGP
(SEQ ID NO: 37)
MKWKALFTAAILQAQLPITEAQSFGLLDPKLCYLLDGILFIYGVILTALFLRSKRSRLLHSD
YMNMTPRRPGPTRKHYQPYAPPRDFAAYRSRVKFSRSADAPAYQQGQNOLYNELNLGRREEY
DVLDKRRGRDPEMGGKPQRRKNPQEGLYNELQKDKMAEAYSEIGMKGERRRGKGHDGLYQGL
STATKDTYDALHMQALPPRQCTNYALLKLAGDVESNPGP
(SEQ ID NO: 38)
MKWKALFTAAILQAQLPITEAQSFGLLDPKLCYLLDGILFIYGVILTALFLRSKRSRLLHSD
YMNMTPRRPGPTRKHYQPYAPPRDFAAYRSLCARPRRSPAQEDGKVYINMPGRGRVKFSRSA
DAPAYQQGQNQLYNELNLGRREEYDVLDKRRGRDPEMGGKPQRRKNPQEGLYNELQKDKMAE
AYSEIGMKGERRRGKGHDGLYQGLSTATKDTYDALHMQALPPRQCTNYALLKLAGDVESNPG
P

In certain embodiments, a DAP10 intracellular co-stimulatory domain is represented by a polynucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 39. In certain embodiments, a CD28 intracellular co-stimulatory domain is represented by a polynucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 40. In certain embodiments, a DAP10 intracellular co-stimulatory domain and CD28 intracellular co-stimulatory domain is represented by a polynucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 41. In certain embodiments, a DAP10 intracellular co-stimulatory domain is represented by an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 42. In certain embodiments, a CD28 intracellular co-stimulatory domain is represented by an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 43. In certain embodiments, a DAP10 intracellular co-stimulatory domain and CD28 intracellular co-stimulatory domain is represented by an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 44.

(SEQ ID NO: 39)
CTTTGCGCACGCCCACGCCGCAGCCCCGCCCAAGAAGATGGCAA
AGTCTACATCAACATGCCAGGCAGGGGC
(SEQ ID NO: 40)
AGGAGTAAGAGGAGCAGGCTCCTGCACAGTGACTACATGAACATG
ACTCCCCGCCGCCCCGGGCCCACCCGCAAGCATTACCAGCCCTAT
GCCCCACCACGCGACTTCGCAGCCTATCGCTCA
(SEQ ID NO: 41)
AGGAGTAAGAGGAGCAGGCTCCTGCACAGTGACTACATGAACATG
ACTCCCCGCCGCCCCGGGCCCACCCGCAAGCATTACCAGCCCTAT
GCCCCACCACGCGACTTCGCAGCCTATCGCTCACTTTGCGCACGC
CCACGCCGCAGCCCCGCCCAAGAAGATGGCAAAGTCTACATCAAC
ATGCCAGGCAGGGGC
(SEQ ID NO: 42)
LCARPRRSPAQEDGKVYINMPGRG
(SEQ ID NO: 43)
RSKRSRLLHSDYMNMTPRRPGPTRKHYQPYAPPRDFAAYRS
(SEQ ID NO: 44)
RSKRSRLLHSDYMNMTPRRPGPTRKHYQPYAPPRDFAAYRSLCA
RPRRSPAQEDGKVYINMPGRG

UTNK15-DAP10: refers to full length CD3zeta comprising a fusion with an intracellular co-stimulatory domain derived from DAP10, full length CD3 gamma, full length CD3 delta, and full length CD3 epsilon linked to IL15, it may be represented by a polynucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 45. In certain embodiments, a UTNK15-DAP10 amino acid sequence may be represented by an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 46.

(SEQ ID NO: 45)
ATGAAGTGGAAGGCGCTTTTCACCGCGGCCATCCTGCAGGCACAGTTGCCGATTACAGAGGC
ACAGAGCTTTGGCCTGCTGGATCCCAAACTCTGCTACCTGCTGGATGGAATCCTCTTCATCT
ATGGTGTCATTCTCACTGCCTTGTTCCTGCTTTGCGCACGCCCACGCCGCAGCCCCGCCCAA
GAAGATGGCAAAGTCTACATCAACATGCCAGGCAGGGGCAGAGTGAAGTTCAGCAGGAGCGC
AGACGCCCCCGCGTACCAGCAGGGCCAGAACCAGCTCTATAACGAGCTCAATCTAGGACGAA
GAGAGGAGTACGATGTTTTGGACAAGAGACGTGGCCGGGACCCTGAGATGGGGGGAAAGCCG
CAGAGAAGGAAGAACCCTCAGGAAGGCCTGTACAATGAACTGCAGAAAGATAAGATGGCGGA
GGCCTACAGTGAGATTGGGATGAAAGGCGAGCGCCGGAGGGGCAAGGGGCACGATGGCCTTT
ACCAGGGTCTCAGTACAGCCACCAAGGACACCTACGACGCCCTTCACATGCAGGCCCTGCCC
CCTCGCCAGTGCACCAACTACGCCCTGCTGAAGCTGGCCGGCGACGTGGAGAGCAACCCCGG
CCCCATGGAACAGGGGAAGGGCCTGGCTGTCCTCATCCTGGCTATCATTCTTCTTCAAGGTA
CTTTGGCCCAGTCAATCAAAGGAAACCACTTGGTTAAGGTGTATGACTATCAAGAAGATGGT
TCGGTACTTCTGACTTGTGATGCAGAAGCCAAAAATATCACATGGTTTAAAGATGGGAAGAT
GATCGGCTTCCTAACTGAAGATAAAAAAAAATGGAATCTGGGAAGTAATGCCAAGGACCCTC
GTGGGATGTATCAGTGTAAAGGATCACAGAACAAGTCAAAACCACTCCAAGTGTATTACAGA
ATGTGTCAGAACTGCATTGAACTAAATGCAGCCACCATATCTGGCTTTCTCTTTGCTGAAAT
CGTCAGCATTTTCGTCCTTGCTGTTGGGGTCTACTTCATTGCTGGACAGGATGGAGTTCGCC
AGTCGAGAGCTTCAGACAAGCAGACTCTGTTGCCCAATGACCAGCTCTACCAGCCCCTCAAG
GATCGAGAAGATGACCAGTACAGCCACCTTCAAGGAAACCAGTTGAGGAGGAATGTGAAGCA
GACCCTGAACTTCGACCTGCTGAAGCTGGCCGGCGACGTGGAGAGCAACCCCGGCCCCATGG
AGCACAGCACCTTCCTGAGCGGCCTGGTGCTGGCCACCCTGCTGAGCCAGGTGAGCCCCTTC
AAGATCCCCATCGAGGAGCTGGAGGACAGAGTGTTCGTGAACTGCAACACCAGCATCACCTG
GGTGGAGGGCACCGTGGGCACCCTGCTGAGCGACATCACCAGACTGGACCTGGGCAAGAGAA
TCCTGGACCCCAGAGGCATCTACAGATGCAACGGCACCGACATCTACAAGGACAAGGAGAGC
ACCGTGCAGGTGCACTACAGAATGTGCCAGAGCTGCGTGGAGCTGGACCCCGCCACCGTGGC
CGGCATCATCGTGACCGACGTGATCGCCACCCTGCTGCTGGCCCTGGGCGTGTTCTGCTTCG
CCGGCCACGAGACCGGCAGACTGAGCGGCGCCGCCGACACCCAGGCCCTGCTGAGAAACGAC
CAGGTGTACCAGCCCCTGAGAGACAGAGACGACGCCCAGTACAGCCACCTGGGCGGCAACTG
GGCCAGAAACAAGGAGGGCAGAGGCAGCCTGCTGACCTGCGGCGACGTGGAGGAGAACCCCG
GCCCCATGCAGAGCGGCACCCACTGGAGAGTGCTGGGCCTGTGCCTGCTGAGCGTGGGCGTG
TGGGGCCAGGACGGCAACGAGGAGATGGGCGGCATCACCCAGACCCCCTACAAGGTGAGCAT
CAGCGGCACCACCGTGATCCTGACCTGCCCCCAGTACCCCGGCAGCGAGATCCTGTGGCAGC
ACAACGACAAGAACATCGGCGGCGACGAGGACGACAAGAACATCGGCAGCGACGAGGACCAC
CTGAGCCTGAAGGAGTTCAGCGAGCTGGAGCAGAGCGGCTACTACGTGTGCTACCCCAGAGG
CAGCAAGCCCGAGGACGCCAACTTCTACCTGTACCTGAGAGCCAGAGTGTGCGAGAACTGCA
TGGAGATGGACGTGATGAGCGTGGCCACCATCGTGATCGTGGACATCTGCATCACCGGCGGC
CTGCTGCTGCTGGTGTACTACTGGAGCAAGAACAGAAAGGCCAAGGCCAAGCCCGTGACCAG
AGGCGCCGGCGCCGGCGGCAGACAGAGAGGCCAGAACAAGGAGAGACCCCCCCCCGTGCCCA
ACCCCGACTACGAGCCCATCAGAAAGGGCCAGAGAGACCTGTACAGCGGCCTGAACCAGAGA
AGAATCGGACCGCAGTGTACTAATTATGCTCTCTTGAAATTGGCTGGAGATGTTGAGAGCAA
TCCCGGGCCCATGCGCATTAGCAAGCCCCACCTGCGGAGCATCAGCATCCAGTGCTACCTGT
GCCTGCTGCTGAACAGCCACTTCCTGACCGAGGCCGGCATCCACGTGTTCATCCTGGGCTGC
TTCAGCGCCGGACTGCCCAAGACCGAGGCCAACTGGGTGAACGTGATCAGCGACCTGAAGAA
GATCGAGGACCTGATCCAGAGCATGCACATCGACGCCACCCTGTACACCGAGAGCGACGTGC
ACCCCAGCTGCAAGGTGACCGCCATGAAGTGCTTTCTGCTGGAACTGCAGGTGATCAGCCTG
GAAAGCGGCGACGCCAGCATCCACGACACCGTGGAGAACCTGATCATCCTGGCCAACAACAG
CCTGAGCAGCAACGGCAACGTGACCGAGAGCGGCTGCAAAGAGTGCGAGGAACTGGAAGAGA
AGAACATCAAAGAGTTTCTGCAGAGCTTCGTGCACATCGTGCAGATGTTCATCAACACCAGC
(SEQ ID NO: 46)
MKWKALFTAAILQAQLPITEAQSFGLLDPKLCYLLDGILFIYGVILTALFLLCARPRRSPAQ
EDGKVYINMPGRGRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATKDTYDALHMQALP
PRQCTNYALLKLAGDVESNPGPMEQGKGLAVLILAIILLQGTLAQSIKGNHLVKVYDYQEDG
SVLLTCDAEAKNITWFKDGKMIGFLTEDKKKWNLGSNAKDPRGMYQCKGSQNKSKPLQVYYR
MCQNCIELNAATISGELFAEIVSIFVLAVGVYFIAGQDGVRQSRASDKQTLLPNDQLYQPLK
DREDDQYSHLQGNQLRRNVKQTLNFDLLKLAGDVESNPGPMEHSTFLSGLVLATLLSQVSPF
KIPIEELEDRVFVNCNTSITWVEGTVGTLLSDITRLDLGKRILDPRGIYRCNGTDIYKDKES
TVQVHYRMCQSCVELDPATVAGIIVTDVIATLLLALGVFCFAGHETGRLSGAADTQALLRND
QVYQPLRDRDDAQYSHLGGNWARNKEGRGSLLTCGDVEENPGPMQSGTHWRVLGLCLLSVGV
WGQDGNEEMGGITQTPYKVSISGTTVILTCPQYPGSEILWQHNDKNIGGDEDDKNIGSDEDH
LSLKEFSELEQSGYYVCYPRGSKPEDANFYLYLRARVCENCMEMDVMSVATIVIVDICITGG
LLLLVYYWSKNRKAKAKPVTRGAGAGGRQRGQNKERPPPVPNPDYEPIRKGQRDLYSGLNQR
RIGPQCTNYALLKLAGDVESNPGPMRISKPHLRSISIQCYLCLLLNSHELTEAGIHVFILGC
FSAGLPKTEANWVNVISDLKKIEDLIQSMHIDATLYTESDVHPSCKVTAMKCFLLELQVISL
ESGDASIHDTVENLIILANNSLSSNGNVTESGCKECEELEEKNIKEFLQSFVHIVQMFINTS

UTNK15-28: refers to full length CD3zeta comprising a fusion with an intracellular co-stimulatory domain derived from CD28, full length CD3 gamma, full length CD3 delta, and full length CD3 epsilon linked to IL15, it may be represented by a polynucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 47. In certain embodiments, a UTNK15-28 amino acid sequence may be represented by an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 48.

(SEQ ID NO: 47)
ATGAAGTGGAAGGCGCTTTTCACCGCGGCCATCCTGCAGGCACAGTTGCCGATTACAGAGGC
ACAGAGCTTTGGCCTGCTGGATCCCAAACTCTGCTACCTGCTGGATGGAATCCTCTTCATCT
ATGGTGTCATTCTCACTGCCTTGTTCCTGAGGAGTAAGAGGAGCAGGCTCCTGCACAGTGAC
TACATGAACATGACTCCCCGCCGCCCCGGGCCCACCCGCAAGCATTACCAGCCCTATGCCCC
ACCACGCGACTTCGCAGCCTATCGCTCAAGAGTGAAGTTCAGCAGGAGCGCAGACGCCCCCG
CGTACCAGCAGGGCCAGAACCAGCTCTATAACGAGCTCAATCTAGGACGAAGAGAGGAGTAC
GATGTTTTGGACAAGAGACGTGGCCGGGACCCTGAGATGGGGGGAAAGCCGCAGAGAAGGAA
GAACCCTCAGGAAGGCCTGTACAATGAACTGCAGAAAGATAAGATGGCGGAGGCCTACAGTG
AGATTGGGATGAAAGGCGAGCGCCGGAGGGGCAAGGGGCACGATGGCCTTTACCAGGGTCTC
AGTACAGCCACCAAGGACACCTACGACGCCCTTCACATGCAGGCCCTGCCCCCTCGCCAGTG
CACCAACTACGCCCTGCTGAAGCTGGCCGGCGACGTGGAGAGCAACCCCGGCCCCATGGAAC
AGGGGAAGGGCCTGGCTGTCCTCATCCTGGCTATCATTCTTCTTCAAGGTACTTTGGCCCAG
TCAATCAAAGGAAACCACTTGGTTAAGGTGTATGACTATCAAGAAGATGGTTCGGTACTTCT
GACTTGTGATGCAGAAGCCAAAAATATCACATGGTTTAAAGATGGGAAGATGATCGGCTTCC
TAACTGAAGATAAAAAAAAATGGAATCTGGGAAGTAATGCCAAGGACCCTCGTGGGATGTAT
CAGTGTAAAGGATCACAGAACAAGTCAAAACCACTCCAAGTGTATTACAGAATGTGTCAGAA
CTGCATTGAACTAAATGCAGCCACCATATCTGGCTTTCTCTTTGCTGAAATCGTCAGCATTT
TCGTCCTTGCTGTTGGGGTCTACTTCATTGCTGGACAGGATGGAGTTCGCCAGTCGAGAGCT
TCAGACAAGCAGACTCTGTTGCCCAATGACCAGCTCTACCAGCCCCTCAAGGATCGAGAAGA
TGACCAGTACAGCCACCTTCAAGGAAACCAGTTGAGGAGGAATGTGAAGCAGACCCTGAACT
TCGACCTGCTGAAGCTGGCCGGCGACGTGGAGAGCAACCCCGGCCCCATGGAGCACAGCACC
TTCCTGAGCGGCCTGGTGCTGGCCACCCTGCTGAGCCAGGTGAGCCCCTTCAAGATCCCCAT
CGAGGAGCTGGAGGACAGAGTGTTCGTGAACTGCAACACCAGCATCACCTGGGTGGAGGGCA
CCGTGGGCACCCTGCTGAGCGACATCACCAGACTGGACCTGGGCAAGAGAATCCTGGACCCC
AGAGGCATCTACAGATGCAACGGCACCGACATCTACAAGGACAAGGAGAGCACCGTGCAGGT
GCACTACAGAATGTGCCAGAGCTGCGTGGAGCTGGACCCCGCCACCGTGGCCGGCATCATCG
TGACCGACGTGATCGCCACCCTGCTGCTGGCCCTGGGCGTGTTCTGCTTCGCCGGCCACGAG
ACCGGCAGACTGAGCGGCGCCGCCGACACCCAGGCCCTGCTGAGAAACGACCAGGTGTACCA
GCCCCTGAGAGACAGAGACGACGCCCAGTACAGCCACCTGGGCGGCAACTGGGCCAGAAACA
AGGAGGGCAGAGGCAGCCTGCTGACCTGCGGCGACGTGGAGGAGAACCCCGGCCCCATGCAG
AGCGGCACCCACTGGAGAGTGCTGGGCCTGTGCCTGCTGAGCGTGGGCGTGTGGGGCCAGGA
CGGCAACGAGGAGATGGGCGGCATCACCCAGACCCCCTACAAGGTGAGCATCAGCGGCACCA
CCGTGATCCTGACCTGCCCCCAGTACCCCGGCAGCGAGATCCTGTGGCAGCACAACGACAAG
AACATCGGCGGCGACGAGGACGACAAGAACATCGGCAGCGACGAGGACCACCTGAGCCTGAA
GGAGTTCAGCGAGCTGGAGCAGAGCGGCTACTACGTGTGCTACCCCAGAGGCAGCAAGCCCG
AGGACGCCAACTTCTACCTGTACCTGAGAGCCAGAGTGTGCGAGAACTGCATGGAGATGGAC
GTGATGAGCGTGGCCACCATCGTGATCGTGGACATCTGCATCACCGGCGGCCTGCTGCTGCT
GGTGTACTACTGGAGCAAGAACAGAAAGGCCAAGGCCAAGCCCGTGACCAGAGGCGCCGGCG
CCGGCGGCAGACAGAGAGGCCAGAACAAGGAGAGACCCCCCCCCGTGCCCAACCCCGACTAC
GAGCCCATCAGAAAGGGCCAGAGAGACCTGTACAGCGGCCTGAACCAGAGAAGAATCGGACC
GCAGTGTACTAATTATGCTCTCTTGAAATTGGCTGGAGATGTTGAGAGCAATCCCGGGCCCA
TGCGCATTAGCAAGCCCCACCTGCGGAGCATCAGCATCCAGTGCTACCTGTGCCTGCTGCTG
AACAGCCACTTCCTGACCGAGGCCGGCATCCACGTGTTCATCCTGGGCTGCTTCAGCGCCGG
ACTGCCCAAGACCGAGGCCAACTGGGTGAACGTGATCAGCGACCTGAAGAAGATCGAGGACC
TGATCCAGAGCATGCACATCGACGCCACCCTGTACACCGAGAGCGACGTGCACCCCAGCTGC
AAGGTGACCGCCATGAAGTGCTTTCTGCTGGAACTGCAGGTGATCAGCCTGGAAAGCGGCGA
CGCCAGCATCCACGACACCGTGGAGAACCTGATCATCCTGGCCAACAACAGCCTGAGCAGCA
ACGGCAACGTGACCGAGAGCGGCTGCAAAGAGTGCGAGGAACTGGAAGAGAAGAACATCAAA
GAGTTTCTGCAGAGCTTCGTGCACATCGTGCAGATGTTCATCAACACCAGC
(SEQ ID NO: 48)
MKWKALFTAAILQAQLPITEAQSFGLLDPKLCYLLDGILFIYGVILTALFLRSKRSRLLHSD
YMNMTPRRPGPTRKHYQPYAPPRDFAAYRSRVKFSRSADAPAYQQGQNQLYNELNLGRREEY
DVLDKRRGRDPEMGGKPQRRKNPQEGLYNELQKDKMAEAYSEIGMKGERRRGKGHDGLYQGL
STATKDTYDALHMQALPPRQCTNYALLKLAGDVESNPGPMEQGKGLAVLILAIILLQGTLAQ
SIKGNHLVKVYDYQEDGSVLLTCDAEAKNITWFKDGKMIGFLTEDKKKWNLGSNAKDPRGMY
QCKGSQNKSKPLQVYYRMCQNCIELNAATISGFLFAEIVSIFVLAVGVYFIAGQDGVRQSRA
SDKQTLLPNDQLYQPLKDREDDQYSHLQGNQLRRNVKQTLNFDLLKLAGDVESNPGPMEHST
FLSGLVLATLLSQVSPFKIPIEELEDRVFVNCNTSITWVEGTVGTLLSDITRLDLGKRILDP
RGIYRCNGTDIYKDKESTVQVHYRMCQSCVELDPATVAGIIVTDVIATLLLALGVFCFAGHE
TGRLSGAADTQALLRNDQVYQPLRDRDDAQYSHLGGNWARNKEGRGSLLTCGDVEENPGPMQ
SGTHWRVLGLCLLSVGVWGQDGNEEMGGITQTPYKVSISGTTVILTCPQYPGSEILWQHNDK
NIGGDEDDKNIGSDEDHLSLKEFSELEQSGYYVCYPRGSKPEDANFYLYLRARVCENCMEMD
VMSVATIVIVDICITGGLLLLVYYWSKNRKAKAKPVTRGAGAGGRQRGQNKERPPPVPNPDY
EPIRKGQRDLYSGLNQRRIGPQCTNYALLKLAGDVESNPGPMRISKPHLRSISIQCYLCLLL
NSHFLTEAGIHVFILGCFSAGLPKTEANWVNVISDLKKIEDLIQSMHIDATLYTESDVHPSC
KVTAMKCFLLELQVISLESGDASIHDTVENLIILANNSLSSNGNVTESGCKECEELEEKNIK
EFLQSFVHIVQMFINTS

UTNK15-28-DAP10: refers to full length CD3zeta comprising a fusion with an intracellular co-stimulatory domain derived from DAP10 and an intracellular co-stimulatory domain derived from CD28, full length CD3 gamma, full length CD3 delta, and full length CD3 epsilon linked to IL15, it may be represented by a polynucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 49. In certain embodiments, a UTNK15-28-DAP10 amino acid sequence may be represented by an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 50.

(SEQ ID NO: 49)
ATGAAGTGGAAGGCGCTTTTCACCGCGGCCATCCTGCAGGCACAGTTGCCGATTACAGAGGC
ACAGAGCTTTGGCCTGCTGGATCCCAAACTCTGCTACCTGCTGGATGGAATCCTCTTCATCT
ATGGTGTCATTCTCACTGCCTTGTTCCTGAGGAGTAAGAGGAGCAGGCTCCTGCACAGTGAC
TACATGAACATGACTCCCCGCCGCCCCGGGCCCACCCGCAAGCATTACCAGCCCTATGCCCC
ACCACGCGACTTCGCAGCCTATCGCTCACTTTGCGCACGCCCACGCCGCAGCCCCGCCCAAG
AAGATGGCAAAGTCTACATCAACATGCCAGGCAGGGGCAGAGTGAAGTTCAGCAGGAGCGCA
GACGCCCCCGCGTACCAGCAGGGCCAGAACCAGCTCTATAACGAGCTCAATCTAGGACGAAG
AGAGGAGTACGATGTTTTGGACAAGAGACGTGGCCGGGACCCTGAGATGGGGGGAAAGCCGC
AGAGAAGGAAGAACCCTCAGGAAGGCCTGTACAATGAACTGCAGAAAGATAAGATGGCGGAG
GCCTACAGTGAGATTGGGATGAAAGGCGAGCGCCGGAGGGGCAAGGGGCACGATGGCCTTTA
CCAGGGTCTCAGTACAGCCACCAAGGACACCTACGACGCCCTTCACATGCAGGCCCTGCCCC
CTCGCCAGTGCACCAACTACGCCCTGCTGAAGCTGGCCGGCGACGTGGAGAGCAACCCCGGC
CCCATGGAACAGGGGAAGGGCCTGGCTGTCCTCATCCTGGCTATCATTCTTCTTCAAGGTAC
TTTGGCCCAGTCAATCAAAGGAAACCACTTGGTTAAGGTGTATGACTATCAAGAAGATGGTT
CGGTACTTCTGACTTGTGATGCAGAAGCCAAAAATATCACATGGTTTAAAGATGGGAAGATG
ATCGGCTTCCTAACTGAAGATAAAAAAAAATGGAATCTGGGAAGTAATGCCAAGGACCCTCG
TGGGATGTATCAGTGTAAAGGATCACAGAACAAGTCAAAACCACTCCAAGTGTATTACAGAA
TGTGTCAGAACTGCATTGAACTAAATGCAGCCACCATATCTGGCTTTCTCTTTGCTGAAATC
GTCAGCATTTTCGTCCTTGCTGTTGGGGTCTACTTCATTGCTGGACAGGATGGAGTTCGCCA
GTCGAGAGCTTCAGACAAGCAGACTCTGTTGCCCAATGACCAGCTCTACCAGCCCCTCAAGG
ATCGAGAAGATGACCAGTACAGCCACCTTCAAGGAAACCAGTTGAGGAGGAATGTGAAGCAG
ACCCTGAACTTCGACCTGCTGAAGCTGGCCGGCGACGTGGAGAGCAACCCCGGCCCCATGGA
GCACAGCACCTTCCTGAGCGGCCTGGTGCTGGCCACCCTGCTGAGCCAGGTGAGCCCCTTCA
AGATCCCCATCGAGGAGCTGGAGGACAGAGTGTTCGTGAACTGCAACACCAGCATCACCTGG
GTGGAGGGCACCGTGGGCACCCTGCTGAGCGACATCACCAGACTGGACCTGGGCAAGAGAAT
CCTGGACCCCAGAGGCATCTACAGATGCAACGGCACCGACATCTACAAGGACAAGGAGAGCA
CCGTGCAGGTGCACTACAGAATGTGCCAGAGCTGCGTGGAGCTGGACCCCGCCACCGTGGCC
GGCATCATCGTGACCGACGTGATCGCCACCCTGCTGCTGGCCCTGGGCGTGTTCTGCTTCGC
CGGCCACGAGACCGGCAGACTGAGCGGCGCCGCCGACACCCAGGCCCTGCTGAGAAACGACC
AGGTGTACCAGCCCCTGAGAGACAGAGACGACGCCCAGTACAGCCACCTGGGCGGCAACTGG
GCCAGAAACAAGGAGGGCAGAGGCAGCCTGCTGACCTGCGGCGACGTGGAGGAGAACCCCGG
CCCCATGCAGAGCGGCACCCACTGGAGAGTGCTGGGCCTGTGCCTGCTGAGCGTGGGCGTGT
GGGGCCAGGACGGCAACGAGGAGATGGGCGGCATCACCCAGACCCCCTACAAGGTGAGCATC
AGCGGCACCACCGTGATCCTGACCTGCCCCCAGTACCCCGGCAGCGAGATCCTGTGGCAGCA
CAACGACAAGAACATCGGCGGCGACGAGGACGACAAGAACATCGGCAGCGACGAGGACCACC
TGAGCCTGAAGGAGTTCAGCGAGCTGGAGCAGAGCGGCTACTACGTGTGCTACCCCAGAGGC
AGCAAGCCCGAGGACGCCAACTTCTACCTGTACCTGAGAGCCAGAGTGTGCGAGAACTGCAT
GGAGATGGACGTGATGAGCGTGGCCACCATCGTGATCGTGGACATCTGCATCACCGGCGGCC
TGCTGCTGCTGGTGTACTACTGGAGCAAGAACAGAAAGGCCAAGGCCAAGCCCGTGACCAGA
GGCGCCGGCGCCGGCGGCAGACAGAGAGGCCAGAACAAGGAGAGACCCCCCCCCGTGCCCAA
CCCCGACTACGAGCCCATCAGAAAGGGCCAGAGAGACCTGTACAGCGGCCTGAACCAGAGAA
GAATCGGACCGCAGTGTACTAATTATGCTCTCTTGAAATTGGCTGGAGATGTTGAGAGCAAT
CCCGGGCCCATGCGCATTAGCAAGCCCCACCTGCGGAGCATCAGCATCCAGTGCTACCTGTG
CCTGCTGCTGAACAGCCACTTCCTGACCGAGGCCGGCATCCACGTGTTCATCCTGGGCTGCT
TCAGCGCCGGACTGCCCAAGACCGAGGCCAACTGGGTGAACGTGATCAGCGACCTGAAGAAG
ATCGAGGACCTGATCCAGAGCATGCACATCGACGCCACCCTGTACACCGAGAGCGACGTGCA
CCCCAGCTGCAAGGTGACCGCCATGAAGTGCTTTCTGCTGGAACTGCAGGTGATCAGCCTGG
AAAGCGGCGACGCCAGCATCCACGACACCGTGGAGAACCTGATCATCCTGGCCAACAACAGC
CTGAGCAGCAACGGCAACGTGACCGAGAGCGGCTGCAAAGAGTGCGAGGAACTGGAAGAGAA
GAACATCAAAGAGTTTCTGCAGAGCTTCGTGCACATCGTGCAGATGTTCATCAACACCAGC
(SEQ ID NO: 50)
MKWKALFTAAILQAQLPITEAQSFGLLDPKLCYLLDGILFIYGVILTALFLRSKRSRLLHSD
YMNMTPRRPGPTRKHYQPYAPPRDFAAYRSLCARPRRSPAQEDGKVYINMPGRGRVKFSRSA
DAPAYQQGQNQLYNELNLGRREEYDVLDKRRGRDPEMGGKPQRRKNPQEGLYNELQKDKMAE
AYSEIGMKGERRRGKGHDGLYQGLSTATKDTYDALHMQALPPRQCTNYALLKLAGDVESNPG
PMEQGKGLAVLILAIILLQGTLAQSIKGNHLVKVYDYQEDGSVLLTCDAEAKNITWFKDGKM
IGFLTEDKKKWNLGSNAKDPRGMYQCKGSQNKSKPLQVYYRMCQNCIELNAATISGELFAEI
VSIFVLAVGVYFIAGQDGVRQSRASDKQTLLPNDQLYQPLKDREDDQYSHLQGNQLRRNVKQ
TLNFDLLKLAGDVESNPGPMEHSTFLSGLVLATLLSQVSPFKIPIEELEDRVFVNCNTSITW
VEGTVGTLLSDITRLDLGKRILDPRGIYRCNGTDIYKDKESTVQVHYRMCQSCVELDPATVA
GIIVTDVIATLLLALGVFCFAGHETGRLSGAADTQALLRNDQVYQPLRDRDDAQYSHLGGNW
ARNKEGRGSLLTCGDVEENPGPMQSGTHWRVLGLCLLSVGVWGQDGNEEMGGITQTPYKVSI
SGTTVILTCPQYPGSEILWQHNDKNIGGDEDDKNIGSDEDHLSLKEFSELEQSGYYVCYPRG
SKPEDANFYLYLRARVCENCMEMDVMSVATIVIVDICITGGLLLLVYYWSKNRKAKAKPVTR
GAGAGGRQRGQNKERPPPVPNPDYEPIRKGQRDLYSGLNQRRIGPQCTNYALLKLAGDVESN
PGPMRISKPHLRSISIQCYLCLLLNSHELTEAGIHVFILGCFSAGLPKTEANWVNVISDLKK
IEDLIQSMHIDATLYTESDVHPSCKVTAMKCFLLELQVISLESGDASIHDTVENLIILANNS
LSSNGNVTESGCKECEELEEKNIKEFLQSFVHIVQMFINTS

As depicted in FIG. 3C and described above, the term “linked” refers to being present on the same polynucleotide vector and does not necessarily mean that the two polypeptides are expressed as one polypeptide. For example, a cytokine produced from a vector of the disclosure may ultimately be produced as a separate molecule from any one or more TCR/CD3 receptor complex components. Whereas, the term “fused” or “fusion” refers to two polypeptides that comprise a peptide bond conjoining the two molecules, i.e. that the two polypeptides are covalently bound by an amide bond and are not separated by a splitting element, such as a 2A element.

In some embodiments, a TCR construct comprises Human papilloma virus (HPV)-specific TCR chains. In some embodiments, a TCR construct comprising an HPV-specific TCR chains comprises TCR alpha and TCR beta chains that target the HPV 18 E6 protein, and/or HPV 18 E7 protein. In some embodiments, an HPV 18 E6 epitope is amino acids 121-135 and/or amino acids 77-91 of the HPV 18 E6 protein. In some embodiments, a TCR construct comprising an HPV-specific TCR chains comprises TCR alpha and TCR beta chains that target the HPV 18 E7 protein. In some embodiments, an HPV 18 E7 epitope is amino acids 11-19. In some embodiments, HPV-specific TCR sequences, TCR variable domain sequences, CDR sequences, and/or TCR constant domain sequences, are described in international patent application publications WO 2015/009604 A1, which is incorporated herein by reference for the purpose described herein.

In some embodiments, a TCR is an invariant natural killer T cell TCR (iTCR). In some embodiments, an iTCR may bind to CD1d expressing cells, such as certain cancer cells, monocytes, and/or macrophages that promote tumor growth. While not being limited by theory, CD1d molecules are not polymorphic, and thus in certain embodiments, recognition of antigens presented by CD1d by transgenic iTCR bearing NK cells does not result in a host-graft mismatch. In certain embodiments, utilization of iTCRs in place of standard T cell derived TCRs can reduce comorbidities associated with MHC-1/2 mismatch.

In some embodiments, an iTCR complex comprises an alpha (a) chain iTCR (iTCRα). In some embodiments, a construct encoding an iTCRα comprises a polynucleotide coding sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to SEQ ID NO: 51 (e.g., iTCRα associated with Vα24-Jα18 in humans). In some embodiments, an iTCRα comprises an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to SEQ ID NO: 52 (e.g., iTCRα associated with Vα24-Jα18 in humans).

iTCRα
(SEQ ID NO: 51)
ATGAAGAAGCACCTGACCACCTTTCTGGTCATCCTGTGGCTGTACTTCTACAGAGGCAACGG
CAAGAACCAGGTGGAACAGAGCCCTCAGAGCCTGATCATCCTGGAAGGCAAGAACTGCACCC
TGCAGTGCAACTACACCGTGTCTCCCTTCAGCAACCTGCGGTGGTACAAGCAGGATACAGGC
AGAGGCCCTGTGTCTCTGACCATCATGACCTTCAGCGAGAACACCAAGAGCAACGGCCGGTA
CACCGCCACACTGGATGCCGATACAAAGCAGAGCAGCCTGCACATCACAGCCAGCCAGCTGA
GCGATAGCGCCAGCTACATCTGCGTGGTGTCCGATAGAGGCAGCACCCTGGGCAGACTGTAC
TTTGGCAGAGGCACCCAGCTGACCGTGTGGCCCGATATTCAGAACCCTGATCCTGCCGTGTA
CCAGCTGAGAGACAGCAAGTCCAGCGACAAGAGCGTGTGCCTGTTCACCGACTTCGACAGCC
AGACCAACGTGTCCCAGAGCAAGGACAGCGACGTGTACATCACCGACAAGtgcGTGCTGGAC
ATGCGGAGCATGGACTTCAAGAGCAATAGCGCCGTGGCCTGGTCCAACAAGAGCGATTTCGC
CTGCGCCAACGCCTTCAACAACAGCATTATCCCCGAGGACACATTCTTCCCAAGTCCTGAGA
GCAGCTGCGACGTGAAGCTGGTGGAAAAGAGCTTCGAGACAGACACCAACCTGAACTTCCAG
AACCTGAGCGTGATCGGCTTCCGGATCCTGCTGCTGAAAGTGGCCGGCTTCAACCTGCTGAT
GACCCTGAGACTGTGGTCTAGC
(SEQ ID NO: 52)
MKKHLTTFLVILWLYFYRGNGKNQVEQSPQSLIILEGKNCTLQCNYTVSPFSNLRWYKQDTG
RGPVSLTIMTFSENTKSNGRYTATLDADTKQSSLHITASQLSDSASYICVVSDRGSTLGRLY
FGRGTQLTVWPDIQNPDPAVYQLRDSKSSDKSVCLFTDFDSQTNVSQSKDSDVYITDKCVLD
MRSMDFKSNSAVAWSNKSDFACANAFNNSIIPEDTFFPSPESSCDVKLVEKSFETDINLNFQ
NLSVIGFRILLLKVAGENLLMTLRLWSS

iTCRβ

In some embodiments, an iTCR complex comprises a beta (β) chain iTCR (iTCRβ). In some embodiments, a construct encoding an iTCRβ comprises a polynucleotide coding sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to SEQ ID NO: 53, 55, or 57. In some embodiments, an iTCRβ comprises an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to SEQ ID NO: 54, 56, 58.

(SEQ ID NO: 53)
ATGAAGAAGCACCTGACCACCTTTCTGGTCATCCTGTGGCTGTACTTCTACAGAGGCAACGG
CAAGAACCAGGTGGAACAGAGCCCTCAGAGCCTGATCATCCTGGAAGGCAAGAACTGCACCC
TGCAGTGCAACTACACCGTGTCTCCCTTCAGCAACCTGCGGTGGTACAAGCAGGATACAGGC
AGAGGCCCTGTGTCTCTGACCATCATGACCTTCAGCGAGAACACCAAGAGCAACGGCCGGTA
CACCGCCACACTGGATGCCGATACAAAGCAGAGCAGCCTGCACATCACAGCCAGCCAGCTGA
GCGATAGCGCCAGCTACATCTGCGTGGTGTCCGATAGAGGCAGCACCCTGGGCAGACTGTAC
TTTGGCAGAGGCACCCAGCTGACCGTGTGGCCCGATATTCAGAACCCTGATCCTGCCGTGTA
CCAGCTGAGAGACAGCAAGTCCAGCGACAAGAGCGTGTGCCTGTTCACCGACTTCGACAGCC
AGACCAACGTGTCCCAGAGCAAGGACAGCGACGTGTACATCACCGACAAGtgcGTGCTGGAC
ATGCGGAGCATGGACTTCAAGAGCAATAGCGCCGTGGCCTGGTCCAACAAGAGCGATTTCGC
CTGCGCCAACGCCTTCAACAACAGCATTATCCCCGAGGACACATTCTTCCCAAGTCCTGAGA
GCAGCTGCGACGTGAAGCTGGTGGAAAAGAGCTTCGAGACAGACACCAACCTGAACTTCCAG
AACCTGAGCGTGATCGGCTTCCGGATCCTGCTGCTGAAAGTGGCCGGCTTCAACCTGCTGAT
GACCCTGAGACTGTGGTCTAGC
(SEQ ID NO: 54)
MTIRLLCYMGFYFLGAGLMEADIYQTPRYLVIGTGKKITLECSQTMGHDKMYWYQQDPGMEL
HLIHYSYGVNSTEKGDLSSESTVSRIRTEHFPLTLESARPSHTSQYLCASSELRALGPSSYN
SPLHFGNGTRLTVTDLNKVFPPEVAVFEPSEAEISHTQKATLVCLATGFFPDHVELSWWVNG
KEVHSGVCTDPQPLKEQPALNDSRYCLSSRLRVSATFWQNPRNHFRCQVQFYGLSENDEWTQ
DRAKPVTQIVSAEAWGRADCGFTSVSYQQGVLSATILYEILLGKATLYAVLVSALVLMAMVK
RKDF
(SEQ ID NO: 55)
ATGACCATCCGGCTGCTGTGCTATATGGGCTTCTACTTCCTCGGAGCCGGCCTGATGGAAGC
CGACATCTACCAGACACCTAGATACCTGGTCATCGGCACCGGCAAAAAGATCACCCTGGAAT
GCTCCCAGACCATGGGCCACGACAAGATGTACTGGTATCAGCAGGACCCCGGCATGGAACTG
CATCTGATCCACTACAGCTACGGCGTGAACAGCACCGAGAAGGGCGATCTGTCTAGCGAGAG
CACCGTGTCCAGAATCCGGACCGAGCACTTCCCACTGACACTGGAAAGCGCCAGACCTAGCC
ACACCAGCCAGTACCTGTGTGCCTCTAGTGAAGGCGGAGGACTCAAGTTGGCGAAAAACATC
CAATACTTTGGTGCTGGCACCCGGCTTTCTGTCCTGGACCTGAACAAGGTGTTCCCTCCAGA
GGTGGCCGTGTTCGAGCCTTCTGAGGCCGAGATCAGCCACACACAGAAAGCCACACTCGTGT
GTCTGGCCACCGGCTTTTTCCCCGATCACGTGGAACTGTCTTGGTGGGTCAACGGCAAAGAG
GTGCACAGCGGCGTCtGCACAGATCCCCAGCCTCTGAAAGAACAGCCCGCTCTGAACGACAG
CCGGTACTGTCTGAGCAGCAGACTGAGAGTGTCCGCCACCTTCTGGCAGAACCCCAGAAACC
ACTTCAGATGCCAGGTGCAGTTTTACGGCCTGAGCGAGAACGACGAGTGGACCCAGGATAGA
GCCAAGCCTGTGACACAGATCGTGTCTGCCGAAGCCTGGGGCAGAGCCGATTGTGGCTTTAC
CAGCGTGTCATACCAGCAGGGCGTGCTGTCTGCCACCATCCTGTATGAGATTCTGCTGGGCA
AAGCCACTCTGTACGCCGTGCTGGTGTCTGCCCTTGTGCTGATGGCCATGGTCAAGAGAAAG
GACTTC
(SEQ ID NO: 56)
MTIRLLCYMGFYFLGAGLMEADIYQTPRYLVIGTGKKITLECSQTMGHDKMYWYQQDPGMEL
HLIHYSYGVNSTEKGDLSSESTVSRIRTEHFPLTLESARPSHTSQYLCASSEGGGLKLAKNI
QYFGAGTRLSVLDLNKVFPPEVAVFEPSEAEISHTQKATLVCLATGFFPDHVELSWWVNGKE
VHSGVCTDPQPLKEQPALNDSRYCLSSRLRVSATFWQNPRNHFRCQVQFYGLSENDEWTQDR
AKPVTQIVSAEAWGRADCGFTSVSYQQGVLSATILYEILLGKATLYAVLVSALVLMAMVKRK
DF
(SEQ ID NO: 57)
ATGACCATCCGGCTGCTGTGCTATATGGGCTTCTACTTCCTCGGAGCCGGCCTGATGGAAGC
CGACATCTACCAGACACCTAGATACCTGGTCATCGGCACCGGCAAAAAGATCACCCTGGAAT
GCTCCCAGACCATGGGCCACGACAAGATGTACTGGTATCAGCAGGACCCCGGCATGGAACTG
CATCTGATCCACTACAGCTACGGCGTGAACAGCACCGAGAAGGGCGATCTGTCTAGCGAGAG
CACCGTGTCCAGAATCCGGACCGAGCACTTCCCACTGACACTGGAAAGCGCCAGACCTAGCC
ACACCAGCCAGTACCTGTGTGCCTCTAGTGAATTCGCCAGCTCCGTGCGAGGGAATACTATC
TACTTTGGTGAGGGATCTTGGCTTACGGTAGTAGACCTGAACAAGGTGTTCCCTCCAGAGGT
GGCCGTGTTCGAGCCTTCTGAGGCCGAGATCAGCCACACACAGAAAGCCACACTCGTGTGTC
TGGCCACCGGCTTTTTCCCCGATCACGTGGAACTGTCTTGGTGGGTCAACGGCAAAGAGGTG
CACAGCGGCGTCtGCACAGATCCCCAGCCTCTGAAAGAACAGCCCGCTCTGAACGACAGCCG
GTACTGTCTGAGCAGCAGACTGAGAGTGTCCGCCACCTTCTGGCAGAACCCCAGAAACCACT
TCAGATGCCAGGTGCAGTTTTACGGCCTGAGCGAGAACGACGAGTGGACCCAGGATAGAGCC
AAGCCTGTGACACAGATCGTGTCTGCCGAAGCCTGGGGCAGAGCCGATTGTGGCTTTACCAG
CGTGTCATACCAGCAGGGCGTGCTGTCTGCCACCATCCTGTATGAGATTCTGCTGGGCAAAG
CCACTCTGTACGCCGTGCTGGTGTCTGCCCTTGTGCTGATGGCCATGGTCAAGAGAAAGGAC
TTC
(SEQ ID NO: 58)
MTIRLLCYMGFYFLGAGLMEADIYQTPRYLVIGTGKKITLECSQTMGHDKMYWYQQDPGMEL
HLIHYSYGVNSTEKGDLSSESTVSRIRTEHFPLTLESARPSHTSQYLCASSEFASSVRGNTI
YFGEGSWLTVVDLNKVFPPEVAVFEPSEAEISHTQKATLVCLATGFFPDHVELSWWVNGKEV
HSGVCTDPQPLKEQPALNDSRYCLSSRLRVSATFWQNPRNHFRCQVQFYGLSENDEWTQDRA
KPVTQIVSAEAWGRADCGFTSVSYQQGVLSATILYEILLGKATLYAVLVSALVLMAMVKRKD
F

In some embodiments, an NK cell comprises an iTCR complex beta (0) chain iTCR (iTCRβ). In some embodiments, a construct encoding an iTCRβ comprises a polynucleotide coding sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to SEQ ID NO: 59, 61, 63, 65, 67, 69, 71, or 73. In some embodiments, an iTCRβ comprises an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to SEQ ID NO: 60, 62, 64, 66, 68, 70, 72, or 74.

In some embodiments, a construct encoding an iTCRβ comprises a polynucleotide coding sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to SEQ ID NO: 59, 65, 69, or 71. In some embodiments, an iTCRβ comprises an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to SEQ ID NO: 60, 66, 70, or 72.

(SEQ ID NO: 59; iTCRβ clone 3)
ATGACCATCCGGCTGCTGTGCTATATGGGCTTCTACTTCCTCGGAGCCGGCCTGATGGAAGC
CGACATCTACCAGACACCTAGATACCTGGTCATCGGCACCGGCAAAAAGATCACCCTGGAAT
GCTCCCAGACCATGGGCCACGACAAGATGTACTGGTATCAGCAGGACCCCGGCATGGAACTG
CATCTGATCCACTACAGCTACGGCGTGAACAGCACCGAGAAGGGCGATCTGTCTAGCGAGAG
CACCGTGTCCAGAATCCGGACCGAGCACTTCCCACTGACACTGGAAAGCGCCAGACCTAGCC
ACACCAGCCAGTACCTGTGTGCCACTGGACAGGGGGCGCAAGATACGCAGTATTTTGGCCCA
GGCACCCGGCTGACAGTGCTCGAGGACCTCAAGAATGTGTTTCCGCCCGAAGTCGCGGTTTT
TGAACCATCAGAAGCCGAGATCTCTCATACACAAAAGGCGACGCTCGTATGCCTCGCGACGG
GATTTTATCCGGACCACGTCGAGCTTTCCTGGTGGGTTAACGGAAAGGAGGTGCATTCCGGA
GTTTGCACGGACCCTCAGCCATTGAAGGAACAGCCCGCACTGAACGACAGTAGGTATTGCCT
TTCATCTCGCCTGCGCGTGTCTGCGACATTCTGGCAAAACCCAAGAAATCACTTCAGATGTC
AAGTTCAGTTCTACGGTCTCAGCGAGAATGATGAGTGGACACAAGATAGGGCTAAACCCGTG
ACTCAAATAGTCTCTGCCGAGGCCTGGGGGAGGGCGGATTGCGGCTTCACATCAGAATCATA
CCAACAAGGAGTATTGAGCGCGACAATTCTTTACGAAATTCTGCTTGGGAAAGCGACTCTGT
ACGCGGTGCTCGTGTCCGCTTTGGTTCTTATGGCAATGGTTAAACGAAAGGATAGTAGGGGC
(SEQ ID NO: 60; iTCRβ clone 3)
MTIRLLCYMGFYFLGAGLMEADIYQTPRYLVIGTGKKITLECSQTMGHDKMYWYQQDPGMEL
HLIHYSYGVNSTEKGDLSSESTVSRIRTEHFPLTLESARPSHTSQYLCATGQGAQDTQYFGP
GTRLTVLEDLKNVEPPEVAVFEPSEAEISHTQKATLVCLATGFYPDHVELSWWVNGKEVHSG
VCTDPQPLKEQPALNDSRYCLSSRLRVSATFWQNPRNHERCQVQFYGLSENDEWTQDRAKPV
TQIVSAEAWGRADCGFTSESYQQGVLSATILYEILLGKATLYAVLVSALVLMAMVKRKDSRG
(SEQ ID NO: 61; iTCRβ clone 18)
ATGACCATCCGGCTGCTGTGCTATATGGGCTTCTACTTCCTCGGAGCCGGCCTGATGGAAGC
CGACATCTACCAGACACCTAGATACCTGGTCATCGGCACCGGCAAAAAGATCACCCTGGAAT
GCTCCCAGACCATGGGCCACGACAAGATGTACTGGTATCAGCAGGACCCCGGCATGGAACTG
CATCTGATCCACTACAGCTACGGCGTGAACAGCACCGAGAAGGGCGATCTGTCTAGCGAGAG
CACCGTGTCCAGAATCCGGACCGAGCACTTCCCACTGACACTGGAAAGCGCCAGACCTAGCC
ACACCAGCCAGTACCTGTGTGCCAGCAGTGATGGGGTGGGGAGCAATCAGCCCCAGCATTTT
GGTGATGGGACTCGACTCTCCATCCTAGAGGACCTCAATAAGGTGTTTCCGCCCGAAGTCGC
GGTTTTTGAACCATCAGAAGCCGAGATCTCTCATACACAAAAGGCGACGCTCGTATGCCTcG
CGACGGGATTTttcCCGGACCACGTCGAGCTTTCCTGGTGGGTTAAcGGAAAGGAGGTGCAT
TCCGGAGTTTGCACGGACCCTCAGCCATTGAAGGAACAGCCCGCACTGAACGACAGTAGGTA
TTGCCTTTCATCTCGCCTGCGCGTGTCTGCGACATTCTGGCAAAACCCAAGAAATCACTTCA
GATGTCAAGTTCAGTTCTACGGTCTCAGCGAGAATGATGAGTGGACACAAGATAGGGCTAAA
CCCGTGACTCAAATAGTCTCTGCCGAGGCCTGGGGGAGGGCGGATTGCGGCTTCACATCAgt
gTCATACCAACAAGGAGTATTGAGCGCGACAATTCTTTACGAAATTCTGCTTGGGAAAGCGA
CTCTGTACGCGGTGCTCGTGTCCGCTTTGGTTCTTATGGCAATGGTTAAACGAAAGGACTTC
(SEQ ID NO: 62; iTCRβ clone 18)
MTIRLLCYMGFYFLGAGLMEADIYQTPRYLVIGTGKKITLECSQTMGHDKMYWYQQDPGMEL
HLIHYSYGVNSTEKGDLSSESTVSRIRTEHFPLTLESARPSHTSQYLCASSDGVGSNQPQHF
GDGTRLSILEDLNKVFPPEVAVFEPSEAEISHTQKATLVCLATGFFPDHVELSWWVNGKEVH
SGVCTDPQPLKEQPALNDSRYCLSSRLRVSATFWQNPRNHERCQVQFYGLSENDEWTQDRAK
PVTQIVSAEAWGRADCGFTSVSYQQGVLSATILYEILLGKATLYAVLVSALVLMAMVKRKDF
(SEQ ID NO: 63; iTCRβ clone 24)
ATGACCATCCGGCTGCTGTGCTATATGGGCTTCTACTTCCTCGGAGCCGGCCTGATGGAAGC
CGACATCTACCAGACACCTAGATACCTGGTCATCGGCACCGGCAAAAAGATCACCCTGGAAT
GCTCCCAGACCATGGGCCACGACAAGATGTACTGGTATCAGCAGGACCCCGGCATGGAACTG
CATCTGATCCACTACAGCTACGGCGTGAACAGCACCGAGAAGGGCGATCTGTCTAGCGAGAG
CACCGTGTCCAGAATCCGGACCGAGCACTTCCCACTGACACTGGAAAGCGCCAGACCTAGCC
ACACCAGCCAGTACCTGTGTGCCAGCAGTGAGGGGGCTGGAAACACCATATATTTTGGAGAG
GGAAGTTGGCTCACTGTTGTAGAGGACCTCAATAAGGTGTTTCCGCCCGAAGTCGCGGTTTT
TGAACCATCAGAAGCCGAGATCTCTCATACACAAAAGGCGACGCTCGTATGCCTcGCGACGG
GATTTttcCCGGACCACGTCGAGCTTTCCTGGTGGGTTAACGGAAAGGAGGTGCATTCCGGA
GTTTGCACGGACCCTCAGCCATTGAAGGAACAGCCCGCACTGAACGACAGTAGGTATTGCCT
TTCATCTCGCCTGCGCGTGTCTGCGACATTCTGGCAAAACCCAAGAAATCACTTCAGATGTC
AAGTTCAGTTCTACGGTCTCAGCGAGAATGATGAGTGGACACAAGATAGGGCTAAACCCGTG
ACTCAAATAGTCTCTGCCGAGGCCTGGGGGAGGGCGGATTGCGGCTTCACATCAgtgTCATA
CCAACAAGGAGTATTGAGCGCGACAATTCTTTACGAAATTCTGCTTGGGAAAGCGACTCTGT
ACGCGGTGCTCGTGTCCGCTTTGGTTCTTATGGCAATGGTTAAACGAAAGGACTTC
(SEQ ID NO: 64; iTCRβ clone 24)
MTIRLLCYMGFYFLGAGLMEADIYQTPRYLVIGTGKKITLECSQTMGHDKMYWYQQDPGMEL
HLIHYSYGVNSTEKGDLSSESTVSRIRTEHFPLTLESARPSHTSQYLCASSEGAGNTIYFGE
GSWLTVVEDLNKVFPPEVAVFEPSEAEISHTQKATLVCLATGFFPDHVELSWWVNGKEVHSG
VCTDPQPLKEQPALNDSRYCLSSRLRVSATFWQNPRNHERCQVQFYGLSENDEWTQDRAKPV
TQIVSAEAWGRADCGFTSVSYQQGVLSATILYEILLGKATLYAVLVSALVLMAMVKRKDF
(SEQ ID NO: 65; iTCRβ clone 51)
ATGACCATCCGGCTGCTGTGCTATATGGGCTTCTACTTCCTCGGAGCCGGCCTGATGGAAGC
CGACATCTACCAGACACCTAGATACCTGGTCATCGGCACCGGCAAAAAGATCACCCTGGAAT
GCTCCCAGACCATGGGCCACGACAAGATGTACTGGTATCAGCAGGACCCCGGCATGGAACTG
CATCTGATCCACTACAGCTACGGCGTGAACAGCACCGAGAAGGGCGATCTGTCTAGCGAGAG
CACCGTGTCCAGAATCCGGACCGAGCACTTCCCACTGACACTGGAAAGCGCCAGACCTAGCC
ACACCAGCCAGTACCTGTGTGCCAGCAGTGACAGGGATAGCAATCAGCCCCAGCATTTTGGT
GATGGGACTCGACTCTCCATCCTAGAGGACCTCAATAAGGTGTTTCCGCCCGAAGTCGCGGT
TTTTGAACCATCAGAAGCCGAGATCTCTCATACACAAAAGGCGACGCTCGTATGCCTCGCGA
CGGGATTTttcCCGGACCACGTCGAGCTTTCCTGGTGGGTTAACGGAAAGGAGGTGCATTCC
GGAGTTTGCACGGACCCTCAGCCATTGAAGGAACAGCCCGCACTGAACGACAGTAGGTATTG
CCTTTCATCTCGCCTGCGCGTGTCTGCGACATTCTGGCAAAACCCAAGAAATCACTTCAGAT
GTCAAGTTCAGTTCTACGGTCTCAGCGAGAATGATGAGTGGACACAAGATAGGGCTAAACCC
GTGACTCAAATAGTCTCTGCCGAGGCCTGGGGGAGGGCGGATTGCGGCTTCACATCAgtgTC
ATACCAACAAGGAGTATTGAGCGCGACAATTCTTTACGAAATTCTGCTTGGGAAAGCGACTC
TGTACGCGGTGCTCGTGTCCGCTTTGGTTCTTATGGCAATGGTTAAACGAAAGGACTTC
(SEQ ID NO: 66; iTCRβ clone 51)
MTIRLLCYMGFYFLGAGLMEADIYQTPRYLVIGTGKKITLECSQTMGHDKMYWYQQDPGMEL
HLIHYSYGVNSTEKGDLSSESTVSRIRTEHFPLTLESARPSHTSQYLCASSDRDSNQPQHFG
DGTRLSILEDLNKVFPPEVAVFEPSEAEISHTQKATLVCLATGFFPDHVELSWWVNGKEVHS
GVCTDPQPLKEQPALNDSRYCLSSRLRVSATFWQNPRNHFRCQVQFYGLSENDEWTQDRAKP
VTQIVSAEAWGRADCGFTSVSYQQGVLSATILYEILLGKATLYAVLVSALVLMAMVKRKDF
(SEQ ID NO: 67; iTCRβ clone 56)
ATGACCATCCGGCTGCTGTGCTATATGGGCTTCTACTTCCTCGGAGCCGGCCTGATGGAAGC
CGACATCTACCAGACACCTAGATACCTGGTCATCGGCACCGGCAAAAAGATCACCCTGGAAT
GCTCCCAGACCATGGGCCACGACAAGATGTACTGGTATCAGCAGGACCCCGGCATGGAACTG
CATCTGATCCACTACAGCTACGGCGTGAACAGCACCGAGAAGGGCGATCTGTCTAGCGAGAG
CACCGTGTCCAGAATCCGGACCGAGCACTTCCCACTGACACTGGAAAGCGCCAGACCTAGCC
ACACCAGCCAGTACCTGTGTGCCAGCGTGGGTCCGGTACCCTCCTACAATGAGCAGTTCTTC
GGGCCAGGGACACGGCTCACCGTGCTAGAGGACCTCAAGAATGTGTTTCCGCCCGAAGTCGC
GGTTTTTGAACCATCAGAAGCCGAGATCTCTCATACACAAAAGGCGACGCTCGTATGCCTCG
CGACGGGATTTTATCCGGACCACGTCGAGCTTTCCTGGTGGGTTAACGGAAAGGAGGTGCAT
TCCGGAGTTTGCACGGACCCTCAGCCATTGAAGGAACAGCCCGCACTGAACGACAGTAGGTA
TTGCCTTTCATCTCGCCTGCGCGTGTCTGCGACATTCTGGCAAAACCCAAGAAATCACTTCA
GATGTCAAGTTCAGTTCTACGGTCTCAGCGAGAATGATGAGTGGACACAAGATAGGGCTAAA
CCCGTGACTCAAATAGTCTCTGCCGAGGCCTGGGGGAGGGCGGATTGCGGCTTCACATCAGA
ATCATACCAACAAGGAGTATTGAGCGCGACAATTCTTTACGAAATTCTGCTTGGGAAAGCGA
CTCTGTACGCGGTGCTCGTGTCCGCTTTGGTTCTTATGGCAATGGTTAAACGAAAGGATAGT
AGGGGC
(SEQ ID NO: 68; iTCRβ clone 56)
MTIRLLCYMGFYFLGAGLMEADIYQTPRYLVIGTGKKITLECSQTMGHDKMYWYQQDPGMEL
HLIHYSYGVNSTEKGDLSSESTVSRIRTEHFPLTLESARPSHTSQYLCASVGPVPSYNEQFF
GPGTRLTVLEDLKNVFPPEVAVFEPSEAEISHTQKATLVCLATGFYPDHVELSWWVNGKEVH
SGVCTDPQPLKEQPALNDSRYCLSSRLRVSATFWQNPRNHFRCQVQFYGLSENDEWTQDRAK
PVTQIVSAEAWGRADCGFTSESYQQGVLSATILYEILLGKATLYAVLVSALVLMAMVKRKDS
RG
(SEQ ID NO: 69; iTCRβ clone 76)
ATGACCATCCGGCTGCTGTGCTATATGGGCTTCTACTTCCTCGGAGCCGGCCTGATGGAAGC
CGACATCTACCAGACACCTAGATACCTGGTCATCGGCACCGGCAAAAAGATCACCCTGGAAT
GCTCCCAGACCATGGGCCACGACAAGATGTACTGGTATCAGCAGGACCCCGGCATGGAACTG
CATCTGATCCACTACAGCTACGGCGTGAACAGCACCGAGAAGGGCGATCTGTCTAGCGAGAG
CACCGTGTCCAGAATCCGGACCGAGCACTTCCCACTGACACTGGAAAGCGCCAGACCTAGCC
ACACCAGCCAGTACCTGTGTGCCAGCAGTGGGGTGACTAGCGCCTCCTACAATGAGCAGTTC
TTCGGGCCAGGGACACGGCTCACCGTGCTAGAGGACCTCAAGAATGTGTTTCCGCCCGAAGT
CGCGGTTTTTGAACCATCAGAAGCCGAGATCTCTCATACACAAAAGGCGACGCTCGTATGCC
TcGCGACGGGATTTTATCCGGACCACGTCGAGCTTTCCTGGTGGGTTAAcGGAAAGGAGGTG
CATTCCGGAGTTTGCACGGACCCTCAGCCATTGAAGGAACAGCCCGCACTGAACGACAGTAG
GTATTGCCTTTCATCTCGCCTGCGCGTGTCTGCGACATTCTGGCAAAACCCAAGAAATCACT
TCAGATGTCAAGTTCAGTTCTACGGTCTCAGCGAGAATGATGAGTGGACACAAGATAGGGCT
AAACCCGTGACTCAAATAGTCTCTGCCGAGGCCTGGGGGAGGGCGGATTGCGGCTTCACATC
AGAATCATACCAACAAGGAGTATTGAGCGCGACAATTCTTTACGAAATTCTGCTTGGGAAAG
CGACTCTGTACGCGGTGCTCGTGTCCGCTTTGGTTCTTATGGCAATGGTTAAACGAAAGGAT
AGTAGGGGC
(SEQ ID NO: 70; iTCRβ clone 76)
MTIRLLCYMGFYFLGAGLMEADIYQTPRYLVIGTGKKITLECSQTMGHDKMYWYQQDPGMEL
HLIHYSYGVNSTEKGDLSSESTVSRIRTEHFPLTLESARPSHTSQYLCASSGVTSASYNEQF
FGPGTRLTVLEDLKNVFPPEVAVFEPSEAEISHTQKATLVCLATGFYPDHVELSWWVNGKEV
HSGVCTDPQPLKEQPALNDSRYCLSSRLRVSATFWQNPRNHERCQVQFYGLSENDEWTQDRA
KPVTQIVSAEAWGRADCGFTSESYQQGVLSATILYEILLGKATLYAVLVSALVLMAMVKRKD
SRG
(SEQ ID NO: 71; iTCRβ clone 93)
ATGACCATCCGGCTGCTGTGCTATATGGGCTTCTACTTCCTCGGAGCCGGCCTGATGGAAGC
CGACATCTACCAGACACCTAGATACCTGGTCATCGGCACCGGCAAAAAGATCACCCTGGAAT
GCTCCCAGACCATGGGCCACGACAAGATGTACTGGTATCAGCAGGACCCCGGCATGGAACTG
CATCTGATCCACTACAGCTACGGCGTGAACAGCACCGAGAAGGGCGATCTGTCTAGCGAGAG
CACCGTGTCCAGAATCCGGACCGAGCACTTCCCACTGACACTGGAAAGCGCCAGACCTAGCC
ACACCAGCCAGTACCTGTGTGCCAGCAGTGGAGGAGGGGAGGAGACCCAGTACTTCGGGCCA
GGCACGCGGCTCCTGGTGCTCGAGGACCTCAAGAATGTGTTTCCGCCCGAAGTCGCGGTTTT
TGAACCATCAGAAGCCGAGATCTCTCATACACAAAAGGCGACGCTCGTATGCCTcGCGACGG
GATTTTATCCGGACCACGTCGAGCTTTCCTGGTGGGTTAAcGGAAAGGAGGTGCATTCCGGA
GTTTGCACGGACCCTCAGCCATTGAAGGAACAGCCCGCACTGAACGACAGTAGGTATTGCCT
TTCATCTCGCCTGCGCGTGTCTGCGACATTCTGGCAAAACCCAAGAAATCACTTCAGATGTC
AAGTTCAGTTCTACGGTCTCAGCGAGAATGATGAGTGGACACAAGATAGGGCTAAACCCGTG
ACTCAAATAGTCTCTGCCGAGGCCTGGGGGAGGGCGGATTGCGGCTTCACATCAGAATCATA
CCAACAAGGAGTATTGAGCGCGACAATTCTTTACGAAATTCTGCTTGGGAAAGCGACTCTGT
ACGCGGTGCTCGTGTCCGCTTTGGTTCTTATGGCAATGGTTAAACGAAAGGATAGTAGGGGC
(SEQ ID NO: 72; iTCRβ clone 93)
MTIRLLCYMGFYFLGAGLMEADIYQTPRYLVIGTGKKITLECSQTMGHDKMYWYQQDPGMEL
HLIHYSYGVNSTEKGDLSSESTVSRIRTEHFPLTLESARPSHTSQYLCASSGGGEETQYFGP
GTRLLVLEDLKNVFPPEVAVFEPSEAEISHTQKATLVCLATGFYPDHVELSWWVNGKEVHSG
VCTDPQPLKEQPALNDSRYCLSSRLRVSATFWQNPRNHFRCQVQFYGLSENDEWTQDRAKPV
TQIVSAEAWGRADCGFTSESYQQGVLSATILYEILLGKATLYAVLVSALVLMAMVKRKDSRG
(SEQ ID NO: 73; iTCRβ clone 96)
ATGACCATCCGGCTGCTGTGCTATATGGGCTTCTACTTCCTCGGAGCCGGCCTGATGGAAGC
CGACATCTACCAGACACCTAGATACCTGGTCATCGGCACCGGCAAAAAGATCACCCTGGAAT
GCTCCCAGACCATGGGCCACGACAAGATGTACTGGTATCAGCAGGACCCCGGCATGGAACTG
CATCTGATCCACTACAGCTACGGCGTGAACAGCACCGAGAAGGGCGATCTGTCTAGCGAGAG
CACCGTGTCCAGAATCCGGACCGAGCACTTCCCACTGACACTGGAAAGCGCCAGACCTAGCC
ACACCAGCCAGTACCTGTGTGCCAGCAGTGCGCAGGGGGTCAGCGAAAAACTGTTTTTTGGC
AGTGGAACCCAGCTCTCTGTCTTGGAGGACCTCAATAAGGTGTTTCCGCCCGAAGTCGCGGT
TTTTGAACCATCAGAAGCCGAGATCTCTCATACACAAAAGGCGACGCTCGTATGCCTCGCGA
CGGGATTTttcCCGGACCACGTCGAGCTTTCCTGGTGGGTTAACGGAAAGGAGGTGCATTCC
GGAGTTTGCACGGACCCTCAGCCATTGAAGGAACAGCCCGCACTGAACGACAGTAGGTATTG
CCTTTCATCTCGCCTGCGCGTGTCTGCGACATTCTGGCAAAACCCAAGAAATCACTTCAGAT
GTCAAGTTCAGTTCTACGGTCTCAGCGAGAATGATGAGTGGACACAAGATAGGGCTAAACCC
GTGACTCAAATAGTCTCTGCCGAGGCCTGGGGGAGGGCGGATTGCGGCTTCACATCAgtgTC
ATACCAACAAGGAGTATTGAGCGCGACAATTCTTTACGAAATTCTGCTTGGGAAAGCGACTC
TGTACGCGGTGCTCGTGTCCGCTTTGGTTCTTATGGCAATGGTTAAACGAAAGGACTTC
(SEQ ID NO: 74; iTCRβ clone 96)
MTIRLLCYMGFYFLGAGLMEADIYQTPRYLVIGTGKKITLECSQTMGHDKMYWYQQDPGMEL
HLIHYSYGVNSTEKGDLSSESTVSRIRTEHFPLTLESARPSHTSQYLCASSAQGVSEKLFFG
SGTQLSVLEDLNKVFPPEVAVFEPSEAEISHTQKATLVCLATGFFPDHVELSWWVNGKEVHS
GVCTDPQPLKEQPALNDSRYCLSSRLRVSATFWQNPRNHFRCQVQFYGLSENDEWTQDRAKP
VTQIVSAEAWGRADCGFTSVSYQQGVLSATILYEILLGKATLYAVLVSALVLMAMVKRKDF

In some embodiments, a construct encoding an iTCRβ chain comprises an iTCRβ VP-DJ region comprising a polynucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to SEQ ID NOs: 75-149.

(SEQ ID NO: 75; iTCRβ clone 3)
ACTGGACAGGGGGCGCAAGATACGCAGTATTTTGGCCCAGGCACCCGGCTGACAGTGCTCGA
G
(SEQ ID NO: 76; iTCRβ clone 4)
AGCAGTGCCCCTGGAGGGTCTGAAGCTTTCTTTGGACAAGGCACCAGACTCACAGTTGTAGA
G
(SEQ ID NO: 77; iTCRβ clone 7)
AGCAGTGAACTCGACAGGGAAGGAAACACCATATATTTTGGAGAGGGAAGTTGGCTCACTGT
TGTAGAG
(SEQ ID NO: 78; iTCRβ clone 9)
AGCAGTGATATGGGACCCGTCTCCTACGAGCAGTACTTCGGGCCGGGCACCAGGCTCACGGT
CACAGAG
(SEQ ID NO: 79; iTCRβ clone 13)
AGCAGTGAGGAAGAGCGGGGCCCCTACGAGCAGTACTTCGGGCCGGGCACCAGGCTCACGGT
CACAGAG
(SEQ ID NO: 80; iTCRβ clone 18)
AGCAGTGATGGGGTGGGGAGCAATCAGCCCCAGCATTTTGGTGATGGGACTCGACTCTCCAT
CCTAGAG
(SEQ ID NO: 81; iTCRβ clone 20)
AGCAGTGACCCCTACGAGCAGTACTTCGGGCCGGGCACCAGGCTCACGGTCACAGAG
(SEQ ID NO: 82; iTCRβ clone 21)
AGCAGTGAGGCCCCAACAGGAACCGGGGCCAACGTCCTGACTTTCGGGGCCGGCAGCAGGCT
GACCGTGCTGGAG
(SEQ ID NO: 83; iTCRβ clone 23)
AGCAGTGATCCTCGACTAGCGGGGGGGGTCGAGCAGTACTTCGGGCCGGGCACCAGGCTCAC
GGTCACAGAG
(SEQ ID NO: 84; iTCRβ clone 24)
AGCAGTGAGGGGGCTGGAAACACCATATATTTTGGAGAGGGAAGTTGGCTCACTGTTGTAGA
G
(SEQ ID NO: 85; iTCRβ clone 26)
AGCAGTTATGCTACAGGCTACAATGAGCAGTTCTTCGGGCCAGGGACACGGCTCACCGTGCT
AGAG
(SEQ ID NO: 86; iTCRβ clone 28)
AGCAGTGAACGGCAGGGCTCCACAGATACGCAGTATTTTGGCCCAGGCACCCGGCTGACAGT
GCTCGAG
(SEQ ID NO: 87; iTCRβ clone 29)
AGCAGTGCTCCGACTAGCGGGAGGGACAATGAGCAGTTCTTCGGGCCAGGGACACGGCTCAC
CGTGCTAGAG
(SEQ ID NO: 88; iTCRβ clone 30)
AGCAGTGAATGGACTAGCGGGGGGCCCAACACCGGGGAGCTGTTTTTTGGAGAAGGCTCTAG
GCTGACCGTACTGGAG
(SEQ ID NO: 89; iTCRβ clone 32)
AGCAGTGAACGGGGGGCTAGCACAGATACGCAGTATTTTGGCCCAGGCACCCGGCTGACAGT
GCTCGAG
(SEQ ID NO: 90; iTCRβ clone 33)
AGCAGTGAAGGGCTAGCGGGAGAACCTCTCTTAGGCAATGAGCAGTTCTTCGGGCCAGGGAC
ACGGCTCACCGTGCTAGAG
(SEQ ID NO: 91; iTCRβ clone 34)
AGCAGTGAGGCAGGCGGCCACACAGATACGCAGTATTTTGGCCCAGGCACCCGGCTGACAGT
GCTCGAG
(SEQ ID NO: 92; iTCRβ clone 36)
AGCAGTGAATACCAAGAGACCCAGTACTTCGGGCCAGGCACGCGGCTCCTGGTGCTCGAG
(SEQ ID NO: 93; iTCRβ clone 37)
AGCACCGACAGGGGATCTTTCGGGGAGCTGTTTTTTGGAGAAGGCTCTAGGCTGACCGTACT
GGAG
(SEQ ID NO: 94; iTCRβ clone 38)
GGAGGAGGGACATCTCAAGAGACCCAGTACTTCGGGCCAGGCACGCGGCTCCTGGTGCTCGA
G
(SEQ ID NO: 95; iTCRβ clone 39)
AGCAGTCCGACTAGCGGGATGGGGGAGACCCAGTACTTCGGGCCAGGCACGCGGCTCCTGGT
GCTCGAG
(SEQ ID NO: 96; iTCRβ clone 41)
AGCAGTGAGTTCGGGGCCAACGTCCTGACTTTCGGGGCCGGCAGCAGGCTGACCGTGCTGGA
G
(SEQ ID NO: 97; iTCRβ clone 42)
AGCAGTGTCCGTAGCGGGAGAGGGGACAATGAGCAGTTCTTCGGGCCAGGGACACGGCTCAC
CGTGCTAGAG
(SEQ ID NO: 98; iTCRβ clone 43)
AGCAGTGTCCAGGAGGAAGATACGCAGTATTTTGGCCCAGGCACCCGGCTGACAGTGCTCGA
G
(SEQ ID NO: 99; iTCRβ clone 44)
AGCAGTGATAGTAGCTACGAGCAGTACTTCGGGCCGGGCACCAGGCTCACGGTCACAGAG
(SEQ ID NO: 100; iTCRβ clone 46)
AGCAGTGGTACTACGGGACAGGAATCCTACGAGCAGTACTTCGGGCCGGGCACCAGGCTCAC
GGTCACAGAG
(SEQ ID NO: 101; iTCRβ clone 47)
AGCAGTGTAAGGGGGAACCACAATGAGCAGTTCTTCGGGCCAGGGACACGGCTCACCGTGCT
AGAG
(SEQ ID NO: 102; iTCRβ clone 48)
AGCAGTGAACTTCAGCGGGAGGGTTCTCCAGATACGCAGTATTTTGGCCCAGGCACCCGGCT
GACAGTGCTCGAG
(SEQ ID NO: 103; iTCRβ clone 49)
AGCAGTGTCCGGGACAGGGATGAAAAACTGTTTTTTGGCAGTGGAACCCAGCTCTCTGTCTT
GGAG
(SEQ ID NO: 104; iTCRβ clone 50)
AGCAGTGAGGGTCAGGGAGGTTACGAGCAGTACTTCGGGCCGGGCACCAGGCTCACGGTCAC
AGAG
(SEQ ID NO: 105; iTCRβ clone 51)
AGCAGTGACAGGGATAGCAATCAGCCCCAGCATTTTGGTGATGGGACTCGACTCTCCATCCT
AGAG
(SEQ ID NO: 106; iTCRβ clone 52)
AGCAGTGATCGGTCTAGCGGAGCCAAAAACATTCAGTACTTCGGCGCCGGGACCCGGCTCTC
AGTGCTGGAG
(SEQ ID NO: 107; iTCRβ clone 53)
AGCAGTGCCACGACTAGCGGGAGGACAGATACGCAGTATTTTGGCCCAGGCACCCGGCTGAC
AGTGCTCGAG
(SEQ ID NO: 108; iTCRβ clone 54)
AGCAGTGAATTTCGGCAGCGGGAGTCCTACGAGCAGTACTTCGGGCCGGGCACCAGGCTCAC
GGTCACAGAG
(SEQ ID NO: 109; iTCRβ clone 55)
AGCAGTGAAATAGCGGGAGTGGCCTACGAGCAGTACTTCGGGCCGGGCACCAGGCTCACGGT
CACAGAG
(SEQ ID NO: 110; iTCRβ clone 56)
AGCGTGGGTCCGGTACCCTCCTACAATGAGCAGTTCTTCGGGCCAGGGACACGGCTCACCGT
GCTAGAG
(SEQ ID NO: 111; iTCRβ clone 57)
AGCAGTGAACGGCGCGGGAGACGGGATACGCAGTATTTTGGCCCAGGCACCCGGCTGACAGT
GCTCGAG
(SEQ ID NO: 112; iTCRβ clone 58)
AGCAGTGGGACAGGGTACGAGCAGTACTTCGGGCCGGGCACCAGGCTCACGGTCACAGAG
(SEQ ID NO: 113; iTCRβ clone 59)
AGCAGTGACCGTAGCGGGAGCTCCTACGAGCAGTACTTCGGGCCGGGCACCAGGCTCACGGT
CACAGAG
(SEQ ID NO: 114; iTCRβ clone 60)
AGCAGTGACAGCACCGGGGAGCTGTTTTTTGGAGAAGGCTCTAGGCTGACCGTACTGGAG
(SEQ ID NO: 115; iTCRβ clone 61)
AGCAGTGCTAGCGGGAGCAATGAGCAGTTCTTCGGGCCAGGGACACGGCTCACCGTGCTAGA
G
(SEQ ID NO: 116; iTCRβ clone 62)
AGCAGTGACGGGACTAGCGGCTACGAGCAGTACTTCGGGCCGGGCACCAGGCTCACGGTCAC
AGAG
(SEQ ID NO: 117; iTCRβ clone 63)
AGCAGTGAATATGAAAAACTGTTTTTTGGCAGTGGAACCCAGCTCTCTGTCTTGGAG
(SEQ ID NO: 118; iTCRβ clone 64)
AGCAGTGAGTCCGGCCCCCGCAATGAGCAGTTCTTCGGGCCAGGGACACGGCTCACCGTGCT
AGAG
(SEQ ID NO: 119; iTCRβ clone 66)
AGCAGTGGCCGACTAGCGGGAGAGGAAGAGACCCAGTACTTCGGGCCAGGCACGCGGCTCCT
GGTGCTCGAG
(SEQ ID NO: 120; iTCRβ clone 67)
AGCAGTGAGGGTGGCAGGGTCGATGAGCAGTTCTTCGGGCCAGGGACACGGCTCACCGTGCT
AGAG
(SEQ ID NO: 121; iTCRβ clone 68)
AGCAGTGAGGCTAACTATGGCTACACCTTCGGTTCGGGGACCAGGTTAACCGTTGTAGAG
(SEQ ID NO: 122; iTCRβ clone 69)
AGCAGTCAGGACGGATTGGGATATGGCTACACCTTCGGTTCGGGGACCAGGTTAACCGTTGT
AGAG
(SEQ ID NO: 123; iTCRβ clone 70)
AGCAGTGGGCGCCTCCACGAGCAGTACTTCGGGCCGGGCACCAGGCTCACGGTCACAGAG
(SEQ ID NO: 124; iTCRβ clone 71)
AGCAGTGAATATAACAGCACAGATACGCAGTATTTTGGCCCAGGCACCCGGCTGACAGTGCT
CGAG
(SEQ ID NO: 125; iTCRβ clone 72)
AGCAGTGAACCCGGATTGGGGGAGCTGTTTTTTGGAGAAGGCTCTAGGCTGACCGTACTGGA
G
(SEQ ID NO: 126; iTCRβ clone 73)
AGCATCCTGGGAGAGGGGCGGAATGAGCAGTTCTTCGGGCCAGGGACACGGCTCACCGTGCT
AGAG
(SEQ ID NO: 127; iTCRβ clone 74)
AGCAGTGCCCCGGGACAGATCTATGGCTACACCTTCGGTTCGGGGACCAGGTTAACCGTTGT
AGAG
(SEQ ID NO: 128; iTCRβ clone 75)
AGCAGTGACAACCAAGAGACCCAGTACTTCGGGCCAGGCACGCGGCTCCTGGTGCTCGAG
(SEQ ID NO: 129; iTCRβ clone 76)
AGCAGTGGGGTGACTAGCGCCTCCTACAATGAGCAGTTCTTCGGGCCAGGGACACGGCTCAC
CGTGCTAGAG
(SEQ ID NO: 130; iTCRβ clone 77)
AGCAGTCCTGAGCCCACCACCCTAGCGGGAGTCCACGAGCAGTACTTCGGGCCGGGCACCAG
GCTCACGGTCACAGAG
(SEQ ID NO: 131; iTCRβ clone 78)
AGCAGTGGGACACAGAGGGCTGAAAAACTGTTTTTTGGCAGTGGAACCCAGCTCTCTGTCTT
GGAG
(SEQ ID NO: 132; iTCRβ clone 79)
AGCAGTGGGACTAGCGGGAGCACAGATACGCAGTATTTTGGCCCAGGCACCCGGCTGACAGT
GCTCGAG
(SEQ ID NO: 133; iTCRβ clone 80)
AGCAGTGAGGCGGGACAGGGTTCCTACGAGCAGTACTTCGGGCCGGGCACCAGGCTCACGGT
CACAGAG
(SEQ ID NO: 134; iTCRβ clone 83)
AGCACCTCTAGCCGCACCGGGGAGCTGTTTTTTGGAGAAGGCTCTAGGCTGACCGTACTGGA
G
(SEQ ID NO: 135; iTCRβ clone 84)
AGCAGTGAACCGGGGGAGCGGAATGAGCAGTTCTTCGGGCCAGGGACACGGCTCACCGTGCT
AGAG
(SEQ ID NO: 136; iTCRβ clone 85)
AGCAGTGAAGGTCGGGTTAACTATGGCTACACCTTCGGTTCGGGGACCAGGTTAACCGTTGT
AGAG
(SEQ ID NO: 137; iTCRβ clone 86)
AGCAGTGAATCAGAAGGGGGCACAGATACGCAGTATTTTGGCCCAGGCACCCGGCTGACAGT
GCTCGAG
(SEQ ID NO: 138; iTCRβ clone 87)
AGCAGTCCCGGGGGGACTAGCGGGAGGGCACGTCCCTACGAGCAGTACTTCGGGCCGGGCAC
CAGGCTCACGGTCACAGAG
(SEQ ID NO: 139; iTCRβ clone 88)
AGCAGTGGGAGGGAGGGGGACCCCTACGAGCAGTACTTCGGGCCGGGCACCAGGCTCACGGT
CACAGAG
(SEQ ID NO: 140; iTCRβ clone 89)
AGCAGTGGACTAGCGAACACCGGGGAGCTGTTTTTTGGAGAAGGCTCTAGGCTGACCGTACT
GGAG
(SEQ ID NO: 141; iTCRβ clone 90)
AGCAGTGGGACGACAGGGGATACACGCACAGATACGCAGTATTTTGGCCCAGGCACCCGGCT
GACAGTGCTCGAG
(SEQ ID NO: 142; iTCRβ clone 91)
AGCAGTGAAGACCGGGACAGGGGTCACGAGCAGTACTTCGGGCCGGGCACCAGGCTCACGGT
CACAGAG
(SEQ ID NO: 143; iTCRβ clone 92)
AGCAGTGAACTAGCGAATGAGCAGTTCTTCGGGCCAGGGACACGGCTCACCGTGCTAGAG
(SEQ ID NO: 144; iTCRβ clone 93)
AGCAGTGGAGGAGGGGAGGAGACCCAGTACTTCGGGCCAGGCACGCGGCTCCTGGTGCTCGA
G
(SEQ ID NO: 145; iTCRβ clone 94)
AGCAGTGAATATGCAGGGTGGGGCGGCAATCAGCCCCAGCATTTTGGTGATGGGACTCGACT
CTCCATCCTAGAG
(SEQ ID NO: 146; iTCRβ clone 95)
AGCAGTGAATTGGACGGGACTAGCGCCTACGAGCAGTACTTCGGGCCGGGCACCAGGCTCAC
GGTCACAGAG
(SEQ ID NO: 147; iTCRβ clone 96)
AGCAGTGCGCAGGGGGTCAGCGAAAAACTGTTTTTTGGCAGTGGAACCCAGCTCTCTGTCTT
GGAG
(SEQ ID NO: 148; iTCRβ clone 97)
AGCAGTGAAGTGGCGGGAGCGGACACCGGGGAGCTGTTTTTTGGAGAAGGCTCTAGGCTGAC
CGTACTGGAG
(SEQ ID NO: 149; iTCRβ clone 98)
AGCAGCGGCAGGGGGCCAGGGGAAAGTGCAGATACGCAGTATTTTGGCCCAGGCACCCGGCT
GACAGTGCTCGAG

### B. NK Cell Fc Binding Modifications

As described herein, engineered NK cells can be modified to express a transgenic construct (heterologous construct) encoding a polypeptide comprising an Fc Receptor extracellular Fc binding domain (e.g., an Fc binding domain). The Fc binding domain can be tethered to a cell membrane, such as through a transmembrane domain (TMD). The Fc binding domain is comprised as part of, or obtained from, an Fc Receptor. An Fc receptor can be an Fcγ receptor, e.g., FcγRI (CD64); FcγRIIA, IIB, and IIC (CD32); FcγRIIIA and/or IIIB (CD16). In certain embodiments, an Fcγ receptor is FcγRIII. In certain embodiments, an FcγRIII is FcγRIIIA, which can be a high affinity variant CD16 (CD16 ha; e.g., F158V). In some embodiments, a cell expresses more than one transgenic Fc binding domain comprising polypeptides.

As described herein, a TCR/FcR construct comprises a coding sequence for an Fc Receptor extracellular binding domain. In exemplary embodiments described herein, a TCR/FcR construct can comprise chimeric polypeptides comprising an Fc binding domain derived from CD16 (e.g., FcγRIII). In some embodiments, an extracellular Fc binding domain derived from CD16 is a high affinity polymorph comprising an F158V substitution. In some embodiments, a CD16 derived Fc binding domain comprising polypeptide comprises substantially no intracellular signaling domain (ICD). In some embodiments, a CD16 extracellular domain (e.g., Fc binding domain) comprising polypeptide comprises an optional heterologous hinge, a heterologous or CD16 derived TMD, and/or a heterologous and/or CD16 derived ICD. In certain embodiments, a CD16 derived Fc binding domain does not comprise mutations which render the binding domain uncleavable and/or refractory to cleavage.

In some embodiments, a polynucleotide encoding a transgenic Fc binding domain comprises a signal peptide. In some embodiments, a signal peptide is a signal peptide that can be found associated with the endogenous Fc Receptor that comprises the noted Fc binding domain. In some embodiments, a signal peptide is a heterologous signal peptide that is not naturally associated with an Fc binding protein.

In some embodiments, a TCR/FcR construct comprises an Fe Receptor extracellular binding domain polypeptide that is encoded by a polynucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 150-154. In certain embodiments, a TCR/FcR construct comprises an Fc Receptor extracellular binding domain polypeptide that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 155-159. In some embodiments, in sequences described herein, underlining refers to signal peptide sequence, which in some embodiments may be modified, omitted, and/or replaced with an alternative signal peptide.

As described herein, in some embodiments a TCR/FcR construct comprises a chimeric Fc Receptor extracellular binding domain polypeptide (e.g., that comprises one or more sequences derived from a non-Fc Receptor polypeptide and/or a different Fc Receptor polypeptide). In some embodiments, a TCR/FcR construct comprises a human CD32 derived hinge region linking an FC Receptor extracellular domain (ECD) sequence and a transmembrane domain (TMD) sequence. In some embodiments, a TCR/FcR construct comprises a CD3ζ derived TMD sequence and/or intracellular domain (ICD) sequence. In some embodiments, a human CD32 derived hinge region is encoded by a polynucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 160. In certain embodiments, a human CD32 derived hinge region comprises a polypeptide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 161. In some embodiments, a CD3ζ derived TMD region is encoded by a polynucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 162. In certain embodiments, a human CD3ζ derived TMD region comprises a polypeptide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 163. In some embodiments, a CD3ζ derived ICD region is encoded by a polynucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 164. In certain embodiments, a human CD3ζ derived ICD region comprises a polypeptide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 165. In some embodiments, a TCR/FcR construct comprises a CD16 derived TMD sequence and/or ICD sequence. In some embodiments, a human CD16 derived TMD region is encoded by a polynucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 166. In certain embodiments, a human CD16 derived TMD region comprises a polypeptide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 167. In some embodiments, a human CD16 derived ICD region is encoded by a polynucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 168. In certain embodiments, a human CD16 derived ICD region comprises a polypeptide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 169.

TCR/FcR #1 CD16 extracellular domain (Fc binding domain) comprising polypeptide:
WT CD16, polynucleotide sequence (SEQ ID NO: 150)
(SEQ ID NO: 150)
ATGTGGCAGCTGCTCCTCCCAACTGCTCTGCTACTTCTAGTTTCAGCTGGCATGCGGACTGA
AGATCTCCCAAAGGCTGTGGTGTTCCTGGAGCCTCAATGGTACAGGGTGCTCGAGAAGGACA
GTGTGACTCTGAAGTGCCAGGGAGCCTACTCCCCTGAGGACAATTCCACACAGTGGTTTCAC
AATGAGAGCCTCATCTCAAGCCAGGCCTCGAGCTACTTCATTGACGCTGCCACAGTCGACGA
CAGTGGAGAGTACAGGTGCCAGACAAACCTCTCCACCCTCAGTGACCCGGTGCAGCTAGAAG
TCCATATCGGCTGGCTGTTGCTCCAGGCCCCTCGGTGGGTGTTCAAGGAGGAAGACCCTATT
CACCTGAGGTGTCACAGCTGGAAGAACACTGCTCTGCATAAGGTCACATATTTACAGAATGG
CAAAGGCAGGAAGTATTTTCATCATAATTCTGACTTCTACATTCCAAAAGCCACACTCAAAG
ACAGCGGCTCCTACTTCTGCAGGGGGCTTTTTGGGAGTAAAAATGTGTCTTCAGAGACTGTG
AACATCACCATCACTCAAGGTTTGGCAGTGTCAACCATCTCATCATTCTTTCCACCTGGGTA
CCAAgtctctttctgcttggtgatggtactcctttttgcagtggacacaggactatatttct
ctgtgaagacaaacattcgaagctcaacaagagactggaaggaccataaatttaaatggaga
aaggaccctcaagacaaaTAA
TCR/FcR #2 CD16 extracellular domain (Fc binding domain) comprising polypeptide:
WT high affinity CD16, polynucleotide sequence (CD16ha, F158V)(SEQ ID NO: 151)
(SEQ ID NO: 151)
ATGTGGCAGCTGCTCCTCCCAACTGCTCTGCTACTTCTAGTTTCAGCTGGCATGCGGACTGA
AGATCTCCCAAAGGCTGTGGTGTTCCTGGAGCCTCAATGGTACAGGGTGCTCGAGAAGGACA
GTGTGACTCTGAAGTGCCAGGGAGCCTACTCCCCTGAGGACAATTCCACACAGTGGTTTCAC
AATGAGAGCCTCATCTCAAGCCAGGCCTCGAGCTACTTCATTGACGCTGCCACAGTCGACGA
CAGTGGAGAGTACAGGTGCCAGACAAACCTCTCCACCCTCAGTGACCCGGTGCAGCTAGAAG
TCCATATCGGCTGGCTGTTGCTCCAGGCCCCTCGGTGGGTGTTCAAGGAGGAAGACCCTATT
CACCTGAGGTGTCACAGCTGGAAGAACACTGCTCTGCATAAGGTCACATATTTACAGAATGG
CAAAGGCAGGAAGTATTTTCATCATAATTCTGACTTCTACATTCCAAAAGCCACACTCAAAG
ACAGCGGCTCCTACTTCTGCAGGGGGCTTgTTGGGAGTAAAAATGTGTCTTCAGAGACTGTG
AACATCACCATCACTCAAGGTTTGGCAGTGTCAACCATCTCATCATTCTTTCCACCTGGGTA
CCAAgtctctttctgcttggtgatggtactcctttttgcagtggacacaggactatatttct
ctgtgaagacaaacattcgaagctcaacaagagactggaaggaccataaatttaaatggaga
aaggaccctcaagacaaaTAA
TCR/FcR #3 CD16 extracellular domain (Fc binding domain) comprising polypeptide:
CD16ha ECD, CD32 hinge, CD16 TMD, and CD16 ICD, polynucleotide sequence (SEQ
ID NO: 152)
(SEQ ID NO: 152)
ATGTGGCAGCTGCTCCTCCCAACTGCTCTGCTACTTCTAGTTTCAGCTGGCATGCGGACTGA
AGATCTCCCAAAGGCTGTGGTGTTCCTGGAGCCTCAATGGTACAGGGTGCTCGAGAAGGACA
GTGTGACTCTGAAGTGCCAGGGAGCCTACTCCCCTGAGGACAATTCCACACAGTGGTTTCAC
AATGAGAGCCTCATCTCAAGCCAGGCCTCGAGCTACTTCATTGACGCTGCCACAGTCGACGA
CAGTGGAGAGTACAGGTGCCAGACAAACCTCTCCACCCTCAGTGACCCGGTGCAGCTAGAAG
TCCATATCGGCTGGCTGTTGCTCCAGGCCCCTCGGTGGGTGTTCAAGGAGGAAGACCCTATT
CACCTGAGGTGTCACAGCTGGAAGAACACTGCTCTGCATAAGGTCACATATTTACAGAATGG
CAAAGGCAGGAAGTATTTTCATCATAATTCTGACTTCTACATTCCAAAAGCCACACTCAAAG
ACAGCGGCTCCTACTTCTGCAGGGGGCTTgTTGGGAGTAAAAATGTGTCTTCAGAGACTGTG
AACATCACCGTCCAAGTGCCCAGCATGGGCAGCTCTTCACCAATGGGGgtctctttctgctt
ggtgatggtactcctttttgcagtggacacaggactatatttctctgtgaagacaaacattc
gaagctcaacaagagactggaaggaccataaatttaaatggagaaaggaccctcaagacaaa
TAA
TCR/FcR #4 CD16 extracellular domain (Fc binding domain) comprising polypeptide:
CD16ha ECD, CD32 hinge, CD3ζ TMD, and CD3ζ ICD, polynucleotide sequence (SEQ
ID NO: 153)
ATGTGGCAGCTGCTCCTCCCAACTGCTCTGCTACTTCTAGTTTCAGCTGGCATGCGGACTGA
AGATCTCCCAAAGGCTGTGGTGTTCCTGGAGCCTCAATGGTACAGGGTGCTCGAGAAGGACA
GTGTGACTCTGAAGTGCCAGGGAGCCTACTCCCCTGAGGACAATTCCACACAGTGGTTTCAC
AATGAGAGCCTCATCTCAAGCCAGGCCTCGAGCTACTTCATTGACGCTGCCACAGTCGACGA
CAGTGGAGAGTACAGGTGCCAGACAAACCTCTCCACCCTCAGTGACCCGGTGCAGCTAGAAG
TCCATATCGGCTGGCTGTTGCTCCAGGCCCCTCGGTGGGTGTTCAAGGAGGAAGACCCTATT
CACCTGAGGTGTCACAGCTGGAAGAACACTGCTCTGCATAAGGTCACATATTTACAGAATGG
CAAAGGCAGGAAGTATTTTCATCATAATTCTGACTTCTACATTCCAAAAGCCACACTCAAAG
ACAGCGGCTCCTACTTCTGCAGGGGGCTTgTTGGGAGTAAAAATGTGTCTTCAGAGACTGTG
AACATCACCGTCCAAGTGCCCAGCATGGGCAGCTCTTCACCAATGGGGCTCTGTTACCTTCT
TGACGGTATTCTTTTTATTTACGGCGTCATCCTCACTGCCCTCTTTTTGAGGGTCAAGTTTT
CAAGATCCGCCGACGCACCTGCTTACCAGCAAGGACAAAATCAACTTTATAACGAACTCAAC
CTTGGTCGGCGGGAAGAATATGATGTACTCGACAAAAGAAGAGGCCGCGACCCGGAGATGGG
TGGCAAACCACAGCGGAGGAAAAACCCCCAGGAAGGTCTCTATAACGAATTGCAGAAAGATA
AAATGGCCGAGGCTTATTCAGAGATCGGCATGAAAGGAGAGCGACGACGAGGAAAGGGGCAT
GATGGTCTGTATCAAGGGCTCAGCACGGCAACAAAGGACACATATGATGCCTTGCATATGCA
GGCGCTTCCGCCGCGCTAA (SEQ ID NO: 153)
TCR/FcR #5 CD16 extracellular domain (Fc binding domain) comprising polypeptide:
CD16ha ECD, CD32 hinge, CD16 TMD, polynucleotide sequence (SEQ ID NO: 154)
(SEQ ID NO: 154)
ATGTGGCAGCTGCTCCTCCCAACTGCTCTGCTACTTCTAGTTTCAGCTGGCATGCGGACTGA
AGATCTCCCAAAGGCTGTGGTGTTCCTGGAGCCTCAATGGTACAGGGTGCTCGAGAAGGACA
GTGTGACTCTGAAGTGCCAGGGAGCCTACTCCCCTGAGGACAATTCCACACAGTGGTTTCAC
AATGAGAGCCTCATCTCAAGCCAGGCCTCGAGCTACTTCATTGACGCTGCCACAGTCGACGA
CAGTGGAGAGTACAGGTGCCAGACAAACCTCTCCACCCTCAGTGACCCGGTGCAGCTAGAAG
TCCATATCGGCTGGCTGTTGCTCCAGGCCCCTCGGTGGGTGTTCAAGGAGGAAGACCCTATT
CACCTGAGGTGTCACAGCTGGAAGAACACTGCTCTGCATAAGGTCACATATTTACAGAATGG
CAAAGGCAGGAAGTATTTTCATCATAATTCTGACTTCTACATTCCAAAAGCCACACTCAAAG
ACAGCGGCTCCTACTTCTGCAGGGGGCTTgTTGGGAGTAAAAATGTGTCTTCAGAGACTGTG
AACATCACCGTCCAAGTGCCCAGCATGGGCAGCTCTTCACCAATGGGGgtctctttctgctt
ggtgatggtactcctttttgcagtggacacaggactatatttctctgtgaagacaTAA
TCR/FcR #1 CD16 extracellular domain (Fc binding domain) comprising polypeptide:
WT CD16, amino acid sequence (SEQ ID NO: 155)
(SEQ ID NO: 155)
MWQLLLPTALLLLVSAGMRTEDLPKAVVFLEPQWYRVLEKDSVTLKCQGAYSPEDNSTQWFH
NESLISSQASSYFIDAATVDDSGEYRCQTNLSTLSDPVQLEVHIGWLLLQAPRWVFKEEDPI
HLRCHSWKNTALHKVTYLQNGKGRKYFHHNSDFYIPKATLKDSGSYFCRGLFGSKNVSSETV
NITITQGLAVSTISSFFPPGYQVSFCLVMVLLFAVDTGLYFSVKTNIRSSTRDWKDHKFKWR
KDPQDK
TCR/FcR #2 CD16 extracellular domain (Fc binding domain) comprising polypeptide:
WT high affinity CD16, amino acid sequence (CD16ha, F158V)(SEQ ID NO: 156)
(SEQ ID NO: 156)
MWQLLLPTALLLLVSAGMRTEDLPKAVVFLEPQWYRVLEKDSVTLKCQGAYSPEDNSTQWFH
NESLISSQASSYFIDAATVDDSGEYRCQTNLSTLSDPVQLEVHIGWLLLQAPRWVFKEEDPI
HLRCHSWKNTALHKVTYLQNGKGRKYFHHNSDFYIPKATLKDSGSYFCRGLVGSKNVSSETV
NITITQGLAVSTISSFFPPGYQVSFCLVMVLLFAVDTGLYFSVKTNIRSSTRDWKDHKFKWR
KDPQDK
TCR/FcR #3 CD16 extracellular domain (Fc binding domain) comprising polypeptide:
CD16ha ECD, CD32 hinge, CD16 TMD, and CD16 ICD, amino acid sequence (SEQ ID
NO: 157)
(SEQ ID NO: 157)
MWQLLLPTALLLLVSAGMRTEDLPKAVVFLEPQWYRVLEKDSVTLKCQGAYSPEDNSTQWFH
NESLISSQASSYFIDAATVDDSGEYRCQTNLSTLSDPVQLEVHIGWLLLQAPRWVFKEEDPI
HLRCHSWKNTALHKVTYLQNGKGRKYFHHNSDFYIPKATLKDSGSYFCRGLVGSKNVSSETV
NITVQVPSMGSSSPMGVSFCLVMVLLFAVDTGLYFSVKTNIRSSTRDWKDHKFKWRKDPQDK
TCR/FcR #4 CD16 extracellular domain (Fc binding domain) comprising polypeptide:
CD16ha ECD, CD32 hinge, CD3ζ TMD, and CD3ζ ICD, amino acid sequence (SEQ ID
NO: 158)
(SEQ ID NO: 158)
MWQLLLPTALLLLVSAGMRTEDLPKAVVFLEPQWYRVLEKDSVTLKCQGAYSPEDNSTQWFH
NESLISSQASSYFIDAATVDDSGEYRCQTNLSTLSDPVQLEVHIGWLLLQAPRWVFKEEDPI
HLRCHSWKNTALHKVTYLQNGKGRKYFHHNSDFYIPKATLKDSGSYFCRGLVGSKNVSSETV
NITVQVPSMGSSSPMGLCYLLDGILFIYGVILTALFLRVKFSRSADAPAYQQGQNQLYNELN
LGRREEYDVLDKRRGRDPEMGGKPQRRKNPQEGLYNELQKDKMAEAYSEIGMKGERRRGKGH
DGLYQGLSTATKDTYDALHMQALPPR
TCR/FcR #5 CD16 extracellular domain (Fc binding domain) comprising polypeptide:
CD16ha ECD, CD32 hinge, CD16 TMD, amino acid sequence (SEQ ID NO: 159)
(SEQ ID NO: 159)
MWQLLLPTALLLLVSAGMRTEDLPKAVVFLEPQWYRVLEKDSVTLKCQGAYSPEDNSTQWFH
NESLISSQASSYFIDAATVDDSGEYRCQTNLSTLSDPVQLEVHIGWLLLQAPRWVFKEEDPI
HLRCHSWKNTALHKVTYLQNGKGRKYFHHNSDFYIPKATLKDSGSYFCRGLVGSKNVSSETV
NITVQVPSMGSSSPMGVSFCLVMVLLFAVDTGLYFSVKT
CD32 hinge polynucleotide (SEQ ID NO: 160)
(SEQ ID NO: 160)
GTCCAAGTGCCCAGCATGGGCAGCTCTTCACCAATGGGG
CD32 hinge polypeptide (SEQ ID NO: 161)
(SEQ ID NO: 161)
VQVPSMGSSSPMG
CD3ζ transmembrane domain polynucleotide (SEQ ID NO: 162)
(SEQ ID NO: 162)
CTCTGTTACCTTCTTGACGGTATTCTTTTTATTTACGGCGTCATCCTCACTGCCCTCTTTTT
G
CD3ζ transmembrane domain polypeptide (SEQ ID NO: 163)
(SEQ ID NO: 163)
LCYLLDGILFIYGVILTALFL
CD3ζ intracellular signaling domain polynucleotide (SEQ ID NO: 164)
(SEQ ID NO: 164)
AGGGTCAAGTTTTCAAGATCCGCCGACGCACCTGCTTACCAGCAAGGACAAAATCAACTTTA
TAACGAACTCAACCTTGGTCGGCGGGAAGAATATGATGTACTCGACAAAAGAAGAGGCCGCG
ACCCGGAGATGGGTGGCAAACCACAGCGGAGGAAAAACCCCCAGGAAGGTCTCTATAACGAA
TTGCAGAAAGATAAAATGGCCGAGGCTTATTCAGAGATCGGCATGAAAGGAGAGCGACGACG
AGGAAAGGGGCATGATGGTCTGTATCAAGGGCTCAGCACGGCAACAAAGGACACATATGATG
CCTTGCATATGCAGGCGCTTCCGCCGCGCTAA
CD3ζ intracellular signaling domain polypeptide (SEQ ID NO: 165)
(SEQ ID NO: 165)
RVKFSRSADAPAYQQGQNQLYNELNLGRREEYDVLDKRRGRDPEMGGKPQRRKNPQEGLYNE
LQKDKMAEAYSEIGMKGERRRGKGHDGLYQGLSTATKDTYDALHMQALPPR
CD16 transmembrane domain polynucleotide (SEQ ID NO: 166)
(SEQ ID NO: 166)
GTCTCTTTCTGCTTGGTGATGGTACTCCTTTTTGCAGTGGACACAGGACTATATTTCTCTGT
G
CD16 transmembrane domain polypeptide (SEQ ID NO: 167)
(SEQ ID NO: 167)
VSFCLVMVLLFAVDTGLYFSV
CD16 intracellular signaling domain polynucleotide (SEQ ID NO: 168)
(SEQ ID NO: 168)
AAGACAAACATTCGAAGCTCAACAAGAGACTGGAAGGACCATAAATTTAAATGGAGAAAGGA
CCCTCAAGACAAATAA
CD16 intracellular signaling domain polypeptide (SEQ ID NO: 169)
(SEQ ID NO: 169)
KTNIRSSTRDWKDHKFKWRKDPQDK

### C. Exemplary Transgenic Open Reading Frames and Vectors

NK cells are modified to express a heterologous (i.e., transgenic) polynucleotide vector (i.e., construct). In some embodiments, a TCR/FcR construct comprises a multicistronic open reading frame polynucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 171-175. In certain embodiments, a TCR/FcR construct comprises a transgenic vector sequence book-ended by LTRs that is at least, or exactly, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 177-181. In some embodiments, in sequences described herein, underlining refers to signal peptide sequence, which in some embodiments may be modified, omitted, and/or replaced with an alternative signal peptide.

In some embodiments, provided herein are heterologous polynucleotide vectors comprising a pair of iTCR chains, while not comprising a CD16 extracellular domain (Fc binding domain) comprising polypeptide (e.g., an “iTCR” construct, e.g., construct iTCR3). In some embodiments, an iTCR construct comprises a multicistronic open reading frame polynucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 170. In certain embodiments, an iTCR construct comprises a transgenic vector sequence book-ended by LTRs that is at least, or exactly, 60%, 61%, 62%, 63%, 64%, 65%, 66%, 67%, 68%, 69%, 70%, 71%, 72%, 73%, 74%, 75%, 76%, 77%, 78%, 79%, 80%, 8100, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 176.

iTCR3 open reading frame (SEQ ID NO: 170)
(SEQ ID NO: 170)
ATGAAGAAGCACCTGACCACCTTTCTGGTCATCCTGTGGCTGTACTTCTACAGAGGCAACGG
CAAGAACCAGGTGGAACAGAGCCCTCAGAGCCTGATCATCCTGGAAGGCAAGAACTGCACCC
TGCAGTGCAACTACACCGTGTCTCCCTTCAGCAACCTGCGGTGGTACAAGCAGGATACAGGC
AGAGGCCCTGTGTCTCTGACCATCATGACCTTCAGCGAGAACACCAAGAGCAACGGCCGGTA
CACCGCCACACTGGATGCCGATACAAAGCAGAGCAGCCTGCACATCACAGCCAGCCAGCTGA
GCGATAGCGCCAGCTACATCTGCGTGGTGTCCGATAGAGGCAGCACCCTGGGCAGACTGTAC
TTTGGCAGAGGCACCCAGCTGACCGTGTGGCCCGATATTCAGAACCCTGATCCTGCCGTGTA
CCAGCTGAGAGACAGCAAGTCCAGCGACAAGAGCGTGTGCCTGTTCACCGACTTCGACAGCC
AGACCAACGTGTCCCAGAGCAAGGACAGCGACGTGTACATCACCGACAAGtgcGTGCTGGAC
ATGCGGAGCATGGACTTCAAGAGCAATAGCGCCGTGGCCTGGTCCAACAAGAGCGATTTCGC
CTGCGCCAACGCCTTCAACAACAGCATTATCCCCGAGGACACATTCTTCCCAAGTCCTGAGA
GCAGCTGCGACGTGAAGCTGGTGGAAAAGAGCTTCGAGACAGACACCAACCTGAACTTCCAG
AACCTGAGCGTGATCGGCTTCCGGATCCTGCTGCTGAAAGTGGCCGGCTTCAACCTGCTGAT
GACCCTGAGACTGTGGTCTAGCGGAAGCGGCGCCACCAATTTCAGCCTGCTGAAACAGGCTG
GCGACGTGGAAGAGAACCCCGGACCTATGACCATCCGGCTGCTGTGCTATATGGGCTTCTAC
TTCCTCGGAGCCGGCCTGATGGAAGCCGACATCTACCAGACACCTAGATACCTGGTCATCGG
CACCGGCAAAAAGATCACCCTGGAATGCTCCCAGACCATGGGCCACGACAAGATGTACTGGT
ATCAGCAGGACCCCGGCATGGAACTGCATCTGATCCACTACAGCTACGGCGTGAACAGCACC
GAGAAGGGCGATCTGTCTAGCGAGAGCACCGTGTCCAGAATCCGGACCGAGCACTTCCCACT
GACACTGGAAAGCGCCAGACCTAGCCACACCAGCCAGTACCTGTGTGCCACTGGACAGGGGG
CGCAAGATACGCAGTATTTTGGCCCAGGCACCCGGCTGACAGTGCTCGAGGACCTCAAGAAT
GTGTTTCCGCCCGAAGTCGCGGTTTTTGAACCATCAGAAGCCGAGATCTCTCATACACAAAA
GGCGACGCTCGTATGCCTcGCGACGGGATTTTATCCGGACCACGTCGAGCTTTCCTGGTGGG
TTAAcGGAAAGGAGGTGCATTCCGGAGTTTGCACGGACCCTCAGCCATTGAAGGAACAGCCC
GCACTGAACGACAGTAGGTATTGCCTTTCATCTCGCCTGCGCGTGTCTGCGACATTCTGGCA
AAACCCAAGAAATCACTTCAGATGTCAAGTTCAGTTCTACGGTCTCAGCGAGAATGATGAGT
GGACACAAGATAGGGCTAAACCCGTGACTCAAATAGTCTCTGCCGAGGCCTGGGGGAGGGCG
GATTGCGGCTTCACATCAGAATCATACCAACAAGGAGTATTGAGCGCGACAATTCTTTACGA
AATTCTGCTTGGGAAAGCGACTCTGTACGCGGTGCTCGTGTCCGCTTTGGTTCTTATGGCAA
TGGTTAAACGAAAGGATAGTAGGGGCTAA
TCR/FcR #1 open reading frame (ORF)(SEQ ID NO: 171)
(SEQ ID NO: 171)
ATGAAGAAGCACCTGACCACCTTTCTGGTCATCCTGTGGCTGTACTTCTACAGAGGCAACGG
CAAGAACCAGGTGGAACAGAGCCCTCAGAGCCTGATCATCCTGGAAGGCAAGAACTGCACCC
TGCAGTGCAACTACACCGTGTCTCCCTTCAGCAACCTGCGGTGGTACAAGCAGGATACAGGC
AGAGGCCCTGTGTCTCTGACCATCATGACCTTCAGCGAGAACACCAAGAGCAACGGCCGGTA
CACCGCCACACTGGATGCCGATACAAAGCAGAGCAGCCTGCACATCACAGCCAGCCAGCTGA
GCGATAGCGCCAGCTACATCTGCGTGGTGTCCGATAGAGGCAGCACCCTGGGCAGACTGTAC
TTTGGCAGAGGCACCCAGCTGACCGTGTGGCCCGATATTCAGAACCCTGATCCTGCCGTGTA
CCAGCTGAGAGACAGCAAGTCCAGCGACAAGAGCGTGTGCCTGTTCACCGACTTCGACAGCC
AGACCAACGTGTCCCAGAGCAAGGACAGCGACGTGTACATCACCGACAAGtgcGTGCTGGAC
ATGCGGAGCATGGACTTCAAGAGCAATAGCGCCGTGGCCTGGTCCAACAAGAGCGATTTCGC
CTGCGCCAACGCCTTCAACAACAGCATTATCCCCGAGGACACATTCTTCCCAAGTCCTGAGA
GCAGCTGCGACGTGAAGCTGGTGGAAAAGAGCTTCGAGACAGACACCAACCTGAACTTCCAG
AACCTGAGCGTGATCGGCTTCCGGATCCTGCTGCTGAAAGTGGCCGGCTTCAACCTGCTGAT
GACCCTGAGACTGTGGTCTAGCGGAAGCGGCGCCACCAATTTCAGCCTGCTGAAACAGGCTG
GCGACGTGGAAGAGAACCCCGGACCTATGACCATCCGGCTGCTGTGCTATATGGGCTTCTAC
TTCCTCGGAGCCGGCCTGATGGAAGCCGACATCTACCAGACACCTAGATACCTGGTCATCGG
CACCGGCAAAAAGATCACCCTGGAATGCTCCCAGACCATGGGCCACGACAAGATGTACTGGT
ATCAGCAGGACCCCGGCATGGAACTGCATCTGATCCACTACAGCTACGGCGTGAACAGCACC
GAGAAGGGCGATCTGTCTAGCGAGAGCACCGTGTCCAGAATCCGGACCGAGCACTTCCCACT
GACACTGGAAAGCGCCAGACCTAGCCACACCAGCCAGTACCTGTGTGCCACTGGACAGGGGG
CGCAAGATACGCAGTATTTTGGCCCAGGCACCCGGCTGACAGTGCTCGAGGACCTCAAGAAT
GTGTTTCCGCCCGAAGTCGCGGTTTTTGAACCATCAGAAGCCGAGATCTCTCATACACAAAA
GGCGACGCTCGTATGCCTcGCGACGGGATTTTATCCGGACCACGTCGAGCTTTCCTGGTGGG
TTAACGGAAAGGAGGTGCATTCCGGAGTTTGCACGGACCCTCAGCCATTGAAGGAACAGCCC
GCACTGAACGACAGTAGGTATTGCCTTTCATCTCGCCTGCGCGTGTCTGCGACATTCTGGCA
AAACCCAAGAAATCACTTCAGATGTCAAGTTCAGTTCTACGGTCTCAGCGAGAATGATGAGT
GGACACAAGATAGGGCTAAACCCGTGACTCAAATAGTCTCTGCCGAGGCCTGGGGGAGGGCG
GATTGCGGCTTCACATCAGAATCATACCAACAAGGAGTATTGAGCGCGACAATTCTTTACGA
AATTCTGCTTGGGAAAGCGACTCTGTACGCGGTGCTCGTGTCCGCTTTGGTTCTTATGGCAA
TGGTTAAACGAAAGGATAGTAGGGGCGGtAGtGGaCAGTGTACTAATTATGCTCTCTTGAAA
TTGGCTGGAGATGTTGAGAGCAATCCCGGGCCCATGTGGCAGCTGCTCCTCCCAACTGCTCT
GCTACTTCTAGTTTCAGCTGGCATGCGGACTGAAGATCTCCCAAAGGCTGTGGTGTTCCTGG
AGCCTCAATGGTACAGGGTGCTCGAGAAGGACAGTGTGACTCTGAAGTGCCAGGGAGCCTAC
TCCCCTGAGGACAATTCCACACAGTGGTTTCACAATGAGAGCCTCATCTCAAGCCAGGCCTC
GAGCTACTTCATTGACGCTGCCACAGTCGACGACAGTGGAGAGTACAGGTGCCAGACAAACC
TCTCCACCCTCAGTGACCCGGTGCAGCTAGAAGTCCATATCGGCTGGCTGTTGCTCCAGGCC
CCTCGGTGGGTGTTCAAGGAGGAAGACCCTATTCACCTGAGGTGTCACAGCTGGAAGAACAC
TGCTCTGCATAAGGTCACATATTTACAGAATGGCAAAGGCAGGAAGTATTTTCATCATAATT
CTGACTTCTACATTCCAAAAGCCACACTCAAAGACAGCGGCTCCTACTTCTGCAGGGGGCTT
TTTGGGAGTAAAAATGTGTCTTCAGAGACTGTGAACATCACCATCACTCAAGGTTTGGCAGT
GTCAACCATCTCATCATTCTTTCCACCTGGGTACCAAgtctctttctgcttggtgatggtac
tcctttttgcagtggacacaggactatatttctctgtgaagacaaacattcgaagctcaaca
agagactggaaggaccataaatttaaatggagaaaggaccctcaagacaaaTAA
TCR/FcR #2 open reading frame (SEQ ID NO: 172)
(SEQ ID NO: 172)
ATGAAGAAGCACCTGACCACCTTTCTGGTCATCCTGTGGCTGTACTTCTACAGAGGCAACGG
CAAGAACCAGGTGGAACAGAGCCCTCAGAGCCTGATCATCCTGGAAGGCAAGAACTGCACCC
TGCAGTGCAACTACACCGTGTCTCCCTTCAGCAACCTGCGGTGGTACAAGCAGGATACAGGC
AGAGGCCCTGTGTCTCTGACCATCATGACCTTCAGCGAGAACACCAAGAGCAACGGCCGGTA
CACCGCCACACTGGATGCCGATACAAAGCAGAGCAGCCTGCACATCACAGCCAGCCAGCTGA
GCGATAGCGCCAGCTACATCTGCGTGGTGTCCGATAGAGGCAGCACCCTGGGCAGACTGTAC
TTTGGCAGAGGCACCCAGCTGACCGTGTGGCCCGATATTCAGAACCCTGATCCTGCCGTGTA
CCAGCTGAGAGACAGCAAGTCCAGCGACAAGAGCGTGTGCCTGTTCACCGACTTCGACAGCC
AGACCAACGTGTCCCAGAGCAAGGACAGCGACGTGTACATCACCGACAAGtgcGTGCTGGAC
ATGCGGAGCATGGACTTCAAGAGCAATAGCGCCGTGGCCTGGTCCAACAAGAGCGATTTCGC
CTGCGCCAACGCCTTCAACAACAGCATTATCCCCGAGGACACATTCTTCCCAAGTCCTGAGA
GCAGCTGCGACGTGAAGCTGGTGGAAAAGAGCTTCGAGACAGACACCAACCTGAACTTCCAG
AACCTGAGCGTGATCGGCTTCCGGATCCTGCTGCTGAAAGTGGCCGGCTTCAACCTGCTGAT
GACCCTGAGACTGTGGTCTAGCGGAAGCGGCGCCACCAATTTCAGCCTGCTGAAACAGGCTG
GCGACGTGGAAGAGAACCCCGGACCTATGACCATCCGGCTGCTGTGCTATATGGGCTTCTAC
TTCCTCGGAGCCGGCCTGATGGAAGCCGACATCTACCAGACACCTAGATACCTGGTCATCGG
CACCGGCAAAAAGATCACCCTGGAATGCTCCCAGACCATGGGCCACGACAAGATGTACTGGT
ATCAGCAGGACCCCGGCATGGAACTGCATCTGATCCACTACAGCTACGGCGTGAACAGCACC
GAGAAGGGCGATCTGTCTAGCGAGAGCACCGTGTCCAGAATCCGGACCGAGCACTTCCCACT
GACACTGGAAAGCGCCAGACCTAGCCACACCAGCCAGTACCTGTGTGCCACTGGACAGGGGG
CGCAAGATACGCAGTATTTTGGCCCAGGCACCCGGCTGACAGTGCTCGAGGACCTCAAGAAT
GTGTTTCCGCCCGAAGTCGCGGTTTTTGAACCATCAGAAGCCGAGATCTCTCATACACAAAA
GGCGACGCTCGTATGCCTcGCGACGGGATTTTATCCGGACCACGTCGAGCTTTCCTGGTGGG
TTAACGGAAAGGAGGTGCATTCCGGAGTTTGCACGGACCCTCAGCCATTGAAGGAACAGCCC
GCACTGAACGACAGTAGGTATTGCCTTTCATCTCGCCTGCGCGTGTCTGCGACATTCTGGCA
AAACCCAAGAAATCACTTCAGATGTCAAGTTCAGTTCTACGGTCTCAGCGAGAATGATGAGT
GGACACAAGATAGGGCTAAACCCGTGACTCAAATAGTCTCTGCCGAGGCCTGGGGGAGGGCG
GATTGCGGCTTCACATCAGAATCATACCAACAAGGAGTATTGAGCGCGACAATTCTTTACGA
AATTCTGCTTGGGAAAGCGACTCTGTACGCGGTGCTCGTGTCCGCTTTGGTTCTTATGGCAA
TGGTTAAACGAAAGGATAGTAGGGGCGGtAGtGGaCAGTGTACTAATTATGCTCTCTTGAAA
TTGGCTGGAGATGTTGAGAGCAATCCCGGGCCCATGTGGCAGCTGCTCCTCCCAACTGCTCT
GCTACTTCTAGTTTCAGCTGGCATGCGGACTGAAGATCTCCCAAAGGCTGTGGTGTTCCTGG
AGCCTCAATGGTACAGGGTGCTCGAGAAGGACAGTGTGACTCTGAAGTGCCAGGGAGCCTAC
TCCCCTGAGGACAATTCCACACAGTGGTTTCACAATGAGAGCCTCATCTCAAGCCAGGCCTC
GAGCTACTTCATTGACGCTGCCACAGTCGACGACAGTGGAGAGTACAGGTGCCAGACAAACC
TCTCCACCCTCAGTGACCCGGTGCAGCTAGAAGTCCATATCGGCTGGCTGTTGCTCCAGGCC
CCTCGGTGGGTGTTCAAGGAGGAAGACCCTATTCACCTGAGGTGTCACAGCTGGAAGAACAC
TGCTCTGCATAAGGTCACATATTTACAGAATGGCAAAGGCAGGAAGTATTTTCATCATAATT
CTGACTTCTACATTCCAAAAGCCACACTCAAAGACAGCGGCTCCTACTTCTGCAGGGGGCTT
gTTGGGAGTAAAAATGTGTCTTCAGAGACTGTGAACATCACCATCACTCAAGGTTTGGCAGT
GTCAACCATCTCATCATTCTTTCCACCTGGGTACCAAgtctctttctgcttggtgatggtac
tcctttttgcagtggacacaggactatatttctctgtgaagacaaacattcgaagctcaaca
agagactggaaggaccataaatttaaatggagaaaggaccctcaagacaaaTAA
TCR/FcR #3 open reading frame (SEQ ID NO: 173)
(SEQ ID NO: 173)
ATGAAGAAGCACCTGACCACCTTTCTGGTCATCCTGTGGCTGTACTTCTACAGAGGCAACGG
CAAGAACCAGGTGGAACAGAGCCCTCAGAGCCTGATCATCCTGGAAGGCAAGAACTGCACCC
TGCAGTGCAACTACACCGTGTCTCCCTTCAGCAACCTGCGGTGGTACAAGCAGGATACAGGC
AGAGGCCCTGTGTCTCTGACCATCATGACCTTCAGCGAGAACACCAAGAGCAACGGCCGGTA
CACCGCCACACTGGATGCCGATACAAAGCAGAGCAGCCTGCACATCACAGCCAGCCAGCTGA
GCGATAGCGCCAGCTACATCTGCGTGGTGTCCGATAGAGGCAGCACCCTGGGCAGACTGTAC
TTTGGCAGAGGCACCCAGCTGACCGTGTGGCCCGATATTCAGAACCCTGATCCTGCCGTGTA
CCAGCTGAGAGACAGCAAGTCCAGCGACAAGAGCGTGTGCCTGTTCACCGACTTCGACAGCC
AGACCAACGTGTCCCAGAGCAAGGACAGCGACGTGTACATCACCGACAAGtgcGTGCTGGAC
ATGCGGAGCATGGACTTCAAGAGCAATAGCGCCGTGGCCTGGTCCAACAAGAGCGATTTCGC
CTGCGCCAACGCCTTCAACAACAGCATTATCCCCGAGGACACATTCTTCCCAAGTCCTGAGA
GCAGCTGCGACGTGAAGCTGGTGGAAAAGAGCTTCGAGACAGACACCAACCTGAACTTCCAG
AACCTGAGCGTGATCGGCTTCCGGATCCTGCTGCTGAAAGTGGCCGGCTTCAACCTGCTGAT
GACCCTGAGACTGTGGTCTAGCGGAAGCGGCGCCACCAATTTCAGCCTGCTGAAACAGGCTG
GCGACGTGGAAGAGAACCCCGGACCTATGACCATCCGGCTGCTGTGCTATATGGGCTTCTAC
TTCCTCGGAGCCGGCCTGATGGAAGCCGACATCTACCAGACACCTAGATACCTGGTCATCGG
CACCGGCAAAAAGATCACCCTGGAATGCTCCCAGACCATGGGCCACGACAAGATGTACTGGT
ATCAGCAGGACCCCGGCATGGAACTGCATCTGATCCACTACAGCTACGGCGTGAACAGCACC
GAGAAGGGCGATCTGTCTAGCGAGAGCACCGTGTCCAGAATCCGGACCGAGCACTTCCCACT
GACACTGGAAAGCGCCAGACCTAGCCACACCAGCCAGTACCTGTGTGCCACTGGACAGGGGG
CGCAAGATACGCAGTATTTTGGCCCAGGCACCCGGCTGACAGTGCTCGAGGACCTCAAGAAT
GTGTTTCCGCCCGAAGTCGCGGTTTTTGAACCATCAGAAGCCGAGATCTCTCATACACAAAA
GGCGACGCTCGTATGCCTcGCGACGGGATTTTATCCGGACCACGTCGAGCTTTCCTGGTGGG
TTAACGGAAAGGAGGTGCATTCCGGAGTTTGCACGGACCCTCAGCCATTGAAGGAACAGCCC
GCACTGAACGACAGTAGGTATTGCCTTTCATCTCGCCTGCGCGTGTCTGCGACATTCTGGCA
AAACCCAAGAAATCACTTCAGATGTCAAGTTCAGTTCTACGGTCTCAGCGAGAATGATGAGT
GGACACAAGATAGGGCTAAACCCGTGACTCAAATAGTCTCTGCCGAGGCCTGGGGGAGGGCG
GATTGCGGCTTCACATCAGAATCATACCAACAAGGAGTATTGAGCGCGACAATTCTTTACGA
AATTCTGCTTGGGAAAGCGACTCTGTACGCGGTGCTCGTGTCCGCTTTGGTTCTTATGGCAA
TGGTTAAACGAAAGGATAGTAGGGGCGGtAGtGGaCAGTGTACTAATTATGCTCTCTTGAAA
TTGGCTGGAGATGTTGAGAGCAATCCCGGGCCCATGTGGCAGCTGCTCCTCCCAACTGCTCT
GCTACTTCTAGTTTCAGCTGGCATGCGGACTGAAGATCTCCCAAAGGCTGTGGTGTTCCTGG
AGCCTCAATGGTACAGGGTGCTCGAGAAGGACAGTGTGACTCTGAAGTGCCAGGGAGCCTAC
TCCCCTGAGGACAATTCCACACAGTGGTTTCACAATGAGAGCCTCATCTCAAGCCAGGCCTC
GAGCTACTTCATTGACGCTGCCACAGTCGACGACAGTGGAGAGTACAGGTGCCAGACAAACC
TCTCCACCCTCAGTGACCCGGTGCAGCTAGAAGTCCATATCGGCTGGCTGTTGCTCCAGGCC
CCTCGGTGGGTGTTCAAGGAGGAAGACCCTATTCACCTGAGGTGTCACAGCTGGAAGAACAC
TGCTCTGCATAAGGTCACATATTTACAGAATGGCAAAGGCAGGAAGTATTTTCATCATAATT
CTGACTTCTACATTCCAAAAGCCACACTCAAAGACAGCGGCTCCTACTTCTGCAGGGGGCTT
gTTGGGAGTAAAAATGTGTCTTCAGAGACTGTGAACATCACCGTCCAAGTGCCCAGCATGGG
CAGCTCTTCACCAATGGGGgtctctttctgcttggtgatggtactcctttttgcagtggaca
caggactatatttctctgtgaagacaaacattcgaagctcaacaagagactggaaggaccat
aaatttaaatggagaaaggaccctcaagacaaaTAA
TCR/FcR #4 open reading frame (SEQ ID NO: 174)
(SEQ ID NO: 174)
ATGAAGAAGCACCTGACCACCTTTCTGGTCATCCTGTGGCTGTACTTCTACAGAGGCAACGG
CAAGAACCAGGTGGAACAGAGCCCTCAGAGCCTGATCATCCTGGAAGGCAAGAACTGCACCC
TGCAGTGCAACTACACCGTGTCTCCCTTCAGCAACCTGCGGTGGTACAAGCAGGATACAGGC
AGAGGCCCTGTGTCTCTGACCATCATGACCTTCAGCGAGAACACCAAGAGCAACGGCCGGTA
CACCGCCACACTGGATGCCGATACAAAGCAGAGCAGCCTGCACATCACAGCCAGCCAGCTGA
GCGATAGCGCCAGCTACATCTGCGTGGTGTCCGATAGAGGCAGCACCCTGGGCAGACTGTAC
TTTGGCAGAGGCACCCAGCTGACCGTGTGGCCCGATATTCAGAACCCTGATCCTGCCGTGTA
CCAGCTGAGAGACAGCAAGTCCAGCGACAAGAGCGTGTGCCTGTTCACCGACTTCGACAGCC
AGACCAACGTGTCCCAGAGCAAGGACAGCGACGTGTACATCACCGACAAGtgcGTGCTGGAC
ATGCGGAGCATGGACTTCAAGAGCAATAGCGCCGTGGCCTGGTCCAACAAGAGCGATTTCGC
CTGCGCCAACGCCTTCAACAACAGCATTATCCCCGAGGACACATTCTTCCCAAGTCCTGAGA
GCAGCTGCGACGTGAAGCTGGTGGAAAAGAGCTTCGAGACAGACACCAACCTGAACTTCCAG
AACCTGAGCGTGATCGGCTTCCGGATCCTGCTGCTGAAAGTGGCCGGCTTCAACCTGCTGAT
GACCCTGAGACTGTGGTCTAGCGGAAGCGGCGCCACCAATTTCAGCCTGCTGAAACAGGCTG
GCGACGTGGAAGAGAACCCCGGACCTATGACCATCCGGCTGCTGTGCTATATGGGCTTCTAC
TTCCTCGGAGCCGGCCTGATGGAAGCCGACATCTACCAGACACCTAGATACCTGGTCATCGG
CACCGGCAAAAAGATCACCCTGGAATGCTCCCAGACCATGGGCCACGACAAGATGTACTGGT
ATCAGCAGGACCCCGGCATGGAACTGCATCTGATCCACTACAGCTACGGCGTGAACAGCACC
GAGAAGGGCGATCTGTCTAGCGAGAGCACCGTGTCCAGAATCCGGACCGAGCACTTCCCACT
GACACTGGAAAGCGCCAGACCTAGCCACACCAGCCAGTACCTGTGTGCCACTGGACAGGGGG
CGCAAGATACGCAGTATTTTGGCCCAGGCACCCGGCTGACAGTGCTCGAGGACCTCAAGAAT
GTGTTTCCGCCCGAAGTCGCGGTTTTTGAACCATCAGAAGCCGAGATCTCTCATACACAAAA
GGCGACGCTCGTATGCCTcGCGACGGGATTTTATCCGGACCACGTCGAGCTTTCCTGGTGGG
TTAACGGAAAGGAGGTGCATTCCGGAGTTTGCACGGACCCTCAGCCATTGAAGGAACAGCCC
GCACTGAACGACAGTAGGTATTGCCTTTCATCTCGCCTGCGCGTGTCTGCGACATTCTGGCA
AAACCCAAGAAATCACTTCAGATGTCAAGTTCAGTTCTACGGTCTCAGCGAGAATGATGAGT
GGACACAAGATAGGGCTAAACCCGTGACTCAAATAGTCTCTGCCGAGGCCTGGGGGAGGGCG
GATTGCGGCTTCACATCAGAATCATACCAACAAGGAGTATTGAGCGCGACAATTCTTTACGA
AATTCTGCTTGGGAAAGCGACTCTGTACGCGGTGCTCGTGTCCGCTTTGGTTCTTATGGCAA
TGGTTAAACGAAAGGATAGTAGGGGCGGtAGtGGaCAGTGTACTAATTATGCTCTCTTGAAA
TTGGCTGGAGATGTTGAGAGCAATCCCGGGCCCATGTGGCAGCTGCTCCTCCCAACTGCTCT
GCTACTTCTAGTTTCAGCTGGCATGCGGACTGAAGATCTCCCAAAGGCTGTGGTGTTCCTGG
AGCCTCAATGGTACAGGGTGCTCGAGAAGGACAGTGTGACTCTGAAGTGCCAGGGAGCCTAC
TCCCCTGAGGACAATTCCACACAGTGGTTTCACAATGAGAGCCTCATCTCAAGCCAGGCCTC
GAGCTACTTCATTGACGCTGCCACAGTCGACGACAGTGGAGAGTACAGGTGCCAGACAAACC
TCTCCACCCTCAGTGACCCGGTGCAGCTAGAAGTCCATATCGGCTGGCTGTTGCTCCAGGCC
CCTCGGTGGGTGTTCAAGGAGGAAGACCCTATTCACCTGAGGTGTCACAGCTGGAAGAACAC
TGCTCTGCATAAGGTCACATATTTACAGAATGGCAAAGGCAGGAAGTATTTTCATCATAATT
CTGACTTCTACATTCCAAAAGCCACACTCAAAGACAGCGGCTCCTACTTCTGCAGGGGGCTT
gTTGGGAGTAAAAATGTGTCTTCAGAGACTGTGAACATCACCGTCCAAGTGCCCAGCATGGG
CAGCTCTTCACCAATGGGGCTCTGTTACCTTCTTGACGGTATTCTTTTTATTTACGGCGTCA
TCCTCACTGCCCTCTTTTTGAGGGTCAAGTTTTCAAGATCCGCCGACGCACCTGCTTACCAG
CAAGGACAAAATCAACTTTATAACGAACTCAACCTTGGTCGGCGGGAAGAATATGATGTACT
CGACAAAAGAAGAGGCCGCGACCCGGAGATGGGTGGCAAACCACAGCGGAGGAAAAACCCCC
AGGAAGGTCTCTATAACGAATTGCAGAAAGATAAAATGGCCGAGGCTTATTCAGAGATCGGC
ATGAAAGGAGAGCGACGACGAGGAAAGGGGCATGATGGTCTGTATCAAGGGCTCAGCACGGC
AACAAAGGACACATATGATGCCTTGCATATGCAGGCGCTTCCGCCGCGCTAA
TCR/FcR #5 open reading frame (SEQ ID NO: 175)
(SEQ ID NO: 175)
ATGAAGAAGCACCTGACCACCTTTCTGGTCATCCTGTGGCTGTACTTCTACAGAGGCAACGG
CAAGAACCAGGTGGAACAGAGCCCTCAGAGCCTGATCATCCTGGAAGGCAAGAACTGCACCC
TGCAGTGCAACTACACCGTGTCTCCCTTCAGCAACCTGCGGTGGTACAAGCAGGATACAGGC
AGAGGCCCTGTGTCTCTGACCATCATGACCTTCAGCGAGAACACCAAGAGCAACGGCCGGTA
CACCGCCACACTGGATGCCGATACAAAGCAGAGCAGCCTGCACATCACAGCCAGCCAGCTGA
GCGATAGCGCCAGCTACATCTGCGTGGTGTCCGATAGAGGCAGCACCCTGGGCAGACTGTAC
TTTGGCAGAGGCACCCAGCTGACCGTGTGGCCCGATATTCAGAACCCTGATCCTGCCGTGTA
CCAGCTGAGAGACAGCAAGTCCAGCGACAAGAGCGTGTGCCTGTTCACCGACTTCGACAGCC
AGACCAACGTGTCCCAGAGCAAGGACAGCGACGTGTACATCACCGACAAGtgcGTGCTGGAC
ATGCGGAGCATGGACTTCAAGAGCAATAGCGCCGTGGCCTGGTCCAACAAGAGCGATTTCGC
CTGCGCCAACGCCTTCAACAACAGCATTATCCCCGAGGACACATTCTTCCCAAGTCCTGAGA
GCAGCTGCGACGTGAAGCTGGTGGAAAAGAGCTTCGAGACAGACACCAACCTGAACTTCCAG
AACCTGAGCGTGATCGGCTTCCGGATCCTGCTGCTGAAAGTGGCCGGCTTCAACCTGCTGAT
GACCCTGAGACTGTGGTCTAGCGGAAGCGGCGCCACCAATTTCAGCCTGCTGAAACAGGCTG
GCGACGTGGAAGAGAACCCCGGACCTATGACCATCCGGCTGCTGTGCTATATGGGCTTCTAC
TTCCTCGGAGCCGGCCTGATGGAAGCCGACATCTACCAGACACCTAGATACCTGGTCATCGG
CACCGGCAAAAAGATCACCCTGGAATGCTCCCAGACCATGGGCCACGACAAGATGTACTGGT
ATCAGCAGGACCCCGGCATGGAACTGCATCTGATCCACTACAGCTACGGCGTGAACAGCACC
GAGAAGGGCGATCTGTCTAGCGAGAGCACCGTGTCCAGAATCCGGACCGAGCACTTCCCACT
GACACTGGAAAGCGCCAGACCTAGCCACACCAGCCAGTACCTGTGTGCCACTGGACAGGGGG
CGCAAGATACGCAGTATTTTGGCCCAGGCACCCGGCTGACAGTGCTCGAGGACCTCAAGAAT
GTGTTTCCGCCCGAAGTCGCGGTTTTTGAACCATCAGAAGCCGAGATCTCTCATACACAAAA
GGCGACGCTCGTATGCCTcGCGACGGGATTTTATCCGGACCACGTCGAGCTTTCCTGGTGGG
TTAACGGAAAGGAGGTGCATTCCGGAGTTTGCACGGACCCTCAGCCATTGAAGGAACAGCCC
GCACTGAACGACAGTAGGTATTGCCTTTCATCTCGCCTGCGCGTGTCTGCGACATTCTGGCA
AAACCCAAGAAATCACTTCAGATGTCAAGTTCAGTTCTACGGTCTCAGCGAGAATGATGAGT
GGACACAAGATAGGGCTAAACCCGTGACTCAAATAGTCTCTGCCGAGGCCTGGGGGAGGGCG
GATTGCGGCTTCACATCAGAATCATACCAACAAGGAGTATTGAGCGCGACAATTCTTTACGA
AATTCTGCTTGGGAAAGCGACTCTGTACGCGGTGCTCGTGTCCGCTTTGGTTCTTATGGCAA
TGGTTAAACGAAAGGATAGTAGGGGCGGtAGtGGaCAGTGTACTAATTATGCTCTCTTGAAA
TTGGCTGGAGATGTTGAGAGCAATCCCGGGCCCATGTGGCAGCTGCTCCTCCCAACTGCTCT
GCTACTTCTAGTTTCAGCTGGCATGCGGACTGAAGATCTCCCAAAGGCTGTGGTGTTCCTGG
AGCCTCAATGGTACAGGGTGCTCGAGAAGGACAGTGTGACTCTGAAGTGCCAGGGAGCCTAC
TCCCCTGAGGACAATTCCACACAGTGGTTTCACAATGAGAGCCTCATCTCAAGCCAGGCCTC
GAGCTACTTCATTGACGCTGCCACAGTCGACGACAGTGGAGAGTACAGGTGCCAGACAAACC
TCTCCACCCTCAGTGACCCGGTGCAGCTAGAAGTCCATATCGGCTGGCTGTTGCTCCAGGCC
CCTCGGTGGGTGTTCAAGGAGGAAGACCCTATTCACCTGAGGTGTCACAGCTGGAAGAACAC
TGCTCTGCATAAGGTCACATATTTACAGAATGGCAAAGGCAGGAAGTATTTTCATCATAATT
CTGACTTCTACATTCCAAAAGCCACACTCAAAGACAGCGGCTCCTACTTCTGCAGGGGGCTT
gTTGGGAGTAAAAATGTGTCTTCAGAGACTGTGAACATCACCGTCCAAGTGCCCAGCATGGG
CAGCTCTTCACCAATGGGGgtctctttctgcttggtgatggtactcctttttgcagtggaca
caggactatatttctctgtgaagacaTAA
iTCR3 transgenic vector (SEQ ID NO: 176)
(SEQ ID NO: 176)
AATGAAAGACCCCACCTGTAGGTTTGGCAAGCTAGCTTAAGTAACGCCATTTTGCAAGGCAT
GGAAAAATACATAACTGAGAATAGAAAAGTTCAGATCAAGGTCAGGAACAGATGGAACAGCT
GAATATGGGCCAAACAGGATATCTGTGGTAAGCAGTTCCTGCCCCGGCTCAGGGCCAAGAAC
AGATGGAACAGCTGAATATGGGCCAAACAGGATATCTGTGGTAAGCAGTTCCTGCCCCGGCT
CAGGGCCAAGAACAGATGGTCCCCAGATGCGGTCCAGCCCTCAGCAGTTTCTAGAGAACCAT
CAGATGTTTCCAGGGTGCCCCAAGGACCTGAAATGACCCTGTGCCTTATTTGAACTAACCAA
TCAGTTCGCTTCTCGCTTCTGTTCGCGCGCTTATGCTCCCCGAGCTCAATAAAAGAGCCCAC
AACCCCTCACTCGGGGCGCCAGTCCTCCGATTGACTGAGTCGCCCGGGTACCCGTGTATCCA
ATAAACCCTCTTGCAGTTGCATCCGACTTGTGGTCTCGCTGTTCCTTGGGAGGGTCTCCTCT
GAGTGATTGACTACCCGTCAGCGGGGGTCTTTCATTTGGGGGCTCGTCCGGGATCGGGAGAC
CCCTGCCCAGGGACCACCGACCCACCACCGGGAGGTAAGCTGGCCAGCAACTTATCTGTGTC
TGTCCGATTGTCTAGTGTCTATGACTGATTTTATGCGCCTGCGTCGGTACTAGTTAGCTAAC
TAGCTCTGTATCTGGCGGACCCGTGGTGGAACTGACGAGTTCGGAACACCCGGCCGCAACCC
TGGGAGACGTCCCAGGGACTTCGGGGGCCGTTTTTGTGGCCCGACCTGAGTCCTAAAATCCC
GATCGTTTAGGACTCTTTGGTGCACCCCCCTTAGAGGAGGGATATGTGGTTCTGGTAGGAGA
CGAGAACCTAAAACAGTTCCCGCCTCCGTCTGAATTTTTGCTTTCGGTTTGGGACCGAAGCC
GCGCCGCGCGTCTTGTCTGCTGCAGCATCGTTCTGTGTTGTCTCTGTCTGACTGTGTTTCTG
TATTTGTCTGAAAATATGGGCCCGGGCTAGCCTGTTACCACTCCCTTAAGTTTGACCTTAGG
TCACTGGAAAGATGTCGAGCGGATCGCTCACAACCAGTCGGTAGATGTCAAGAAGAGACGTT
GGGTTACCTTCTGCTCTGCAGAATGGCCAACCTTTAACGTCGGATGGCCGCGAGACGGCACC
TTTAACCGAGACCTCATCACCCAGGTTAAGATCAAGGTCTTTTCACCTGGCCCGCATGGACA
CCCAGACCAGGTGGGGTACATCGTGACCTGGGAAGCCTTGGCTTTTGACCCCCCTCCCTGGG
TCAAGCCCTTTGTACACCCTAAGCCTCCGCCTCCTCTTCCTCCATCCGCCCCGTCTCTCCCC
CTTGAACCTCCTCGTTCGACCCCGCCTCGATCCTCCCTTTATCCAGCCCTCACTCCTTCTCT
AGGCGCCCCCATATGGCCATATGAGATCTTATATGGGGCACCCCCGCCCCTTGTAAACTTCC
CTGACCCTGACATGACAAGAGTTACTAACAGCCCCTCTCTCCAAGCTCACTTACAGGCTCTC
TACTTAGTCCAGCACGAAGTCTGGAGACCTCTGGCGGCAGCCTACCAAGAACAACTGGACCG
ACCGGTGGTACCTCACCCTTACCGAGTCGGCGACACAGTGTGGGTCCGCCGACACCAGACTA
AGAACCTAGAACCTCGCTGGAAAGGACCTTACACAGTCCTGCTGACCACCCCCACCGCCCTC
AAAGTAGACGGCATCGCAGCTTGGATACACGCCGCCCACGTGAAGGCTGCCGACCCCGGGGG
TGGACCATCCTCTAGACTGCCATGAAGAAGCACCTGACCACCTTTCTGGTCATCCTGTGGCT
GTACTTCTACAGAGGCAACGGCAAGAACCAGGTGGAACAGAGCCCTCAGAGCCTGATCATCC
TGGAAGGCAAGAACTGCACCCTGCAGTGCAACTACACCGTGTCTCCCTTCAGCAACCTGCGG
TGGTACAAGCAGGATACAGGCAGAGGCCCTGTGTCTCTGACCATCATGACCTTCAGCGAGAA
CACCAAGAGCAACGGCCGGTACACCGCCACACTGGATGCCGATACAAAGCAGAGCAGCCTGC
ACATCACAGCCAGCCAGCTGAGCGATAGCGCCAGCTACATCTGCGTGGTGTCCGATAGAGGC
AGCACCCTGGGCAGACTGTACTTTGGCAGAGGCACCCAGCTGACCGTGTGGCCCGATATTCA
GAACCCTGATCCTGCCGTGTACCAGCTGAGAGACAGCAAGTCCAGCGACAAGAGCGTGTGCC
TGTTCACCGACTTCGACAGCCAGACCAACGTGTCCCAGAGCAAGGACAGCGACGTGTACATC
ACCGACAAGtgcGTGCTGGACATGCGGAGCATGGACTTCAAGAGCAATAGCGCCGTGGCCTG
GTCCAACAAGAGCGATTTCGCCTGCGCCAACGCCTTCAACAACAGCATTATCCCCGAGGACA
CATTCTTCCCAAGTCCTGAGAGCAGCTGCGACGTGAAGCTGGTGGAAAAGAGCTTCGAGACA
GACACCAACCTGAACTTCCAGAACCTGAGCGTGATCGGCTTCCGGATCCTGCTGCTGAAAGT
GGCCGGCTTCAACCTGCTGATGACCCTGAGACTGTGGTCTAGCGGAAGCGGCGCCACCAATT
TCAGCCTGCTGAAACAGGCTGGCGACGTGGAAGAGAACCCCGGACCTATGACCATCCGGCTG
CTGTGCTATATGGGCTTCTACTTCCTCGGAGCCGGCCTGATGGAAGCCGACATCTACCAGAC
ACCTAGATACCTGGTCATCGGCACCGGCAAAAAGATCACCCTGGAATGCTCCCAGACCATGG
GCCACGACAAGATGTACTGGTATCAGCAGGACCCCGGCATGGAACTGCATCTGATCCACTAC
AGCTACGGCGTGAACAGCACCGAGAAGGGCGATCTGTCTAGCGAGAGCACCGTGTCCAGAAT
CCGGACCGAGCACTTCCCACTGACACTGGAAAGCGCCAGACCTAGCCACACCAGCCAGTACC
TGTGTGCCACTGGACAGGGGGCGCAAGATACGCAGTATTTTGGCCCAGGCACCCGGCTGACA
GTGCTCGAGGACCTCAAGAATGTGTTTCCGCCCGAAGTCGCGGTTTTTGAACCATCAGAAGC
CGAGATCTCTCATACACAAAAGGCGACGCTCGTATGCCTcGCGACGGGATTTTATCCGGACC
ACGTCGAGCTTTCCTGGTGGGTTAACGGAAAGGAGGTGCATTCCGGAGTTTGCACGGACCCT
CAGCCATTGAAGGAACAGCCCGCACTGAACGACAGTAGGTATTGCCTTTCATCTCGCCTGCG
CGTGTCTGCGACATTCTGGCAAAACCCAAGAAATCACTTCAGATGTCAAGTTCAGTTCTACG
GTCTCAGCGAGAATGATGAGTGGACACAAGATAGGGCTAAACCCGTGACTCAAATAGTCTCT
GCCGAGGCCTGGGGGAGGGCGGATTGCGGCTTCACATCAGAATCATACCAACAAGGAGTATT
GAGCGCGACAATTCTTTACGAAATTCTGCTTGGGAAAGCGACTCTGTACGCGGTGCTCGTGT
CCGCTTTGGTTCTTATGGCAATGGTTAAACGAAAGGATAGTAGGGGCTAACAATTGCGCGTC
ATCATCGATCCGGATTAGTCCAATTTGTTAAAGACAGGATATCAGTGGTCCAGGCTCTAGTT
TTGACTCAACAATATCACCAGCTGAAGCCTATAGAGTACGAGCCATAGATAAAATAAAAGAT
TTTATTTAGTCTCCAGAAAAAGGGGGGAATGAAAGACCCCACCTGTAGGTTTGGCAAGCTAG
CTTAAGTAACGCCATTTTGCAAGGCATGGAAAAATACATAACTGAGAATAGAGAAGTTCAGA
TCAAGGTCAGGAACAGATGGAACAGCTGAATATGGGCCAAACAGGATATCTGTGGTAAGCAG
TTCCTGCCCCGGCTCAGGGCCAAGAACAGATGGAACAGCTGAATATGGGCCAAACAGGATAT
CTGTGGTAAGCAGTTCCTGCCCCGGCTCAGGGCCAAGAACAGATGGTCCCCAGATGCGGTCC
AGCCCTCAGCAGTTTCTAGAGAACCATCAGATGTTTCCAGGGTGCCCCAAGGACCTGAAATG
ACCCTGTGCCTTATTTGAACTAACCAATCAGTTCGCTTCTCGCTTCTGTTCGCGCGCTTCTG
CTCCCCGAGCTCAATAAAAGAGCCCACAACCCCTCACTCGGGGCGCCAGTCCTCCGATTGAC
TGAGTCGCCCGGGTACCCGTGTATCCAATAAACCCTCTTGCAGTTGCATCCGACTTGTGGTC
TCGCTGTTCCTTGGGAGGGTCTCCTCTGAGTGATTGACTACCCGTCAGCGGGGGTCTTTCA
TCR/FcR #1 transgenic vector (SEQ ID NO: 177)
(SEQ ID NO: 177)
AATGAAAGACCCCACCTGTAGGTTTGGCAAGCTAGCTTAAGTAACGCCATTTTGCAAGGCAT
GGAAAAATACATAACTGAGAATAGAAAAGTTCAGATCAAGGTCAGGAACAGATGGAACAGCT
GAATATGGGCCAAACAGGATATCTGTGGTAAGCAGTTCCTGCCCCGGCTCAGGGCCAAGAAC
AGATGGAACAGCTGAATATGGGCCAAACAGGATATCTGTGGTAAGCAGTTCCTGCCCCGGCT
CAGGGCCAAGAACAGATGGTCCCCAGATGCGGTCCAGCCCTCAGCAGTTTCTAGAGAACCAT
CAGATGTTTCCAGGGTGCCCCAAGGACCTGAAATGACCCTGTGCCTTATTTGAACTAACCAA
TCAGTTCGCTTCTCGCTTCTGTTCGCGCGCTTATGCTCCCCGAGCTCAATAAAAGAGCCCAC
AACCCCTCACTCGGGGCGCCAGTCCTCCGATTGACTGAGTCGCCCGGGTACCCGTGTATCCA
ATAAACCCTCTTGCAGTTGCATCCGACTTGTGGTCTCGCTGTTCCTTGGGAGGGTCTCCTCT
GAGTGATTGACTACCCGTCAGCGGGGGTCTTTCATTTGGGGGCTCGTCCGGGATCGGGAGAC
CCCTGCCCAGGGACCACCGACCCACCACCGGGAGGTAAGCTGGCCAGCAACTTATCTGTGTC
TGTCCGATTGTCTAGTGTCTATGACTGATTTTATGCGCCTGCGTCGGTACTAGTTAGCTAAC
TAGCTCTGTATCTGGCGGACCCGTGGTGGAACTGACGAGTTCGGAACACCCGGCCGCAACCC
TGGGAGACGTCCCAGGGACTTCGGGGGCCGTTTTTGTGGCCCGACCTGAGTCCTAAAATCCC
GATCGTTTAGGACTCTTTGGTGCACCCCCCTTAGAGGAGGGATATGTGGTTCTGGTAGGAGA
CGAGAACCTAAAACAGTTCCCGCCTCCGTCTGAATTTTTGCTTTCGGTTTGGGACCGAAGCC
GCGCCGCGCGTCTTGTCTGCTGCAGCATCGTTCTGTGTTGTCTCTGTCTGACTGTGTTTCTG
TATTTGTCTGAAAATATGGGCCCGGGCTAGCCTGTTACCACTCCCTTAAGTTTGACCTTAGG
TCACTGGAAAGATGTCGAGCGGATCGCTCACAACCAGTCGGTAGATGTCAAGAAGAGACGTT
GGGTTACCTTCTGCTCTGCAGAATGGCCAACCTTTAACGTCGGATGGCCGCGAGACGGCACC
TTTAACCGAGACCTCATCACCCAGGTTAAGATCAAGGTCTTTTCACCTGGCCCGCATGGACA
CCCAGACCAGGTGGGGTACATCGTGACCTGGGAAGCCTTGGCTTTTGACCCCCCTCCCTGGG
TCAAGCCCTTTGTACACCCTAAGCCTCCGCCTCCTCTTCCTCCATCCGCCCCGTCTCTCCCC
CTTGAACCTCCTCGTTCGACCCCGCCTCGATCCTCCCTTTATCCAGCCCTCACTCCTTCTCT
AGGCGCCCCCATATGGCCATATGAGATCTTATATGGGGCACCCCCGCCCCTTGTAAACTTCC
CTGACCCTGACATGACAAGAGTTACTAACAGCCCCTCTCTCCAAGCTCACTTACAGGCTCTC
TACTTAGTCCAGCACGAAGTCTGGAGACCTCTGGCGGCAGCCTACCAAGAACAACTGGACCG
ACCGGTGGTACCTCACCCTTACCGAGTCGGCGACACAGTGTGGGTCCGCCGACACCAGACTA
AGAACCTAGAACCTCGCTGGAAAGGACCTTACACAGTCCTGCTGACCACCCCCACCGCCCTC
AAAGTAGACGGCATCGCAGCTTGGATACACGCCGCCCACGTGAAGGCTGCCGACCCCGGGGG
TGGACCATCCTCTAGACTGCCATGAAGAAGCACCTGACCACCTTTCTGGTCATCCTGTGGCT
GTACTTCTACAGAGGCAACGGCAAGAACCAGGTGGAACAGAGCCCTCAGAGCCTGATCATCC
TGGAAGGCAAGAACTGCACCCTGCAGTGCAACTACACCGTGTCTCCCTTCAGCAACCTGCGG
TGGTACAAGCAGGATACAGGCAGAGGCCCTGTGTCTCTGACCATCATGACCTTCAGCGAGAA
CACCAAGAGCAACGGCCGGTACACCGCCACACTGGATGCCGATACAAAGCAGAGCAGCCTGC
ACATCACAGCCAGCCAGCTGAGCGATAGCGCCAGCTACATCTGCGTGGTGTCCGATAGAGGC
AGCACCCTGGGCAGACTGTACTTTGGCAGAGGCACCCAGCTGACCGTGTGGCCCGATATTCA
GAACCCTGATCCTGCCGTGTACCAGCTGAGAGACAGCAAGTCCAGCGACAAGAGCGTGTGCC
TGTTCACCGACTTCGACAGCCAGACCAACGTGTCCCAGAGCAAGGACAGCGACGTGTACATC
ACCGACAAGtgcGTGCTGGACATGCGGAGCATGGACTTCAAGAGCAATAGCGCCGTGGCCTG
GTCCAACAAGAGCGATTTCGCCTGCGCCAACGCCTTCAACAACAGCATTATCCCCGAGGACA
CATTCTTCCCAAGTCCTGAGAGCAGCTGCGACGTGAAGCTGGTGGAAAAGAGCTTCGAGACA
GACACCAACCTGAACTTCCAGAACCTGAGCGTGATCGGCTTCCGGATCCTGCTGCTGAAAGT
GGCCGGCTTCAACCTGCTGATGACCCTGAGACTGTGGTCTAGCGGAAGCGGCGCCACCAATT
TCAGCCTGCTGAAACAGGCTGGCGACGTGGAAGAGAACCCCGGACCTATGACCATCCGGCTG
CTGTGCTATATGGGCTTCTACTTCCTCGGAGCCGGCCTGATGGAAGCCGACATCTACCAGAC
ACCTAGATACCTGGTCATCGGCACCGGCAAAAAGATCACCCTGGAATGCTCCCAGACCATGG
GCCACGACAAGATGTACTGGTATCAGCAGGACCCCGGCATGGAACTGCATCTGATCCACTAC
AGCTACGGCGTGAACAGCACCGAGAAGGGCGATCTGTCTAGCGAGAGCACCGTGTCCAGAAT
CCGGACCGAGCACTTCCCACTGACACTGGAAAGCGCCAGACCTAGCCACACCAGCCAGTACC
TGTGTGCCACTGGACAGGGGGCGCAAGATACGCAGTATTTTGGCCCAGGCACCCGGCTGACA
GTGCTCGAGGACCTCAAGAATGTGTTTCCGCCCGAAGTCGCGGTTTTTGAACCATCAGAAGC
CGAGATCTCTCATACACAAAAGGCGACGCTCGTATGCCTcGCGACGGGATTTTATCCGGACC
ACGTCGAGCTTTCCTGGTGGGTTAACGGAAAGGAGGTGCATTCCGGAGTTTGCACGGACCCT
CAGCCATTGAAGGAACAGCCCGCACTGAACGACAGTAGGTATTGCCTTTCATCTCGCCTGCG
CGTGTCTGCGACATTCTGGCAAAACCCAAGAAATCACTTCAGATGTCAAGTTCAGTTCTACG
GTCTCAGCGAGAATGATGAGTGGACACAAGATAGGGCTAAACCCGTGACTCAAATAGTCTCT
GCCGAGGCCTGGGGGAGGGCGGATTGCGGCTTCACATCAGAATCATACCAACAAGGAGTATT
GAGCGCGACAATTCTTTACGAAATTCTGCTTGGGAAAGCGACTCTGTACGCGGTGCTCGTGT
CCGCTTTGGTTCTTATGGCAATGGTTAAACGAAAGGATAGTAGGGGCGGtAGtGGaCAGTGT
ACTAATTATGCTCTCTTGAAATTGGCTGGAGATGTTGAGAGCAATCCCGGGCCCATGTGGCA
GCTGCTCCTCCCAACTGCTCTGCTACTTCTAGTTTCAGCTGGCATGCGGACTGAAGATCTCC
CAAAGGCTGTGGTGTTCCTGGAGCCTCAATGGTACAGGGTGCTCGAGAAGGACAGTGTGACT
CTGAAGTGCCAGGGAGCCTACTCCCCTGAGGACAATTCCACACAGTGGTTTCACAATGAGAG
CCTCATCTCAAGCCAGGCCTCGAGCTACTTCATTGACGCTGCCACAGTCGACGACAGTGGAG
AGTACAGGTGCCAGACAAACCTCTCCACCCTCAGTGACCCGGTGCAGCTAGAAGTCCATATC
GGCTGGCTGTTGCTCCAGGCCCCTCGGTGGGTGTTCAAGGAGGAAGACCCTATTCACCTGAG
GTGTCACAGCTGGAAGAACACTGCTCTGCATAAGGTCACATATTTACAGAATGGCAAAGGCA
GGAAGTATTTTCATCATAATTCTGACTTCTACATTCCAAAAGCCACACTCAAAGACAGCGGC
TCCTACTTCTGCAGGGGGCTTTTTGGGAGTAAAAATGTGTCTTCAGAGACTGTGAACATCAC
CATCACTCAAGGTTTGGCAGTGTCAACCATCTCATCATTCTTTCCACCTGGGTACCAAgtct
ctttctgcttggtgatggtactcctttttgcagtggacacaggactatatttctctgtgaag
acaaacattcgaagctcaacaagagactggaaggaccataaatttaaatggagaaaggaccc
tcaagacaaaTAACAATTGCGCGTCATCATCGATCCGGATTAGTCCAATTTGTTAAAGACAG
GATATCAGTGGTCCAGGCTCTAGTTTTGACTCAACAATATCACCAGCTGAAGCCTATAGAGT
ACGAGCCATAGATAAAATAAAAGATTTTATTTAGTCTCCAGAAAAAGGGGGGAATGAAAGAC
CCCACCTGTAGGTTTGGCAAGCTAGCTTAAGTAACGCCATTTTGCAAGGCATGGAAAAATAC
ATAACTGAGAATAGAGAAGTTCAGATCAAGGTCAGGAACAGATGGAACAGCTGAATATGGGC
CAAACAGGATATCTGTGGTAAGCAGTTCCTGCCCCGGCTCAGGGCCAAGAACAGATGGAACA
GCTGAATATGGGCCAAACAGGATATCTGTGGTAAGCAGTTCCTGCCCCGGCTCAGGGCCAAG
AACAGATGGTCCCCAGATGCGGTCCAGCCCTCAGCAGTTTCTAGAGAACCATCAGATGTTTC
CAGGGTGCCCCAAGGACCTGAAATGACCCTGTGCCTTATTTGAACTAACCAATCAGTTCGCT
TCTCGCTTCTGTTCGCGCGCTTCTGCTCCCCGAGCTCAATAAAAGAGCCCACAACCCCTCAC
TCGGGGCGCCAGTCCTCCGATTGACTGAGTCGCCCGGGTACCCGTGTATCCAATAAACCCTC
TTGCAGTTGCATCCGACTTGTGGTCTCGCTGTTCCTTGGGAGGGTCTCCTCTGAGTGATTGA
CTACCCGTCAGCGGGGGTCTTTCA
TCR/FcR #2 transgenic vector (SEQ ID NO: 178)
(SEQ ID NO: 178)
AATGAAAGACCCCACCTGTAGGTTTGGCAAGCTAGCTTAAGTAACGCCATTTTGCAAGGCAT
GGAAAAATACATAACTGAGAATAGAAAAGTTCAGATCAAGGTCAGGAACAGATGGAACAGCT
GAATATGGGCCAAACAGGATATCTGTGGTAAGCAGTTCCTGCCCCGGCTCAGGGCCAAGAAC
AGATGGAACAGCTGAATATGGGCCAAACAGGATATCTGTGGTAAGCAGTTCCTGCCCCGGCT
CAGGGCCAAGAACAGATGGTCCCCAGATGCGGTCCAGCCCTCAGCAGTTTCTAGAGAACCAT
CAGATGTTTCCAGGGTGCCCCAAGGACCTGAAATGACCCTGTGCCTTATTTGAACTAACCAA
TCAGTTCGCTTCTCGCTTCTGTTCGCGCGCTTATGCTCCCCGAGCTCAATAAAAGAGCCCAC
AACCCCTCACTCGGGGCGCCAGTCCTCCGATTGACTGAGTCGCCCGGGTACCCGTGTATCCA
ATAAACCCTCTTGCAGTTGCATCCGACTTGTGGTCTCGCTGTTCCTTGGGAGGGTCTCCTCT
GAGTGATTGACTACCCGTCAGCGGGGGTCTTTCATTTGGGGGCTCGTCCGGGATCGGGAGAC
CCCTGCCCAGGGACCACCGACCCACCACCGGGAGGTAAGCTGGCCAGCAACTTATCTGTGTC
TGTCCGATTGTCTAGTGTCTATGACTGATTTTATGCGCCTGCGTCGGTACTAGTTAGCTAAC
TAGCTCTGTATCTGGCGGACCCGTGGTGGAACTGACGAGTTCGGAACACCCGGCCGCAACCC
TGGGAGACGTCCCAGGGACTTCGGGGGCCGTTTTTGTGGCCCGACCTGAGTCCTAAAATCCC
GATCGTTTAGGACTCTTTGGTGCACCCCCCTTAGAGGAGGGATATGTGGTTCTGGTAGGAGA
CGAGAACCTAAAACAGTTCCCGCCTCCGTCTGAATTTTTGCTTTCGGTTTGGGACCGAAGCC
GCGCCGCGCGTCTTGTCTGCTGCAGCATCGTTCTGTGTTGTCTCTGTCTGACTGTGTTTCTG
TATTTGTCTGAAAATATGGGCCCGGGCTAGCCTGTTACCACTCCCTTAAGTTTGACCTTAGG
TCACTGGAAAGATGTCGAGCGGATCGCTCACAACCAGTCGGTAGATGTCAAGAAGAGACGTT
GGGTTACCTTCTGCTCTGCAGAATGGCCAACCTTTAACGTCGGATGGCCGCGAGACGGCACC
TTTAACCGAGACCTCATCACCCAGGTTAAGATCAAGGTCTTTTCACCTGGCCCGCATGGACA
CCCAGACCAGGTGGGGTACATCGTGACCTGGGAAGCCTTGGCTTTTGACCCCCCTCCCTGGG
TCAAGCCCTTTGTACACCCTAAGCCTCCGCCTCCTCTTCCTCCATCCGCCCCGTCTCTCCCC
CTTGAACCTCCTCGTTCGACCCCGCCTCGATCCTCCCTTTATCCAGCCCTCACTCCTTCTCT
AGGCGCCCCCATATGGCCATATGAGATCTTATATGGGGCACCCCCGCCCCTTGTAAACTTCC
CTGACCCTGACATGACAAGAGTTACTAACAGCCCCTCTCTCCAAGCTCACTTACAGGCTCTC
TACTTAGTCCAGCACGAAGTCTGGAGACCTCTGGCGGCAGCCTACCAAGAACAACTGGACCG
ACCGGTGGTACCTCACCCTTACCGAGTCGGCGACACAGTGTGGGTCCGCCGACACCAGACTA
AGAACCTAGAACCTCGCTGGAAAGGACCTTACACAGTCCTGCTGACCACCCCCACCGCCCTC
AAAGTAGACGGCATCGCAGCTTGGATACACGCCGCCCACGTGAAGGCTGCCGACCCCGGGGG
TGGACCATCCTCTAGACTGCCATGAAGAAGCACCTGACCACCTTTCTGGTCATCCTGTGGCT
GTACTTCTACAGAGGCAACGGCAAGAACCAGGTGGAACAGAGCCCTCAGAGCCTGATCATCC
TGGAAGGCAAGAACTGCACCCTGCAGTGCAACTACACCGTGTCTCCCTTCAGCAACCTGCGG
TGGTACAAGCAGGATACAGGCAGAGGCCCTGTGTCTCTGACCATCATGACCTTCAGCGAGAA
CACCAAGAGCAACGGCCGGTACACCGCCACACTGGATGCCGATACAAAGCAGAGCAGCCTGC
ACATCACAGCCAGCCAGCTGAGCGATAGCGCCAGCTACATCTGCGTGGTGTCCGATAGAGGC
AGCACCCTGGGCAGACTGTACTTTGGCAGAGGCACCCAGCTGACCGTGTGGCCCGATATTCA
GAACCCTGATCCTGCCGTGTACCAGCTGAGAGACAGCAAGTCCAGCGACAAGAGCGTGTGCC
TGTTCACCGACTTCGACAGCCAGACCAACGTGTCCCAGAGCAAGGACAGCGACGTGTACATC
ACCGACAAGtgcGTGCTGGACATGCGGAGCATGGACTTCAAGAGCAATAGCGCCGTGGCCTG
GTCCAACAAGAGCGATTTCGCCTGCGCCAACGCCTTCAACAACAGCATTATCCCCGAGGACA
CATTCTTCCCAAGTCCTGAGAGCAGCTGCGACGTGAAGCTGGTGGAAAAGAGCTTCGAGACA
GACACCAACCTGAACTTCCAGAACCTGAGCGTGATCGGCTTCCGGATCCTGCTGCTGAAAGT
GGCCGGCTTCAACCTGCTGATGACCCTGAGACTGTGGTCTAGCGGAAGCGGCGCCACCAATT
TCAGCCTGCTGAAACAGGCTGGCGACGTGGAAGAGAACCCCGGACCTATGACCATCCGGCTG
CTGTGCTATATGGGCTTCTACTTCCTCGGAGCCGGCCTGATGGAAGCCGACATCTACCAGAC
ACCTAGATACCTGGTCATCGGCACCGGCAAAAAGATCACCCTGGAATGCTCCCAGACCATGG
GCCACGACAAGATGTACTGGTATCAGCAGGACCCCGGCATGGAACTGCATCTGATCCACTAC
AGCTACGGCGTGAACAGCACCGAGAAGGGCGATCTGTCTAGCGAGAGCACCGTGTCCAGAAT
CCGGACCGAGCACTTCCCACTGACACTGGAAAGCGCCAGACCTAGCCACACCAGCCAGTACC
TGTGTGCCACTGGACAGGGGGCGCAAGATACGCAGTATTTTGGCCCAGGCACCCGGCTGACA
GTGCTCGAGGACCTCAAGAATGTGTTTCCGCCCGAAGTCGCGGTTTTTGAACCATCAGAAGC
CGAGATCTCTCATACACAAAAGGCGACGCTCGTATGCCTCGCGACGGGATTTTATCCGGACC
ACGTCGAGCTTTCCTGGTGGGTTAACGGAAAGGAGGTGCATTCCGGAGTTTGCACGGACCCT
CAGCCATTGAAGGAACAGCCCGCACTGAACGACAGTAGGTATTGCCTTTCATCTCGCCTGCG
CGTGTCTGCGACATTCTGGCAAAACCCAAGAAATCACTTCAGATGTCAAGTTCAGTTCTACG
GTCTCAGCGAGAATGATGAGTGGACACAAGATAGGGCTAAACCCGTGACTCAAATAGTCTCT
GCCGAGGCCTGGGGGAGGGCGGATTGCGGCTTCACATCAGAATCATACCAACAAGGAGTATT
GAGCGCGACAATTCTTTACGAAATTCTGCTTGGGAAAGCGACTCTGTACGCGGTGCTCGTGT
CCGCTTTGGTTCTTATGGCAATGGTTAAACGAAAGGATAGTAGGGGCGGtAGtGGaCAGTGT
ACTAATTATGCTCTCTTGAAATTGGCTGGAGATGTTGAGAGCAATCCCGGGCCCATGTGGCA
GCTGCTCCTCCCAACTGCTCTGCTACTTCTAGTTTCAGCTGGCATGCGGACTGAAGATCTCC
CAAAGGCTGTGGTGTTCCTGGAGCCTCAATGGTACAGGGTGCTCGAGAAGGACAGTGTGACT
CTGAAGTGCCAGGGAGCCTACTCCCCTGAGGACAATTCCACACAGTGGTTTCACAATGAGAG
CCTCATCTCAAGCCAGGCCTCGAGCTACTTCATTGACGCTGCCACAGTCGACGACAGTGGAG
AGTACAGGTGCCAGACAAACCTCTCCACCCTCAGTGACCCGGTGCAGCTAGAAGTCCATATC
GGCTGGCTGTTGCTCCAGGCCCCTCGGTGGGTGTTCAAGGAGGAAGACCCTATTCACCTGAG
GTGTCACAGCTGGAAGAACACTGCTCTGCATAAGGTCACATATTTACAGAATGGCAAAGGCA
GGAAGTATTTTCATCATAATTCTGACTTCTACATTCCAAAAGCCACACTCAAAGACAGCGGC
TCCTACTTCTGCAGGGGGCTTgTTGGGAGTAAAAATGTGTCTTCAGAGACTGTGAACATCAC
CATCACTCAAGGTTTGGCAGTGTCAACCATCTCATCATTCTTTCCACCTGGGTACCAAgtct
ctttctgcttggtgatggtactcctttttgcagtggacacaggactatatttctctgtgaag
acaaacattcgaagctcaacaagagactggaaggaccataaatttaaatggagaaaggaccc
tcaagacaaaTAACAATTGCGCGTCATCATCGATCCGGATTAGTCCAATTTGTTAAAGACAG
GATATCAGTGGTCCAGGCTCTAGTTTTGACTCAACAATATCACCAGCTGAAGCCTATAGAGT
ACGAGCCATAGATAAAATAAAAGATTTTATTTAGTCTCCAGAAAAAGGGGGGAATGAAAGAC
CCCACCTGTAGGTTTGGCAAGCTAGCTTAAGTAACGCCATTTTGCAAGGCATGGAAAAATAC
ATAACTGAGAATAGAGAAGTTCAGATCAAGGTCAGGAACAGATGGAACAGCTGAATATGGGC
CAAACAGGATATCTGTGGTAAGCAGTTCCTGCCCCGGCTCAGGGCCAAGAACAGATGGAACA
GCTGAATATGGGCCAAACAGGATATCTGTGGTAAGCAGTTCCTGCCCCGGCTCAGGGCCAAG
AACAGATGGTCCCCAGATGCGGTCCAGCCCTCAGCAGTTTCTAGAGAACCATCAGATGTTTC
CAGGGTGCCCCAAGGACCTGAAATGACCCTGTGCCTTATTTGAACTAACCAATCAGTTCGCT
TCTCGCTTCTGTTCGCGCGCTTCTGCTCCCCGAGCTCAATAAAAGAGCCCACAACCCCTCAC
TCGGGGCGCCAGTCCTCCGATTGACTGAGTCGCCCGGGTACCCGTGTATCCAATAAACCCTC
TTGCAGTTGCATCCGACTTGTGGTCTCGCTGTTCCTTGGGAGGGTCTCCTCTGAGTGATTGA
CTACCCGTCAGCGGGGGTCTTTCA
TCR/FcR #3 transgenic vector (SEQ ID NO: 179)
(SEQ ID NO: 179)
AATGAAAGACCCCACCTGTAGGTTTGGCAAGCTAGCTTAAGTAACGCCATTTTGCAAGGCAT
GGAAAAATACATAACTGAGAATAGAAAAGTTCAGATCAAGGTCAGGAACAGATGGAACAGCT
GAATATGGGCCAAACAGGATATCTGTGGTAAGCAGTTCCTGCCCCGGCTCAGGGCCAAGAAC
AGATGGAACAGCTGAATATGGGCCAAACAGGATATCTGTGGTAAGCAGTTCCTGCCCCGGCT
CAGGGCCAAGAACAGATGGTCCCCAGATGCGGTCCAGCCCTCAGCAGTTTCTAGAGAACCAT
CAGATGTTTCCAGGGTGCCCCAAGGACCTGAAATGACCCTGTGCCTTATTTGAACTAACCAA
TCAGTTCGCTTCTCGCTTCTGTTCGCGCGCTTATGCTCCCCGAGCTCAATAAAAGAGCCCAC
AACCCCTCACTCGGGGCGCCAGTCCTCCGATTGACTGAGTCGCCCGGGTACCCGTGTATCCA
ATAAACCCTCTTGCAGTTGCATCCGACTTGTGGTCTCGCTGTTCCTTGGGAGGGTCTCCTCT
GAGTGATTGACTACCCGTCAGCGGGGGTCTTTCATTTGGGGGCTCGTCCGGGATCGGGAGAC
CCCTGCCCAGGGACCACCGACCCACCACCGGGAGGTAAGCTGGCCAGCAACTTATCTGTGTC
TGTCCGATTGTCTAGTGTCTATGACTGATTTTATGCGCCTGCGTCGGTACTAGTTAGCTAAC
TAGCTCTGTATCTGGCGGACCCGTGGTGGAACTGACGAGTTCGGAACACCCGGCCGCAACCC
TGGGAGACGTCCCAGGGACTTCGGGGGCCGTTTTTGTGGCCCGACCTGAGTCCTAAAATCCC
GATCGTTTAGGACTCTTTGGTGCACCCCCCTTAGAGGAGGGATATGTGGTTCTGGTAGGAGA
CGAGAACCTAAAACAGTTCCCGCCTCCGTCTGAATTTTTGCTTTCGGTTTGGGACCGAAGCC
GCGCCGCGCGTCTTGTCTGCTGCAGCATCGTTCTGTGTTGTCTCTGTCTGACTGTGTTTCTG
TATTTGTCTGAAAATATGGGCCCGGGCTAGCCTGTTACCACTCCCTTAAGTTTGACCTTAGG
TCACTGGAAAGATGTCGAGCGGATCGCTCACAACCAGTCGGTAGATGTCAAGAAGAGACGTT
GGGTTACCTTCTGCTCTGCAGAATGGCCAACCTTTAACGTCGGATGGCCGCGAGACGGCACC
TTTAACCGAGACCTCATCACCCAGGTTAAGATCAAGGTCTTTTCACCTGGCCCGCATGGACA
CCCAGACCAGGTGGGGTACATCGTGACCTGGGAAGCCTTGGCTTTTGACCCCCCTCCCTGGG
TCAAGCCCTTTGTACACCCTAAGCCTCCGCCTCCTCTTCCTCCATCCGCCCCGTCTCTCCCC
CTTGAACCTCCTCGTTCGACCCCGCCTCGATCCTCCCTTTATCCAGCCCTCACTCCTTCTCT
AGGCGCCCCCATATGGCCATATGAGATCTTATATGGGGCACCCCCGCCCCTTGTAAACTTCC
CTGACCCTGACATGACAAGAGTTACTAACAGCCCCTCTCTCCAAGCTCACTTACAGGCTCTC
TACTTAGTCCAGCACGAAGTCTGGAGACCTCTGGCGGCAGCCTACCAAGAACAACTGGACCG
ACCGGTGGTACCTCACCCTTACCGAGTCGGCGACACAGTGTGGGTCCGCCGACACCAGACTA
AGAACCTAGAACCTCGCTGGAAAGGACCTTACACAGTCCTGCTGACCACCCCCACCGCCCTC
AAAGTAGACGGCATCGCAGCTTGGATACACGCCGCCCACGTGAAGGCTGCCGACCCCGGGGG
TGGACCATCCTCTAGACTGCCATGAAGAAGCACCTGACCACCTTTCTGGTCATCCTGTGGCT
GTACTTCTACAGAGGCAACGGCAAGAACCAGGTGGAACAGAGCCCTCAGAGCCTGATCATCC
TGGAAGGCAAGAACTGCACCCTGCAGTGCAACTACACCGTGTCTCCCTTCAGCAACCTGCGG
TGGTACAAGCAGGATACAGGCAGAGGCCCTGTGTCTCTGACCATCATGACCTTCAGCGAGAA
CACCAAGAGCAACGGCCGGTACACCGCCACACTGGATGCCGATACAAAGCAGAGCAGCCTGC
ACATCACAGCCAGCCAGCTGAGCGATAGCGCCAGCTACATCTGCGTGGTGTCCGATAGAGGC
AGCACCCTGGGCAGACTGTACTTTGGCAGAGGCACCCAGCTGACCGTGTGGCCCGATATTCA
GAACCCTGATCCTGCCGTGTACCAGCTGAGAGACAGCAAGTCCAGCGACAAGAGCGTGTGCC
TGTTCACCGACTTCGACAGCCAGACCAACGTGTCCCAGAGCAAGGACAGCGACGTGTACATC
ACCGACAAGtgcGTGCTGGACATGCGGAGCATGGACTTCAAGAGCAATAGCGCCGTGGCCTG
GTCCAACAAGAGCGATTTCGCCTGCGCCAACGCCTTCAACAACAGCATTATCCCCGAGGACA
CATTCTTCCCAAGTCCTGAGAGCAGCTGCGACGTGAAGCTGGTGGAAAAGAGCTTCGAGACA
GACACCAACCTGAACTTCCAGAACCTGAGCGTGATCGGCTTCCGGATCCTGCTGCTGAAAGT
GGCCGGCTTCAACCTGCTGATGACCCTGAGACTGTGGTCTAGCGGAAGCGGCGCCACCAATT
TCAGCCTGCTGAAACAGGCTGGCGACGTGGAAGAGAACCCCGGACCTATGACCATCCGGCTG
CTGTGCTATATGGGCTTCTACTTCCTCGGAGCCGGCCTGATGGAAGCCGACATCTACCAGAC
ACCTAGATACCTGGTCATCGGCACCGGCAAAAAGATCACCCTGGAATGCTCCCAGACCATGG
GCCACGACAAGATGTACTGGTATCAGCAGGACCCCGGCATGGAACTGCATCTGATCCACTAC
AGCTACGGCGTGAACAGCACCGAGAAGGGCGATCTGTCTAGCGAGAGCACCGTGTCCAGAAT
CCGGACCGAGCACTTCCCACTGACACTGGAAAGCGCCAGACCTAGCCACACCAGCCAGTACC
TGTGTGCCACTGGACAGGGGGCGCAAGATACGCAGTATTTTGGCCCAGGCACCCGGCTGACA
GTGCTCGAGGACCTCAAGAATGTGTTTCCGCCCGAAGTCGCGGTTTTTGAACCATCAGAAGC
CGAGATCTCTCATACACAAAAGGCGACGCTCGTATGCCTCGCGACGGGATTTTATCCGGACC
ACGTCGAGCTTTCCTGGTGGGTTAACGGAAAGGAGGTGCATTCCGGAGTTTGCACGGACCCT
CAGCCATTGAAGGAACAGCCCGCACTGAACGACAGTAGGTATTGCCTTTCATCTCGCCTGCG
CGTGTCTGCGACATTCTGGCAAAACCCAAGAAATCACTTCAGATGTCAAGTTCAGTTCTACG
GTCTCAGCGAGAATGATGAGTGGACACAAGATAGGGCTAAACCCGTGACTCAAATAGTCTCT
GCCGAGGCCTGGGGGAGGGCGGATTGCGGCTTCACATCAGAATCATACCAACAAGGAGTATT
GAGCGCGACAATTCTTTACGAAATTCTGCTTGGGAAAGCGACTCTGTACGCGGTGCTCGTGT
CCGCTTTGGTTCTTATGGCAATGGTTAAACGAAAGGATAGTAGGGGCGGtAGtGGaCAGTGT
ACTAATTATGCTCTCTTGAAATTGGCTGGAGATGTTGAGAGCAATCCCGGGCCCATGTGGCA
GCTGCTCCTCCCAACTGCTCTGCTACTTCTAGTTTCAGCTGGCATGCGGACTGAAGATCTCC
CAAAGGCTGTGGTGTTCCTGGAGCCTCAATGGTACAGGGTGCTCGAGAAGGACAGTGTGACT
CTGAAGTGCCAGGGAGCCTACTCCCCTGAGGACAATTCCACACAGTGGTTTCACAATGAGAG
CCTCATCTCAAGCCAGGCCTCGAGCTACTTCATTGACGCTGCCACAGTCGACGACAGTGGAG
AGTACAGGTGCCAGACAAACCTCTCCACCCTCAGTGACCCGGTGCAGCTAGAAGTCCATATC
GGCTGGCTGTTGCTCCAGGCCCCTCGGTGGGTGTTCAAGGAGGAAGACCCTATTCACCTGAG
GTGTCACAGCTGGAAGAACACTGCTCTGCATAAGGTCACATATTTACAGAATGGCAAAGGCA
GGAAGTATTTTCATCATAATTCTGACTTCTACATTCCAAAAGCCACACTCAAAGACAGCGGC
TCCTACTTCTGCAGGGGGCTTgTTGGGAGTAAAAATGTGTCTTCAGAGACTGTGAACATCAC
CGTCCAAGTGCCCAGCATGGGCAGCTCTTCACCAATGGGGgtctctttctgcttggtgatgg
tactcctttttgcagtggacacaggactatatttctctgtgaagacaaacattcgaagctca
acaagagactggaaggaccataaatttaaatggagaaaggaccctcaagacaaaTAACAATT
GCGCGTCATCATCGATCCGGATTAGTCCAATTTGTTAAAGACAGGATATCAGTGGTCCAGGC
TCTAGTTTTGACTCAACAATATCACCAGCTGAAGCCTATAGAGTACGAGCCATAGATAAAAT
AAAAGATTTTATTTAGTCTCCAGAAAAAGGGGGGAATGAAAGACCCCACCTGTAGGTTTGGC
AAGCTAGCTTAAGTAACGCCATTTTGCAAGGCATGGAAAAATACATAACTGAGAATAGAGAA
GTTCAGATCAAGGTCAGGAACAGATGGAACAGCTGAATATGGGCCAAACAGGATATCTGTGG
TAAGCAGTTCCTGCCCCGGCTCAGGGCCAAGAACAGATGGAACAGCTGAATATGGGCCAAAC
AGGATATCTGTGGTAAGCAGTTCCTGCCCCGGCTCAGGGCCAAGAACAGATGGTCCCCAGAT
GCGGTCCAGCCCTCAGCAGTTTCTAGAGAACCATCAGATGTTTCCAGGGTGCCCCAAGGACC
TGAAATGACCCTGTGCCTTATTTGAACTAACCAATCAGTTCGCTTCTCGCTTCTGTTCGCGC
GCTTCTGCTCCCCGAGCTCAATAAAAGAGCCCACAACCCCTCACTCGGGGCGCCAGTCCTCC
GATTGACTGAGTCGCCCGGGTACCCGTGTATCCAATAAACCCTCTTGCAGTTGCATCCGACT
TGTGGTCTCGCTGTTCCTTGGGAGGGTCTCCTCTGAGTGATTGACTACCCGTCAGCGGGGGT
CTTTCA
TCR/FcR #4 transgenic vector (SEQ ID NO: 180)
(SEQ ID NO: 180)
AATGAAAGACCCCACCTGTAGGTTTGGCAAGCTAGCTTAAGTAACGCCATTTTGCAAGGCAT
GGAAAAATACATAACTGAGAATAGAAAAGTTCAGATCAAGGTCAGGAACAGATGGAACAGCT
GAATATGGGCCAAACAGGATATCTGTGGTAAGCAGTTCCTGCCCCGGCTCAGGGCCAAGAAC
AGATGGAACAGCTGAATATGGGCCAAACAGGATATCTGTGGTAAGCAGTTCCTGCCCCGGCT
CAGGGCCAAGAACAGATGGTCCCCAGATGCGGTCCAGCCCTCAGCAGTTTCTAGAGAACCAT
CAGATGTTTCCAGGGTGCCCCAAGGACCTGAAATGACCCTGTGCCTTATTTGAACTAACCAA
TCAGTTCGCTTCTCGCTTCTGTTCGCGCGCTTATGCTCCCCGAGCTCAATAAAAGAGCCCAC
AACCCCTCACTCGGGGCGCCAGTCCTCCGATTGACTGAGTCGCCCGGGTACCCGTGTATCCA
ATAAACCCTCTTGCAGTTGCATCCGACTTGTGGTCTCGCTGTTCCTTGGGAGGGTCTCCTCT
GAGTGATTGACTACCCGTCAGCGGGGGTCTTTCATTTGGGGGCTCGTCCGGGATCGGGAGAC
CCCTGCCCAGGGACCACCGACCCACCACCGGGAGGTAAGCTGGCCAGCAACTTATCTGTGTC
TGTCCGATTGTCTAGTGTCTATGACTGATTTTATGCGCCTGCGTCGGTACTAGTTAGCTAAC
TAGCTCTGTATCTGGCGGACCCGTGGTGGAACTGACGAGTTCGGAACACCCGGCCGCAACCC
TGGGAGACGTCCCAGGGACTTCGGGGGCCGTTTTTGTGGCCCGACCTGAGTCCTAAAATCCC
GATCGTTTAGGACTCTTTGGTGCACCCCCCTTAGAGGAGGGATATGTGGTTCTGGTAGGAGA
CGAGAACCTAAAACAGTTCCCGCCTCCGTCTGAATTTTTGCTTTCGGTTTGGGACCGAAGCC
GCGCCGCGCGTCTTGTCTGCTGCAGCATCGTTCTGTGTTGTCTCTGTCTGACTGTGTTTCTG
TATTTGTCTGAAAATATGGGCCCGGGCTAGCCTGTTACCACTCCCTTAAGTTTGACCTTAGG
TCACTGGAAAGATGTCGAGCGGATCGCTCACAACCAGTCGGTAGATGTCAAGAAGAGACGTT
GGGTTACCTTCTGCTCTGCAGAATGGCCAACCTTTAACGTCGGATGGCCGCGAGACGGCACC
TTTAACCGAGACCTCATCACCCAGGTTAAGATCAAGGTCTTTTCACCTGGCCCGCATGGACA
CCCAGACCAGGTGGGGTACATCGTGACCTGGGAAGCCTTGGCTTTTGACCCCCCTCCCTGGG
TCAAGCCCTTTGTACACCCTAAGCCTCCGCCTCCTCTTCCTCCATCCGCCCCGTCTCTCCCC
CTTGAACCTCCTCGTTCGACCCCGCCTCGATCCTCCCTTTATCCAGCCCTCACTCCTTCTCT
AGGCGCCCCCATATGGCCATATGAGATCTTATATGGGGCACCCCCGCCCCTTGTAAACTTCC
CTGACCCTGACATGACAAGAGTTACTAACAGCCCCTCTCTCCAAGCTCACTTACAGGCTCTC
TACTTAGTCCAGCACGAAGTCTGGAGACCTCTGGCGGCAGCCTACCAAGAACAACTGGACCG
ACCGGTGGTACCTCACCCTTACCGAGTCGGCGACACAGTGTGGGTCCGCCGACACCAGACTA
AGAACCTAGAACCTCGCTGGAAAGGACCTTACACAGTCCTGCTGACCACCCCCACCGCCCTC
AAAGTAGACGGCATCGCAGCTTGGATACACGCCGCCCACGTGAAGGCTGCCGACCCCGGGGG
TGGACCATCCTCTAGACTGCCATGAAGAAGCACCTGACCACCTTTCTGGTCATCCTGTGGCT
GTACTTCTACAGAGGCAACGGCAAGAACCAGGTGGAACAGAGCCCTCAGAGCCTGATCATCC
TGGAAGGCAAGAACTGCACCCTGCAGTGCAACTACACCGTGTCTCCCTTCAGCAACCTGCGG
TGGTACAAGCAGGATACAGGCAGAGGCCCTGTGTCTCTGACCATCATGACCTTCAGCGAGAA
CACCAAGAGCAACGGCCGGTACACCGCCACACTGGATGCCGATACAAAGCAGAGCAGCCTGC
ACATCACAGCCAGCCAGCTGAGCGATAGCGCCAGCTACATCTGCGTGGTGTCCGATAGAGGC
AGCACCCTGGGCAGACTGTACTTTGGCAGAGGCACCCAGCTGACCGTGTGGCCCGATATTCA
GAACCCTGATCCTGCCGTGTACCAGCTGAGAGACAGCAAGTCCAGCGACAAGAGCGTGTGCC
TGTTCACCGACTTCGACAGCCAGACCAACGTGTCCCAGAGCAAGGACAGCGACGTGTACATC
ACCGACAAGtgcGTGCTGGACATGCGGAGCATGGACTTCAAGAGCAATAGCGCCGTGGCCTG
GTCCAACAAGAGCGATTTCGCCTGCGCCAACGCCTTCAACAACAGCATTATCCCCGAGGACA
CATTCTTCCCAAGTCCTGAGAGCAGCTGCGACGTGAAGCTGGTGGAAAAGAGCTTCGAGACA
GACACCAACCTGAACTTCCAGAACCTGAGCGTGATCGGCTTCCGGATCCTGCTGCTGAAAGT
GGCCGGCTTCAACCTGCTGATGACCCTGAGACTGTGGTCTAGCGGAAGCGGCGCCACCAATT
TCAGCCTGCTGAAACAGGCTGGCGACGTGGAAGAGAACCCCGGACCTATGACCATCCGGCTG
CTGTGCTATATGGGCTTCTACTTCCTCGGAGCCGGCCTGATGGAAGCCGACATCTACCAGAC
ACCTAGATACCTGGTCATCGGCACCGGCAAAAAGATCACCCTGGAATGCTCCCAGACCATGG
GCCACGACAAGATGTACTGGTATCAGCAGGACCCCGGCATGGAACTGCATCTGATCCACTAC
AGCTACGGCGTGAACAGCACCGAGAAGGGCGATCTGTCTAGCGAGAGCACCGTGTCCAGAAT
CCGGACCGAGCACTTCCCACTGACACTGGAAAGCGCCAGACCTAGCCACACCAGCCAGTACC
TGTGTGCCACTGGACAGGGGGCGCAAGATACGCAGTATTTTGGCCCAGGCACCCGGCTGACA
GTGCTCGAGGACCTCAAGAATGTGTTTCCGCCCGAAGTCGCGGTTTTTGAACCATCAGAAGC
CGAGATCTCTCATACACAAAAGGCGACGCTCGTATGCCTcGCGACGGGATTTTATCCGGACC
ACGTCGAGCTTTCCTGGTGGGTTAACGGAAAGGAGGTGCATTCCGGAGTTTGCACGGACCCT
CAGCCATTGAAGGAACAGCCCGCACTGAACGACAGTAGGTATTGCCTTTCATCTCGCCTGCG
CGTGTCTGCGACATTCTGGCAAAACCCAAGAAATCACTTCAGATGTCAAGTTCAGTTCTACG
GTCTCAGCGAGAATGATGAGTGGACACAAGATAGGGCTAAACCCGTGACTCAAATAGTCTCT
GCCGAGGCCTGGGGGAGGGCGGATTGCGGCTTCACATCAGAATCATACCAACAAGGAGTATT
GAGCGCGACAATTCTTTACGAAATTCTGCTTGGGAAAGCGACTCTGTACGCGGTGCTCGTGT
CCGCTTTGGTTCTTATGGCAATGGTTAAACGAAAGGATAGTAGGGGCGGtAGtGGaCAGTGT
ACTAATTATGCTCTCTTGAAATTGGCTGGAGATGTTGAGAGCAATCCCGGGCCCATGTGGCA
GCTGCTCCTCCCAACTGCTCTGCTACTTCTAGTTTCAGCTGGCATGCGGACTGAAGATCTCC
CAAAGGCTGTGGTGTTCCTGGAGCCTCAATGGTACAGGGTGCTCGAGAAGGACAGTGTGACT
CTGAAGTGCCAGGGAGCCTACTCCCCTGAGGACAATTCCACACAGTGGTTTCACAATGAGAG
CCTCATCTCAAGCCAGGCCTCGAGCTACTTCATTGACGCTGCCACAGTCGACGACAGTGGAG
AGTACAGGTGCCAGACAAACCTCTCCACCCTCAGTGACCCGGTGCAGCTAGAAGTCCATATC
GGCTGGCTGTTGCTCCAGGCCCCTCGGTGGGTGTTCAAGGAGGAAGACCCTATTCACCTGAG
GTGTCACAGCTGGAAGAACACTGCTCTGCATAAGGTCACATATTTACAGAATGGCAAAGGCA
GGAAGTATTTTCATCATAATTCTGACTTCTACATTCCAAAAGCCACACTCAAAGACAGCGGC
TCCTACTTCTGCAGGGGGCTTgTTGGGAGTAAAAATGTGTCTTCAGAGACTGTGAACATCAC
CGTCCAAGTGCCCAGCATGGGCAGCTCTTCACCAATGGGGCTCTGTTACCTTCTTGACGGTA
TTCTTTTTATTTACGGCGTCATCCTCACTGCCCTCTTTTTGAGGGTCAAGTTTTCAAGATCC
GCCGACGCACCTGCTTACCAGCAAGGACAAAATCAACTTTATAACGAACTCAACCTTGGTCG
GCGGGAAGAATATGATGTACTCGACAAAAGAAGAGGCCGCGACCCGGAGATGGGTGGCAAAC
CACAGCGGAGGAAAAACCCCCAGGAAGGTCTCTATAACGAATTGCAGAAAGATAAAATGGCC
GAGGCTTATTCAGAGATCGGCATGAAAGGAGAGCGACGACGAGGAAAGGGGCATGATGGTCT
GTATCAAGGGCTCAGCACGGCAACAAAGGACACATATGATGCCTTGCATATGCAGGCGCTTC
CGCCGCGCTAATAACAATTGCGCGTCATCATCGATCCGGATTAGTCCAATTTGTTAAAGACA
GGATATCAGTGGTCCAGGCTCTAGTTTTGACTCAACAATATCACCAGCTGAAGCCTATAGAG
TACGAGCCATAGATAAAATAAAAGATTTTATTTAGTCTCCAGAAAAAGGGGGGAATGAAAGA
CCCCACCTGTAGGTTTGGCAAGCTAGCTTAAGTAACGCCATTTTGCAAGGCATGGAAAAATA
CATAACTGAGAATAGAGAAGTTCAGATCAAGGTCAGGAACAGATGGAACAGCTGAATATGGG
CCAAACAGGATATCTGTGGTAAGCAGTTCCTGCCCCGGCTCAGGGCCAAGAACAGATGGAAC
AGCTGAATATGGGCCAAACAGGATATCTGTGGTAAGCAGTTCCTGCCCCGGCTCAGGGCCAA
GAACAGATGGTCCCCAGATGCGGTCCAGCCCTCAGCAGTTTCTAGAGAACCATCAGATGTTT
CCAGGGTGCCCCAAGGACCTGAAATGACCCTGTGCCTTATTTGAACTAACCAATCAGTTCGC
TTCTCGCTTCTGTTCGCGCGCTTCTGCTCCCCGAGCTCAATAAAAGAGCCCACAACCCCTCA
CTCGGGGCGCCAGTCCTCCGATTGACTGAGTCGCCCGGGTACCCGTGTATCCAATAAACCCT
CTTGCAGTTGCATCCGACTTGTGGTCTCGCTGTTCCTTGGGAGGGTCTCCTCTGAGTGATTG
ACTACCCGTCAGCGGGGGTCTTTCA
TCR/FcR #5 transgenic vector (SEQ ID NO: 181])
(SEQ ID NO: 181)
AATGAAAGACCCCACCTGTAGGTTTGGCAAGCTAGCTTAAGTAACGCCATTTTGCAAGGCAT
GGAAAAATACATAACTGAGAATAGAAAAGTTCAGATCAAGGTCAGGAACAGATGGAACAGCT
GAATATGGGCCAAACAGGATATCTGTGGTAAGCAGTTCCTGCCCCGGCTCAGGGCCAAGAAC
AGATGGAACAGCTGAATATGGGCCAAACAGGATATCTGTGGTAAGCAGTTCCTGCCCCGGCT
CAGGGCCAAGAACAGATGGTCCCCAGATGCGGTCCAGCCCTCAGCAGTTTCTAGAGAACCAT
CAGATGTTTCCAGGGTGCCCCAAGGACCTGAAATGACCCTGTGCCTTATTTGAACTAACCAA
TCAGTTCGCTTCTCGCTTCTGTTCGCGCGCTTATGCTCCCCGAGCTCAATAAAAGAGCCCAC
AACCCCTCACTCGGGGCGCCAGTCCTCCGATTGACTGAGTCGCCCGGGTACCCGTGTATCCA
ATAAACCCTCTTGCAGTTGCATCCGACTTGTGGTCTCGCTGTTCCTTGGGAGGGTCTCCTCT
GAGTGATTGACTACCCGTCAGCGGGGGTCTTTCATTTGGGGGCTCGTCCGGGATCGGGAGAC
CCCTGCCCAGGGACCACCGACCCACCACCGGGAGGTAAGCTGGCCAGCAACTTATCTGTGTC
TGTCCGATTGTCTAGTGTCTATGACTGATTTTATGCGCCTGCGTCGGTACTAGTTAGCTAAC
TAGCTCTGTATCTGGCGGACCCGTGGTGGAACTGACGAGTTCGGAACACCCGGCCGCAACCC
TGGGAGACGTCCCAGGGACTTCGGGGGCCGTTTTTGTGGCCCGACCTGAGTCCTAAAATCCC
GATCGTTTAGGACTCTTTGGTGCACCCCCCTTAGAGGAGGGATATGTGGTTCTGGTAGGAGA
CGAGAACCTAAAACAGTTCCCGCCTCCGTCTGAATTTTTGCTTTCGGTTTGGGACCGAAGCC
GCGCCGCGCGTCTTGTCTGCTGCAGCATCGTTCTGTGTTGTCTCTGTCTGACTGTGTTTCTG
TATTTGTCTGAAAATATGGGCCCGGGCTAGCCTGTTACCACTCCCTTAAGTTTGACCTTAGG
TCACTGGAAAGATGTCGAGCGGATCGCTCACAACCAGTCGGTAGATGTCAAGAAGAGACGTT
GGGTTACCTTCTGCTCTGCAGAATGGCCAACCTTTAACGTCGGATGGCCGCGAGACGGCACC
TTTAACCGAGACCTCATCACCCAGGTTAAGATCAAGGTCTTTTCACCTGGCCCGCATGGACA
CCCAGACCAGGTGGGGTACATCGTGACCTGGGAAGCCTTGGCTTTTGACCCCCCTCCCTGGG
TCAAGCCCTTTGTACACCCTAAGCCTCCGCCTCCTCTTCCTCCATCCGCCCCGTCTCTCCCC
CTTGAACCTCCTCGTTCGACCCCGCCTCGATCCTCCCTTTATCCAGCCCTCACTCCTTCTCT
AGGCGCCCCCATATGGCCATATGAGATCTTATATGGGGCACCCCCGCCCCTTGTAAACTTCC
CTGACCCTGACATGACAAGAGTTACTAACAGCCCCTCTCTCCAAGCTCACTTACAGGCTCTC
TACTTAGTCCAGCACGAAGTCTGGAGACCTCTGGCGGCAGCCTACCAAGAACAACTGGACCG
ACCGGTGGTACCTCACCCTTACCGAGTCGGCGACACAGTGTGGGTCCGCCGACACCAGACTA
AGAACCTAGAACCTCGCTGGAAAGGACCTTACACAGTCCTGCTGACCACCCCCACCGCCCTC
AAAGTAGACGGCATCGCAGCTTGGATACACGCCGCCCACGTGAAGGCTGCCGACCCCGGGGG
TGGACCATCCTCTAGACTGCCATGAAGAAGCACCTGACCACCTTTCTGGTCATCCTGTGGCT
GTACTTCTACAGAGGCAACGGCAAGAACCAGGTGGAACAGAGCCCTCAGAGCCTGATCATCC
TGGAAGGCAAGAACTGCACCCTGCAGTGCAACTACACCGTGTCTCCCTTCAGCAACCTGCGG
TGGTACAAGCAGGATACAGGCAGAGGCCCTGTGTCTCTGACCATCATGACCTTCAGCGAGAA
CACCAAGAGCAACGGCCGGTACACCGCCACACTGGATGCCGATACAAAGCAGAGCAGCCTGC
ACATCACAGCCAGCCAGCTGAGCGATAGCGCCAGCTACATCTGCGTGGTGTCCGATAGAGGC
AGCACCCTGGGCAGACTGTACTTTGGCAGAGGCACCCAGCTGACCGTGTGGCCCGATATTCA
GAACCCTGATCCTGCCGTGTACCAGCTGAGAGACAGCAAGTCCAGCGACAAGAGCGTGTGCC
TGTTCACCGACTTCGACAGCCAGACCAACGTGTCCCAGAGCAAGGACAGCGACGTGTACATC
ACCGACAAGtgcGTGCTGGACATGCGGAGCATGGACTTCAAGAGCAATAGCGCCGTGGCCTG
GTCCAACAAGAGCGATTTCGCCTGCGCCAACGCCTTCAACAACAGCATTATCCCCGAGGACA
CATTCTTCCCAAGTCCTGAGAGCAGCTGCGACGTGAAGCTGGTGGAAAAGAGCTTCGAGACA
GACACCAACCTGAACTTCCAGAACCTGAGCGTGATCGGCTTCCGGATCCTGCTGCTGAAAGT
GGCCGGCTTCAACCTGCTGATGACCCTGAGACTGTGGTCTAGCGGAAGCGGCGCCACCAATT
TCAGCCTGCTGAAACAGGCTGGCGACGTGGAAGAGAACCCCGGACCTATGACCATCCGGCTG
CTGTGCTATATGGGCTTCTACTTCCTCGGAGCCGGCCTGATGGAAGCCGACATCTACCAGAC
ACCTAGATACCTGGTCATCGGCACCGGCAAAAAGATCACCCTGGAATGCTCCCAGACCATGG
GCCACGACAAGATGTACTGGTATCAGCAGGACCCCGGCATGGAACTGCATCTGATCCACTAC
AGCTACGGCGTGAACAGCACCGAGAAGGGCGATCTGTCTAGCGAGAGCACCGTGTCCAGAAT
CCGGACCGAGCACTTCCCACTGACACTGGAAAGCGCCAGACCTAGCCACACCAGCCAGTACC
TGTGTGCCACTGGACAGGGGGCGCAAGATACGCAGTATTTTGGCCCAGGCACCCGGCTGACA
GTGCTCGAGGACCTCAAGAATGTGTTTCCGCCCGAAGTCGCGGTTTTTGAACCATCAGAAGC
CGAGATCTCTCATACACAAAAGGCGACGCTCGTATGCCTcGCGACGGGATTTTATCCGGACC
ACGTCGAGCTTTCCTGGTGGGTTAACGGAAAGGAGGTGCATTCCGGAGTTTGCACGGACCCT
CAGCCATTGAAGGAACAGCCCGCACTGAACGACAGTAGGTATTGCCTTTCATCTCGCCTGCG
CGTGTCTGCGACATTCTGGCAAAACCCAAGAAATCACTTCAGATGTCAAGTTCAGTTCTACG
GTCTCAGCGAGAATGATGAGTGGACACAAGATAGGGCTAAACCCGTGACTCAAATAGTCTCT
GCCGAGGCCTGGGGGAGGGCGGATTGCGGCTTCACATCAGAATCATACCAACAAGGAGTATT
GAGCGCGACAATTCTTTACGAAATTCTGCTTGGGAAAGCGACTCTGTACGCGGTGCTCGTGT
CCGCTTTGGTTCTTATGGCAATGGTTAAACGAAAGGATAGTAGGGGCGGtAGtGGaCAGTGT
ACTAATTATGCTCTCTTGAAATTGGCTGGAGATGTTGAGAGCAATCCCGGGCCCATGTGGCA
GCTGCTCCTCCCAACTGCTCTGCTACTTCTAGTTTCAGCTGGCATGCGGACTGAAGATCTCC
CAAAGGCTGTGGTGTTCCTGGAGCCTCAATGGTACAGGGTGCTCGAGAAGGACAGTGTGACT
CTGAAGTGCCAGGGAGCCTACTCCCCTGAGGACAATTCCACACAGTGGTTTCACAATGAGAG
CCTCATCTCAAGCCAGGCCTCGAGCTACTTCATTGACGCTGCCACAGTCGACGACAGTGGAG
AGTACAGGTGCCAGACAAACCTCTCCACCCTCAGTGACCCGGTGCAGCTAGAAGTCCATATC
GGCTGGCTGTTGCTCCAGGCCCCTCGGTGGGTGTTCAAGGAGGAAGACCCTATTCACCTGAG
GTGTCACAGCTGGAAGAACACTGCTCTGCATAAGGTCACATATTTACAGAATGGCAAAGGCA
GGAAGTATTTTCATCATAATTCTGACTTCTACATTCCAAAAGCCACACTCAAAGACAGCGGC
TCCTACTTCTGCAGGGGGCTTgTTGGGAGTAAAAATGTGTCTTCAGAGACTGTGAACATCAC
CGTCCAAGTGCCCAGCATGGGCAGCTCTTCACCAATGGGGgtctctttctgcttggtgatgg
tactcctttttgcagtggacacaggactatatttctctgtgaagacaTAACAATTGCGCGTC
ATCATCGATCCGGATTAGTCCAATTTGTTAAAGACAGGATATCAGTGGTCCAGGCTCTAGTT
TTGACTCAACAATATCACCAGCTGAAGCCTATAGAGTACGAGCCATAGATAAAATAAAAGAT
TTTATTTAGTCTCCAGAAAAAGGGGGGAATGAAAGACCCCACCTGTAGGTTTGGCAAGCTAG
CTTAAGTAACGCCATTTTGCAAGGCATGGAAAAATACATAACTGAGAATAGAGAAGTTCAGA
TCAAGGTCAGGAACAGATGGAACAGCTGAATATGGGCCAAACAGGATATCTGTGGTAAGCAG
TTCCTGCCCCGGCTCAGGGCCAAGAACAGATGGAACAGCTGAATATGGGCCAAACAGGATAT
CTGTGGTAAGCAGTTCCTGCCCCGGCTCAGGGCCAAGAACAGATGGTCCCCAGATGCGGTCC
AGCCCTCAGCAGTTTCTAGAGAACCATCAGATGTTTCCAGGGTGCCCCAAGGACCTGAAATG
ACCCTGTGCCTTATTTGAACTAACCAATCAGTTCGCTTCTCGCTTCTGTTCGCGCGCTTCTG
CTCCCCGAGCTCAATAAAAGAGCCCACAACCCCTCACTCGGGGCGCCAGTCCTCCGATTGAC
TGAGTCGCCCGGGTACCCGTGTATCCAATAAACCCTCTTGCAGTTGCATCCGACTTGTGGTC
TCGCTGTTCCTTGGGAGGGTCTCCTCTGAGTGATTGACTACCCGTCAGCGGGGGTCTTTCA

### D. NK Cells

The NK cells that are modified to express the TCR/CD3 receptor complex may be obtained from any suitable source, including fresh or frozen sources. In certain embodiments, NK cells are not NK cells obtained from iPSC differentiation. In certain embodiments, NK cells are not from an NK cell line (e.g., NK-92). In certain embodiments, NK cells are derived from human peripheral blood mononuclear cells (PBMC), unstimulated leukapheresis products (PBSC), NK cell lines (e.g., NK-92), human embryonic stem cells (hESCs), induced pluripotent stem cells (iPSCs), bone marrow, or umbilical cord blood by methods well known in the art. Specifically, the NK cells may be isolated from cord blood (CB), peripheral blood (PB), bone marrow, stem cells, NK cell lines, or a mixture thereof. In particular embodiments, the NK cells are isolated from pooled CB. The CB may be pooled from 2, 3, 4, 5, 6, 7, 8, 9, 10, or more units. The NK cells may be autologous or allogeneic with respect to a recipient individual. The isolated NK cells may or may not be haplotype matched for the subject to be administered the cell therapy. NK cells can be detected by specific surface markers, such as CD16 and CD56 in humans, for example. In some cases, the source of the NK cells is cord blood and the NK cells may be in the cord blood in a heterogeneous mixture of cells and may be depleted of certain cells expressing CD3. In other methods, umbilical CB is used to derive NK cells by the isolation of CD34+cells.

The NK cells may be pre-activated with one or more inflammatory cytokines, and they may be expanded or non-expanded. In some cases, the NK cells are pre-activated either prior to modification (e.g., engineering) or after modification. In specific embodiments, pre-activation of the NK cells may comprise culturing the isolated NK cells in the presence of one or more cytokines. The NK cells may be stimulated with IL-2, or other cytokines that bind the common gamma-chain (e.g., IL-7, IL-12, IL-15, IL-18, IL-21, and others). In particular embodiments, the pre-activation cytokines may be selected from the group consisting of IL-12, IL-15, IL-18, and a combination thereof. One or more additional cytokines may be used for the pre-activation step. The pre-activation may be for a short period of time such as 5-72 hours, such as 10-50 hours, particularly 10-20 hours, such as 12, 13, 14, 15, 16, 17, 18, 19, or 20 hours, specifically about 16 hours. The pre-activation culture may comprise IL-12 at a concentration of 0.1-150 ng/mL, such as 0.5-50 ng/mL, particularly 1-20 ng/mL, such as 2, 3, 4, 5, 6, 7, 8, 9, 10, 11, 12, 13, 14, or 15 ng/mL, specifically about 10 ng/mL. The pre-activation culture may comprise IL-18 and/or IL-15 at a concentration of 10-100 ng/mL, such as 40-60 ng/mL, particularly 45, 46, 47, 48, 49, 50, 51, 52, 53, 54, or 55 ng/mL, specifically about 50 ng/mL.

In some cases, the NK cells are expanded either prior to modification to express constructs described herein (e.g., uTNK15 and/or TCR/FcR constructs). Pre-activated NK cells may be expanded in the presence of artificial antigen presenting cells (aAPCs) and/or feeders/fragments or NK activating beads. The pre-activated NK cells may be washed prior to expansion, such as 2, 3, 4, or 5 times, specifically 3 times. The aAPCs may be engineered to express CD137 ligand and/or a membrane-bound cytokine. The membrane-bound cytokine may be membrane-bound IL-21 (mIL-21) or membrane-bound IL-15 (mIL-15). In particular embodiments, the aAPCs are engineered to express CD137 ligand and mIL-21. The aAPCs may be derived from cancer cells, such as leukemia cells. The aAPCs may not express endogenous HLA class I, II, or CD1d molecules. They may express ICAM-1 (CD54) and LFA-3 (CD58). In particular, the aAPCs may be K562 cells, such as K562 cells engineered to express CD137 ligand and mIL-21. The aAPCs may be irradiated. In some embodiments, fragments of APC can be used to expand the NK cells. The engineering may be by any method known in the art, such as retroviral transduction. Retroviral transduction may be at least, at most, or about 1, 2, 3, 4, 5, 6, 7, 8, 9, 10, 11, 12, 13, 14, 15, 16, 17, 18, 19, or 20 days following NK co-culturing with an antigen presenting cell. In some embodiments, retroviral transduction comprises co-transduction of more than one construct. In some embodiments, retroviral transduction occurs after or at about 5 days of co-culturing with an antigen presenting cell. In some embodiments, co-culturing with an antigen presenting cell continues following transduction of an NK cell. The expansion may be for about 2-30 days, such as 3-20 days, particularly 12-16 days, such as 12, 13, 14, 15, 16, 17, 18, or 19 days, specifically about 14 days. The pre-activated NK cells and aAPCs may be present at a ratio of about 3:1-1:3, such as 2:1, 1:1, 1:2, specifically about 1:2. The expansion culture may further comprise cytokines to promote expansion, such as IL-2. The IL-2 may be present at a concentration of about 10-500 U/mL, such as 100-300 U/mL, particularly about 200 U/mL. The IL-2 may be replenished in the expansion culture, such as every 2-3 days. The aAPCs may be added to the culture at least a second time, such as at about 7 days of expansion.

In particular embodiments, the NK cells are transfected or transduced with one or more membrane bound cytokines, including IL-21, IL-12, IL-18, IL-23, IL-7, or IL-15, either secreted by NK cells or tethered to the NK cell membrane. In such cases, the membrane bound cytokine may be tethered to the NK cell membrane with a particular transmembrane domain, such as the transmembrane domain of CD8, CD28, CD27, B7H3, IgG1, IgG4, CD4, DAP10, DAP12, for example.

Following preparation, the modified NK cells may be immediately infused (including with an effective amount of one or more bispecific or multi-specific antibodies, or the NK cells may be stored, such as by cryopreservation. In some cases, when the NK cells are source from cryopreservation, the NK cells were deactivated pre-cryopreservation using a deactivating agent (e.g., a kinase inhibitor, e.g., Dasatinib, nilotinib, rapamycin, etc.). In certain aspects, the cells may be propagated for days, weeks, or months ex vivo as a bulk population within about 1, 2, 3, 4, or 5 days.

### E. Loading of NK Cells

In particular embodiments, the NK cells are loaded with antibodies prior to use. The NK cells may be loaded in any specific manner, including in culture (e.g., incubation) or immediately before infusion, for example, to produce a complex of NK cells with the antibodies. The culture (e.g., incubation) conditions are suitable enough to allow for an effective amount of antibody to bind to the surface of the NK cells. In the case of use of monospecific antibodies, the Fc region of the monospecific antibody binds the NK cell while the antigen binding domain of the monospecific antibody is free to bind its target antigen. In certain embodiments of cases of use of multispecific antibodies, one or more antigen binding domains of the antibody can bind the surface of the NK cells, such as through an antigen on the surface of the NK cells (for example but not limited to, NKp30, NKp44, NKp46, CD16, CD32, CD64, KIRs, and the like), and the other antigen binding domain is free to bind its target antigen. In certain embodiments of cases of use of multispecific antibodies, one or more antigen binding domains of the antibody can bind one or more target antigens. In certain embodiments of cases of use of multispecific antibodies, the Fc region of the antibody binds the NK cell while the antigen binding domains of the antibody are free to bind target antigens. In certain embodiments of cases of use of multispecific antibodies, the Fc region of the antibody binds the NK cell through a transgenic construct (heterologous construct) encoded polypeptide comprising an Fc Receptor extracellular Fc binding domain (e.g., an Fc binding domain).

The culture conditions by which the NK cells become loaded may or may not be of a particular type having one or more specific parameters. In particular embodiments, the loading of the NK cells occurs in culture at a specific temperature, such as 37° C., although in alternative embodiments the temperature is 36° C. or 38° C., or lower or higher. The duration of the loading step may be for any suitable amount of time, such as in a range of one minute to 24 hours or longer. For example, the range may be in the range of 1 min to 24 hrs, 1 min to 18 hrs, 1 min to 12 hours, 1 min to 6 hrs, 1 min to 1 hr, 30 min to 24 hrs, 30 min to 18 hrs, 30 min to 12 hrs, 30 min to 6 hrs, 30 min to 1 hr, 1-24 hrs, 1-18 hrs, 1-12 hrs, 1-6 hrs, 6-24 hrs, 6-18 hrs, 6-12 hrs, 12-24 hrs, 12-18 hrs, or 18-24 hrs. In some embodiments, the duration of the loading step may be greater than or equal to approximately 6, 8, 10, 12, 14, 16, 18, 20, 22, 24, 26, 28, 30, 32, 34, 36, 38, 40, 42, 44, 46, or 48 hours, or any range derivable therein. In specific embodiments, the cell culture media is basal media or complex media. In some cases, the culture comprises one or more reagents that were utilized during pre-activation and/or expansion steps, while in other cases the culture does not. In specific embodiments, the culture comprises one or more cytokines, including one or more of IL-12, IL-15, IL-2, and IL-18, for example. In some embodiments, the culture comprises APCs of any kind.

In some embodiments, loading of the NK cells is conducted such that antibodies remain bound to the NK cell surface following washing of the NK cells. In some embodiments, NK cells are loaded with antibodies under conditions that are suitable for retention of at least a detectable fraction of the antibody on the NK cell surface for at least or exactly 12 hours, 24 hours, 36 hours, 48 hours, 60 hours, 72 hours, 84 hours, 96 hours, 108 hours, 120 hours, 132 hours, 144 hours, 156 hours, 168 hours, 180 hours, 192 hours, 204 hours, 216 hours, 228 hours, 240 hours, 252 hours, 264 hours, 276 hours, 288 hours, 300 hours, 312 hours, or greater than 312 hours, or any range derivable therein. In some embodiments, NK cells are loaded with antibodies under conditions that are suitable for retention of the antibody on the NK cell surface for at least 1, 2, 3, 4, 5, 6, 7, 8, 9, 10, 11, 12, 13, 14, or 15, or longer than 15 days, or any range derivable therein. In some embodiments, NK cells are loaded with antibodies under conditions that are suitable for retention of the antibody on the NK cell surface following cryopreservation and/or thawing. In some embodiments, confirmation of loading of an antibody to an NK cell surface can be determined using flow cytometry.

In some embodiments, NK cells are loaded with at least, exactly, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μg/ml final concentration, or greater than 100 μg/ml final concentration, or any range derivable therein, of antibody. In some embodiments, NK cells are loaded with at least, exactly, or about 1, 10, 20, 30, 40, 50, 60, 70, 80, 90, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, or 1000 μg/ml final concentration, or greater than 1000 μg/ml final concentration, or any range derivable therein, of antibody.

In some embodiments, NK cells and antibodies are incubated such that at least or exactly about 1%, 2%, 3%, 4%, 5%, 6%, 7% 8%, 9% 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, of NK cells are complexed with an antibody.

In some embodiments, NK cells and antibodies are incubated such that at least or exactly about 1%, 2%, 3%, 4%, 5%, 6%, 7%, 8%, 9% 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, of the antibody remains bound to the NK cells for at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 8 days, 9 days, 10 days, 11 days, or 12 days, or any range derivable therein, following incubation of the NK cells and antibody.

In certain embodiments, relative to the amount of antibodies complexed to engineered NK cells measured about 1 hour after incubation and washing, at least about 1%, 2%, 3%, 4%, 5%, 6%, 7%, 8%, 9%, 10%, 11%, 12%, 13%, 14%, 15%, 16%, 17%, 18%, 19%, 20%, or greater than 20%, or any range derivable therein, of the antibodies are bound to the NK cell surface at least 3 days after incubation and washing. In certain embodiments, relative to the amount of antibodies complexed to engineered NK cells measured about 1 hour after incubation and washing, at least about 9%, of the antibodies are bound to the NK cell surface at least 3 days after incubation and washing.

In certain embodiments, relative to the amount of antibodies complexed to engineered NK cells measured about 1 hour after incubation and washing, at least about 1%, 2%, 3%, 4%, 5%, 6%, 7%, 8%, 9%, 10%, 11%, 12%, 13%, 14%, 15%, 16%, 17%, 18%, 19%, 20%, or greater than 20%, or any range derivable therein, of the antibodies are bound to the NK cell surface at least 5 days after incubation and washing. In certain embodiments, relative to the amount of antibodies complexed to engineered NK cells measured about 1 hour after incubation and washing, at least about 6% of the antibodies are bound to the NK cell surface at least 5 days after incubation and washing.

In certain embodiments, relative to the amount of antibodies complexed to engineered NK cells measured about 1 hour after incubation and washing, at least about 1%, 2%, 3%, 4%, 5%, 6%, 7%, 8%, 9%, 10%, 11%, 12%, 13%, 14%, 15%, 16%, 17%, 18%, 19%, 20%, or greater than 20%, or any range derivable therein, of the antibodies are bound to the NK cell surface at least 7 days after incubation and washing. In certain embodiments, relative to the amount of antibodies complexed to the engineered NK cells measured about 1 hour after incubation and washing, at least about 4% of the antibodies are bound to the NK cell surface at least 7 days after incubation and washing.

In certain embodiments, relative to the amount of antibodies complexed to engineered NK cells measured about 1 hour after incubation and washing, at least about 1%, 2%, 3%, 4%, 5%, 6%, 7%, 8%, 9%, 10%, 11%, 12%, 13%, 14%, 15%, 16%, 17%, 18%, 19%, 20%, or greater than 20%, or any range derivable therein, of the antibodies are bound to the NK cell surface at least 12 days after incubation and washing. In certain embodiments, relative to the amount of antibodies complexed to the engineered NK cells measured about 1 hour after incubation and washing, at least about 3% of the antibodies are bound to the NK cell surface at least 12 days after incubation and washing.

In certain embodiments, a composition comprising engineered NK cells and antibodies is cryopreserved. In certain embodiments, a composition comprising engineered NK cells and antibodies is thawed from cryopreservation, and at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90% of the engineered NK cells are complexed to an antibody.

In certain embodiments, relative to the amount of antibodies complexed to non-cryopreserved engineered NK cells measured about 1 hour after incubation and washing, at least about 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, 90%, or greater than 90%, or any range derivable therein, of the antibodies are bound to the NK cell surface after thawing from cryopreservation. In certain embodiments, relative to the amount of antibodies complexed to non-cryopreserved engineered NK cells measured about 1 hour after incubation and washing, at least about 30% of the antibodies are bound to the NK cell surface after thawing from cryopreservation. In certain embodiments, relative to the amount of antibodies complexed to non-cryopreserved engineered NK cells measured about 1 hour after incubation and washing, at least about 40% of the antibodies are bound to the NK cell surface after thawing from cryopreservation. In certain embodiments, relative to the amount of antibodies complexed to non-cryopreserved engineered NK cells measured about 1 hour after incubation and washing, at least about 50% of the antibodies are bound to the NK cell surface after thawing from cryopreservation. In certain embodiments, relative to the amount of antibodies complexed to non-cryopreserved engineered NK cells measured about 1 hour after incubation and washing, at least about 60% of the antibodies are bound to the NK cell surface after thawing from cryopreservation.

In some embodiments, incubation of the NK cells and antibody occurs in any suitable NK cell media known to one of skill in the art. In certain embodiments, incubation of the NK cells and antibody is performed in media comprising, consisting of, or consisting essentially of Click's/RPMI media. In certain embodiments, incubation of the NK cells and antibody is performed in media comprising, consisting of, or consisting essentially of Click's media. In certain embodiments, incubation of the NK cells and antibody is performed in media comprising, consisting of, or consisting essentially of RPMI media. In certain embodiments, incubation of the NK cells and antibody is performed in media comprising, consisting of, or consisting essentially of SCGM media. In certain embodiments, incubation of the NK cells and antibody is performed in vivo, such as in the blood, lymph, and/or tumor of a subject.

In certain embodiments, loading of an NK cell can comprise in-vivo loading of the NK cells. In some embodiments, a subject can be administered one or more doses of an antibody prior to administration of an NK cell. In some embodiments, a subject can be administered one or more doses of an antibody after administration of an NK cell. In some embodiments, a subject can be administered one or more doses of an antibody before administration of a loaded NK cell, and a subject can be administered one or more doses of an antibody after administration of a loaded NK cell.

In some embodiments, loading of an NK cell can comprise providing a subject with at least, exactly, or about, 1 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg of an antibody, one or more times prior to administration of an NK cell. In some embodiments, loading of an NK cell can comprise providing a subject with at least, exactly, or about 1, 10, 20, 30, 40, 50, 60, 70, 80, 90, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, or 1000 mg of an antibody, one or more times prior to administration of an NK cell.

In some embodiments, loading of an NK cell can comprise providing a subject with at least, exactly, or about, 1 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg of an antibody, one or more times after administration of an NK cell. In some embodiments, loading of an NK cell can comprise providing a subject with at least, exactly, or about 1, 10, 20, 30, 40, 50, 60, 70, 80, 90, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, or 1000 mg of an antibody, one or more times after administration of an NK cell.

In some embodiments, loading of an NK cell can comprise providing a subject with at least, exactly, or about, 1 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg of an antibody, at the same time as administration of an NK cell. In some embodiments, loading of an NK cell can comprise providing a subject with at least, exactly, or about 1, 10, 20, 30, 40, 50, 60, 70, 80, 90, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, or 1000 mg of an antibody, at the same time as administration of an NK cell.

The antibodies of the compositions are subjected in an effective amount to an effective amount of NK cells of the disclosure, thereby producing a complex that is “chimeric antigen receptor-like.” In particular, an antigen binding domain of the antibody binds to the NK cells, such as through the antigen that is a cell surface protein. A plurality of antibodies may be subjected to a plurality of NK cells such that there are multiple complexes of cell/antibody. The antibodies may be of any time, including monospecific, bispecific, or multispecific, and in specific cases the antibody engages both the NK cell and a target antigen through an antigen binding domain of the antibody (such as with engagers in the art that are fusion proteins consisting of two single-chain variable fragments (scFvs) of different antibodies). In examples wherein the antibody is monospecific, an antigen binding domain of the antibody binds a target antigen, such as a cancer antigen, and another part of the antibody binds the NK cells, such as an Fc region of the antibody. In cases wherein the antibody is multispecific, one or more antigen binding domains of the antibody may bind the NK cell (such as through an NK cell surface antigen) and one or more antigen binding domains of the antibody binds one or more target antigens. In certain embodiments of cases of use of multispecific antibodies, one or more antigen binding domains of the antibody and/or the Fc region of the antibody may bind the NK cell. In certain embodiments, one or more Fc regions of the antibody may bind the NK cell through a polypeptide encoded by a transgenic construct. The multispecific antibody may be bispecific, trispecific, or tetraspecific, for example. In cases wherein the antibody is trispecific or tetraspecific, the additional antigen binding domains may bind other cells, such as stem cells.

In particular embodiments, the antibodies may bind any NK cell surface antigen (that may or may not be receptors) on NK cells, such as CD16 (including CD16a or CD16b), CD32, CD56, CD64, a c-type lectin such as NKG2D, NKG2C, a costimulatory molecule such as CS1, DNAM, 2B4, CD2, an NCR, NKp30, NKp44, NKp46, or KIR, and redirect the NK cells to a target, thus increasing the response and specificity against different tumors.

In some embodiments, the antibodies may bind any suitable antigen (e.g., antigens described herein, such as those that are described as targets of TCRs, etc.). In particular embodiments, an antibody targets CD123. In particular embodiments, an antibody targets EGFR. In particular embodiments, an antibody targets EGFR2. In particular embodiments, an antibody is bi-specific and targets EGFR and c-MET. In particular embodiments, an antibody is Imgatuzumab, Amivantamab, and/or Margetuximab.

Generation of the complexes may be by any suitable means, such that the conditions are sufficient for the appropriate region of the antibody to bind the appropriate surface region of the NK cell. Any particular medium may be utilized, in certain instances. In specific cases, Plasma-Lyte A and/or human serum albumin are utilized, wherein in other cases they are not. Once the complexes are formed in culture, they may or may not be washed prior to administration to the subject, such as through infusion. In some embodiments, the NK cells and the antibodies are administered separately, and the complexes form in vivo. In certain embodiments, the NK cells and the antibodies are administered separately, and are also administered together, and complexes form in vitro and in vivo. In certain embodiments, a composition comprising NK cells and antibody is washed with PBS to remove unbound antibody. In certain embodiments, a composition comprising NK cells and antibody is washed at least 1, 2, 3, 4, or 5, or greater than 5 times to remove unbound antibody. In certain embodiments, a composition comprising NK cells and antibody is was for at least 1, 2, 3, 4, 5, 6, 7, 8, 9, or 10, or greater than 10 minutes to remove unbound antibody. In certain embodiments, a composition comprising NK cells and antibody is washed twice to remove unbound antibody. In certain embodiments, a wash is for exactly, or about 5 minutes. In certain embodiments, a wash comprises agitation of the composition, e.g., using a cell shaker.

### F. Pre-Activation

In some embodiments, the NK cells are pre-activated prior to administration to a recipient individual. The pre-activation step may or may not occur before any expansion step. In specific embodiments, the NK cells are pre-activated with one or more cytokines, and in specific embodiments, the NK cells are pre-activated with one or more of IL-12, IL-15, IL-2, and IL-18 and including two, three, or more. In cases wherein less than all three of IL-12, IL-15, IL-2, and IL-18 are utilized, it may be that IL-12 and IL-15 but not IL-18; or IL-12 and IL-18 but not IL-15; or IL-15 and IL-18 but not IL-12. IL-2 may or may not be substituted for IL-15.

In particular embodiments, the pre-activation cytokines may be IL-12, IL-15, and IL-18. One or more additional cytokines may be used for the pre-activation step. The pre-activation may be for a short period of time such as 5-72 hours, such as 10-50 hours, particularly 10-20 hours, such as 12, 13, 14, 15, 16, 17, 18, 19, or 20 hours, and specifically about 16 hours in some cases. The pre-activation culture may comprise IL-18 and/or IL-15 at a concentration of 10-100 ng/mL, such as 40-60 ng/mL, particular 45, 46, 47, 48, 49, 50, 51, 52, 53, 54, or 55 ng/mL, specifically about 50 ng/mL. In some cases, the pre-activation culture comprises IL-12 at a concentration of 0.1-150 ng/mL, including at a concentration of 1-20 ng/mL, such as a concentration of 10 ng/mL. In alternative embodiments the NK cells may be stimulated with IL-2, or other cytokines that bind the common gamma-chain (e.g., IL-7, IL-21, and others), and this may be in addition to IL-12, IL-15, and IL-18 or as an alternative to one or more of them. In such cases, the pre-activation culture may comprise IL-12 at a concentration of 0.1-150 ng/mL, such as 0.5-50 ng/mL, particularly 1-20 ng/mL, such as 2, 3, 4, 5, 6, 7, 8, 9, 10, 11, 12, 13, 14, or 15 ng/mL, specifically about 10 ng/mL.

### G. Expansion

In particular embodiments, NK cells are expanded to increase their quantity prior to administration to an individual in need thereof. The expanded cells may or may not be derived from pre-activated NK cells such that a pre-activation step may occur before an expansion step. The NK cell expansion step may be of any suitable such that the NK cell population is expanded, but in specific cases the expansion step utilizes particular one or more reagents, such as in culture, to enhance their expansion. In certain cases the NK cells may not be expanded. IL-2 or IL-15 or IL-18 or any combination of the cytokines may be added to the expansion culture before or during expansion. The NK cells can be expanded ex vivo in flasks or in one of several different bioreactor configurations with continuous perfusion of media/additives, in specific embodiments.

In specific cases, the NK cells (whether pre-activated or not) may be washed (e.g., with PBS or Plasma Lyte or human serum albumin or culture media or combinations thereof) prior to and/or after expansion, such as 1, 2, 3, 4, or 5 times. In some embodiments, cells are washed specifically 3 times. In particular embodiments, the NK cells are expanded in the presence of artificial antigen presenting cells (aAPCs). In particular embodiments, the NK cells are expanded in the presence of fragments of aAPCs. The aAPCs may be engineered to express CD137 ligand and/or a membrane-bound cytokine. The membrane-bound cytokine may be membrane-bound IL-21 (mIL-21) or membrane-bound IL-15 (mIL-15). In particular embodiments, the aAPCs are engineered to express CD137 ligand and mIL-21. The aAPCs may be derived from cancer cells, such as leukemia cells. The aAPCs may not express endogenous HLA class I, II, or CD1d molecules. They may express ICAM-1 (CD54) and LFA-3 (CD58) or CD48. In particular, the aAPCs may be K562 cells, such as K562 cells engineered to express CD137 ligand and mIL-21. The engineering may be by any method known in the art, such as retroviral transduction, although any viral or non-viral vector may be utilized. The aAPCs may or may not be irradiated. The expansion may be for a particular duration in time, such as for about 2-30 days, such as 3-20 days, particularly 12-16 days, such as 12, 13, 14, 15, 16, 17, 18, or 19 days, specifically about 14 days. The pre-activated NK cells and aAPCs may be present at a ratio of about 3:1-1:3, such as 2:1, 1:1, 1:2, specifically about 1:2. The expansion culture may further comprise one or more cytokines to promote expansion, such as IL-2. The IL-2 may be present at a concentration of about 10-500 U/mL, such as 100-300 U/mL, particularly about 200 U/mL. The IL-2 may be replenished in the expansion culture, including at a certain frequency, such as every 2-3 days. The aAPCs may be added to the culture at least a second time, such as at about 7 days of expansion. Any cytokine(s) used in the pre-activation and/or expansion steps may be recombinant human cytokines.

In some embodiments, following expansion, the NK cells may be immediately utilized in any manner, such as complexed with one or more antibodies, or they may be stored, such as by cryopreservation. In certain aspects, the cells may be propagated for days, weeks, or months ex vivo as a bulk population within about 1, 2, 3, 4, or 5 days.

Activated and/or expanded NK cells can secrete type I cytokines, such as interferon-7, tumor necrosis factor-α and granulocyte-macrophage colony-stimulating factor (GM-CSF), which activate both innate and adaptive immune cells as well as other cytokines and chemokines. The measurement of these cytokines can be used to determine the activation status of NK cells. In addition, other methods known in the art for determination of NK cell activation may be used for characterization of the NK cells of the present disclosure.

Thus, with respect to particular pre-activation and expansion aspects of the disclosure, in specific embodiments the NK cells pre-activated with any combination of IL-12, IL15, and/or IL-18 followed by expansion with aAPCs, such as K562 cells expressing mIL-21 and CD137 ligand, provide a highly potent cellular product. Thus, methods are provided using the present NK cells for the treatment of various diseases, such as immunotherapy of patients with cancer. In an exemplary method, the isolated NK cells may be subjected to a brief period, such as about 16 hours, of pre-activation with a combination of cytokines, such as interleukin-12 (IL-12), IL-15, and/or IL-18, followed by expansion using artificial antigen presenting cells (aAPCs), such as K562 feeder cells expressing membrane-bound IL-21 and CD137 ligand, and/or exogenous IL-2. IL-2 or IL-15 or IL-18 or any combination of the cytokines may be added to the expansion culture at least a second time.

### H. Cryopreservation

In particular cases, NK cells and/or antibodies of the disclosure are preserved in a cryopreservation medium composition comprising at least one cryoprotectant, a serum (human or animal serum) or a non-serum alternative to serum (not human serum or animal serum), and at least one cytokine and/or at least one growth factor. In some cases, the cryoprotectant is dimethyl sulfoxide (DMSO), glycerin, glycerol, hydroxyethyl starch, or a combination thereof. The non-serum alternative may be of any kind, including at least platelet lysate and/or a blood product lysate (for example, human serum albumin). In embodiments of the composition wherein one or more (including two or more) cytokines are utilized, the cytokine may be a natural or a recombinant or a synthetic protein. At least one of the cytokines may be an Food and Drug Administration (FDA)-approved cytokine. Examples of cytokines and growth factors include at least IL-1, IL-2, IL-3, IL-4, IL-6, IL-7, IL-9, IL-10, IL-12, IL-13, IL-15, IL-17, IL-18, IL-21, IL-22, interferon, tumor necrosis factor, stem cell factor, FLT3-ligand, APRIL, thrombopoietin, erythropoietin, or a combination thereof. For serum embodiments, the serum may be an animal-derived serum, such as human serum (including human AB serum) or bovine serum. DMSO and other cryoprotectants, when utilized may comprise 4-10%, 4-6%, 4-8%, 5-10%, 5-8%, 6-10%, 6-8%, 8-10%, and so forth, of the composition. For embodiments wherein serum is employed, the serum may comprise 5-99%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-99%, 10-95%, 10-90%, 10-85%, 10-80%, 10-75%, 10-70%, 10-65%, 10-60%, 10-55%, 10-50%, 10-45%, 10-40%, 10-35%, 10-30%, 10-25%, 10-20%, 10-15%, 20-99%, 20-95%, 20-90%, 20-85%, 20-80%, 20-75%, 20-70%, 20-65%, 20-60%, 20-55%, 20-50%, 20-45%, 20-40%, 20-35%, 20-30%, 20-25%, 30-99%, 30-95%, 30-90%, 30-85%, 30-80%, 30-75%, 30-70%, 30-65%, 30-60%, 30-55%, 30-50%, 30-45%, 30-40%, 30-35%, 40-99%, 40-95%, 40-90%, 40-85%, 40-80%, 40-75%, 40-70%, 40-65%, 40-60%, 40-55%, 40-50%, 40-45%, 50-99%, 50-95%, 50-90%, 50-85%, 50-80%, 50-75%, 50-70%, 50-65%, 50-60%, 50-55%, 60-99%, 60-95%, 60-90%, 60-85%, 60-80%, 60-75%, 60-70%, 60-65%, 70-99%, 70-95%, 70-90%, 70-85%, 70-80%, 70-75%, 80-99%, 80-95%, 80-90%, 80-85%, 90-99%, 90-95%, or 95-99% of the composition. The composition may comprise at least or no more than 10, 15, 20, 25, 30, 35, 40, 45, 50, 55, 60, 65, 70, 75, 80, 85, 90, 91, 92, 93, 94, 95, 96, 97, 98, or 99% of serum. In specific embodiments, the composition comprises platelet lysate that may be at any concentration in the composition, but in certain embodiments the platelet lysate comprises 5-99%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-99%, 10-95%, 10-90%, 10-85%, 10-80%, 10-75%, 10-70%, 10-65%, 10-60%, 10-55%, 10-50%, 10-45%, 10-40%, 10-35%, 10-30%, 10-25%, 10-20%, 10-15%, 20-99%, 20-95%, 20-90%, 20-85%, 20-80%, 20-75%, 20-70%, 20-65%, 20-60%, 20-55%, 20-50%, 20-45%, 20-40%, 20-35%, 20-30%, 20-25%, 30-99%, 30-95%, 30-90%, 30-85%, 30-80%, 30-75%, 30-70%, 30-65%, 30-60%, 30-55%, 30-50%, 30-45%, 30-40%, 30-35%, 40-99%, 40-95%, 40-90%, 40-85%, 40-80%, 40-75%, 40-70%, 40-65%, 40-60%, 40-55%, 40-50%, 40-45%, 50-99%, 50-95%, 50-90%, 50-85%, 50-80%, 50-75%, 50-70%, 50-65%, 50-60%, 50-55%, 60-99%, 60-95%, 60-90%, 60-85%, 60-80%, 60-75%, 60-70%, 60-65%, 70-99%, 70-95%, 70-90%, 70-85%, 70-80%, 70-75%, 80-99%, 80-95%, 80-90%, 80-85%, 90-99%, 90-95%, or 95-99% of the composition. The composition may comprise at least or no more than 10, 15, 20, 25, 30, 35, 40, 45, 50, 55, 60, 65, 70, 75, 80, 85, 90, 91, 92, 93, 94, 95, 96, 97, 98, or 99% of platelet lysate.

The composition may have certain concentrations of components, including cytokines and/or growth factors. In specific cases, any cytokine, including IL-2, IL-21, and/or IL-15, for example, are present in the composition in a particular concentration. The IL-2 may be present at a concentration of 1-5000, 1-1000, 1-500, 1-100, 100-5000, 100-500, 500-5000, 500-1000, or 1000-5000 U/mL, for example. In a specific case, the IL-2 is present at a concentration in the composition of at least or no more than 100, 200, 300, 400, 500, 600, 700, 800, 900, or 1000 U/mL. In specific embodiments, IL-21 is present in the composition at a concentration of 10-3000, 10-2000, 10-1000, 10-500, 10-100, 100-3000, 100-2000, 100-1000, 500-3000, 500-2000, 500-1000, 1000-3000, 1000-2000, or 2000-3000 ng/mL. The IL-21 may be in a concentration in the composition of at least or nor more than 1, 5, 10, 20, 30, 40, 50, 60, 70, 80, 90, 100, 150, 200, 250, 300, 350, 400, 450, 500, 750, 1000, 1250, 1500, 1750, 2000, 2250, 2500, 2750, or 3000 ng/mL. IL-15 may be present in the composition at a concentration of 1-2000, 1-1000, 1-500, 1-100, 100-2000, 100-1000, 100-500, 500-2000, 500-1000, or 1000-2000 ng/mL. IL-15 may be present in the composition at a concentration of at least or no more than 10, 50, 100, 500, 1000, 1500, or 2000 ng/mL.

Compositions as encompassed herein that comprise at least one cryoprotectant, a serum or a non-serum alternative to serum, and at least one cytokine and/or at least one growth factor may further comprise a plurality of immune cells and/or stem cells, each of any kind. In specific embodiments, the cells are NK cells, T cells, B cells, NKT cells derived from mature bone marrow or peripheral blood cells, cell lines such as tumor cell lines (e.g., NK92 or other NK lines), hematopoietic stem cells, induced pluripotent stem cells, MSCs (a population of cells alternatively called “mesenchymal stem cells” and “mesenchymal stromal cells” in the literature), or a mixture thereof, which can be derived from bone marrow, peripheral blood, skin, adipose tissue, or a combination thereof. In embodiments wherein NK cells are utilized, the NK cells may or may not be expanded NK cells. Embodiments of the disclosure also encompass pharmaceutical compositions that comprise any composition of the disclosure and a suitable pharmaceutically acceptable carrier.

In certain embodiments, cells and/or antibodies are treated with one or more deactivating agents (e.g., a kinase inhibitor, e.g., Dasatinib, Nilotinib, Rapamycin, etc.) pre-cryopreservation.

In some embodiments, technologies described herein comprise deactivating a NK cell, comprising treating an NK cell with an effective amount of one or more deactivating agents under conditions to produce a deactivated NK cell. In some embodiments, a deactivating agent is a kinase inhibitor. In some embodiments, a deactivating agent is a mechanistic target of rapamycin (mTOR) inhibitor. In some embodiments, the mTOR inhibitor is rapamycin, everolimus, and/or temsirolimus. In some embodiments, the mTOR inhibitor is rapamycin. In some embodiments, the deactivating agent is a tyrosine kinase (TK) inhibitor. In some embodiments, the TK inhibitor is Lorlatinib, Brigatinib, Ceritinib, Alectinib, Crizotinib, Bosutinib, Ponatinib, Nilotinib, Dasatinib, Imatinib, Zanubrutinib, Acalabrutinib, Ibrutinib, Capmatinib, Pexidartinib, Dacomitinib, Osimertinib, Erlotinib, Gefitinib, Lapatinib, Afatinib, Pemigatinib, Erdafitinib, Nintedanib, Gilteritinib, Midostaurin, Tucatinib, Neratinib, Baricitinib, Ruxolitinib, Fedratinib, Tofacitinib, Ripretinib, Selumetinib, Binimetinib, Cobimetinib, Trametinib, Upadacitinib, Avapritinib, Selpercatinib, Cabozantinib, Fostamatinib, Larotrectinib, Entrectinib, Axitinib, Regorafenib, Pazopanib, Sorafenib, Lenvatinib, Vandetanib, and/or Sunitinib. In some embodiments, the TK inhibitor is a BCR-Abl inhibitor. In some embodiments, the TK inhibitor is Bosutinib, Ponatinib, Nilotinib, Dasatinib, and/or Imatinib. In some embodiments, the TK inhibitor is Dasatinib and/or Nilotinib. In some embodiments, the TK inhibitor is Dasatinib.

In some embodiments, treatment with a deactivating agent is at any point during culturing of the NK cell. In some embodiments, the treatment is for about 24 to about 96 hours, about 36 to about 84 hours, or about 48 to about 72 hours. In some embodiments, the treatment is for about 24 hours, about 48 hours, or about 72 hours. In some embodiments, the NK cell is treated with the deactivating agent at a concentration of about 1 to about 1000 nM. In some embodiments, the NK cell is treated with the deactivating agent at a concentration of about 5 to about 500 nM. In some embodiments, the NK cell is treated with the deactivating agent at a concentration of about 20 to about 200 nM. In some embodiments, the NK cell is treated with the deactivating agent at a concentration of about 30 to about 100 nM. In some embodiments, the deactivated NK cell has an increased expression of one or more of C-kit, CCR-5, CD62L and/or CXCR4, and/or decreased expression of one or more of NKG2D, DNAM, OX-40, TRAIL, HLA-DR, CD2, CD25, ICOS, and/or CD95 relative to an activated NK cell. In some embodiments, technologies described herein comprise methods of maintaining the viability of a population of cells over at least 50% percent following cryopreservation of the population, comprising the step of subjecting the population to an effective amount of one or more deactivating agents (e.g., a tyrosine kinase inhibitor) to deactivate the cells prior to cryopreservation, cryopreserving the cells, and thawing the population, wherein upon thawing the viability of the population is over at least 50%. In some cases, upon thawing of the cells the viability of the population of cells is over at least 55, 60, 65, 70, 75, 80, 85, 90, 91, 92, 93, 94, 95, 96, 97, 98, or 99% following cryopreservation of the population.

### III. HETEROLOGOUS PROTEINS AND MUTATIONS

In specific embodiments, the NK cells are modified not only to express one or more components of the TCR/CD3 complex and an Fc binding protein, but they are also modified to express one or more other heterologous proteins. The heterologous proteins may facilitate activity of the NK cells in any manner, including at least their activation, persistence, expansion, homing, and/or cytotoxicity.

### A. Monospecific, Bispecific, or Multi-Specific Antibodies

Aspects of the disclosure relate to use of antibodies or functional fragments thereof in a composition also comprising certain NK cells. The term “antibody” refers to an intact immunoglobulin of any isotype, or a fragment thereof that can compete with the intact antibody for specific binding to the target antigen, such as through the antigen binding domain, and includes chimeric, humanized, fully human, monospecific, and multispecific (including at least bispecific and trispecific, and more) antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity. In specific embodiments, the antibody comprises an scFv. In certain embodiments, the antibodies may be any antibody or antibody-like structure known in the art, including antibody fragments, single-domain antibodies, scFv, bispecific antibodies, bispecific diabodies, trispecific antibodies, scFv-Fc and other antibody constructs and engagers.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986 See, e.g., Epitope Mapping Protocols, supra. Additionally, antigenic regions of proteins can also be predicted and identified using standard antigenicity and hydropathy plots.

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al. Front Immunol. 2013; 4: 302; 2013)

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (λ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the —COH end. The isotype of an antibody can be IgM, IgD, IgG, IgA, or IgE and is defined by the heavy chains present of which there are five classifications: mu (μ), delta (δ), gamma (γ), alpha (α), or epsilon (ε) chains, respectively. IgG has several subtypes, including, but not limited to, IgG1, IgG2, IgG3, and IgG4. IgM subtypes include IgM1 and IgM2. IgA subtypes include IgAQ1 and IgA2.

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins, such as the following:

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bi-specific, meaning the two antigen-binding sites have different antigen specificities.

Bispecific antibodies are a class of antibodies that have two paratopes with different binding sites for two or more distinct epitopes. In some embodiments, bispecific antibodies can be biparatopic, wherein a bispecific antibody may specifically recognize a different epitope from the same antigen. In some embodiments, bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

In certain aspects, the antigen-binding domain may be multispecific or heterospecific by multimerizing with VH and VL region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

In some embodiments, multispecific antibodies can be used and directly linked via a short flexible polypeptide chain, using routine methods known in the art. One such example is diabodies that are bivalent, bispecific antibodies in which the VH and VL domains are expressed on a single polypeptide chain, and utilize a linker that is too short to allow for pairing between domains on the same chain, thereby forcing the domains to pair with complementary domains of another chain creating two antigen binding sites. The linker functionality is applicable for embodiments of triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, VH and VL, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the VL domain are identified as L1, L2, and L3, with L1 occurring at the most distal end and L3 occurring closest to the CL domain. The CDRs may also be given the names CDR-1, CDR-2, and CDR-3. The L3 (CDR-3) is generally the region of highest variability among all antibody molecules produced by a given organism. The CDRs are regions of the polypeptide chain arranged linearly in the primary structure, and separated from each other by Framework Regions. The amino terminal (N-terminal) end of the VL chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the VH chain, which includes three CDRs identified as H1, H2 and H3. The majority of amino acid residues in the variable domains, or Fv fragments (VH and VL), are part of the framework regions (approximately 85%). The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970); Chothia (as described in C. Chothia et al., “Conformations of immunoglobulin hypervariable regions,” Nature, vol. 342, no. 6252, pp. 877-883, December 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include: 1) Computational predictions of the tertiary structure of the antibody/epitope binding interactions based on the chemical nature of the amino acid sequence of the antibody variable region and composition of the epitope; 2) Hydrogen-deuterium exchange and mass spectroscopy; 3) Polypeptide fragmentation and peptide mapping approaches in which one generates multiple overlapping peptide fragments from the full length of the polypeptide and evaluates the binding affinity of these peptides for the epitope; 4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific VH and VL domains as appropriate.

In certain aspects, affinity matured antibodies are enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by VH and VL domain shuffling, random mutagenesis of CDR and/or framework residues employed in phage display is described by Rajpal et al., PNAS. 24: 8466-8471 (2005) and Thie et al., Methods Mol Biol. 525:309-22 (2009) in conjugation with computation methods as demonstrated in Tiller et al., Front. Immunol. 8:986 (2017).

Chimeric immunoglobulins are the products of fused genes derived from different species; “humanized” chimeras generally have the framework region (FR) from human immunoglobulins and one or more CDRs are from a non-human source.

In certain aspects, portions of the heavy and/or light chain are identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference for all purposes.

In some embodiments, minimizing the antibody polypeptide sequence from the non-human species optimizes chimeric antibody function and reduces immunogenicity. Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhoeyen et al., Science 239:1534-36 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

Functional antibody fragments and antigen-binding fragments may be utilized. Certain aspects relate to antibody fragments, such as antibody fragments that bind to and/or neutralize inflammatory mediators. The term functional antibody fragment includes antigen-binding fragments of an antibody that retain the ability to specifically bind to an antigen. These fragments are constituted of various arrangements of the variable region heavy chain (VH) and/or light chain (VL); and in some embodiments, include constant region heavy chain 1 (CH1) and light chain (CL). In some embodiments, they lack the Fc region constituted of heavy chain 2 (CH2) and 3 (CH3) domains. Embodiments of antigen binding fragments and the modifications thereof may include: (i) the Fab fragment type constituted with the VL, VH, CL, and CH1 domains; (ii) the Fd fragment type constituted with the VH and CH1 domains; (iii) the Fv fragment type constituted with the VH and VL domains; (iv) the single domain fragment type, dAb, (Ward, 1989; McCafferty et al., 1990; Holt et al., 2003) constituted with a single VH or VL domain; (v) isolated complementarity determining region (CDR) regions. Such terms are described, for example, in Harlow and Lane, Antibodies: A Laboratory Manual, Cold Spring Harbor Laboratory, NY (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015). The citations in this paragraph are all incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment means a monovalent antigen-binding fragment of an antibody containing the VL, VH, CL and CH1 domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two VH and VL domains, and can further include all or part of the two CL and CH1 domains.

The term Fd fragment means a fragment of the heavy chain of a monoclonal antibody, which includes all or part of the VH, including the CDRs. An Fd fragment can further include CH1 region sequences.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)2 means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv)2 fragments are also known as “miniantibodies” or “minibodies.”

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

In some cases, fragment crystallizable regions, Fc, are utilized. An Fc region contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included. In some embodiments, antibodies that incorporate cytokines (including those referred to herein) may be utilized (such as with TRIKEs).

In some embodiments, an antibody is an antibody-drug conjugate. In some embodiments, an antibody may specifically be one or more of the following, although an antibody is not limited to these: [fam]-trastuzumab deruxtecan, Abciximab, Adalimumab, Ado-trastuzumab emtansine, Aducanumab, Alemtuzumab, Alirocumab, Amivantamab, Anifrolumab, Ansuvimab, Atezolizumab, Atoltivimab with Maftivimab and Odesivimab-ebgn (aka Inmazeb), Avelumab, Basiliximab, Belantamab mafodotin, Belimumab, Benralizumab, Bevacizumab, Bezlotoxumab, Bimekizumab, Blinatumomab, Brentuximab vedotin, Brodalumab, Brolucizumab, Burosumab, Canakinumab, Caplacizumab, Casirivimab+imdevimab, Catumaxomab, Cemiplimab, Certolizumab pegol, Cetuximab, Cevostamab, Crizanlizumab, Daclizumab, Daratumumab, Denosumab, Dinutuximab, Donanemab, Dostarlimab, Dupilumab, Durvalumab, Eculizumab, Edrecolomab, Efalizumab, Elotuzumab, Emapalumab, Emicizumab, Enfortumab vedotin, Eptinezumab, Erenumab, Evinacumab, Evolocumab, Faricimab, Fremanezumab, Galcanezumab, Gemtuzumab, Gemtuzumab-Ozogamicin, Golimumab, Guselkumab, Ibalizumab, Ibritumomab tiuxetan, Idarucizumab, Imgatuzumab, Inebilizumab, Infliximab, Inolimomab, Inotuzumab, Inotuzumab-Ozogamicin, IPH61, Ipilimumab, Isatuximab, Ixekizumab, Lanadelumab, Lecanemab, Loncastuximab tesirine, Margetuximab, Mepolizumab, Mirvetuximab soravtansine, Mogamulizumab, Mosunetuzumab, Moxetumomab pasudotox, Muromonab-CD3, Narsoplimab, Natalizumab, Naxitamab, Nebacumab, Necitumumab, Nirsevimab, Nivolumab, Obiltoxaximab, Obinutuzumab, Ocrelizumab, Ofatumumab, Olaratumab, Omalizumab, Omburtamab, Oportuzumab monatox, Palivizumab, Panitumumab, Pembrolizumab, Penpulimab, Pertuzumab, Polatuzumab vedotin, Ramucirumab, Ranibizumab, Ravulizumab, Raxibacumab, Regdanvimab, Relatlimab, Reslizumab, Retifanlimab, Risankizumab, Rituximab, Romosozumab, Sacituzumab govitecan, Sarilumab, Satralizumab, Secukinumab, Siltuximab, Sintilimab, Sotrovimab, Spesolimab, Sutimlimab, Tafasitamab, Tebentafusp, Teclistamab, Teplizumab, Teprotumumab, Tezepelumab, Tildrakizumab, Tislelizumab, Tisotumab vedotin, Tixagevimab, cilgavimab, Tocilizumab, Toripalimab, Tositumomab-I131, Tralokinumab, Trastuzumab, Tremelimumab, Ublituximab, Ustekinumab, Vedolizumab, or any combination thereof. In some embodiments, an antibody is Elranatamab. In some embodiments, an antibody is Imgatuzumab. In some embodiments, an antibody is Margetuximab. In some embodiments, an antibody is Amivantamab. In some embodiments, an antibody is Blinatumomab. In some embodiments, an antibody is Obinutuzumab. In some embodiments, an antibody is 1PH61 (also known as IPH6101 or SAR443579). In some embodiments, an antibody is Teclistamab. In some embodiments, an antibody is Cetuximab. In some embodiments, an antibody is Rituximab.

In some embodiments, the NK cells are modified to express one or more monospecific, bispecific, and/or multi-specific antibodies, although in other cases the NK cells do not express the antibodies but the antibodies are utilized in conjunction with the NK cells (i.e., the NK cells are loaded with the antibodies and/or utilized in co-therapy regimens with the antibodies).

In some embodiments, the antibodies may be engagers that bridge a particular immune effector cell with a particular target cell for destruction of the target cell. The present disclosure allows the modified NK cells to be used with standard T-cell engagers (BiTEs) because they have been modified to express CD3 that in many cases is the T cell antigen to which the BiTE engager binds. In such cases, the BiTE used in the invention may also target a cancer or viral antigen that may be tailored to the medical condition of an intended recipient individual. For example, the BiTE may be tailored to bind a cancer antigen that is characteristic of the cancer cells of a cancer of the individual, and/or may be tailored to bind an antigen that is characteristic of a cell associated with another disorder (e.g., an autoimmune disorder). The anti-CD3 antibody of the BiTE may target the CD3γ chain, CD3δ chain, CD3ε chain, or CD3ζ chain. In some embodiments, a BiTE may target other T cell associated proteins, such as but not limited to CD28.

In some embodiments, an antibody is a BiTE, and may specifically be one or more of the following, although a BiTE is not limited to these: AMG 160/Acapatamab, AMG 199/TNB 585, AMG 330, AMG 427/EMIRODATAMAB, AMG 509, AMG 701, AMG 910, APVO414/ES414/MOR209, APVO436, Blinatumomab/Blincyto, Catumaxomab/Removab, CC-1, CC-93269/EM801, Cibisatamab/RG7802/RO6958688, CLN-049, Elranatamab/PF-06863135, EMB-06, Epcoritamab/GEN3013, ERY974, Flotetuzumab/MGD006, Glofitamab/RG6026/RO7082859, ISB 1342/GBR 1342, JNJ-63709178, JNJ-63898081, JNJ-67571244, JNJ-75348780, Linvoseltamab/REGN 5458, M701, M802, MGD007, Mosunetuzumab/RG7828, Nivatrotamab/Hu3F8-BsAb, Odronextamab/REGN1979, REGN4018, REGN5459, REGN7075, REGN5678, Talquetamab/JNJ-64407564, Tarlatamab/AMG 757, Teclistamab/JNJ-64007957, Tepoditamab/MCLA-117, TNB-383B, TNB-486, TNB-585, XmAbl3676/Plamotamab, XmAbl4045/Vibecotamab, and/or XmAb18087/Tidutamab.

In some embodiments, a BiTE can target CD30, PSMA, MUC17, CD33, FLT3, STEAP1, BCMA, CLDN18.2, CD123, CD19, CD20, EpCAM, CEA, GPC3, CD38, CD33, CD22, HER2, GPA33, GD2, MUC16, GPRC5D, DLL-3, CLEC12A, FcRH5, and/or SSTR.

In some cases, in addition to expressing a CD3/TCR complex that allows the NK cells to be utilized as a therapy with BiTEs, the NK cells may be utilized in conjunction with and/or modified to express one or more bispecific NK engagers (BiKEs). A BiKE comprises an antibody that binds a surface protein on the NK cell, including a naturally expressed surface protein on NK cells (for example but not limited to, NKp30, NKp44, NKp46, CD16, CD32, CD64, KIRs, and the like), and also comprises an antibody that binds a desired target antigen. The BiKE may target the NK cells through an antibody binding domain directed to an NK surface protein such as CD16, CS1, CD32, CD64, CD56, NKG2D, NKG2C, DNAM, 2B4, CD2, an NCR, NKp30, NKp44, NKp46, or KIR, for example. In such cases, the BiKE used in the invention may also target a cancer or viral antigen that may be tailored to the medical condition of an intended recipient individual. For example, the BiKE may be tailored to bind a cancer antigen that is characteristic of the cancer cells of a cancer of the individual, and/or may be tailored to bind an antigen that is characteristic of a cell associated with another disorder (e.g., an autoimmune disorder). In some embodiments, a BiKE is AFM13 and targets CD16 and CD30.

In certain embodiments, an antibody is Blinatumomab. In certain embodiments, an antibody is Tebentafusp. In certain embodiments, an antibody is Mosunetuzumab. In certain embodiments, an antibody is Teclistamab. In certain embodiments, an antibody is Glofitamab. In certain embodiments, an antibody is Epcoritamab. In some embodiments, an antibody is Flotetuzumab. In some embodiments, an antibody is APV0436. In some embodiments, an antibody is TNB383B. In certain embodiments of cases of use of multispecific antibodies, one or more antigen binding domains of the antibody can bind one or more target antigens.

In embodiments wherein an NK cell expresses the CD3/TCR complex (including iTCRs) and one or more other heterologous proteins (e.g., an antibody), one or more vectors may be utilized to transfect or transduce the cells with the CD3/TCR complex components and one or more other heterologous proteins. In some cases, one or more of the CD3/TCR complex components and the one or more heterologous proteins may or may not be on the same multicistronic vector.

In certain embodiments, an antibody comprises an IgG1 derived Fc domain. In certain embodiments, an antibody comprises an IgG4 derived Fc domain. In certain embodiments, an antibody comprises an Fc domain that has been glycoengineered (e.g., afucosylated, etc.). In certain embodiments, an antibody comprises an Fc domain that has not been glycoengineered. In certain embodiments, an antibody comprises an Fc domain that does comprise or expressly does not comprise Fc domain modifications, such as those described in Xinhua Wang et al., “IgG Fc engineering to modulate antibody effector functions” Protein Cell, 2018 January; 9(1): 63-73; which is incorporated herein by reference for the purposes described herein.

### B. Engineered Receptors

In specific embodiments, the NK cells are engineered to express one or more engineered receptors. In some cases, the engineered receptors are engineered antigen receptors that target a cancer or viral antigen of any kind. The receptor may be tailored to target a desired antigen based on a medical condition of an intended recipient individual.

In some embodiments, engineered NK cells may be modified to express one or more chemokines, chemokine receptors, cytokines, cytokine receptors, and/or suicide genes. In certain embodiments, NK cells are engineered to express a CD16, CD32, CD64, or other Fc region binding proteins. In certain embodiments, NK cells are engineered to express a CD16 gene and/or a characteristic Fc binding region thereof. In certain embodiments, NK cells are engineered to express a wild type CD16 gene and/or a characteristic Fc binding region thereof. In certain embodiments, NK cells are engineered to express a high affinity wild type human CD16 gene and/or a characteristic Fc binding region thereof. In certain embodiments, NK cells are engineered to express a CD32 gene and/or a characteristic Fc binding region thereof. In certain embodiments, NK cells are engineered to express a CD64 gene and/or a characteristic Fc binding region thereof. In certain embodiments, NK cells are engineered to express a transgenic receptor that is a target of a multi-specific antibody.

### C. Cytokines

In some embodiments, the cells expressing the NK cells are engineered to express one or more heterologous cytokines and/or are engineered to upregulate normal expression of one or more heterologous cytokines. The cells may or may not be transduced or transfected for one or more cytokines on the same vector as other genes. In certain embodiments, NK cells may be modified to express one or more cytokines, cytokine receptors, chemokines, chemokine receptors, and/or suicide genes.

One or more cytokines may be co-expressed from a vector, including as a separate polypeptide from any component of the TCR/CD3 complex. Interleukin-15 (IL-15), for example, is tissue restricted and only under pathologic conditions is it observed at any level in the serum, or systemically. IL-15 possesses several attributes that are desirable for adoptive therapy. IL-15 is a homeostatic cytokine that induces development and cell proliferation of natural killer cells, promotes the eradication of established tumors via alleviating functional suppression of tumor-resident cells, and inhibits activation-induced cell death (AICD). In addition to IL-15, other cytokines are envisioned. These include, but are not limited to, cytokines (e.g., IL-2, IL-12, IL-18, and/or IL-21), chemokines, and other molecules that contribute to the activation and proliferation of cells used for human application. NK cells expressing IL-15 are capable of continued supportive cytokine signaling, which is useful for their survival post-infusion. In certain embodiments, NK cells expressing IL-21 are capable of continued supportive cytokine signaling, which is useful for their survival post-infusion. In certain embodiments, a cytokine is expressed as part of a multicistronic construct with one or more functional proteins and/or marker proteins.

In some embodiments, the cells express one or more exogenously provided engineered receptors, wherein the engineered receptor comprises a chemokine receptor and/or a cytokine receptor. In some embodiments, a cytokine receptor is an IL-15 receptor. In some embodiments, a cytokine receptor is a non-naturally occurring variant of a cytokine receptor. In some embodiments, a cytokine receptor is an IL-15, IL-12, IL-2, IL-18, IL-21, IL-23, or GMCSF receptor, or a combination thereof.

In specific embodiments, the cells express one or more exogenously provided cytokines. As one example, the cytokine is IL-15, IL-12, IL-2, IL-18, IL-21, IL-23, GMCSF, or a combination thereof. The cytokine may be exogenously provided to the NK cells because it is expressed from an expression vector within the cell. In an alternative case, an endogenous cytokine in the cell is upregulated upon manipulation of regulation of expression of the endogenous cytokine, such as genetic recombination at the promoter site(s) of the cytokine. In cases wherein the cytokine is provided on an expression construct to the cell, the cytokine may be encoded from the same vector as one or more components of the CD3/TCR complex and/or Fc Receptor extracellular domain comprising polypeptide.

In some embodiments, a specific sequence of IL-15 is utilized, such as those that follow (underlining refers to signal peptide sequence, which may be included, modified, or omitted):

(SEQ ID NO: 182)
ATGCGCATTAGCAAGCCCCACCTGCGGAGCATCAGCATCCAGTGCTACCT
GTGCCTGCTGCTGAACAGCCACTTCCTGACCGAGGCCGGCATCCACGTGT
TCATCCTGGGCTGCTTCAGCGCCGGACTGCCCAAGACCGAGGCCAACTGG
GTGAACGTGATCAGCGACCTGAAGAAGATCGAGGACCTGATCCAGAGCAT
GCACATCGACGCCACCCTGTACACCGAGAGCGACGTGCACCCCAGCTGCA
AGGTGACCGCCATGAAGTGCTTTCTGCTGGAACTGCAGGTGATCAGCCTG
GAAAGCGGCGACGCCAGCATCCACGACACCGTGGAGAACCTGATCATCCT
GGCCAACAACAGCCTGAGCAGCAACGGCAACGTGACCGAGAGCGGCTGCA
AAGAGTGCGAGGAACTGGAAGAGAAGAACATCAAAGAGTTTCTGCAGAGC
TTCGTGCACATCGTGCAGATGTTCATCAACACCAGC
(SEQ ID NO: 183)
MRISKPHLRSISIQCYLCLLLNSHFLTEAGIHVFILGCFSAGLPKTEANW
VNVISDLKKIEDLIQSMHIDATLYTESDVHPSCKVTAMKCFLLELQVISL
ESGDASIHDTVENLIILANNSLSSNGNVTESGCKECEELEEKNIKEFLQS
FVHIVQMFINTS

In some embodiments, a specific sequence of IL-21 is utilized, such as those that follow (underlining refers to signal peptide sequence, which may be included or omitted): SEQ ID NO: 184—Exemplary codon optimized polynucleotide sequence comprising IL-21 (signal peptide underlined).

SEQ ID NO: 184 - Exemplary codon optimized poly-
nucleotide sequence comprising IL-21 (signal
peptide underlined).
(SEQ ID NO: 184)
ATGAGGAGCAGTCCAGGCAATATGGAGCGGATAGTAATTTGTCTCATGGT
AATATTCCTCGGTACTCTGGTACATAAATCTTCCTCTCAAGGTCAGGACC
GCCATATGATTCGAATGCGGCAGCTGATTGACATAGTCGATCAACTGAAG
AACTATGTGAATGATCTTGTGCCCGAGTTTTTGCCAGCCCCTGAAGACGT
AGAAACTAATTGTGAGTGGAGTGCCTTTTCCTGCTTTCAAAAGGCACAGC
TGAAATCCGCCAACACGGGCAATAACGAACGGATAATTAACGTATCCATT
AAGAAGCTGAAGCGGAAGCCGCCCTCAACCAATGCGGGACGGCGGCAAAA
GCATCGCTTGACCTGTCCGTCATGCGACAGCTACGAGAAAAAGCCCCCGA
AGGAGTTCTTGGAACGCTTCAAGAGTCTCCTTCAGAAAATGATTCACCAG
CACCTGTCCTCACGGACGCACGGAAGCGAGGACAGT
SEQ ID NO: 185 - Exemplary codon optimized amino
acid sequence comprising a comprising IL-21
(signal peptide underlined).
(SEQ ID NO: 185)
MRSSPGNMERIVICLMVIFLGTLVHKSSSQGQDRHMIRMRQLIDIVDQLK
NYVNDLVPEFLPAPEDVETNCEWSAFSCFQKAQLKSANTGNNERIINVSI
KKLKRKPPSTNAGRRQKHRLTCPSCDSYEKKPPKEFLERFKSLLQKMIHQ
HLSSRTHGSEDS
SEQ ID NO: 186 - Exemplary polynucleotide sequence
comprising IL-21.
(SEQ ID NO: 186)
GGCCAGGACCGGCACATGATCCGGATGAGACAGCTGATCGACATCGTGGA
CCAGCTGAAGAACTACGTGAACGACCTGGTGCCCGAGTTCCTGCCTGCCC
CCGAGGACGTGGAAACAAACTGCGAGTGGAGCGCCTTCAGCTGCTTCCAG
AAGGCCCAGCTGAAAAGCGCCAACACCGGCAACAACGAGCGGATCATCAA
CGTGTCCATCAAGAAGCTGAAGCGGAAGCCCCCCAGCACCAACGCCGGAA
GAAGGCAGAAGCACAGACTGACCTGCCCCAGCTGCGACAGCTACGAGAAG
AAGCCCCCTAAAGAGTTCCTGGAACGGTTCAAGAGCCTGCTGCAGAAGAT
GATCCACCAGCACCTGAGCAGCCGGACCCACGGCTCTGAGGACAGC
SEQ ID NO: 187 - Exemplary amino acid sequence
comprising a comprising IL-21.
(SEQ ID NO: 187)
GQDRHMIRMRQLIDIVDQLKNYVNDLVPEFLPAPEDVETNCEWSAFSCFQ
KAQLKSANTGNNERIINVSIKKLKRKPPSTNAGRRQKHRLTCPSCDSYEK
KPPKEFLERFKSLLQKMIHQHLSSRTHGSEDS

### D. Antigens

The modified NK cells of the disclosure are utilized with bispecific or multi-specific antibodies that target one or more particular antigens. In addition, the NK cells may be modified with engineered antigen receptors that target one or more particular antigens. In cases wherein the NK cells are modified with one or more engineered antigen receptors, the antigen targeted by the bispecific or multi-specific antibody, and the antigen targeted by the one or more engineered antigen receptors may or may not be the same antigen. In some cases, the antigen targeted by the bispecific or multi-specific antibody, and the antigen targeted by the one or more engineered antigen receptors are different antigens but are associated with the same type of cancer.

Among the antigens targeted by the antibodies and/or engineered antigen receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Any suitable antigen may be targeted in the present method. The antigen may be associated with certain cancer cells but not associated with non-cancerous cells, in some cases. In some embodiments, an antigen may be associated with cells characteristic of an autoimmune disorder, but not healthy/non-disorder associated cells. Exemplary antigens include, but are not limited to, antigenic molecules from infectious agents, auto-/self-antigens, tumor-/cancer-associated antigens, and tumor neoantigens (Linnemann et al., 2015). In particular aspects, the antigens include NY-ESO, CD19, EBNA, CD123, HER2, CA-125, TRAIL/DR4, CD20, CD22, CD70, CD38, CD123, CLL1, carcinoembryonic antigen, alphafetoprotein, CD56, AKT, Her3, epithelial tumor antigen, CD319 (CS1), ROR1, folate binding protein, HIV-1 envelope glycoprotein gp120, HIV-1 envelope glycoprotein gp41, CD5, CD23, CD30, HERV-K, IL-11Ralpha, kappa chain, lambda chain, CSPG4, CD33, CD47, CLL-1, U5snRNP200, CD200, BAFF-R, BCMA, FcRH5, CD99, p53, mutated p53, Ras, mutated ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, melanoma-associated antigen, BAGE, DAM-6, -10, GAGE-1, -2, -8, GAGE-3, -4, -5, -6, -7B, NA88-A, MC1R, mda-7, gp75, Gp100, PSA, PSM, Tyrosinase, tyrosinase-related protein, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), VEGFR2, cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notch1-4), NY ESO 1, c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SAGE, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, and LRRN1. Examples of sequences for antigens are known in the art, for example, in the GENBANK® database: CD19 (Accession No. NG_007275.1), EBNA (Accession No. NG_002392.2), WT1 (Accession No. NG_009272.1), CD123 (Accession No. NC_000023.11), NY-ESO (Accession No. NC_000023.11), EGFRvIII (Accession No. NG_007726.3), MUC1 (Accession No. NG_029383.1), HER2 (Accession No. NG_007503.1), CA-125 (Accession No. NG_055257.1), WT1 (Accession No. NG_009272.1), Mage-A3 (Accession No. NG_013244.1), Mage-A4 (Accession No. NG_013245.1), Mage-A10 (Accession No. NC_000023.11), TRAIL/DR4 (Accession No. NC_000003.12), and/or CEA (Accession No. NC_000019.10).

Tumor-associated antigens may be derived from prostate, breast, colorectal, lung, pancreatic, renal, mesothelioma, ovarian, liver, brain, bone, stomach, spleen, testicular, cervical, anal, gall bladder, thyroid, or melanoma cancers, as examples. Exemplary tumor-associated antigens or tumor cell-derived antigens include MAGE 1, 3, and MAGE 4 (or other MAGE antigens such as those disclosed in International Patent Publication No. WO 99/40188); PRAME; BAGE; RAGE, Lage (also known as NY ESO 1); SAGE; and HAGE or GAGE. These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

Other tumor associated antigens include Plu-1, HASH-1, HasH-2, Cripto and Criptin. Additionally, a tumor antigen may be a self-peptide hormone, such as whole length gonadotrophin hormone releasing hormone (GnRH), a short 10 amino acid long peptide, useful in the treatment of many cancers.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

### E. Suicide Gene

In particular embodiments, a suicide gene is utilized in conjunction with the NK cell therapy to control its use and allow for termination of the cell therapy at a desired event and/or time. The suicide gene is employed in transduced cells for the purpose of eliciting death for the transduced cells when needed. The cells of the present disclosure that have been modified to harbor one or more vectors encompassed by the disclosure that may comprise one or more suicide genes. In some embodiments, the term “suicide gene” as used herein is defined as a gene which, upon administration of a prodrug or other agent, effects transition of a gene product to a compound which kills its host cell. In other embodiments, a suicide gene encodes a gene product that is, when desired, targeted by an agent (such as an antibody) that targets the suicide gene product.

In some cases, the cell therapy may be subject to utilization of one or more suicide genes of any kind when an individual receiving the cell therapy and/or having received the cell therapy shows one or more symptoms of one or more adverse events, such as cytokine release syndrome, neurotoxicity, anaphylaxis/allergy, and/or on-target/off tumor toxicities (as examples) or is considered at risk for having the one or more symptoms, including imminently. The use of the suicide gene may be part of a planned protocol for a therapy or may be used only upon a recognized need for its use. In some cases the cell therapy is terminated by use of agent(s) that targets the suicide gene or a gene product therefrom because the therapy is no longer required.

Utilization of the suicide gene may be instigated upon onset of at least one adverse event for the individual, and that adverse event may be recognized by any means, including upon routine monitoring that may or may not be continuous from the beginning of the cell therapy. The adverse event(s) may be detected upon examination and/or testing. In cases wherein the individual has cytokine release syndrome (which may also be referred to as cytokine storm), the individual may have elevated inflammatory cytokine(s) (merely as examples: interferon-gamma, granulocyte macrophage colony-stimulating factor, IL-10, IL-6 and TNF-alpha); fever; fatigue; hypotension; hypoxia, tachycardia; nausea; capillary leak; cardiac/renal/hepatic dysfunction; or a combination thereof, for example. In cases wherein the individual has neurotoxicity, the individual may have confusion, delirium, aplasia, and/or seizures. In some cases, the individual is tested for a marker associated with onset and/or severity of cytokine release syndrome, such as C-reactive protein, IL-6, TNF-alpha, and/or ferritin.

Examples of suicide genes include engineered nonsecretable (including membrane bound) tumor necrosis factor (TNF)-alpha mutant polypeptides (see PCT/US19/62009, which is incorporated by reference herein in its entirety), and they may be affected by delivery of an antibody that binds the TNF-alpha mutant. Examples of suicide gene/prodrug combinations that may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidylate kinase (Tdk::Tmk) and AZT; and deoxycytidine kinase and cytosine arabinoside. The E. coli purine nucleoside phosphorylase, a so-called suicide gene that converts the prodrug 6-methylpurine deoxyriboside to toxic purine 6-methylpurine, may be utilized. Other suicide genes include CD20, CD52, inducible caspase 9, purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), EGFRv3, and Thymidine phosphorylase (TP), as examples.

### F. Knockout or Knockdown of Endogenous Genes

In certain embodiments, NK cells of the disclosure may include gene editing of the NK cells to remove 1, 2, 3, 4, 5, 6, 7, 8, 9, 10, or more endogenous genes in the NK cells. In some cases the gene editing occurs in NK cells expressing one or more heterologous transgenes (e.g., CD3, TCR, etc.), whereas in other cases the gene editing occurs in NK cells that do not express a heterologous transgene but that ultimately will express one or more heterologous transgenes, in at least some cases. In particular embodiments, the NK cells that are gene edited are expanded NK cells.

In particular cases, one or more endogenous genes of the NK cells are modified, such as disrupted in expression where the expression is reduced in part or in full. In specific cases, one or more genes are knocked down or knocked out using processes of the disclosure. In specific cases, multiple genes are knocked down or knocked out in the same step as processes of the disclosure. The genes that are edited in the NK cells may be of any kind, but in specific embodiments the genes are genes whose gene products inhibit activity and/or proliferation of NK cells. In specific cases the genes that are edited in the NK cells allow the NK cells to work more effectively in a tumor microenvironment. In specific cases, the genes are one or more of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, TDAG8, CD5, CD7, SLAMF7, CD38, LAG3, TCR, beta2-microglobulin, HLA, CD73, CREB, CREM, ICER, and CD39. In specific embodiments, the TGFBR2 gene is knocked out or knocked down in the NK cells. In specific embodiments, the CISH gene is knocked out or knocked down in the NK cells. In specific embodiments, the CD38 gene is knocked out or knocked down in the NK cells. In specific embodiments, the Glucocorticoid receptor (GR) gene is knocked out or knocked down in the NK cells.

In some embodiments, the gene editing is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus. Methods of utilizing a CRISPR system are well known in the art

### IV. ADMINISTRATION OF THERAPEUTIC COMPOSITIONS AND METHODS OF USE

In some embodiments, TCR/FcR comprising NK cells are administered to an individual in need thereof. In some embodiments, TCR/FcR comprising NK cells are administered to an individual in need there of in combination with monospecific, bispecific, and/or multi-specific antibodies. In some embodiments, combination administration to an individual in need thereof occurs in such a way as to have the TCR/FcR NK cells and antibodies be in proximity, and thus the antibody and NK cells are able to interact through one or more receptors (e.g., CD3/TCR complex, CD16, etc.). In some cases, the two components are administered separately to an individual, whereas in other cases the two components are complexed together (i.e., NK cells are loaded with one, two, or more components) prior to administration, such as in an ex vivo manner. In another embodiment, the NK cells express the antibodies. In some cases, the two components are not pre-complexed prior to administration, but are co-administered by any suitable route of administration, such as by co-infusion to the patient. In some embodiments one or more components (e.g., monospecific, bispecific, and/or multispecific antibodies) could be loaded onto NK cells ex vivo, while other components, which may be the same components or different components, may be co-administered.

In some embodiments, methods of using TCR/FcR comprising NK cells in conjunction with antibodies results in synergistic effects. In some embodiments, combination of TCR/FcR comprising NK cells with Blinatumomab results in a synergistic effect and increased NK cell cytotoxicity against CD19 bearing cells (e.g., cancer cells). In some embodiments, combination of TCR/FcR comprising NK cells with Obinutuzumab results in a synergistic effect and increased NK cell cytotoxicity against CD20 bearing cells (e.g., cancer cells). In some embodiments, combination of TCR/FcR comprising NK cells with Rituximab results in a synergistic effect and increased NK cell cytotoxicity against CD19 bearing cells (e.g., cancer cells). In some embodiments, combination of TCR/FcR comprising NK cells with Amivantamab results in a synergistic effect and increased NK cell cytotoxicity against EGFR and/or c-MET bearing cells (e.g., cancer cells). In some embodiments, combination of TCR/FcR comprising NK cells with Imgatuzumab results in a synergistic effect and increased NK cell cytotoxicity against EGFR bearing cells (e.g., cancer cells). In some embodiments, combination of TCR/FcR comprising NK cells with Cetuximab results in a synergistic effect and increased NK cell cytotoxicity against EGFR bearing cells (e.g., cancer cells). In some embodiments, combination of TCR/FcR comprising NK cells with Teclistamab results in a synergistic effect and increased NK cell cytotoxicity against BCMA bearing cells (e.g., cancer cells). In some embodiments, combination of TCR/FcR comprising NK cells with Elranatamab results in a synergistic effect and increased NK cell cytotoxicity against BCMA bearing cells (e.g., cancer cells). In some embodiments, combination of TCR/FcR comprising NK cells with Talquetamab results in a synergistic effect and increased NK cell cytotoxicity against GPRC5D bearing cells (e.g., cancer cells). In some embodiments, combination of TCR/FcR comprising NK cells with Pertuzumab results in a synergistic effect and increased NK cell cytotoxicity against HER2 bearing cells (e.g., cancer cells). In some embodiments, combination of TCR/FcR comprising NK cells with Trastuzumab results in a synergistic effect and increased NK cell cytotoxicity against HER2 bearing cells (e.g., cancer cells). In some embodiments, combination of TCR/FcR comprising NK cells with Tafasitamab results in a synergistic effect and increased NK cell cytotoxicity against CD19 bearing cells (e.g., cancer cells). In some embodiments, combination of TCR/FcR comprising NK cells with Brentuximab results in a synergistic effect and increased NK cell cytotoxicity against CD30 bearing cells (e.g., cancer cells).

Embodiments of the present disclosure concern methods for the use of the compositions comprising NK cells and antibodies provided herein for treating or preventing a medical disease or disorder. In some embodiments, methods include administering to the subject a therapeutically effective amount of a TCR/FcR-modified NK cell with one or more antibodies, thereby treating or preventing the disease in the subject, including reducing the risk of, reducing the severity of, and/or delaying the onset of the disease. In certain embodiments of the present disclosure, cancer or infection is treated by transfer of a composition comprising the NK cell population and corresponding antibodies. In certain embodiments of the present disclosure, an autoimmune disorder is treated by transfer of a composition comprising the NK cell population and corresponding antibodies. In at least some cases, because of their release of pro-inflammatory cytokines, NK cells may reverse the anti-inflammatory tumor microenvironment and increase adaptive immune responses by promoting differentiation, activation, and/or recruitment of accessory immune cell to sites of malignancy. In certain embodiments, a providing step may comprise culturing the NK cells with antibody molecules for a specific duration of time (e.g., about 5 minutes to about 24 hours or more) and storing the NK cells and the antibody molecules for a period of time (e.g., about 1, 2, 3, 4, 5 days, or greater than 5 days) prior to infusion/administration.

In certain embodiments of the present disclosure, an autoimmune disorder comprises a disorder characterized by abnormal B-cells. In certain embodiments, an autoimmune disorder comprises B cell related autoimmunities. In certain embodiments, an autoimmune disorder comprises systemic lupus erythematosus (SLE). In certain embodiments, an autoimmune disorder comprises systemic scleroderma (SSc). In certain embodiments, an autoimmune disorder comprises multiple sclerosis (MS). In certain embodiments, an autoimmune disorder comprises Grave's disease. In certain embodiments, an autoimmune disorder comprises rheumatoid arthritis. In certain embodiments, an autoimmune disorder comprises myositis. In certain embodiments, an autoimmune disorder comprises dermatomyositis. In certain embodiments, an autoimmune disorder comprises myasthenia gravis. In certain embodiments, an autoimmune disorder comprises Sjogren's syndrome. In certain embodiments, an autoimmune disorder comprises diffuse scleroderma. In certain embodiments, an autoimmune disorder comprises inflammatory myopathy. In certain embodiments, an autoimmune disorder comprises ANCA-associated systemic vasculitis. In certain embodiments, an autoimmune disorder comprises antiphospholipid syndrome. In certain embodiments, an autoimmune disorder comprises immune nephritis. In certain embodiments, an autoimmune disorder comprises immune thrombocytopenia (ITP). In certain embodiments, an autoimmune disorder comprises refractory POEMS (polyneuropathy, organomegaly, endocrinopathy/edema, monoclonal-protein, skin changes) syndrome. In certain embodiments, an autoimmune disorder comprises amyloidosis. In certain embodiments, an autoimmune disorder comprises autoimmune hemolytic anemia. In certain embodiments, an autoimmune disorder comprises vasculitis.

Cancers for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition. Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed. Examples of therapies other than those of the present disclosure include surgery, chemotherapy, drug therapy, radiation, hormone therapy, immunotherapy (other than that of the present disclosure), or a combination thereof.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 M or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

Embodiments of the present disclosure concern methods for the use of the compositions comprising NK cells (e.g., engineered NK cells) and antibodies provided herein for treating or preventing a medical disease or disorder. The method includes administering to the subject a therapeutically effective amount of the loaded, optionally pre-activated, and optionally expanded NK cells with the antibodies, thereby treating or preventing the disease in the subject, including reducing the risk of, reducing the severity of, and/or delaying the onset of the disease. In certain embodiments of the present disclosure, cancer or infection is treated by transfer of a composition comprising the NK cell population and antibodies. In at least some cases, because of their release of pro-inflammatory cytokines, NK cells may reverse the anti-inflammatory tumor microenvironment and increase adaptive immune responses by promoting differentiation, activation, and/or recruitment of accessory immune cell to sites of malignancy.

In certain embodiments, cancers for which the present compositions and methods described herein are useful for treatment, prevention, and/or amelioration of symptoms include at least CD19, CD20, CD30, HER2, GPRC5D, EGFR, EGFR2, BCMA, and/or c-MET expressing cancers. In certain embodiments, cancers for which the present compositions and methods described herein are useful for treatment, prevention, and/or amelioration of symptoms include at least CD19 expressing cancers. In certain embodiments, cancers for which the present compositions and methods described herein are useful for treatment, prevention, and/or amelioration of symptoms include at least CD20 expressing cancers. In certain embodiments, cancers for which the present compositions and methods described herein are useful for treatment, prevention, and/or amelioration of symptoms include at least EGFR expressing cancers. In certain embodiments, cancers for which the present compositions and methods described herein are useful for treatment, prevention, and/or amelioration of symptoms include at least BCMA expressing cancers. In certain embodiments, cancers for which the present compositions and methods described herein are useful for treatment, prevention, and/or amelioration of symptoms include at least c-MET expressing cancers. In certain embodiments, cancers for which the present compositions and methods described herein are useful for treatment, prevention, and/or amelioration of symptoms include at least CD70, and/or TROP2 expressing cancers.

In certain embodiments, compositions and methods described herein are utilized for treatment, prevention, and/or amelioration of symptoms associated with PDAC. In certain embodiments, compositions and methods described herein are utilized for treatment, prevention, and/or amelioration of symptoms associated with CRC. In certain embodiments, compositions and methods described herein are utilized for treatment, prevention, and/or amelioration of symptoms associated with ovarian cancer. In certain embodiments, compositions and methods described herein are utilized for treatment, prevention, and/or amelioration of symptoms associated with leukemias. In certain embodiments, compositions and methods described herein are utilized for treatment, prevention, and/or amelioration of symptoms associated with kidney cancer. In certain embodiments, compositions and methods described herein are utilized for treatment, prevention, and/or amelioration of symptoms associated with glioblastoma. In certain embodiments, compositions and methods described herein are utilized for treatment, prevention, and/or amelioration of symptoms associated with breast cancer. In certain embodiments, compositions and methods described herein are utilized for treatment, prevention, and/or amelioration of symptoms associated with RCC. In certain embodiments, compositions and methods described herein are utilized for treatment, prevention, and/or amelioration of symptoms associated with myeloma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

Particular embodiments concern methods of treatment of leukemia. Leukemia is a cancer of the blood or bone marrow and is characterized by an abnormal proliferation (production by multiplication) of blood cells, usually white blood cells (leukocytes). It is part of the broad group of diseases called hematological neoplasms. Leukemia is a broad term covering a spectrum of diseases. Leukemia is clinically and pathologically split into its acute and chronic forms.

Acute leukemia is characterized by the rapid proliferation of immature blood cells. This crowding makes the bone marrow unable to produce healthy blood cells. Acute forms of leukemia can occur in children and young adults. In fact, it is a more common cause of death for children in the U.S. than any other type of malignant disease. Immediate treatment is required in acute leukemia due to the rapid progression and accumulation of the malignant cells, which then spill over into the bloodstream and spread to other organs of the body. Central nervous system (CNS) involvement is uncommon, although the disease can occasionally cause cranial nerve palsies. Chronic leukemia is distinguished by the excessive buildup of relatively mature, but still abnormal, blood cells. Typically taking months to years to progress, the cells are produced at a much higher rate than normal cells, resulting in many abnormal white blood cells in the blood. Chronic leukemia mostly occurs in older people, but can theoretically occur in any age group. Whereas acute leukemia must be treated immediately, chronic forms are sometimes monitored for some time before treatment to ensure maximum effectiveness of therapy.

Furthermore, the diseases are classified into lymphocytic or lymphoblastic, which indicate that the cancerous change took place in a type of marrow cell that normally goes on to form lymphocytes, and myelogenous or myeloid, which indicate that the cancerous change took place in a type of marrow cell that normally goes on to form red cells, some types of white cells, and platelets (see lymphoid cells vs. myeloid cells).

Acute lymphocytic leukemia (also known as acute lymphoblastic leukemia, or ALL) is the most common type of leukemia in young children. This disease also affects adults, especially those aged 65 and older. Chronic lymphocytic leukemia (CLL) most often affects adults over the age of 55. It sometimes occurs in younger adults, but it almost never affects children. Acute myelogenous leukemia (also known as acute myeloid leukemia, or AML) occurs more commonly in adults than in children. This type of leukemia was previously called “acute nonlymphocytic leukemia.” Chronic myelogenous leukemia (CIL) occurs mainly in adults. A very small number of children also develop this disease.

Lymphoma is a type of cancer that originates in lymphocytes (a type of white blood cell in the vertebrate immune system). There are many types of lymphoma. According to the U.S. National Institutes of Health, lymphomas account for about five percent of all cases of cancer in the United States, and Hodgkin's lymphoma in particular accounts for less than one percent of all cases of cancer in the United States. Because the lymphatic system is part of the body's immune system, patients with a weakened immune system, such as from HIV infection or from certain drugs or medication, also have a higher incidence of lymphoma.

In certain embodiments of the disclosure, compositions comprising the NK cells and antibodies are delivered to an individual in need thereof, such as an individual that has cancer or an infection. In at least some cases, the cells can enhance the individual's immune system to attack the respective cancer or pathogenic cells. In some cases, the individual is provided with one or more doses of the compositions comprising the NK cells and antibodies. In cases where the individual is provided with two or more doses of the NK cells/antibodies, the duration between the administrations should be sufficient to allow time for propagation in the individual, and in specific embodiments the duration between doses is 1, 2, 3, 4, 5, 6, 7, or more days.

The source of NK cells that are pre-activated (optionally) and expanded may be of any kind, but in specific embodiments the cells are obtained from a bank of umbilical cord blood, peripheral blood, human embryonic stem cells, or induced pluripotent stem cells, for example. Suitable doses for a therapeutic effect would be at least 10⁵ or between about 10⁵ and about 10¹² cells per dose, for example, preferably in a series of dosing cycles. An exemplary dosing regimen consists of four one-week dosing cycles of escalating doses, starting at least at about 10⁵ cells on Day 0, for example increasing incrementally up to a target dose of about 10¹² cells within several weeks of initiating an intra-patient dose escalation scheme. Suitable modes of administration include intravenous, subcutaneous, intracavitary (for example by reservoir-access device), intraperitoneal, and direct injection into a tumor mass.

Compositions comprising NK cells (with or without antibodies) generated according to the present methods have many potential uses, including experimental and therapeutic uses. In particular, it is envisaged that such cell populations are useful in suppressing undesirable or inappropriate immune responses. In some methods, a small number of NK cells can be removed from a patient and then manipulated and expanded ex vivo before reinfusing them into the patient. Examples of diseases which may be treated in this way are autoimmune diseases and conditions in which suppressed immune activity is desirable, e.g., for allo-transplantation tolerance. A therapeutic method could comprise obtaining NK cells from a mammal; expanding the NK cells ex vivo in accordance with the methods of the present methods as described herein; engineering NK cells as described herein, exposing under sufficient conditions the NK cells to antibodies; and administering the compositions comprising expanded NK cells/antibodies to the mammal to be treated.

A pharmaceutical composition of the present disclosure can be used alone or in combination with other well-established agents useful for treating cancer. Whether delivered alone or in combination with other agents, the pharmaceutical composition of the present disclosure can be delivered via various routes and to various sites in a mammalian, particularly human, body to achieve a particular effect. One skilled in the art will recognize that, although more than one route can be used for administration, a particular route can provide a more immediate and more effective reaction than another route. For example, intradermal delivery may be advantageously used over inhalation for the treatment of melanoma. Local or systemic delivery can be accomplished by administration comprising application or instillation of the formulation into body cavities, inhalation or insufflation of an aerosol, or by parenteral introduction, comprising intramuscular, intravenous, intraportal, intrahepatic, peritoneal, subcutaneous, or intradermal administration.

In one embodiment, the subject has an autoimmune disease. Non-limiting examples of autoimmune diseases include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac spate-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, nephrotic syndrome (such as minimal change disease, focal glomerulosclerosis, or mebranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, and Wegener's granulomatosis. Thus, some examples of an autoimmune disease that can be treated using the methods disclosed herein include, but are not limited to, multiple sclerosis, rheumatoid arthritis, systemic lupus erythematosis, type I diabetes mellitus, Crohn's disease; ulcerative colitis, myasthenia gravis, glomerulonephritis, ankylosing spondylitis, vasculitis, or psoriasis. The subject can also have an allergic disorder such as Asthma.

In yet another embodiment, the subject is the recipient of a transplanted organ or stem cells and NK cells of the disclosure are used to prevent and/or treat rejection. In particular embodiments, the subject has or is at risk of developing graft versus host disease. GVHD is a possible complication of any transplant that uses or contains stem cells from either a related or an unrelated donor. There are two kinds of GVHD, acute and chronic. Acute GVHD appears within the first three months following transplantation. Signs of acute GVHD include a reddish skin rash on the hands and feet that may spread and become more severe, with peeling or blistering skin. Acute GVHD can also affect the stomach and intestines, in which case cramping, nausea, and diarrhea are present. Yellowing of the skin and eyes (jaundice) indicates that acute GVHD has affected the liver. Chronic GVHD is ranked based on its severity: stage/grade 1 is mild; stage/grade 4 is severe. Chronic GVHD develops three months or later following transplantation. The symptoms of chronic GVHD are similar to those of acute GVHD, but in addition, chronic GVHD may also affect the mucous glands in the eyes, salivary glands in the mouth, and glands that lubricate the stomach lining and intestines. Any of the populations of NK cells disclosed herein can be utilized. Examples of a transplanted organ include a solid organ transplant, such as kidney, liver, skin, pancreas, lung and/or heart, or a cellular transplant such as islets, hepatocytes, myoblasts, bone marrow, or hematopoietic or other stem cells. The transplant can be a composite transplant, such as tissues of the face. NK cells, such as immunosuppressive CD19+cells, can be administered (with or without antibodies) prior to transplantation, concurrently with transplantation, and/or following transplantation. In some embodiments, compositions comprising NK cells (with or without antibodies) are administered prior to the transplant, such as at least 1 hour, at least 12 hours, at least 1 day, at least 2 days, at least 3 days, at least 4 days, at least 5 days, at least 6 days, at least 1 week, at least 2 weeks, at least 3 weeks, at least 4 weeks, or at least 1 month prior to the transplant. In one specific, non-limiting example, administration of the therapeutically effective amount of the compositions comprising the NK cells (with or without antibodies) occurs 3-5 days prior to transplantation.

In some embodiments, NK cells (with or without antibodies) administered to a patient that is receiving a transplant can be sensitized with antigens specific to the transplanted material prior to administration. According to this embodiment, the transplant recipient will have a decreased immune/inflammatory response to the transplanted material and, as such, the likelihood of rejection of the transplanted tissue is minimized. Similarly, with regard to the treatment of graft versus host disease, the NK cells can be sensitized with antigens specific to the host. According to this embodiment, the recipient will have a decreased immune/inflammatory response to self-antigens.

In a further embodiment, administration of a therapeutically effective amount of compositions comprising NK cells (with or without antibodies) to a subject treats or inhibits inflammation in the subject. Thus, the method includes administering a therapeutically effective amount of compositions comprising NK cells (with or without antibodies) to the subject to inhibit the inflammatory process. Examples of inflammatory disorders include, but are not limited to, asthma, encephalitis, inflammatory bowel disease, chronic obstructive pulmonary disease (COPD), allergic disorders, septic shock, pulmonary fibrosis, undifferentiated spondyloarthropathy, undifferentiated arthropathy, arthritis, inflammatory osteolysis, and chronic inflammation resulting from chronic viral or bacterial infections. The methods disclosed herein can also be used to treat allergic disorders.

Administration of compositions comprising NK cells (with or without antibodies) can be utilized whenever immunosuppression or inhibition of inflammation is desired, for example, at the first sign or symptoms of a disease or inflammation. These may be general, such as pain, edema, elevated temperature, or may be specific signs or symptoms related to dysfunction of affected organ(s). For example, in renal transplant rejection there may be an elevated serum creatinine level, whereas in GVHD, there may be a rash, and in asthma, there may be shortness of breath and wheezing.

Administration of compositions comprising NK cells (with or without antibodies) can also be utilized to prevent immune-mediated disease in a subject of interest. For example, compositions comprising NK cells (with or without antibodies) can be administered to a subject that will be a transplant recipient prior to the transplantation. In another example, compositions comprising NK cells (with or without antibodies) are administered to a subject receiving allogeneic bone marrow transplants without T cell depletion. In a further example, compositions comprising NK cells (with or without antibodies) can be administered to a subject with a family history of diabetes. In other example, compositions comprising NK cells (with or without antibodies) are administered to a subject with asthma in order to prevent an asthma attack. In some embodiments, a therapeutically effective amount of compositions comprising NK cells (with or without antibodies) is administered to the subject in advance of a symptom. In some embodiments, administration of the compositions comprising NK cells (with or without antibodies) results in decreased incidence or severity of subsequent immunological event or symptom (such as an asthma attack), or improved patient survival, compared to patients who received other therapy not including compositions comprising NK cells (with or without antibodies).

In certain embodiments, the compositions comprising NK cells (with or without antibodies) are administered in combination with one or more additional therapeutic agents. For example, the therapeutic agent may comprise T cells, an immunomodulatory agent, a monoclonal antibody, a chemotherapeutic agent, hormone(s), drugs of any kind, surgery, radiation, etc. In non-limiting examples, the immunomodulatory agent is lenalidomide, the monoclonal antibody is rituximab, ofatumumab, or lumiliximab, and the chemotherapeutic agent is fludarabine or cyclophosphamide. In non-limiting examples, the immunomodulatory agent is Elranatamab, Blinatumomab, Obinutuzumab, Rituximab, Amivantamab, Imgatuzumab, Cetuximab, and/or Teclistamab.

A composition of the present disclosure can be provided in unit dosage form wherein each dosage unit, e.g., an injection, contains a predetermined amount of the composition, alone or in appropriate combination with other active agents. The term unit dosage form as used herein refers to physically discrete units suitable as unitary dosages for human and animal subjects, each unit containing a predetermined quantity of the composition of the present disclosure, alone or in combination with other active agents, calculated in an amount sufficient to produce the desired effect, in association with a pharmaceutically acceptable diluent, carrier, or vehicle, where appropriate. The specifications for the unit dosage forms of the present disclosure depend on the particular pharmacodynamics associated with the pharmaceutical composition in the particular subject.

Desirably an effective amount or sufficient number of the isolated transduced NK cells is present in the composition and introduced into the subject such that long-term, specific, anti-tumor responses are established to reduce the size of a tumor or eliminate tumor growth or regrowth than would otherwise result in the absence of such treatment. Desirably, the amount of NK cells reintroduced into the subject causes a 10%, 20%, 30%, 40%, 50%, 60%, 70%, 80%, 90%, 95%, 98%, or 100% decrease in tumor size when compared to otherwise same conditions wherein the NK cells are not present.

Accordingly, the amount of compositions comprising NK cells (with or without antibodies) administered should take into account the route of administration and should be such that a sufficient number of the compositions comprising NK cells (with or without antibodies) will be introduced so as to achieve the desired therapeutic response. Furthermore, the amounts of each active agent included in the compositions described herein (e.g., the amount per each cell to be contacted or the amount per certain body weight) can vary in different applications. In general, the concentration of NK cells desirably should be sufficient to provide in the subject being treated at least from about 1×10⁶ to about 1×10¹² NK cells, even more desirably, from about 1×10⁷ to about 5×10¹⁰ NK cells, although any suitable amount can be utilized either above, e.g., greater than 5×10⁸ cells, or below, e.g., less than 1×10⁷ cells. The dosing schedule can be based on well-established cell-based therapies (see, e.g., U.S. Pat. No. 4,690,915), or an alternate continuous infusion strategy can be employed. In certain embodiments, a subject being treated is provided with at least, exactly, or about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, or 9×10⁸ NK cells. In some embodiments, a subject being treated is provided with one or more doses of NK cells, at one or more dose levels. In some embodiments, a subject being treated is provided with at least, exactly, or about 4×10⁷ NK cells. In some embodiments, a subject being treated is provided with at least, exactly, or about 8×10⁷ NK cells. In some embodiments, a subject being treated is provided with at least, exactly, or about 4×10⁸ NK cells. In some embodiments, a subject being treated is provided with at least, exactly, or about 8×10⁸ NK cells.

These values provide general guidance of the range of compositions comprising NK cells and antibodies to be utilized by the practitioner upon optimizing the method of the present disclosure for practice of the present methods. The recitation herein of such ranges by no means precludes the use of a higher or lower amount of a component, as might be warranted in a particular application. For example, the actual dose and schedule can vary depending on whether the compositions are administered in combination with other pharmaceutical compositions, or depending on interindividual differences in pharmacokinetics, drug disposition, and metabolism. One skilled in the art readily can make any necessary adjustments in accordance with the exigencies of the particular situation.

### A. Combination Therapies

In certain embodiments, the compositions and methods of the present embodiments involve a cancer therapy that is additional to the compositions comprising the NK cells and antibodies. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, hormone therapy, or a combination of the foregoing. The additional therapy may be in the form of adjuvant or neoadjuvant therapy.

In some embodiments, the additional therapy is the administration of small molecule enzymatic inhibitor(s) or anti-metastatic agent(s). In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent(s). The additional therapy may be one or more of the chemotherapeutic agents known in the art.

An immune cell therapy (in addition to the compositions of the disclosure) may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from the composition(s) of the disclosure, one would generally ensure that a significant period of time did not expire between the time of each delivery, such that the two compounds would still be able to exert an advantageously combined effect on the patient. In such instances, it is contemplated that one may provide a patient with the immunotherapy therapy and the disclosed compositions within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

### 1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclophosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaIl); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein transferase inhibitors, transplatinum, and pharmaceutically acceptable salts, acids, or derivatives of any of the above.

### 2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### 3. Immunotherapy

The skilled artisan will understand that additional immunotherapies (outside of the disclosed cell therapy) may be used in combination or in conjunction with methods of the embodiments.

In certain embodiments, additional immunotherapies comprise administration of one or more antibodies to a subject. In certain embodiments, the additional one or more antibodies that are administered to a subject may be the same one or more antibodies loaded onto an NK cell. In some embodiments, the additional one or more antibodies that are administered to a subject may target one or more of the same antigens as the one or more antibodies loaded onto an NK cell. In some embodiments, the additional one or more antibodies that are administered to a subject are administered prior to administration of immune cells (e.g., NK cells), for example but not limited to, 1, 2, 3, 4, 5, 6, 7, 8, 9, 10, 11, 12, 13, 14, or 15 days prior to administration of immune cells. In some embodiments, the additional one or more antibodies that are administered to a subject are administered after administration of immune cells (e.g., NK cells), for example but not limited to, 1, 2, 3, 4, 5, 6, 7, 8, 9, 10, 11, 12, 13, 14, 15, 16, 17, 18, 19, 20, 21, 22, 23, 24, 25, 26, 27, 28 or longer than 28 days, after administration of immune cells. In some embodiments, the additional one or more antibodies that are administered to a subject are administered concurrently with administration of immune cells (e.g., NK cells), for example but not limited to, 1, 2, 3, 4, 5, or more than 5 times.

In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells other than those having knockdown or knockout of TGF-beta R2.

Antibody-drug conjugates have emerged as a breakthrough approach to the development of cancer therapeutics. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons of any kind, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

### 4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### 5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### V. KITS

Certain aspects of the present disclosure also concern kits comprising compositions of the invention or compositions to implement methods of the invention. In particular embodiments, the kit comprises NK cells, fresh or frozen, and that may or may not have been pre-activated or expanded. The NK cells may or may not already express one or more components of the TCR/CD3 complex and/or transgenic FcTR derived Fc binding domain comprising polypeptides (e.g., comprising an extracellular domain from CD16, CD32, and/or CD64). In cases wherein the NK cells do not already express one or more components of the TCR/CD3 complex and/or transgenic FcTR derived Fc binding domain comprising polypeptides, the kit may comprise reagents for corresponding transfection or transduction of the NK cells, including reagents such as vectors that express the component(s), primers for amplification of the component(s), and so forth. In some cases, the NK cells may or may not also express one or more heterologous proteins as defined herein, and when they do not, the kit may comprise vectors that express the heterologous protein(s), primers for amplification of the heterologous protein(s), and so forth.

Kits may comprise components which may be individually packaged or placed in a container, such as a tube, bottle, vial, syringe, or other suitable container means. Individual components (such as but not limited to, modified NK cells, non-modified NK cells, means to modify NK cells, antibodies, cytokines, etc.) may also be provided in a kit in concentrated amounts; in some embodiments, a component is provided individually in the same concentration as it would be in a solution with other components. Concentrations of components may be provided as 1×, 2×, 5×, 10×, or 20×or more. In some embodiments the components are provided in separate solutions or separate containers, in some embodiments the components are provided in a single container.

### VI. EXAMPLES

The following examples are included to demonstrate particular embodiments of the invention. It should be appreciated by those of skill in the art that the techniques disclosed in the examples which follow represent techniques discovered by the inventor to function well in the practice of the invention, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the invention.

### Example 1—Invariant Tcr and Cd3 Expressing Nk Cells

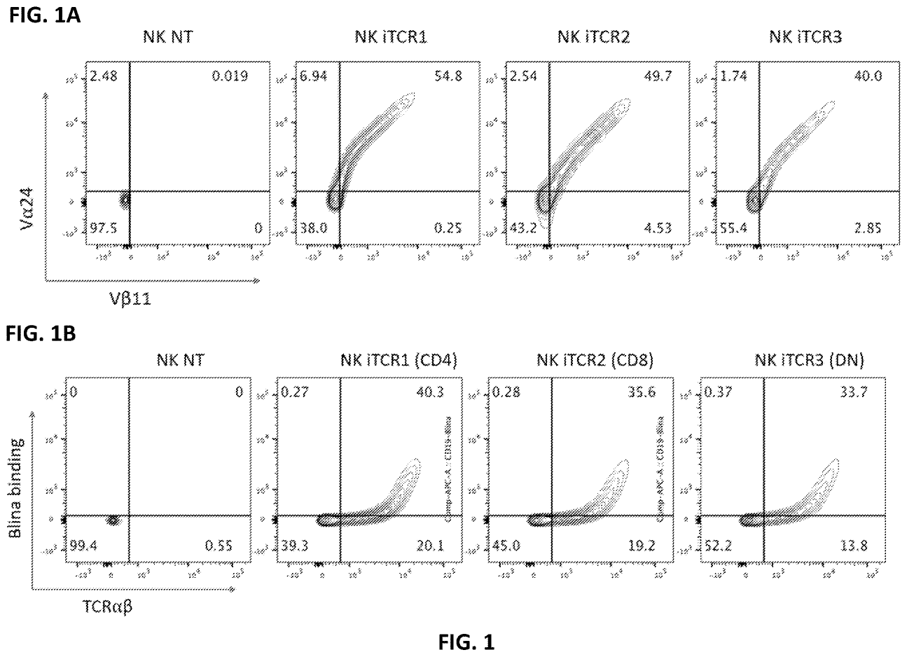

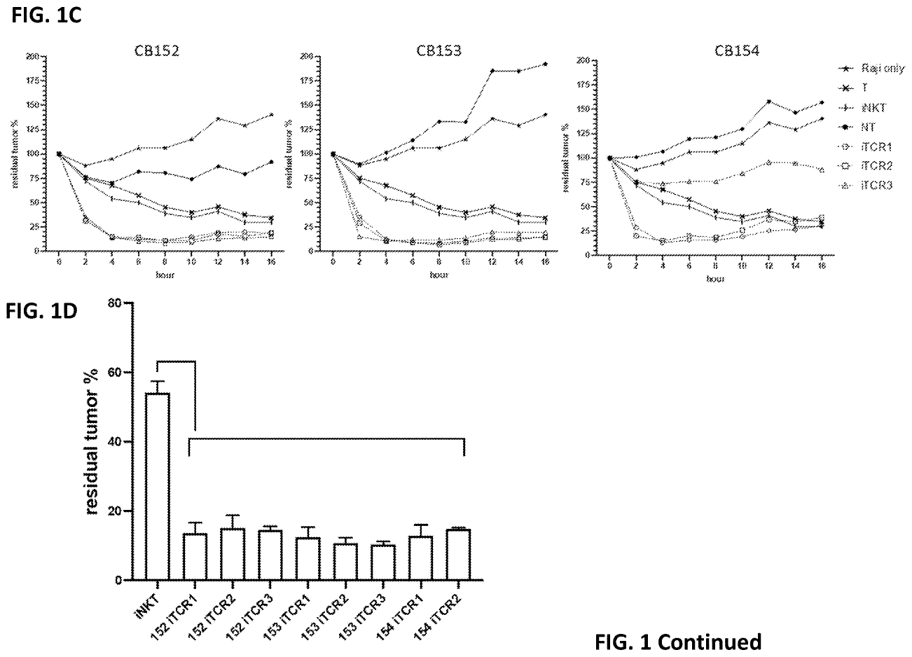

As shown in FIGS. 1A-1D, invariant Natural Killer T-cell Receptors (iTCRs) were transduced into cord blood derived NK cells, and the iTCR expression profile was determined. NK cells were derived from cord blood (three donors, CD4, CD8, and DN respectively), and were expanded with irradiated (100 Gy) universal antigen presenting cells (uAPC) feeder cells (2:1 feeder cell:NK ratio) and recombinant human IL-2 (200 U/ml) in complete media. To create a universal T cell-like NK cell that could secrete IL-15 and signal via CD3 upon CD3 antibody engagement, NK cells were purified and co-transduced with a retroviral construct containing a CD3 complex with NK co-stimulatory molecules and an IL-15 gene, and with an invariant Natural Killer T Cell receptor (iTCR) (alpha and beta chains) on day 5. Following transduction, cells underwent another round of expansion with uAPCs. As shown in FIG. 1A, exemplary flow cytometry plots showed expression of three different iTCR pairs in transduced NK cell populations, with each population expressing proteins iTCRα (SEQ ID NO: 52), and either iTCRβ chain 1 (SEQ ID NO: 54), iTCRβ chain 2 (SEQ ID NO: 56), or iTCRβ chain 3 (SEQ ID NO: 58), and CD3 complex. Confirmation of iTCR expression was determined using antibodies specific for iTCR target Va24 and V3 11 regions (y and x axis respectively). To prepare cells for analysis, NK cells were isolated from cord blood and pre-treated with IL15, IL18, and IL12 cytokine cocktail on day −1. The next day (day 0) irradiated uAPC feeder cells were added to stimulate and expand the NK cells. Transduction was done on day 5 with both uTNK15 and the exampled iNKT iTCR retroviral compositions. iTCR expression on NK cells was confirmed with antibodies specific for iTCR that targets Va24 and V311 regions. As shown in FIG. 1B, exemplary flow cytometry plot results showed binding of Blinatumomab (y axis) to three different CD3/iTCR complex (comprising iTCR1, iTCR2, and iTCR3, respectively) expressing NK cells derived from three donors (cell donor 4 (CD4), cell donor 8 (CD8), and cell donor N (CDN), respectively). Blinatumomab was pre-loaded on the 3 different iTCR-expressing NK cells by incubation of NK cells with Blinatumomab (4 μg/μl final concentration) for 1 hour at 37° C., followed by washing. The binding of Blinatumomab on CD3 was confirmed by flow cytometry using the CAR19 detection kit from Miltenyi Biotech™, which uses the CD19 protein to detect Blinatumomab (y axis) binding on NK cell surface. As shown in FIGS. 1C-1D IncuCyte® live cell imaging was used to measure the cytotoxicity of NK cells pre-loaded with Blinatumomab (as detailed above), co-transduced with iTCR constructs and UT-NK15, against GFP-expressing Raji cells. GFP-expressing Raji cells were co-cultured with CD3/iTCR-expressing NK cells at 3:1 effector to target ratios (150,000 effector and 50,000 target cells were seeded in each well of a 96 well plate). T cells and iNKT cells were used as positive and negative controls, respectively. A reduction in GFP expression indicated cell death. Three NK donor lines were utilized, CB152, CB153, and CB154, respectively, transduced with iTCRs (iTCRβ chain 1, 2, or 3 (SEQ ID NOs: 53, 55, or 57, respectively) and iTCRα (SEQ ID NO: 51)) or left non-transduced (NT) (as negative control).

The results depicted in FIG. 1 show that iTCRs can be expressed in cord blood derived NK cells, and that these iTCRs can be utilized to stabilize the expression of transgenic CD3. Furthermore, that the transgenic CD3 can be bound by CD3 targeting antibodies, such as Blinatumomab, to create antibody loaded CD3/iTCR-expressing NK cells. Loading of CD3/iTCR-expressing NK cells with an antibody (e.g., Blinatumomab) rendered the cells highly cytotoxic against tumor cells (e.g., Raji cells), with residual tumor cell numbers in the trials being lower for CD3/iTCR expressing NK cells when compared to control cells such as T cells, iNKT cells, and/or non-transduced NK cells.

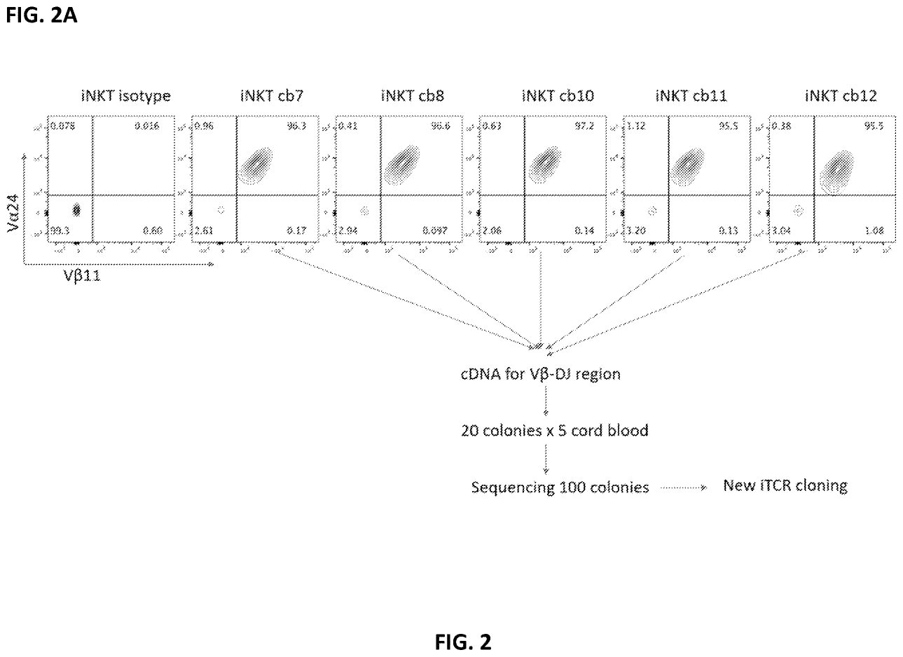

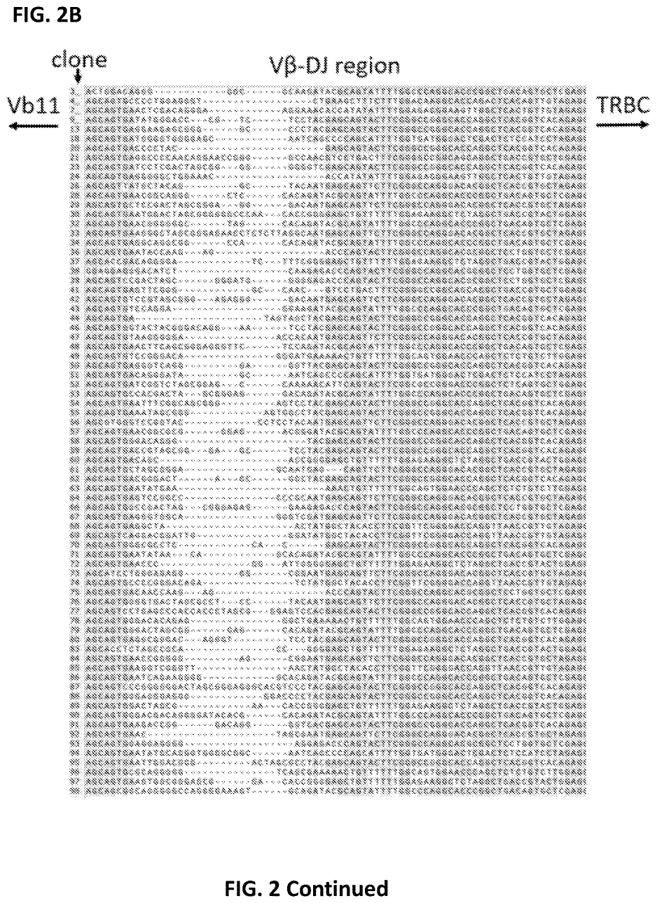

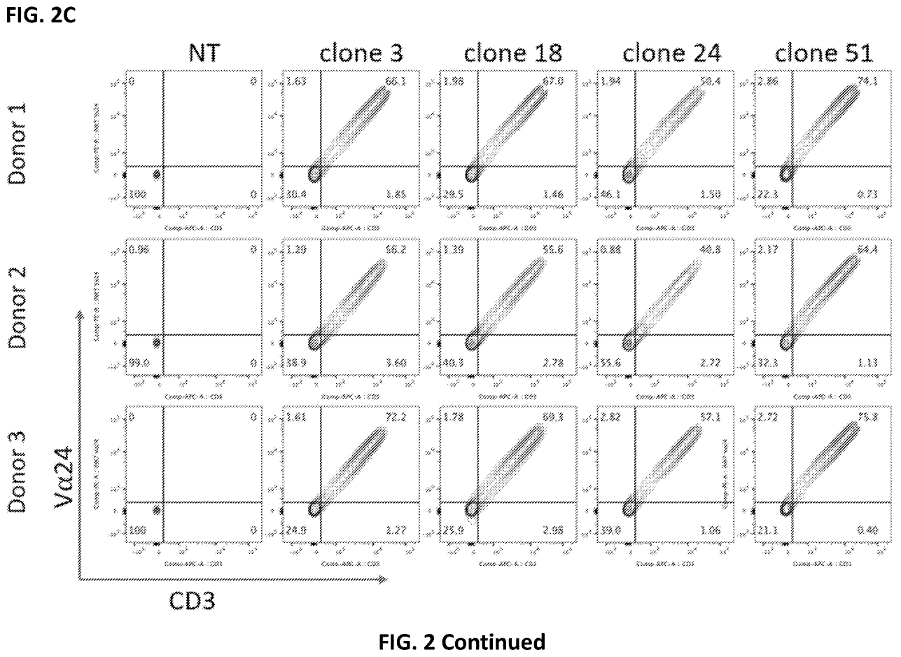

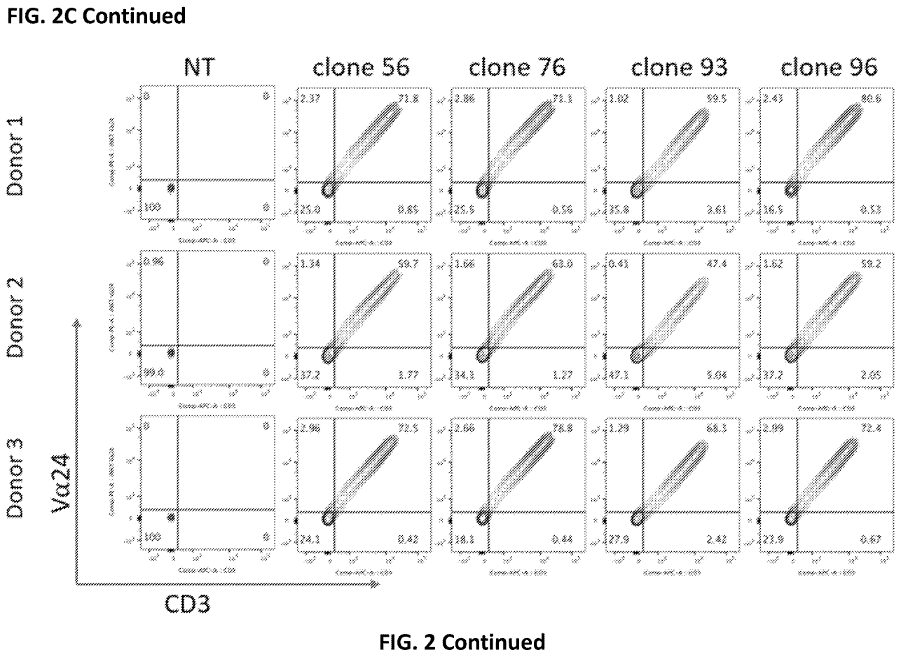

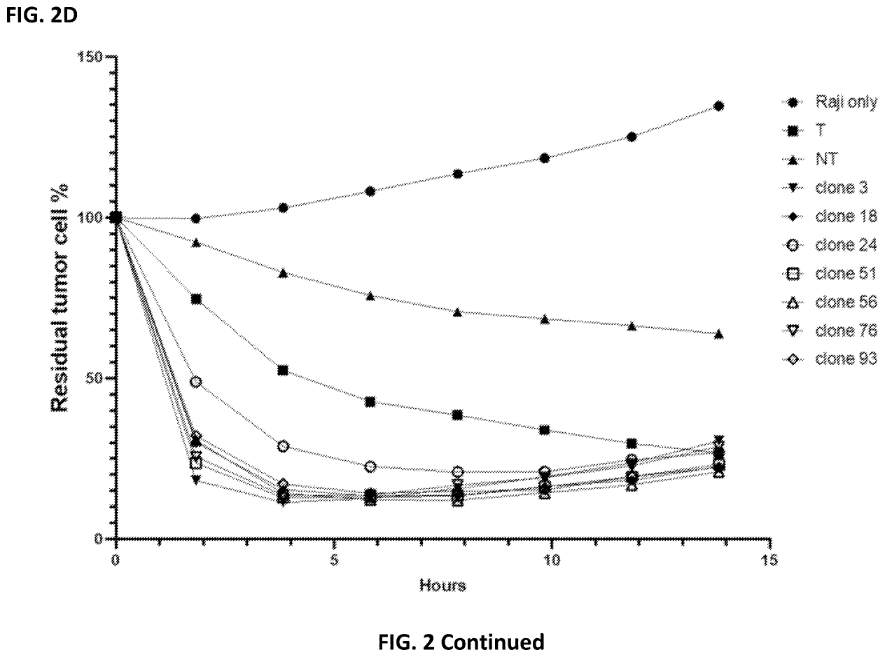

As shown in FIGS. 2A-2C, newly cloned iTCRβ sequences were obtained from cord blood derived iNKT cells, these iTCRβ chains (in addition to iTCRα) were successfully transgenically expressed in cord blood derived NK cells, and facilitated improved cytotoxicity of the NK cells against tumor cells. As depicted in FIG. 2A, iNKT cells from five cord blood donors were identified and shown to express both iTCRβ and iTCRα. The iNKT cells were isolated from 5 cord blood donors using the iNKT isolation kit from Miltenyi Biotech™ Isolated cells were stimulated and expanded with irradiated cord blood PBMC (40 Gry) in the presence of 100 ng/ml of alpha-Galactosylceramide and 200 U/ml of IL-2. On day 7 after iNKT expansion, the purity of iNKT cultures was confirmed by iNKT specific antibodies against Va24 and VP 11. Total mRNA was extracted from iNKT cells, and cDNAs of the VP-DJ regions were cloned and sequenced (100 individual clones). Shown in FIG. 2B are VP-DJ polynucleotide sequences of cloned iTCRβ chains. As shown in FIG. 2C transgenic expression of Va24 and CD3 in transgenic CD3/iTCR expressing NK cell populations from three cord blood donors was confirmed. The NK cells were co-transduced with iTCRs (eight randomly selected iTCRβ clones with coding sequences represented by SEQ ID NOs: 59, 61, 63, 65, 67, 69, 71, or 73, representing clones 3, 18, 24, 51, 56, 76, 93, and 96 respectively, and iTCRα coding sequence represented by SEQ ID NO: 51) and UT-NK15 on day 5. iTCR expression was confirmed by flow cytometry using iTCR specific antibodies against Va24 and antibodies against CD3. The cytotoxicity of these transduced cells was then tested. As shown in FIG. 2D, IncuCyte® live cell imaging was utilized to measure of cytotoxicity of NK cells co-transduced with iTCRs (as described in FIG. 2B) and UT-NK15, and pre-loaded with Blinatumomab (1 hour at 37° C.), against GFP-expressing Raji cells at 3:1 target to effector cell ratios (150,000 effector and 50,000 target cells were seeded in each well of a 96 well plate). T cells were used as positive control and non-transduced (NT) cells were utilized as negative controls. In general, the cytotoxicity of T cells expressing transgenic TCRs was found to be the same as cord blood derived in iNKT cells expressing transgenic TCRs. A reduction in GFP expression indicated tumor cell death. iTCR/CD3 expressing NK cells displayed significantly greater tumor cell cytotoxicity, as evidenced by lower relative residual tumor cell populations.

The results depicted in FIG. 2 showed that newly isolated iTCRs can be expressed in cord blood derived NK cells, and that these iTCRs can be utilized to stabilize the expression of transgenic CD3. Furthermore, that the transgenic CD3 can be bound by CD3 targeting antibodies, such as Blinatumomab, to create antibody loaded CD3/iTCR-expressing NK cells. Loading of CD3/iTCR-expressing NK cells with an antibody (e.g., Blinatumomab) rendered the majority of CD3/iTCR-expressing NK cells highly cytotoxic against tumor cells (e.g., Raji cells), with residual tumor cell numbers in the trials being lower for CD3/iTCR expressing NK cells when compared to control cells such as T cells and/or non-transduced NK cells.

### Example 2—Tcr/Fcr Nk Cells

Polynucleotide constructs comprising CD16 Fc binding domain polypeptides and invariant TCRs (iTCRs) were generated. FIGS. 3A-3C show polynucleotide construct layouts and graphical schematics for exemplary uTNK15 and TCR/FcR vectors. Shown in FIG. 3A are vector maps for multicistronic construct uTNK15-28 (SEQ ID NO: 120) comprising an open reading frame (ORF) coding region for CD3 complex proteins and IL-15, while in FIG. 3B shown are vector maps for multicistronic TCR/FcR constructs described herein comprising open reading frames for an alpha (a; SEQ ID NO: 51) and beta (0, clone 3; SEQ ID NO: 59) invariant T Cell Receptor (iTCR) chains and a CD16 Fc binding domain comprising polypeptide. In FIG. 3C provided are schematic representations for uTNK15-28, and five exemplary iTCR/CD16 Fc binding domain polypeptide bearing constructs: TCR/FcR #1 (open reading frame (ORF) SEQ ID NO: 171, in vector SEQ ID NO: 177), TCR/FcR #2 (ORF SEQ ID NO: 172, in vector SEQ ID NO: 178), TCR/FcR #3 (ORF SEQ ID NO: 173, in vector SEQ ID NO: 179), TCR/FcR #4 (ORF SEQ ID NO: 174, in vector SEQ ID NO: 180, and TCR/FcR #5 (ORF SEQ ID NO: 175, in vector SEQ ID NO: 181).

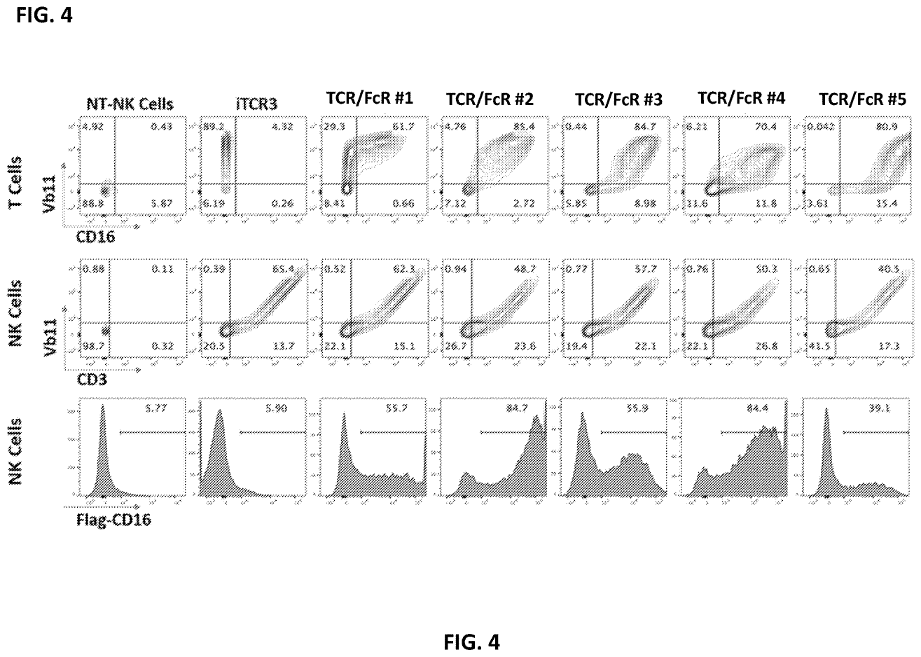

As shown in FIG. 4, cord blood (CB) derived NK cells (CB-NK) and T cells were transduced with TCR/FcR constructs and effectively expressed the encoded proteins. The top row of FIG. 4 depicts T cells expanded with anti-CD3/28 beads in the presence of IL-2 (50 Iu/ML) that were transduced with the different TCR/FcR constructs at day 3. Transduction efficiency was determined 48 hours later by measuring the surface expression of iTCRvb11 and CD16. The middle row depicts cord blood derived NK cells expanded with irradiated (100 Gy) UAPC feeder cells (2:1 feeder cell: NK ratio) and recombinant human IL-2 (200 U/ml) in complete NK cell growth medium. Seven days after expansion, NK cells were transduced with the noted iTCR3 or one of TCR/FcR constructs #1-#5, comprising CD16 Fc binding domain polypeptide variant sequences SEQ ID NOs: 155-159 respectively. Transduction efficiency was determined 48 hours later by measuring surface expression of iTCRvb11, CD3 complex, and as depicted in the bottom row, heterologous CD16 Fc binding domain comprising polypeptides (measured as anti-flag antibody against flag tagged CD16 to avoid endogenous CD16 background signal).

### Example 3—Tcr/Fcr Nk Cells Targeting Cd19 and/or Cd20 Displayed Enhanced Cytotoxicity

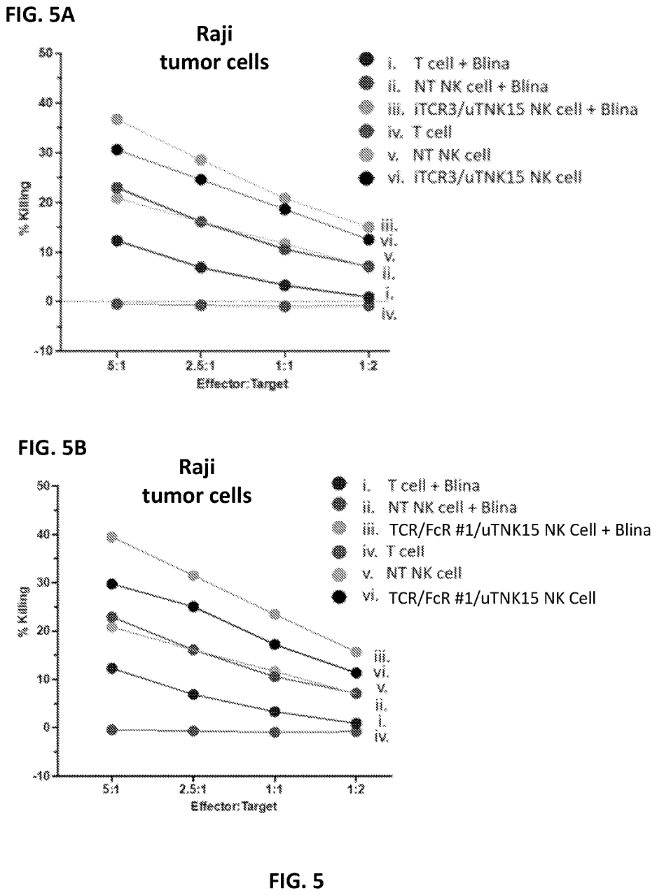

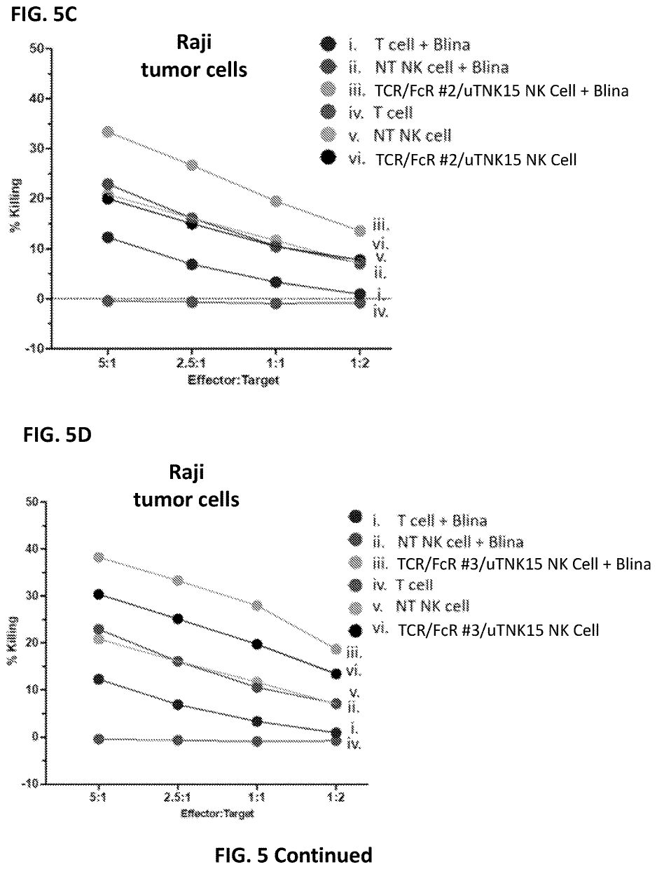

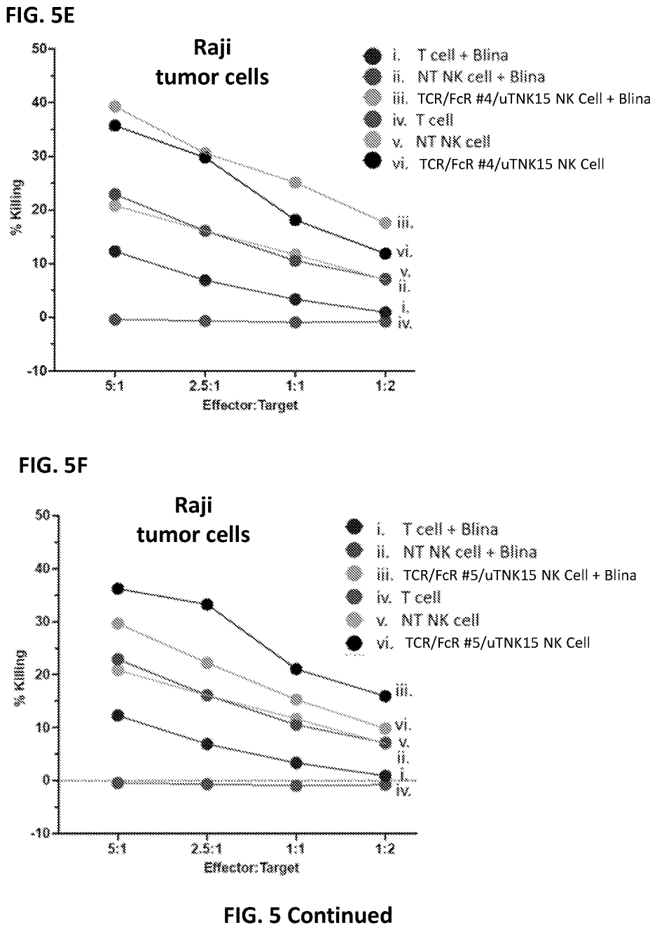

As shown in FIGS. 5A-5F, TCR/FcR transduced NK cells were effectively loaded with CD19 targeting antibody (e.g., Blinatumomab (Blina)), and displayed enhanced killing of CD19+(e.g., Raji cell line) tumor cells. Cord blood derived NK cells were transduced with uTNK15-28, and iTCR3 (5A) or one of TCR/FcR constructs #1-#5 (5B-5F). Non-transduced (NT) cord blood derived NK cells (NT NK cell) served as negative controls, while non-transduced T cells were used as positive controls. Cells were or were not loaded with Blinatumomab (100 μg/ml final concentration) for one hour at room temperature in PBS, cells were then washed prior to co-culturing with Raji cells. The Raji cells were labelled with chromium-51 and co-cultured with the immune cells at various effector to target (E:T) ratios (X axis). Cells were co-cultured for four hours and chromium release (corresponding to NK cell cytotoxicity against the cancer cells) was measured (y-axis). Compared to non-loaded TCR/FcR-NK cells, loaded or non-loaded T cells, or loaded non-transduced (NT) NK Cells, Blinatumomab-loaded TCR/FcR transduced NK cells showed increased cytotoxicity against CD19+Raji cells at E:T ratios of 5:1, 2.5:1, 1:1, and 1:2.

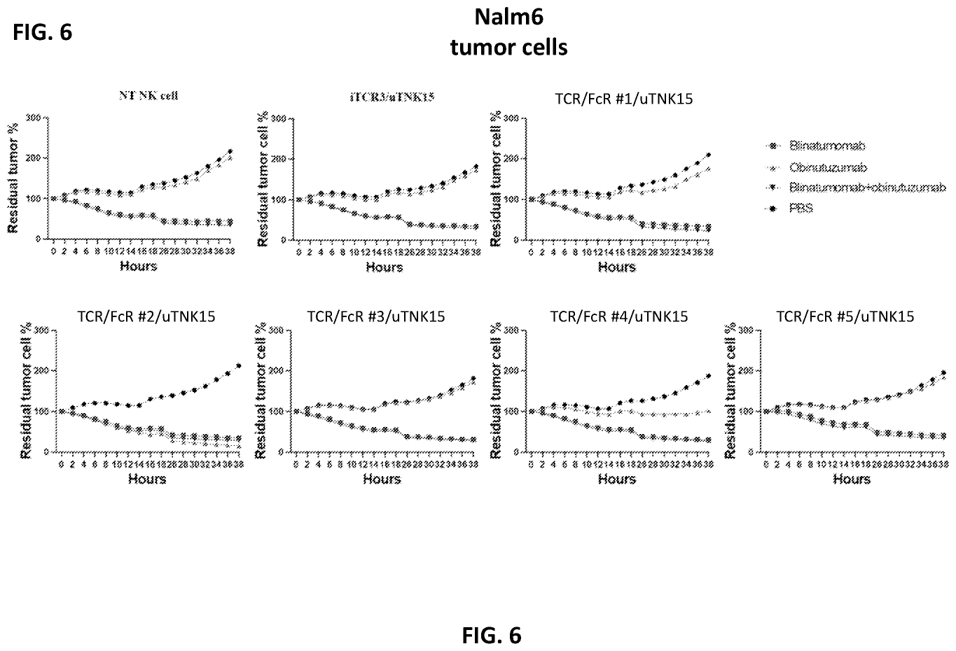

As shown in FIG. 6, TCR/FcR transduced NK cells loaded with Blinatumomab and/or Obinutuzumab displayed enhanced killing of CD19+++/CD20+(e.g., Nalm6) tumor cells. Cord blood derived NK cells were transduced with uTNK15, and iTCR3 or one of TCR/FcR constructs #1-#5. Non-transduced (NT) cord blood derived NK cells (NT NK cell) served as negative controls. NK cells were left unloaded (PBS), loaded with Blinatumomab (100 μg/ml final concentration), loaded with Obinutuzumab (500 μg/ml final concentration), or loaded with Blinatumomab (100 μg/ml final concentration) and Obinutuzumab (500 μg/ml final concentration) for one hour at room temperature in PBS and then washed prior to co-culturing with tumor cells. Immune cells and Nalm6 cells were co-cultured at a 1:1 E:T ratio and real-time cytotoxicity of effector cells against the Nalm6 cells was measured every 2 hours over a 40 hour period. Compared to non-loaded TCR/FcR-NK cells or loaded NT NK Cells, Blinatumomab and/or Obinutuzumab loaded TCR/FcR transduced NK cells showed increased cytotoxicity against CD19+++/CD20+ (e.g., Nalm6) tumor cells.

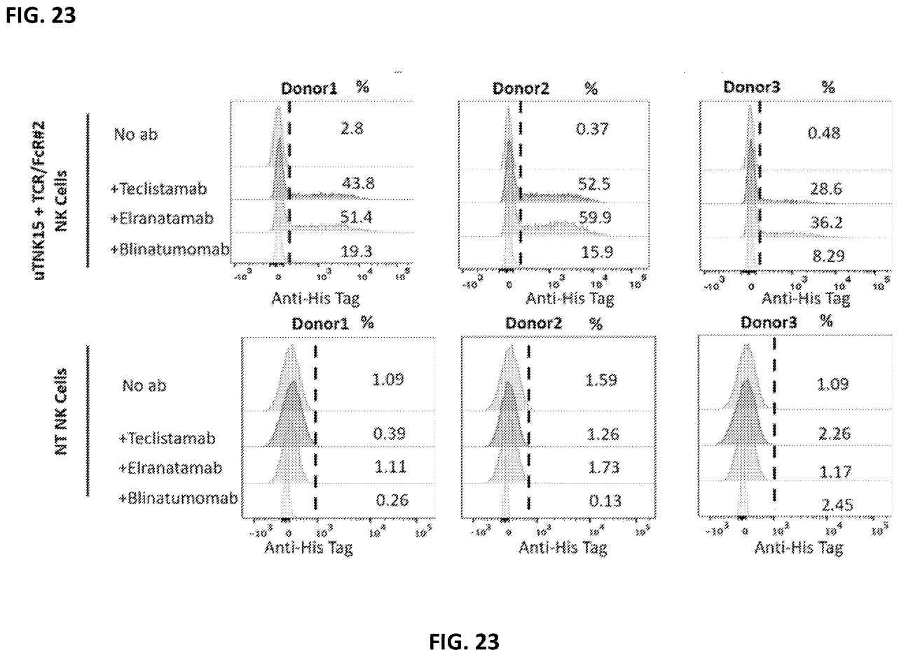

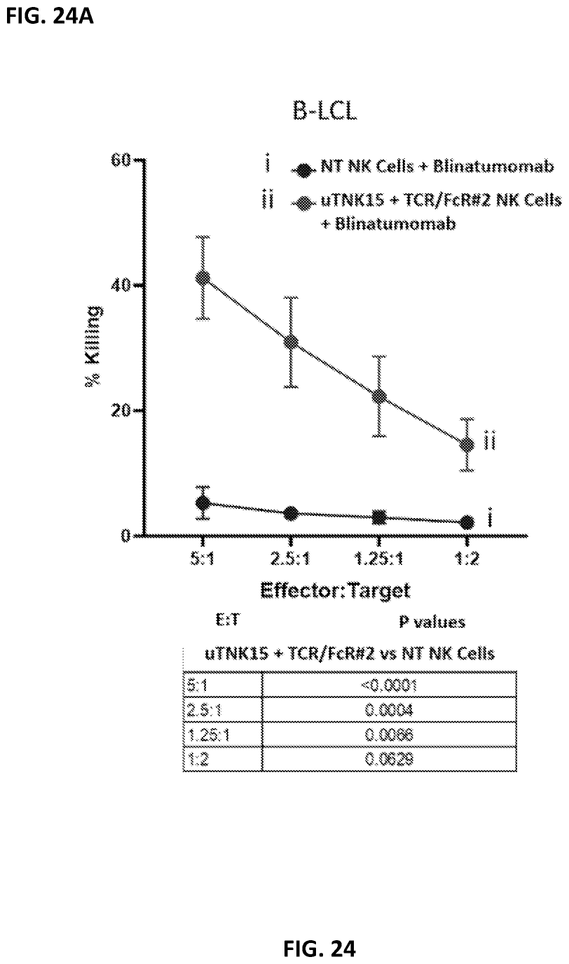

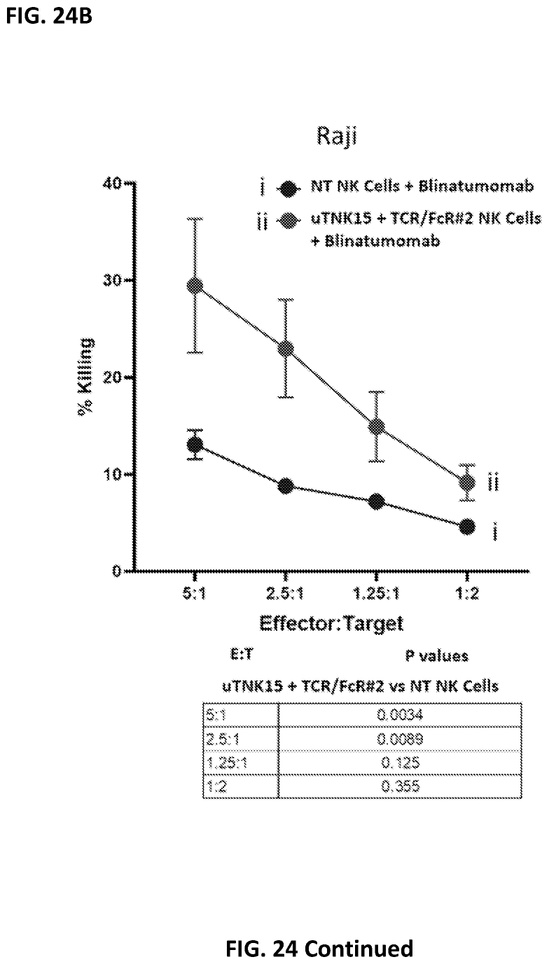

As shown in FIG. 23, TCR/FcR #2 transduced NK cells were successfully loaded with the bispecific antibodies Blinatumomab, Teclistamab, or Elranatamab, while non-transduced (NT) NK cells were not bound by the antibodies. As shown in FIGS. 24A-24B, uTNK15 and TCR/FcR #2 transduced NK cells loaded with bispecific anti-CD19/CD3 antibody blinatumomab (10 g/ml for 1 hour at 37° C. in click/RPMI media & washed prior to co-culture) demonstrated enhanced antitumor activity against CD19+tumor cells (e.g., Raji cells or B-LCL cells) at various E:T ratios in short term Cr51 assays when compared to blinatumomab loaded NT NK cells. FIG. 24A shows the results against B-LCL cells, where there was significantly more killing by engineered NK cells at 5:1, 2.5:1, and 1.25:1 E:T ratios. FIG. 24B shows the results against Raji cells, where there was significantly more killing by engineered NK cells at 5:1 and 2.5:1 E:T ratios. Compared to blinatumomab-loaded NT NK cells, blinatumomab-loaded engineered NK cells showed enhanced cytotoxicity against CD19+tumor cells.

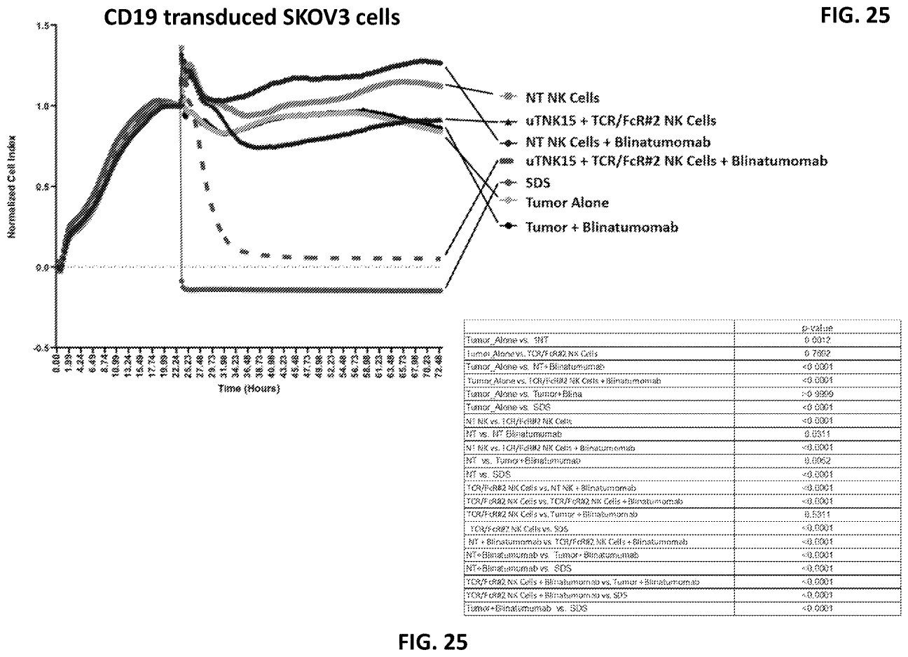

As shown in FIG. 25, Blinatumomab loaded uTNK15 and TCR/FcR #2 transduced NK cells showed enhanced killing compared to unloaded uTNK15 and TCR/FcR #2 transduced NK cells or loaded/unloaded non-transduced (NT) NK Cells. NK cells were either NT (NT or NT NK shorthand for stats) or transduced with uTNK15 and TCR/FcR #2 (“TCR/FcR #2” shorthand for stats). NK cells were either left unloaded or loaded with blinatumomab (10 g/ml for 1 hour at 37° C. in click/RPMI media & washed prior to co-culture) before co-culturing with tumor cells at 1:1 effector to target (E:T) ratio. Compared to loaded or unloaded NT NK Cells, unloaded engineered NK cells, or Blinatumumab alone, Blinatumumab-loaded engineered NK cells showed significantly increased cytotoxic activity against CD19 transduced SKOV3 cancer cells.

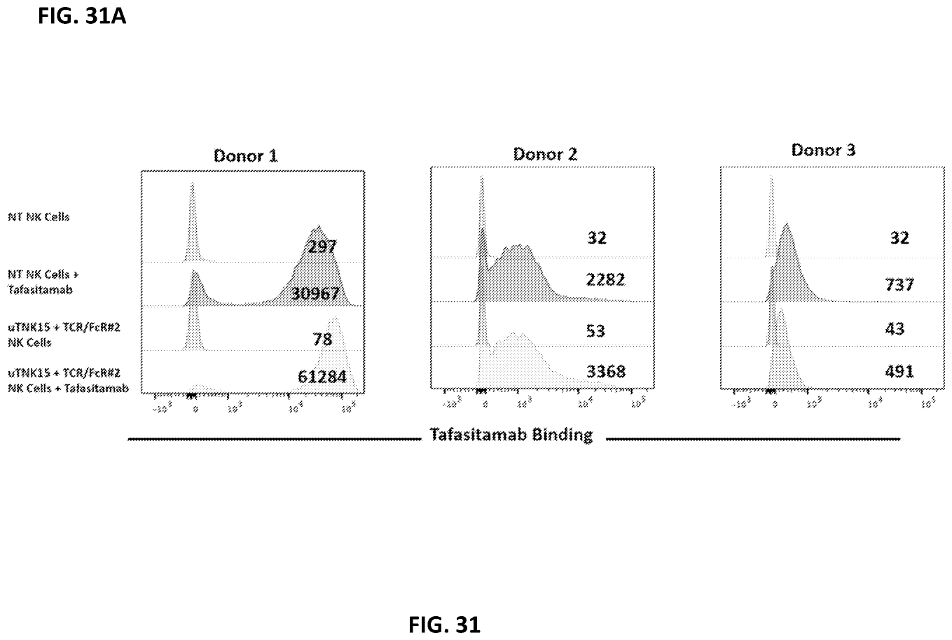

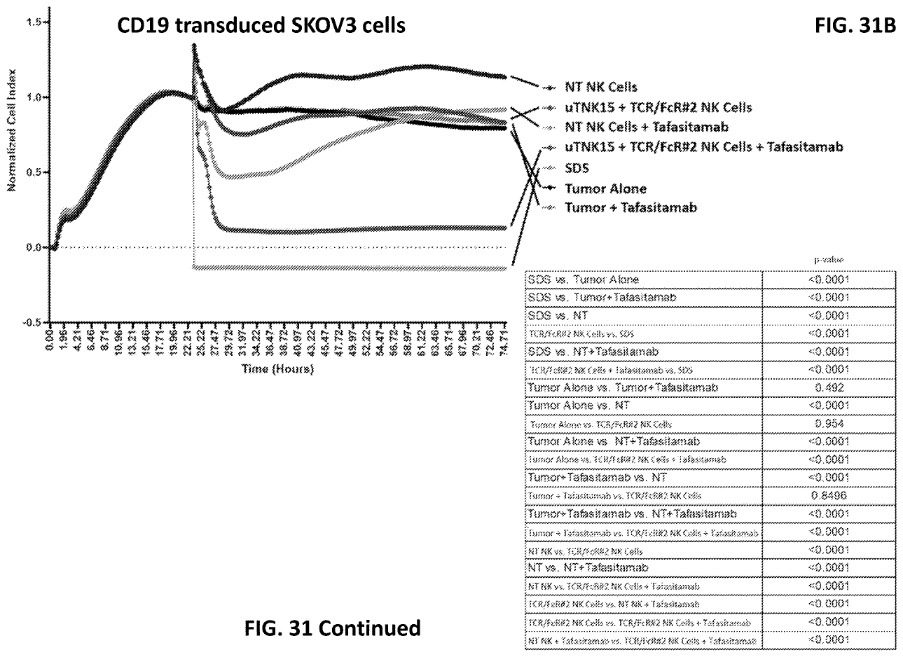

As shown in FIGS. 31A-31B, Tafasitamab (anti-CD19) bound uTNK15 and TCR/FcR #2 transduced NK cells with higher affinity than non-transduced (NT) NK cells, and Tafasitamab loaded engineered NK cells displayed superior antitumor activity against CD19 transduced SKOV3 (CD19+SKOV3) ovarian cancer cells in a long term xCELLigence killing assay. FIG. 31A, NK cells were extracted from cord blood and cultured in complete medium supplemented with irradiated (100 Gy) uAPC feeder cells at a ratio of 2:1 (feeder cell:NK). Additionally, recombinant human IL-2 was added at a concentration of 200 U/ml to facilitate expansion of the NK cells. Five days after expansion, natural killer (NK) cells were either left non-transduced, or transduced with the uTNK15 and TCR/FcR #2 constructs. These cells were then treated with Tafasitamab (10 g/ml for 1 hour at 37° C. in complete media). After incubation, the cells were washed and subjected to flow cytometric analysis to validate the binding. Flow cytometric analysis revealed that Tafasitamab exhibited higher and stronger binding affinity towards engineered NK cells compared to NT NK cells. FIG. 31B, Tafasitamab loaded uTNK15 and TCR/FcR #2 transduced NK cells showed enhanced killing compared to unloaded uTNK15 and TCR/FcR #2 transduced NK cells or loaded/unloaded NT NK Cells. NK cells were either NT (“NT” or “NT NK” shorthand for stats) or transduced with uTNK15 and TCR/FcR #2 (“TCR/FcR #2” shorthand for stats). NK cells were either left unloaded or loaded with Tafasitamab (200 g/ml for 1 hour at 37° C. in click/RPMI media & washed prior to co-culture) before co-culturing with tumor cells at 1:1 effector to target (E:T) ratio. Compared to loaded or unloaded NT NK Cells, unloaded engineered NK cells, or Tafasitamab alone, Tafasitamab-loaded engineered NK cells showed significantly increased cytotoxic activity against transduced CD19+SKOV3 ovarian cancer cells.

### Example 4—Tcr/Fcr Nk Cells Targeting Bcma Displayed Enhanced Cytotoxicity

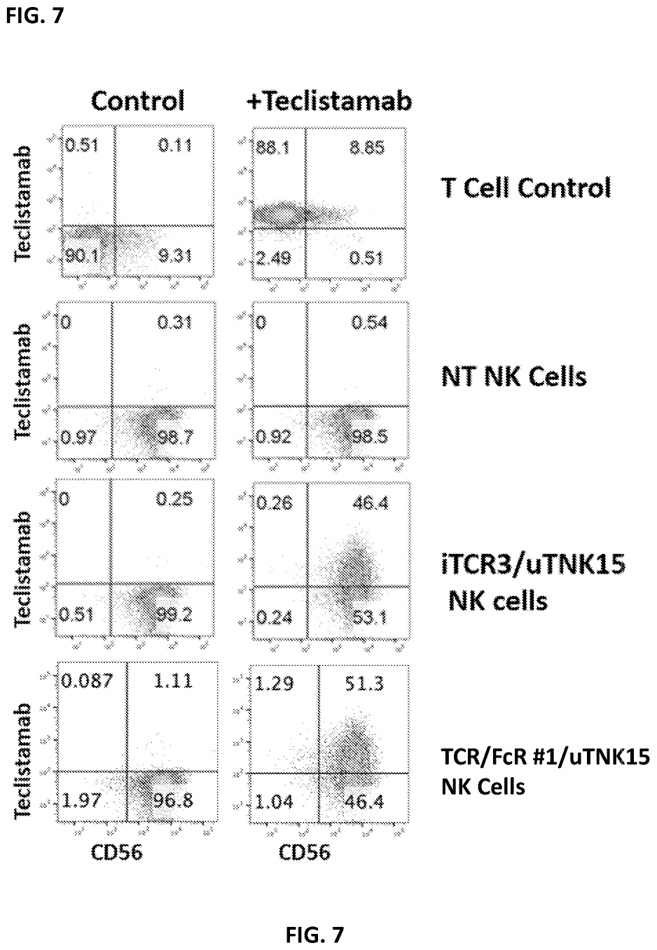

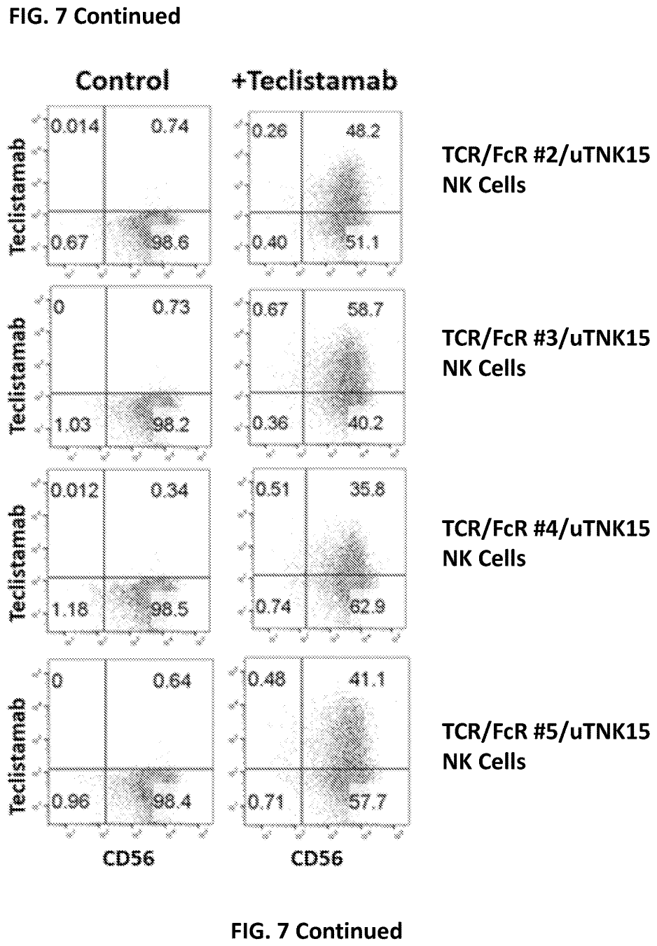

As shown in FIG. 7, TCR/FcR transduced NK cells and T cells were effectively loaded with the Bispecific T cell Engager (BiTE) Teclistamab, while non-transduced NK cells were unable to be loaded with Teclistamab. NK cells were isolated from cord blood and expanded in complete media in the presence of irradiated (100 Gy) uAPC feeder cells (2:1 feeder cell:NK ratio) and recombinant human IL-2 (200 U/ml). Seven days following expansion, NK cells were transduced with uTNK15, and iTCR3 or one of TCR/FcR constructs #1-#5 constructs, or left non-transduced (NT). T cells were used as a positive control and loaded with Teclistamab (200 μg/ml final concentration) for one hour at room temperature in PBS and then washed prior to validation of Teclistamab binding. Flow cytometric analysis against anti-human IgG stained cells showed Teclistamab was bound to NK cells transduced with uTNK15, and iTCR3 or TCR/FcR #1-#5, and to T cells, but not NT NK cells.

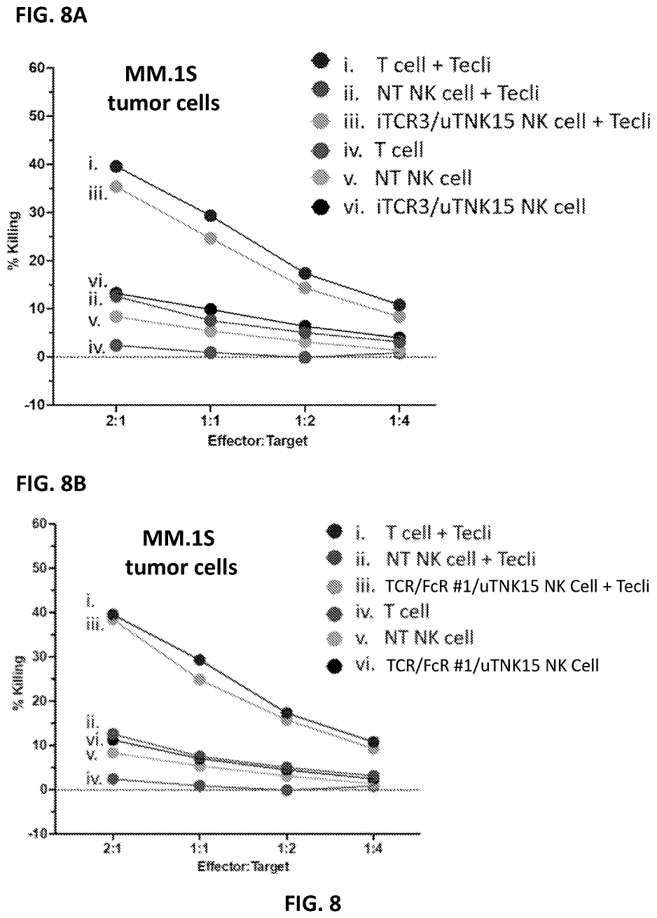

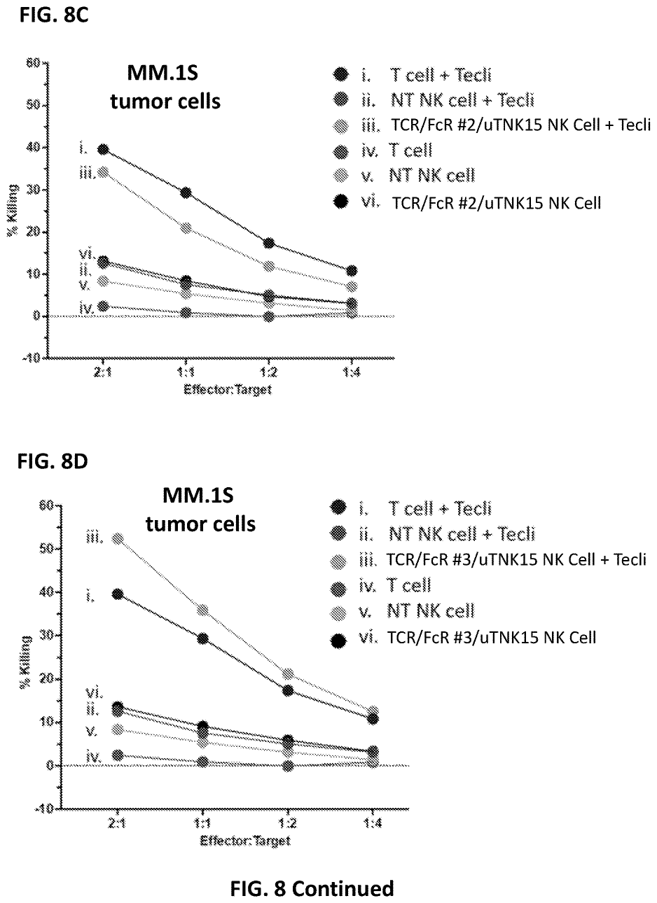

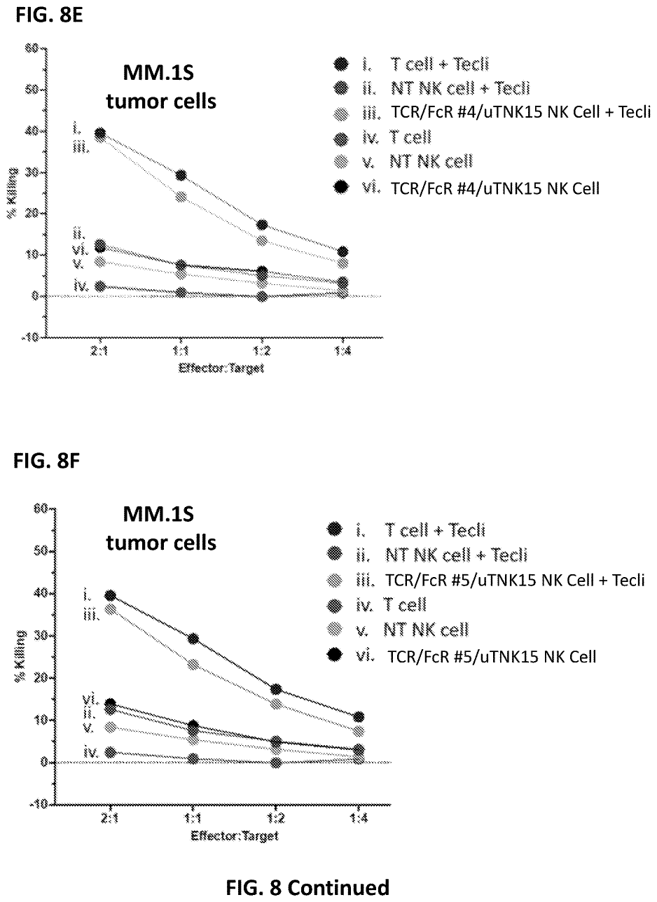

As shown in FIGS. 8A-8F, TCR/FcR transduced NK cells loaded with Teclistamab (Tecli) displayed enhanced killing of BCMA+(MM.1S, myeloma) tumor cells. Cord blood derived NK cells were transduced with uTNK15, and iTCR3 (FIG. 8A) or one of TCR/FcR constructs #1-#5 (FIGS. 8B-8F). Non-transduced (NT) cord blood derived NK cells (NT NK cell) served as negative controls, while non-transduced T cells were used as positive controls. Cells were or were not loaded with Teclistamab (200 μg/ml final concentration) for one hour at room temperature in PBS, cells were then washed prior to co-culturing with MM.1S cells. The MM.1S cells were labelled with chromium-51 and co-cultured with the immune cells at various effector to target (E:T) ratios (X axis). Cells were co-cultured for four hours and chromium release (corresponding to NK cell cytotoxicity against the cancer cells) was measured (y-axis). Compared to non-loaded TCR/FcR-NK cells, or loaded non-transduced (NT) NK Cells, Teclistamab-loaded TCR/FcR transduced NK cells showed increased cytotoxicity against BCMA+MM.1S cells.

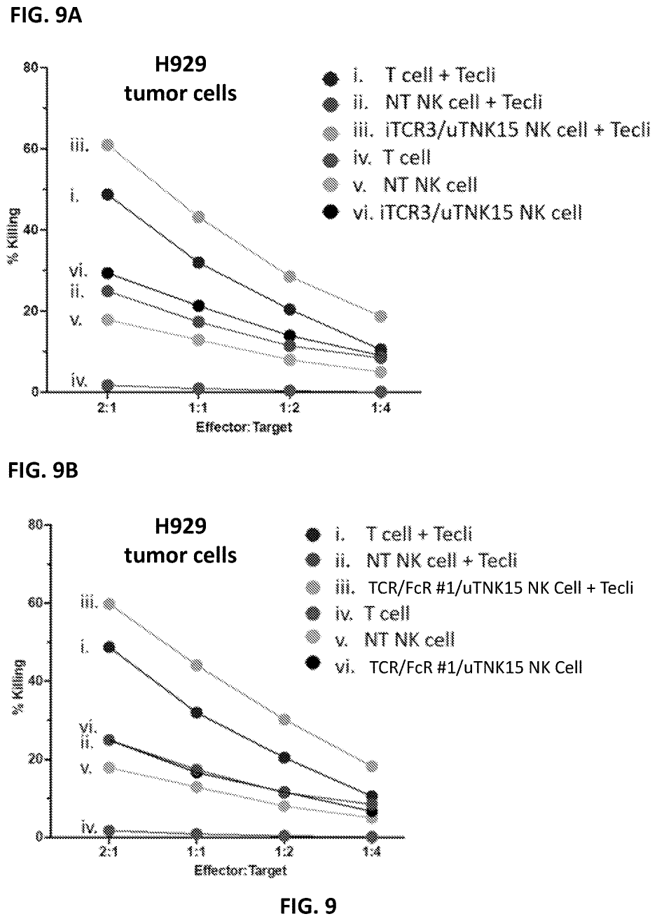

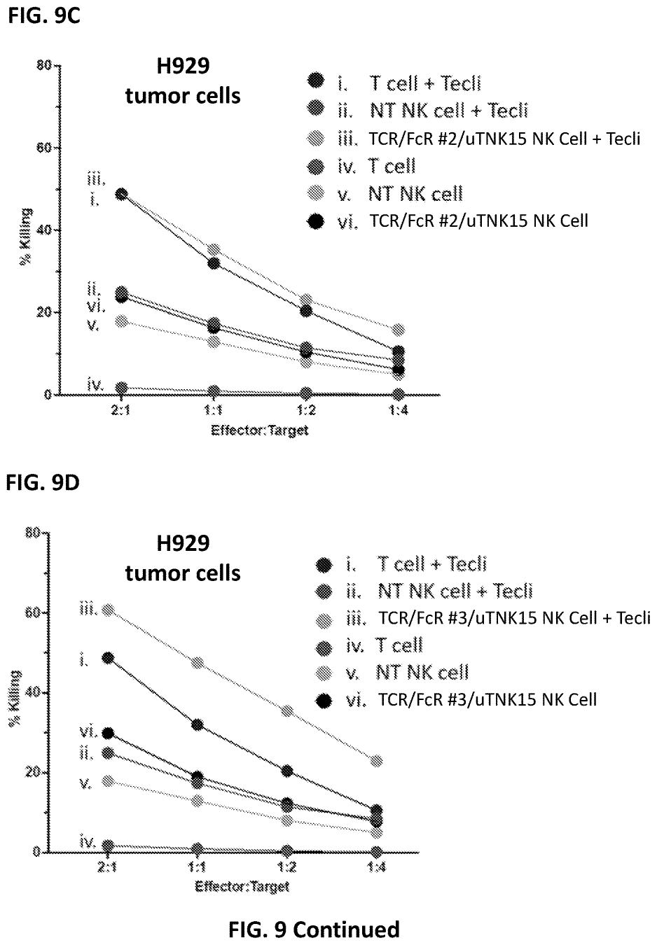

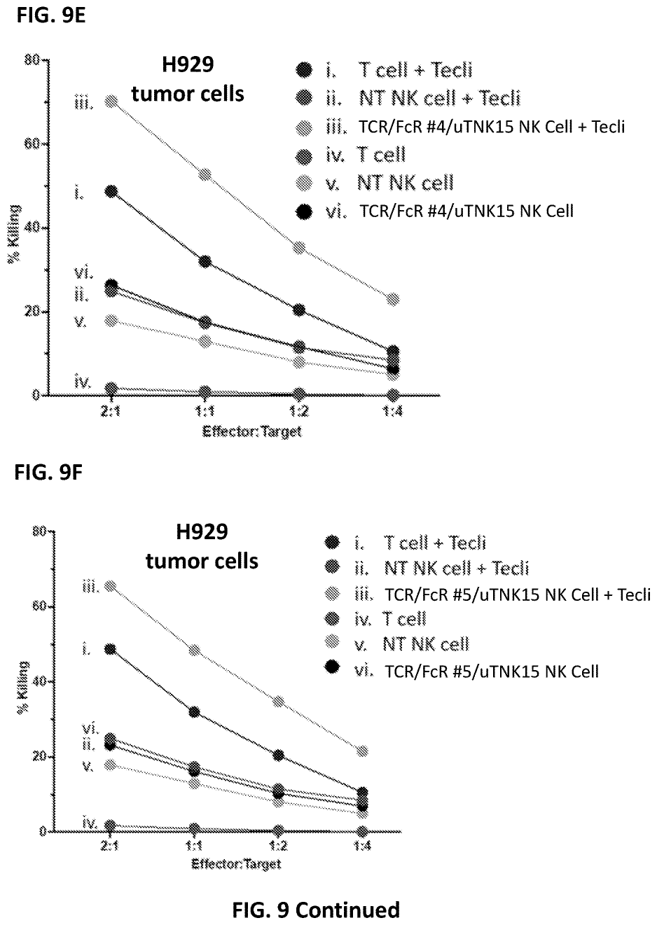

As shown in FIGS. 9A-9F, TCR/FcR transduced NK cells loaded with Teclistamab (Tecli) displayed enhanced killing of BCMA+(H929, myeloma) tumor cells. Cord blood derived NK cells were transduced with uTNK15, and iTCR3 (FIG. 9A) or one of TCR/FcR constructs #1-#5 (FIGS. 9B-9F). Non-transduced (NT) cord blood derived NK cells (NT NK cell) served as negative controls, while non-transduced T cells were used as positive controls. Cells were or were not loaded with Teclistamab (200 μg/ml final concentration) for one hour at room temperature in PBS, cells were then washed prior to co-culturing with MM.1S cells. The H929 cells were labelled with chromium-51 and co-cultured with the immune cells at various effector to target (E:T) ratios (X axis). Cells were co-cultured for four hours and chromium release (corresponding to NK cell cytotoxicity against the cancer cells) was measured (y-axis). Compared to non-loaded TCR/FcR-NK cells, or loaded non-transduced (NT) NK Cells, Teclistamab-loaded TCR/FcR transduced NK cells showed increased cytotoxicity against BCMA+H929 cells.

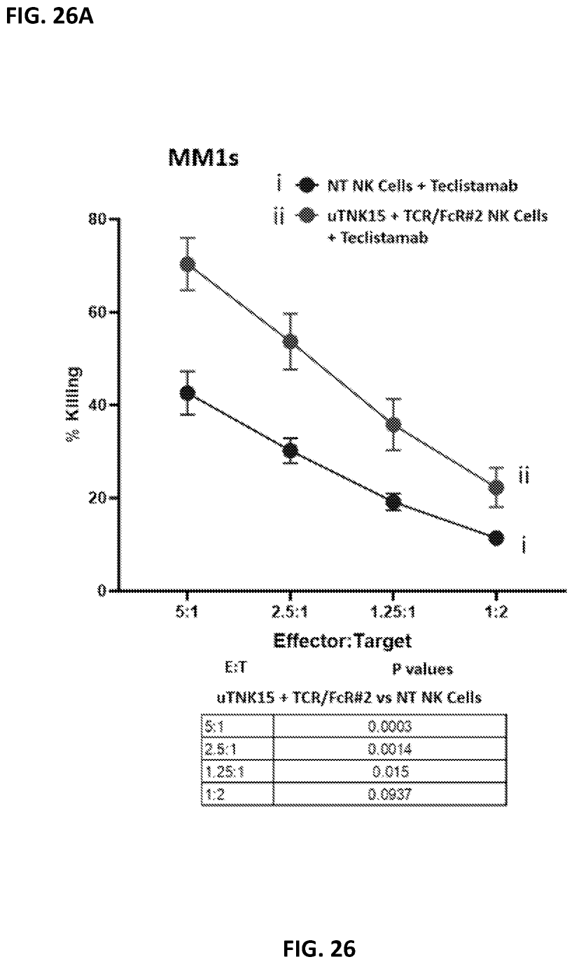

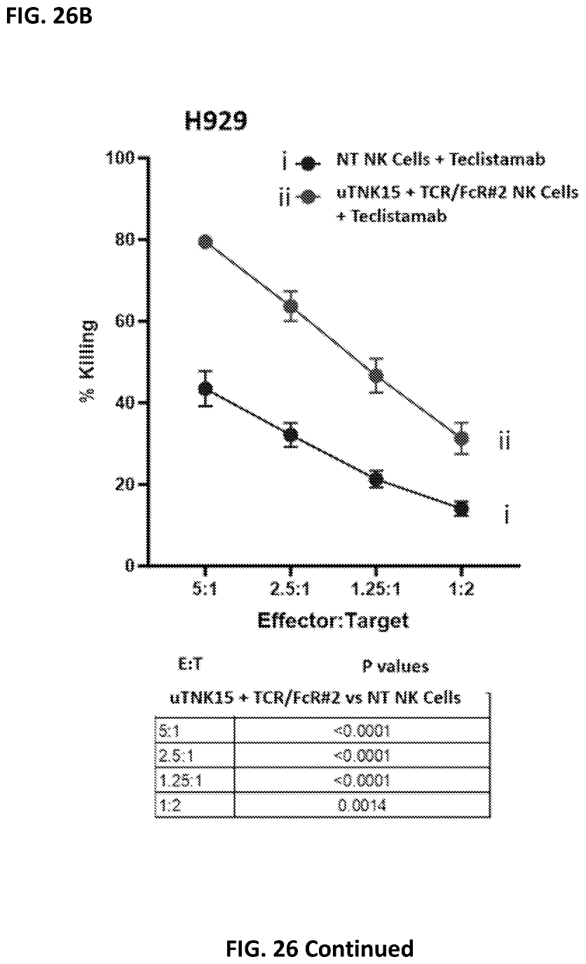

As shown in FIG. 23, TCR/FcR #2 transduced NK cells were successfully loaded with the bispecific antibodies Blinatumomab, Teclistamab, or Elranatamab, while non-transduced (NT) NK cells were not bound by the antibodies. As shown in FIGS. 26A-26B, uTNK15 and TCR/FcR #2 transduced NK cells loaded with bispecific anti-BCMA/CD3 antibody Teclistamab (20 g/ml for 1 hour at 37° C. in click/RPMI media & washed prior to co-culture) demonstrated enhanced antitumor activity against BCMA+ tumor cells (e.g., MM1s cells, H929 cells) at various E:T ratios in short term Cr51 assays when compared to Teclistamab loaded non-transduced (NT) NK cells. FIG. 26A shows the results against MM1s cells, where there was significantly more killing by engineered NK cells at 5:1, 2.5:1, and 1.25:1 E:T ratios. FIG. 26B shows the results against H929 cells, where there was significantly more killing by engineered NK cells at 5:1, 2.5:1, 1.25:1, and 1:2 E:T ratios. Compared to Teclistamab-loaded NT NK cells, Teclistamab-loaded engineered NK cells showed enhanced cytotoxicity against BCMA+tumor cells.

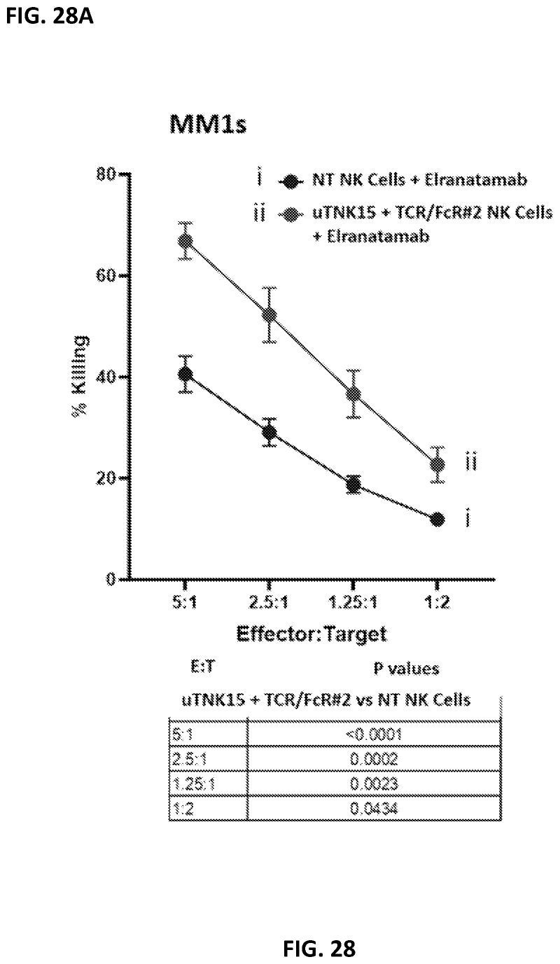

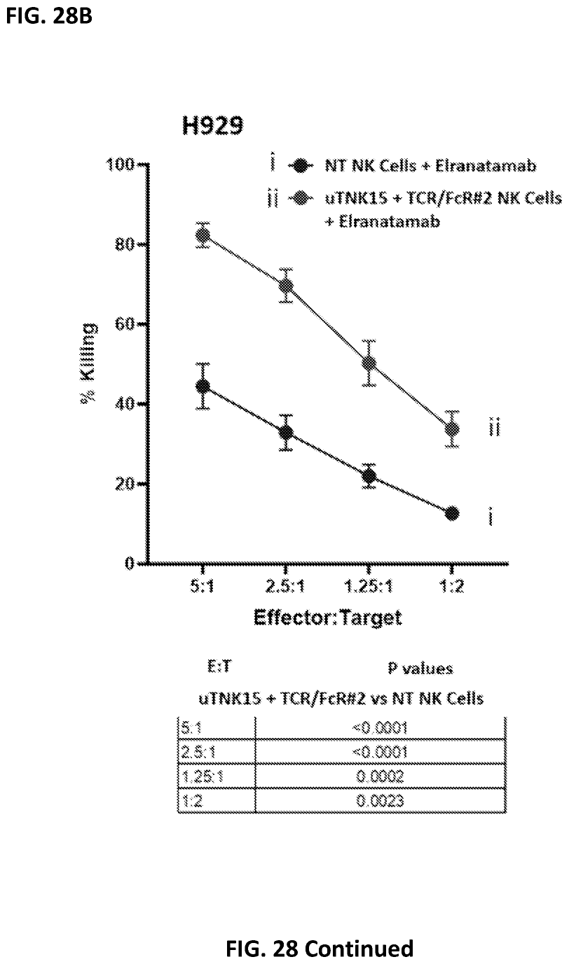

As shown in FIGS. 28A-28B, uTNK15 and TCR/FcR #2 transduced NK cells loaded with bispecific anti-BCMA/CD3 antibody Elranatamab (10 g/ml for 1 hour at 37° C. in click/RPMI media & washed prior to co-culture) demonstrated enhanced antitumor activity against BCMA+tumor cells (e.g., MM1s cells, H929 cells) at various E:T ratios in short term Cr51 assays when compared to Elranatamab loaded non-transduced (NT) NK cells. FIG. 28A shows the results against MM1s cells, where there was significantly more killing by engineered NK cells at 5:1, 2.5:1, 1.25:1, and 1:2 E:T ratios. FIG. 28B shows the results against H929 cells, where there was significantly more killing by engineered NK cells at 5:1, 2.5:1, 1.25:1, and 1:2 E:T ratios. Compared to Elranatamab-loaded NT NK cells, Elranatamab-loaded engineered NK cells showed enhanced cytotoxicity against BCMA+tumor cells.

### Example 5—Tcr/Fcr Nk Cells can Bind Low Fcyr Affinity Fc Domains and Displayed Enhanced Cytotoxicity

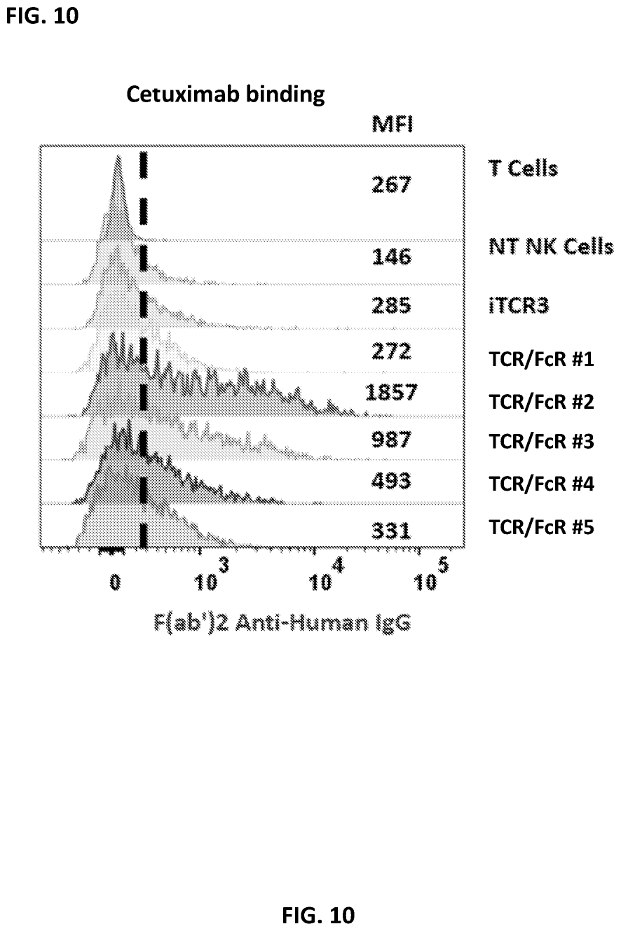

As shown in FIG. 10, TCR/FcR transduced NK cells can be loaded with low Fcγ Receptor affinity antibodies (e.g., Cetuximab). NK cells were derived from cord blood and expanded with irradiated (100 Gy) UAPC feeder cells (2:1 feeder cell:NK ratio) and recombinant human IL-2 (200 U/ml) in complete NK cell growth medium (Click's/RPMI). NK cells were loaded with low Fcγ Receptor affinity antibody (e.g., Cetuximab at 100 μg/ml final concentration) for one hour at 37° C. in Click's/RPMI media followed by washing before validating the binding of the low Fcγ Receptor affinity antibody with flow cytometry. NK cells were stained with Alexa-Fluor647 affinity-purified F(ab′)2 fragment goat anti-human IgG (H+L) antibody and analyzed by flow cytometry. The results showed that antibodies with low Fcγ receptor affinity Fc domains (e.g., Cetuximab) bound to high affinity CD16 (CD16 ha, (F158V)) TCR/FcR transduced NK cells at greater levels when compared to ITCR3 transduced NK cells, NT NK cells, or T cells.

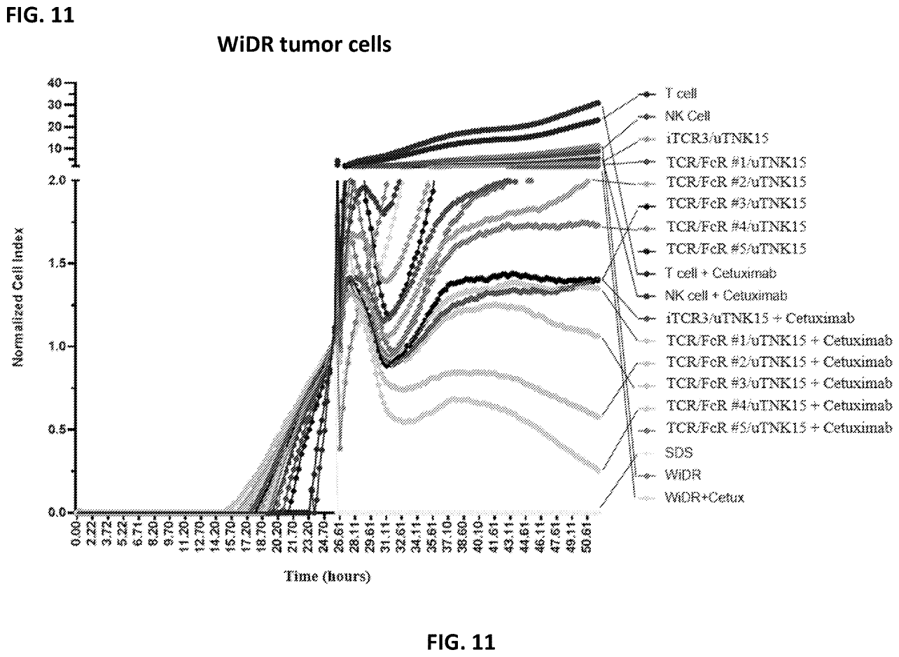

As shown in FIG. 11, TCR/FcR transduced NK cells loaded with Cetuximab displayed enhanced killing of WiDR (colorectal cancer cells; CRC) compared to non-loaded TCR/FcR NK cells or loaded/non-loaded non-transduced (NT) NK Cells. NK cells were transduced with uTNK15, and iTCR3 or one of TCR/FcR constructs #1-#5. WiDR cells alone, Non-transduced (NT) cord blood derived NK cells (NT NK cell), and T cells served as served as controls. Cells were or were not loaded with Cetuximab (100 μg/ml final concentration) for one hour at 37° C. in Click's/RPMI media and then washed before co-culturing with tumor cells at 1:1 E:T ratio. Compared to Cetuximab alone, or to loaded/non-loaded NT NK cells or to non-loaded TCR/FcR NK cells, Cetuximab loaded TCR/FcR NK cells showed the highest cytotoxic activities against the EGFR+ CRC (WiDR) cell line. The lower the normalized cell index (Y axis), the higher the degree of cytotoxicity. SDS was used a positive control for cytotoxicity. NK cells transduced with TCR/FcR #4 or TCR/FcR #2 and loaded with cetuximab exerted the greatest levels of cytotoxicity against WiDR cells.

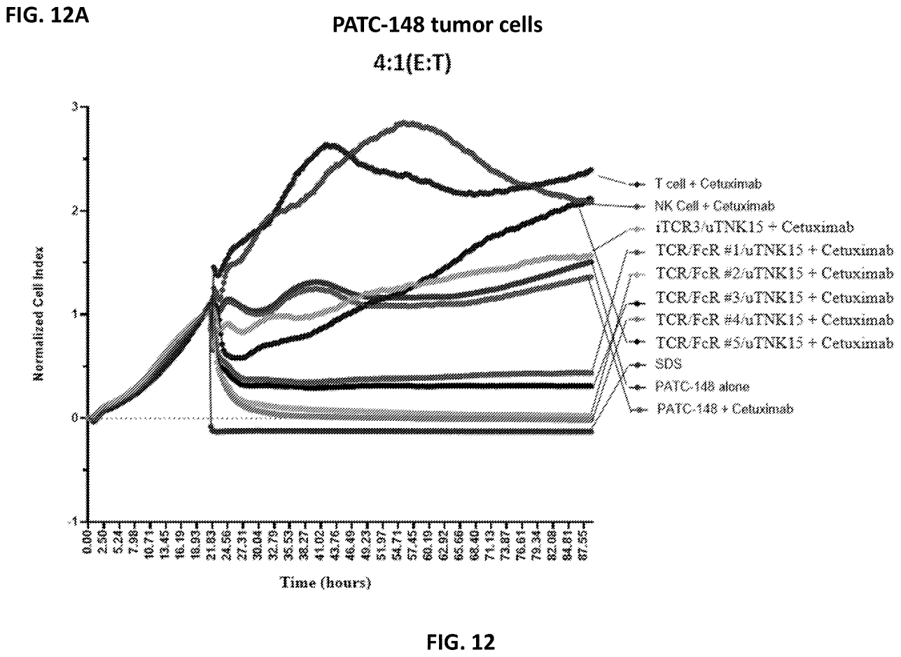

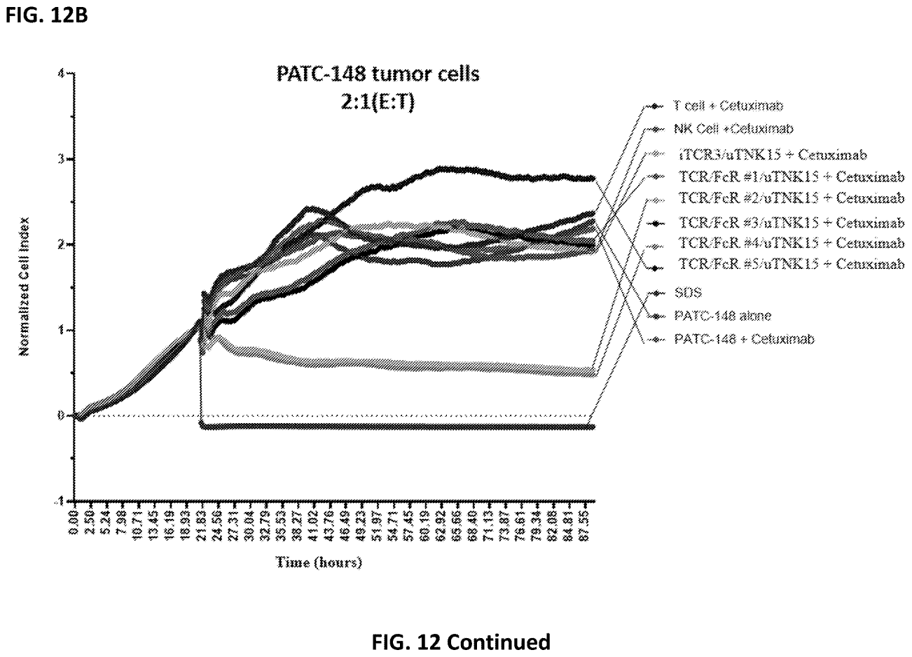

As shown in FIGS. 12A-12B, TCR/FcR transduced NK cells loaded with Cetuximab displayed enhanced killing of PATC148 cells (pancreatic ductal adenocarcinoma, PDAC) compared to non-loaded TCR/FcR NK cells or loaded/non-loaded non-transduced (NT) NK Cells. NK cells were transduced with uTNK15, and iTCR3 or one of TCR/FcR constructs #1-#5. PATC148 cells alone, Non-transduced (NT) cord blood derived NK cells (NT NK cell), and T cells served as controls. Cells were or were not loaded with Cetuximab (100 μg/ml final concentration) for one hour at 37° C. in Click's/RPMI media and then washed before co-culturing with tumor cells at 4:1 E:T ratio (FIG. 12A), or at 2:1 ET ratio (FIG. 12B). Compared to Cetuximab alone, or with loaded/non-loaded non-transduced (NT) NK cells or with non-loaded TCR/FcR NK cells, Cetuximab loaded TCR/FcR transduced NK cells showed the greatest cytotoxic activity against EGFR+PATC148 (PDAC) cell lines. The lower the normalized cell index (Y axis), the higher the degree of cytotoxicity. SDS was used a positive control for cytotoxicity. NK cells transduced with TCR/FcR #4 or TCR/FcR #2 and loaded with cetuximab exerted the greatest levels of cytotoxicity against PATC148 (PDAC) cells.

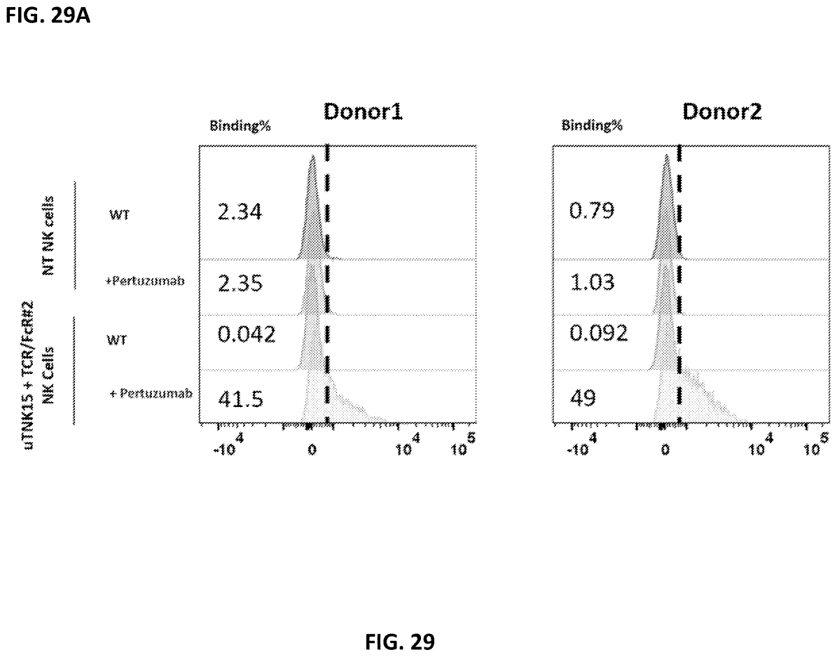

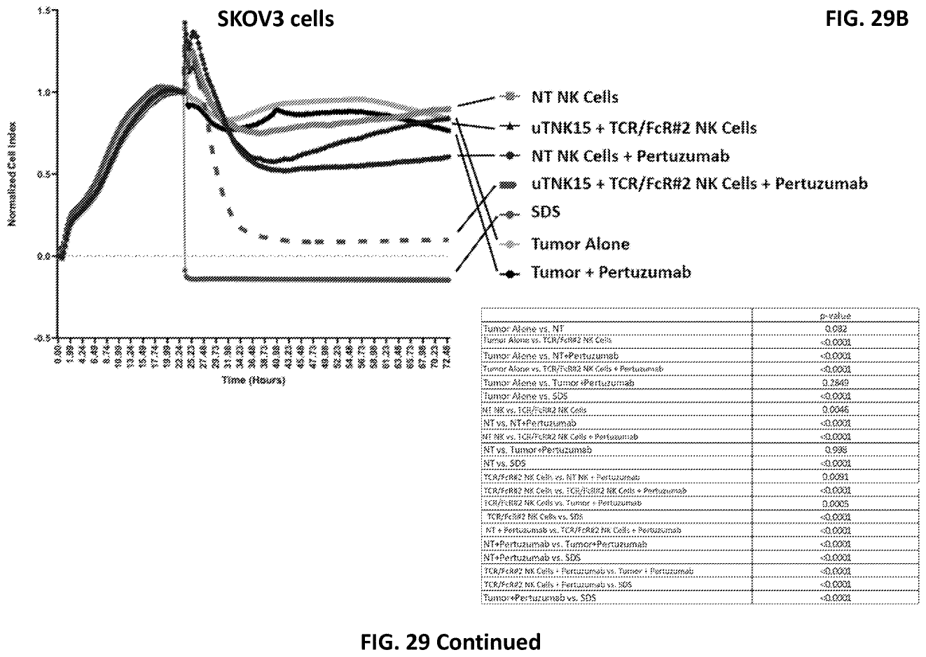

As shown in FIG. 29A-29B, Pertuzumab (anti-HER2 Ab with low affinity binding to wild type CD16) bound to uTNK15 and TCR/FcR #2 transduced NK cells, but did not bind to non-transduced (NT) NK cells, and Pertuzumab loaded engineered NK cells displayed superior antitumor activity against HER2+SKOV3 ovarian cancer cells in a long term xCELLigence killing assay. FIG. 29A, shows Pertuzumab binding to engineered NK cells. NK cells were extracted from cord blood and cultured in complete medium supplemented with irradiated (100 Gy) uAPC feeder cells at a ratio of 2:1 (feeder cell:NK). Additionally, recombinant human TL-2 was added at a concentration of 200 U/ml to facilitate expansion of the NK cells. Five days after expansion, natural killer (NK) cells were either transduced with the uTNK15 and TCR/FcR #2 constructs, or left non-transduced (NT). These cells were then loaded with Pertuzumab (200 g/ml for one hour at 37° C. in a complete medium). After incubation, the cells were washed and subjected to flow cytometric analysis to validate the binding. Flow cytometric analysis revealed that Pertuzumab could bind engineered NK cells, but no binding of Pertuzumab was observed with NT NK cells. FIG. 29B, shows Pertuzumab loaded uTNK15 and TCR/FcR #2 transduced NK cells had enhanced killing of HER2+ tumor cells when compared to unloaded uTNK15 and TCR/FcR #2 transduced NK cells or loaded/unloaded non-transduced (NT) NK Cells. NK cells were either NT (NT or NT NK shorthand for stats) or transduced with uTNK15 and TCR/FcR #2 (“TCR/FcR #2” shorthand for stats). NK cells were either left unloaded or loaded with Pertuzumab (200 g/ml for 1 hour at 37° C. in click/RPMI media & washed prior to co-culture) before co-culturing with tumor cells at 1:1 effector to target (E:T) ratio. Compared to loaded or unloaded NT NK Cells, unloaded engineered NK cells, or Pertuzumab alone, Pertuzumab-loaded engineered NK cells showed significantly increased cytotoxic activity against HER2+SKOV3 ovarian cancer cells.

### Example 6—Tcr/Fcr Nk Cells Targeting Egfr Displayed Enhanced Cytotoxicity

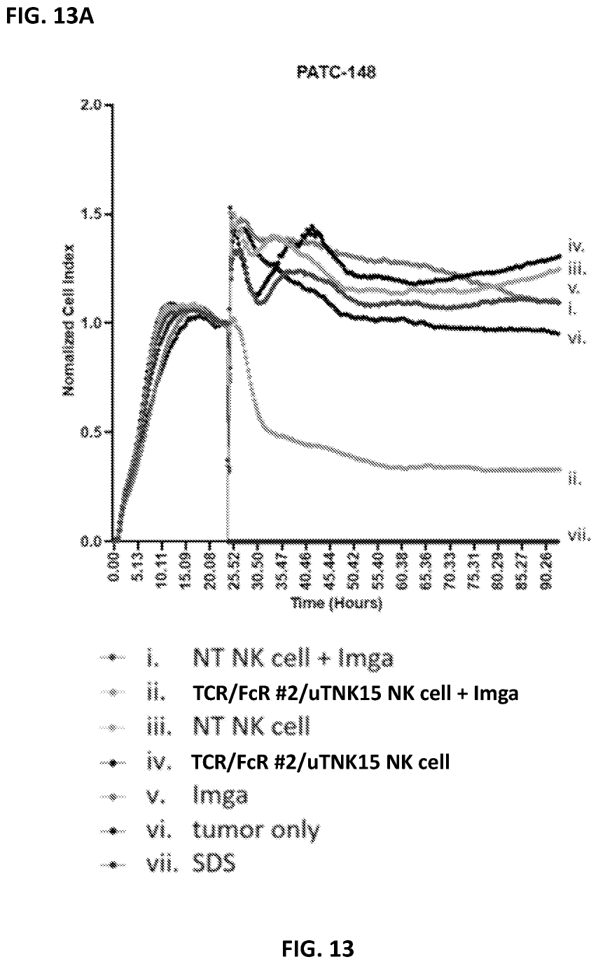

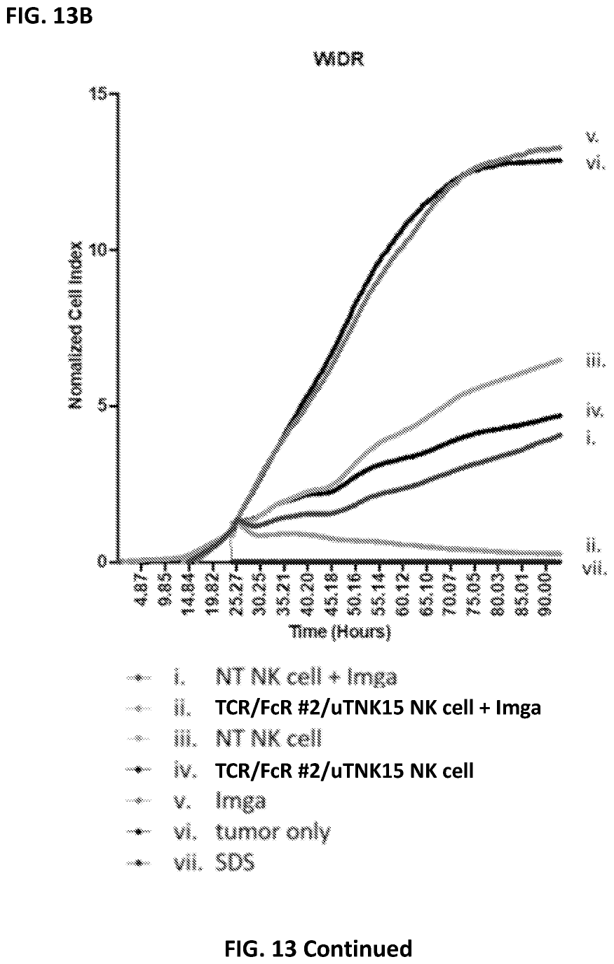

As shown in FIGS. 13A-13B, TCR/FcR transduced NK cells loaded with Imgatuzumab displayed enhanced killing of PDAC or CRC cells when compared to non-loaded TCR/FcR NK cells or loaded/un-loaded non-transduced (NT) NK Cells. NT NK cells or NK cells transduced with uTNK15 and TCR/FcR construct #2 were loaded with Imgatuzumab (10 μg/ml final concentration) for one hour at room temperature in PBS and then washed before co-culture with EGFR+PDAC (PATC-148, FIG. 13A) or EGFR+colorectal cancer (WiDR, FIG. 13B) tumor cells at a 1:1 E:T ratio. Compared to Imgatuzumab alone, loaded/un-loaded NT-NK cells, or non-loaded TCR/FcR NK cells, Imgatuzumab loaded TCR/FcR transduced NK cells showed increased cytotoxic activity against EGFR+PDAC (PATC 148) and colorectal (WiDR) cell lines.

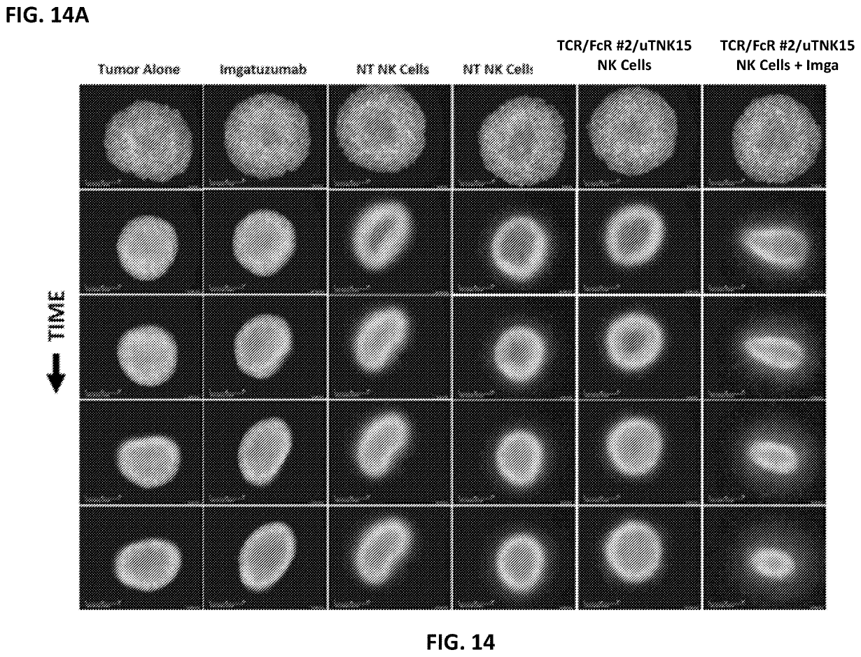

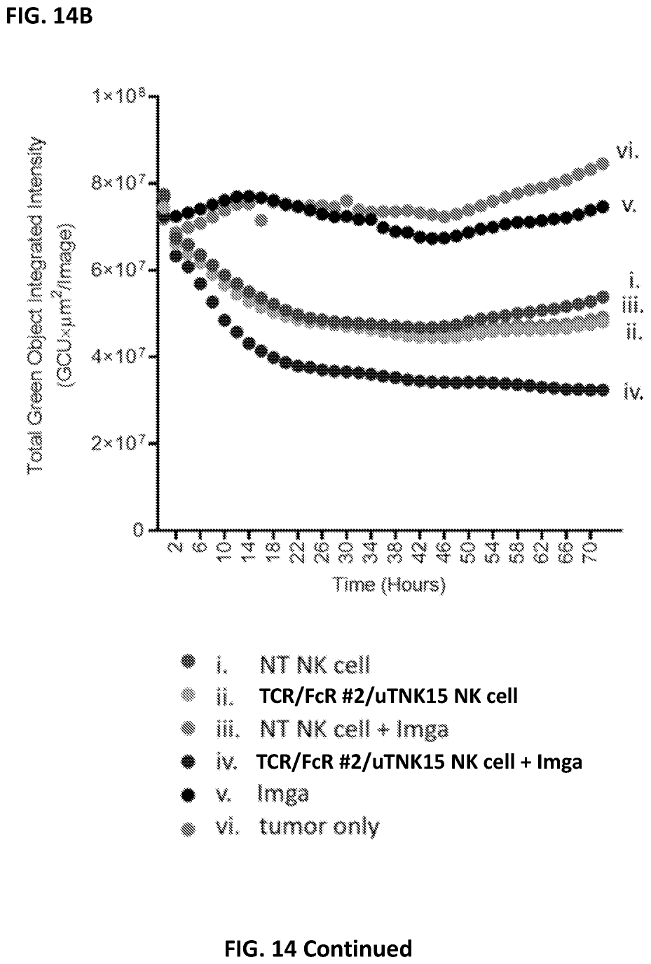

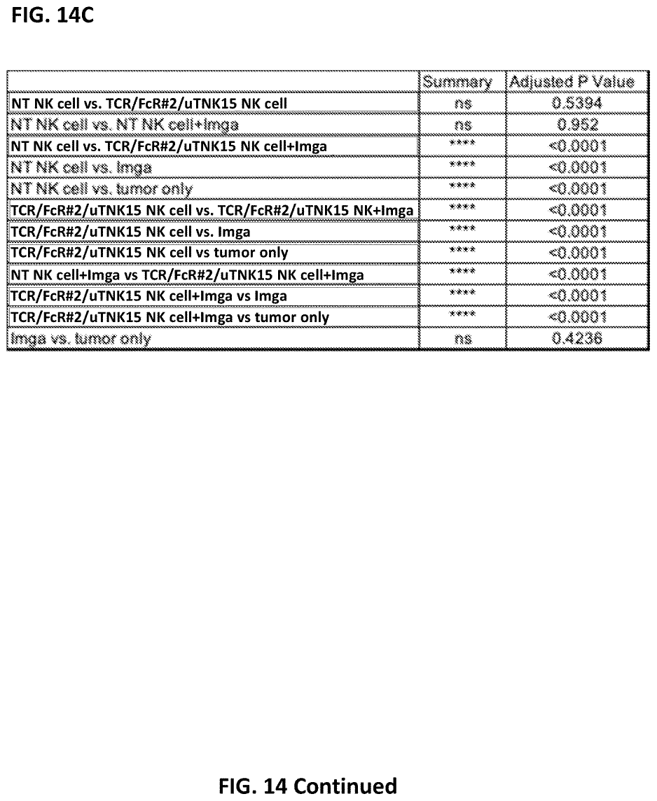

As shown in FIGS. 14A-14B, TCR/FcR transduced NK cells loaded with Imgatuzumab displayed enhanced killing of 3D PDAC (PATC-148) tumor spheroids. 14A) displays representative images of PATC148 spheroids (PDAC tumor cell line transduced with GFP) either left alone, treated with NT NK Cells, or treated with NK cells transduced with uTNK15 and TCR/FcR construct #2, the NK cells were either non-loaded or loaded with Imgatuzumab (10 μg/ml final concentration) for one hour at room temperature in PBS and then washed before co-culture. The data showed significant decreases in spheroid size in the wells treated with Imgatuzumab loaded TCR/FcR transduced NK cells. 14B) depicts quantification of the total integrated green intensity over time observed in 14A, the data demonstrated a significant decrease in total integrated green intensity when spheroids were treated with TCR/FcR transduced NK cells loaded with Imgatuzumab.

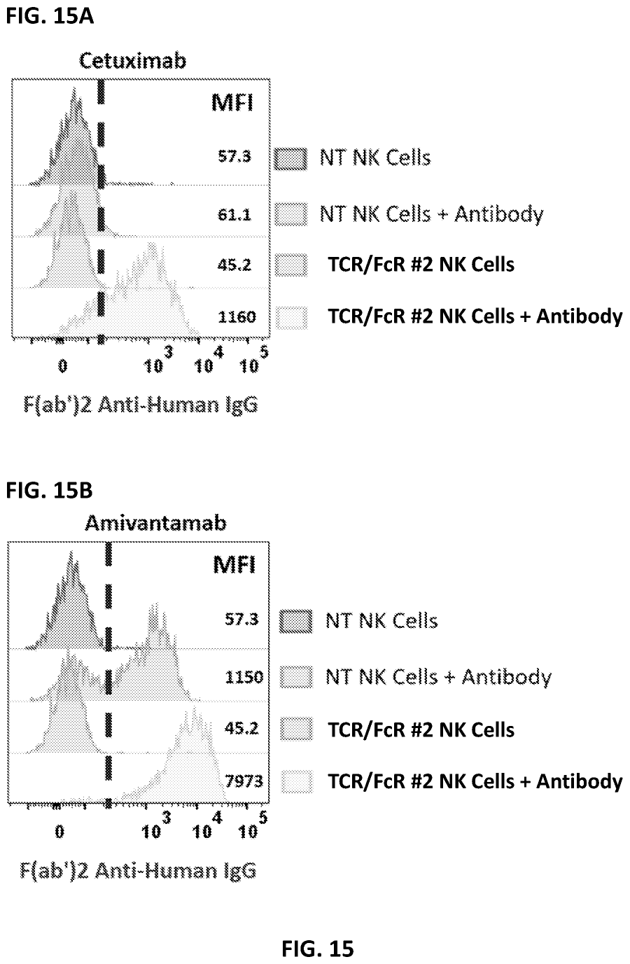

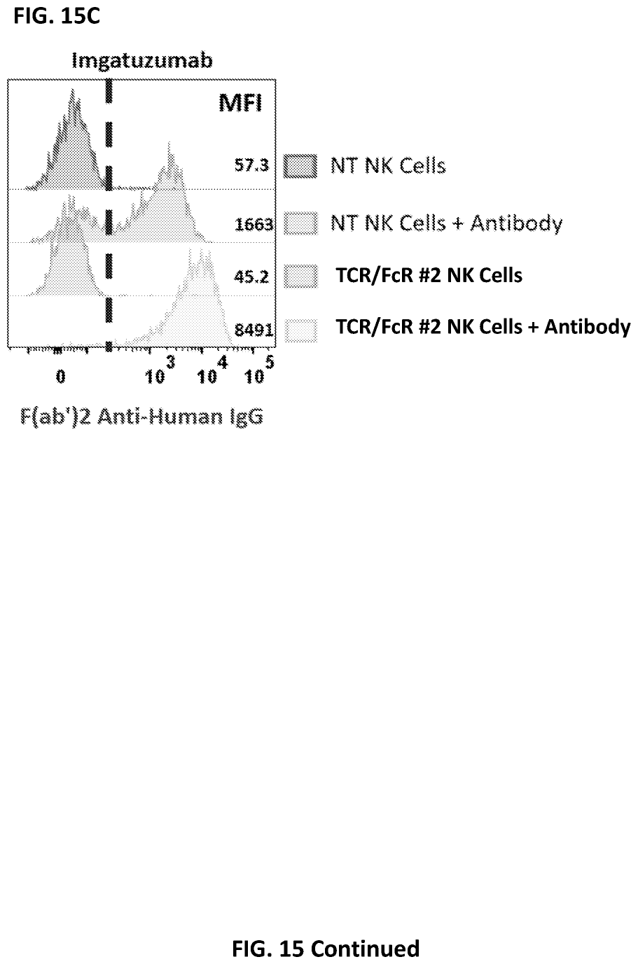

As shown in FIGS. 15A-15B, antibodies Cetuximab, Amivantamab, or Imgatuzumab can bind to TCR/FcR #2 transduced NK cells. NK cells were derived from cord blood and were expanded with irradiated (100 Gy) UAPC feeder cells (2:1 feeder cell:NK ratio) and recombinant human IL-2 (200 U/ml) in complete NK cell growth medium (Click's/RPMI). Non-transduced NK cells or NK cells transduced with TCR/FcR #2 were loaded with Cetuximab (250 μg/ml final concentration) (FIG. 15A), Amivantamab (100 μg/ml final concentration) (FIG. 15B), or Imgatuzumab (100 μg/ml final concentration) (FIG. 15C), for one hour at 37° C. in Click's/RPMI media before the cells were washed. NK cells were stained with Alexa-Fluor647 affinity-purified F(ab′)2 fragment goat anti-human IgG (H+L) antibody and analyzed by flow cytometry to confirm the binding of Cetuximab, Amivantamab, or Imgatuzumab. The results showed that antibodies were bound to TCR/FcR transduced NK cells with higher affinity when compared to NT NK cells. Of note, unlike Amivantamab or Imgatuzumab, Cetuximab (which comprises an Fc domain with low binding affinity to WT CD16) was found to bind only to the CD16 ha TCR/FcR transduced NK cells.

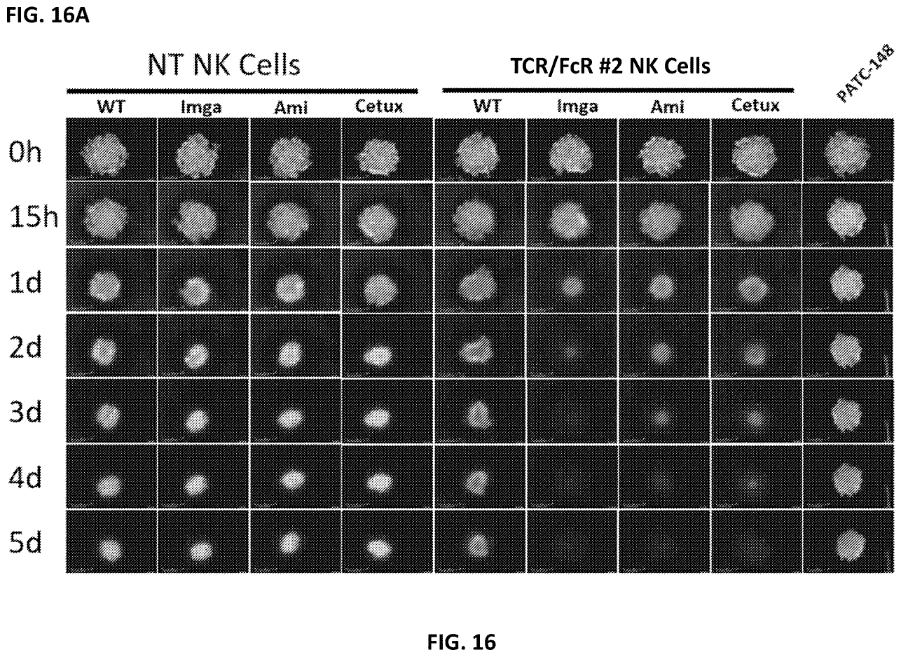

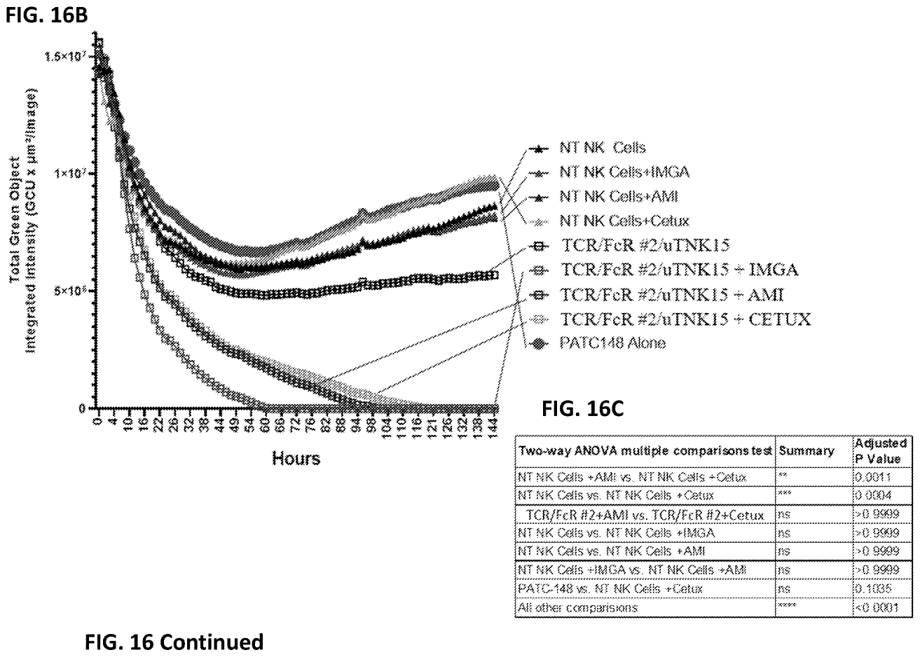

As shown in FIGS. 16A-16C, TCR/FcR #2 transduced NK cells demonstrated enhanced antitumor activity against PATC-148 (PDAC) cell spheroids when loaded with Cetuximab (Cetux), Imgatuzumab (Imga), or Amivantamab (Ami) relative to NT NK cells. FIG. 16A shows representative images of GFP transduced PDAC (PATC-148) spheroids that were either left alone, treated with non-loaded or loaded NT NK, or treated with non-loaded or loaded TCR/FcR #2 transduced NK cells. Loaded cells were prepared with Imgatuzumab (100 μg/ml final concentration), Amivantamab (100 μg/ml final concentration), or Cetuximab (250 μg/ml final concentration). A readily observable and significant decrease in spheroid size was observed in the wells treated with antibody-loaded TCR/FcR transduced NK cells. FIG. 16B is a graphical quantification of the total integrated green intensity over time from experiments as performed in FIG. 16A, the data shows a significant decrease (FIG. 16C) in total integrated green intensity when spheroids were treated with TCR/FcR transduced NK cells loaded with antibodies.

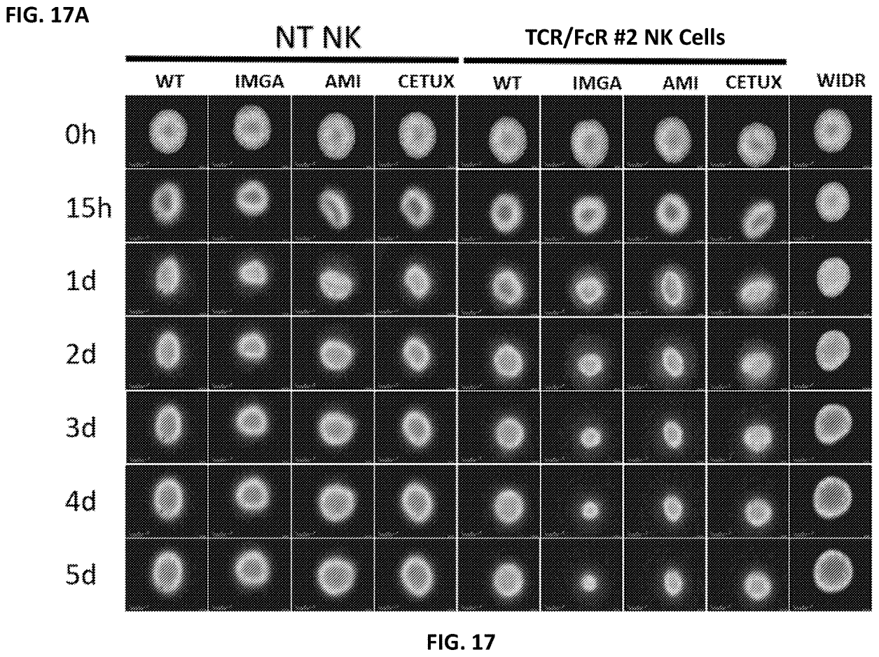

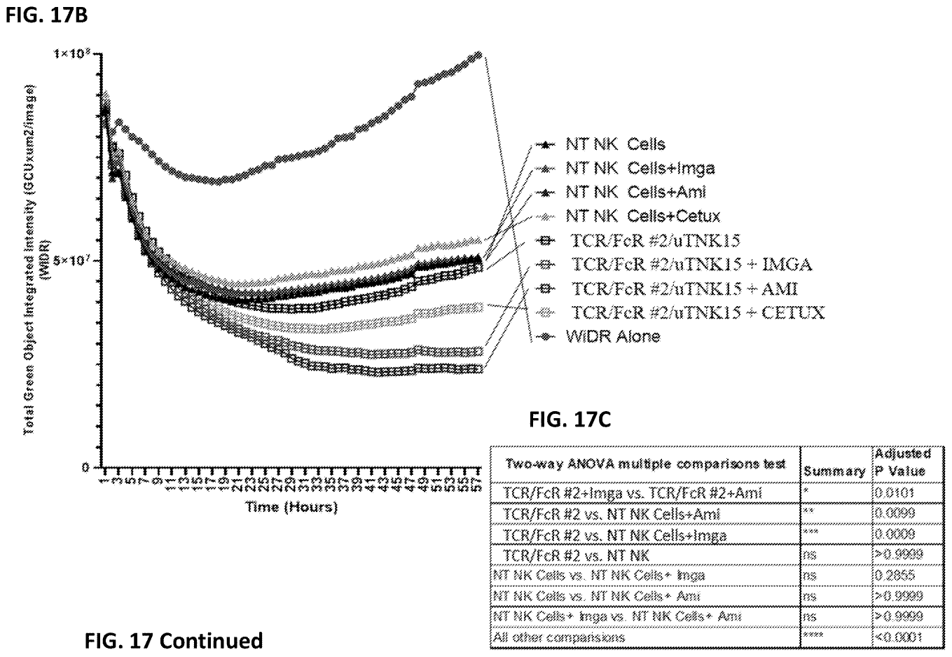

As shown in FIGS. 17A-17C, TCR/FcR #2 transduced NK cells demonstrated enhanced antitumor activity against WiDR (CRC) cell spheroids when loaded with Cetuximab (Cetux), Imgatuzumab (Imga), or Amivantamab (Ami) relative to NT NK cells. Antibody loaded TCR/FcR transduced NK cells showed enhanced killing of CRC cells when compared to non-loaded TCR/FcR NK cells or loaded/non-loaded non-transduced (NT) NK Cells. FIG. 17A shows representative images of GFP transduced WiDR spheroids that were either left alone, treated with non-loaded or loaded NT NK, or treated with non-loaded or loaded TCR/FcR #2 transduced NK cells. Loaded cells were prepared with Imgatuzumab (100 μg/ml final concentration), Amivantamab (100 μg/ml final concentration), or Cetuximab (250 μg/ml final concentration). The data show significant decreases in spheroid size in the wells treated with antibody-loaded TCR/FcR transduced NK cells. FIG. 17B is a graphical quantification of the total integrated green intensity over time from experiments as performed in FIG. 16A, the data shows a significant decrease (FIG. 17C) in total integrated green intensity when spheroids were treated with TCR/FcR transduced NK cells loaded with antibodies.

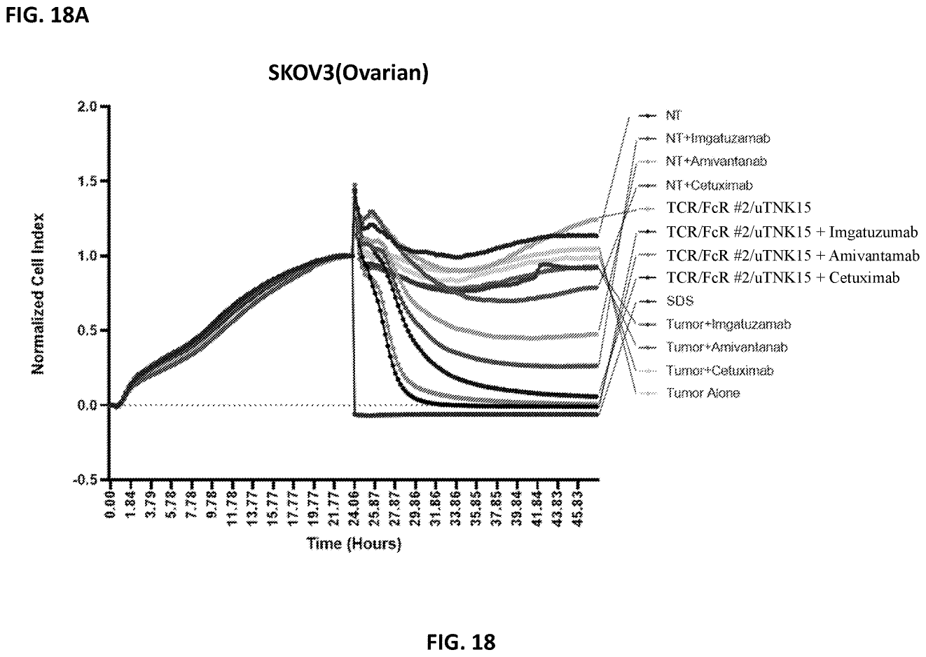

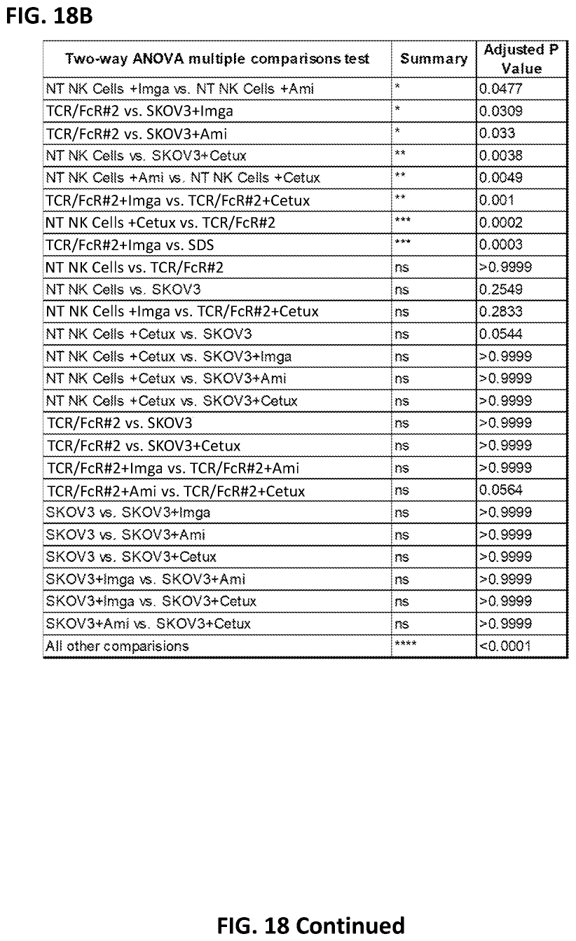

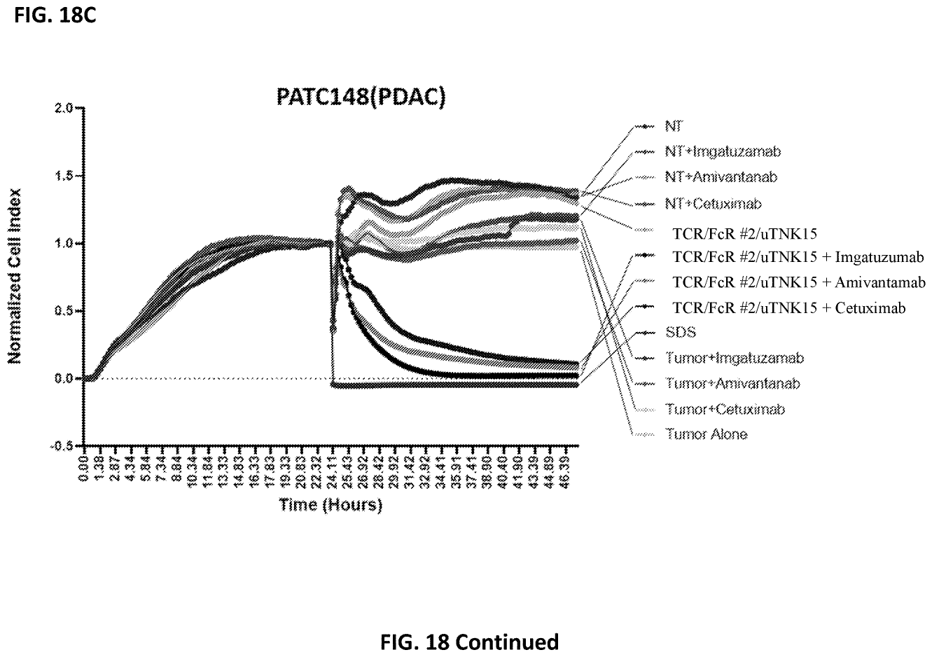

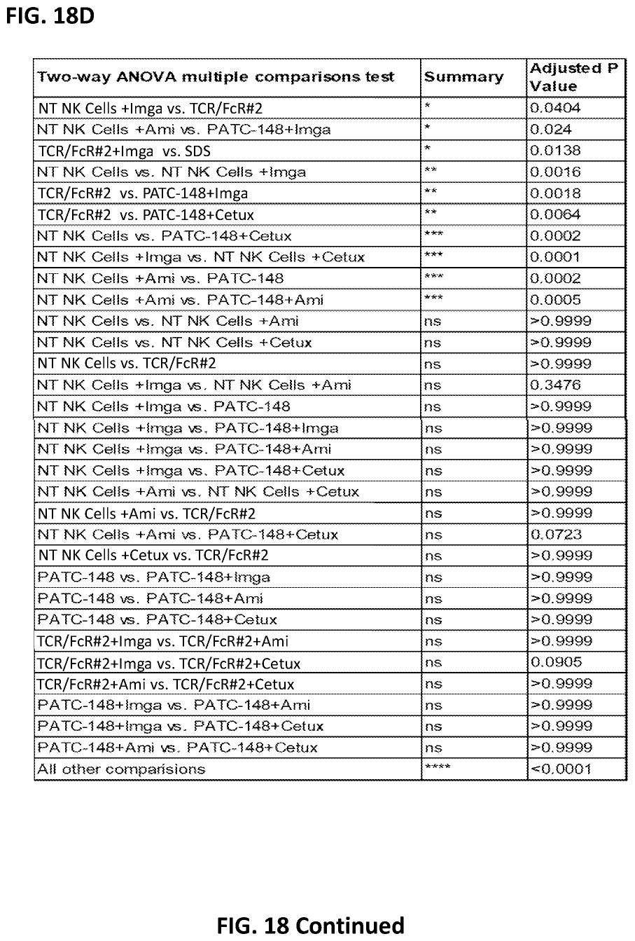

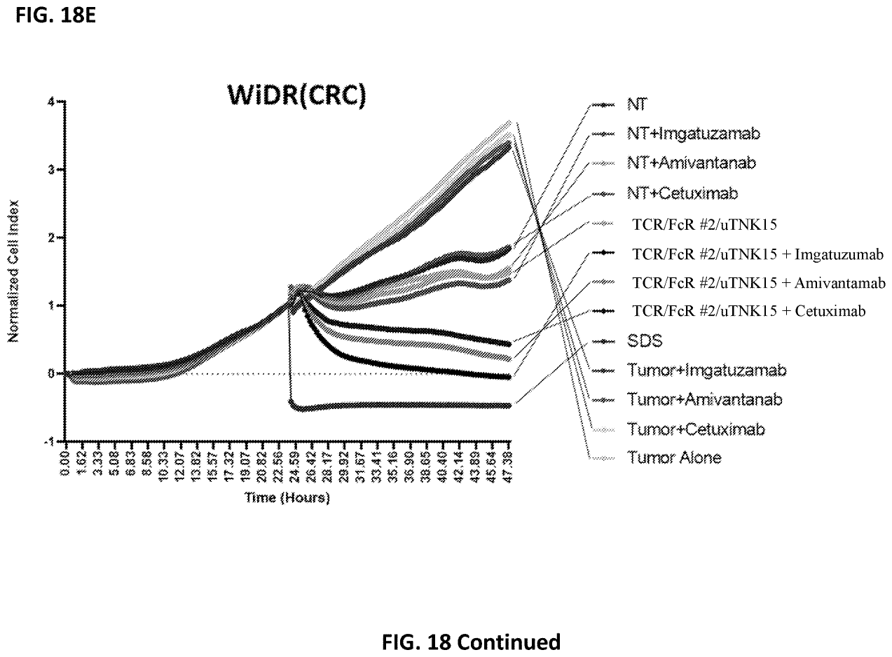

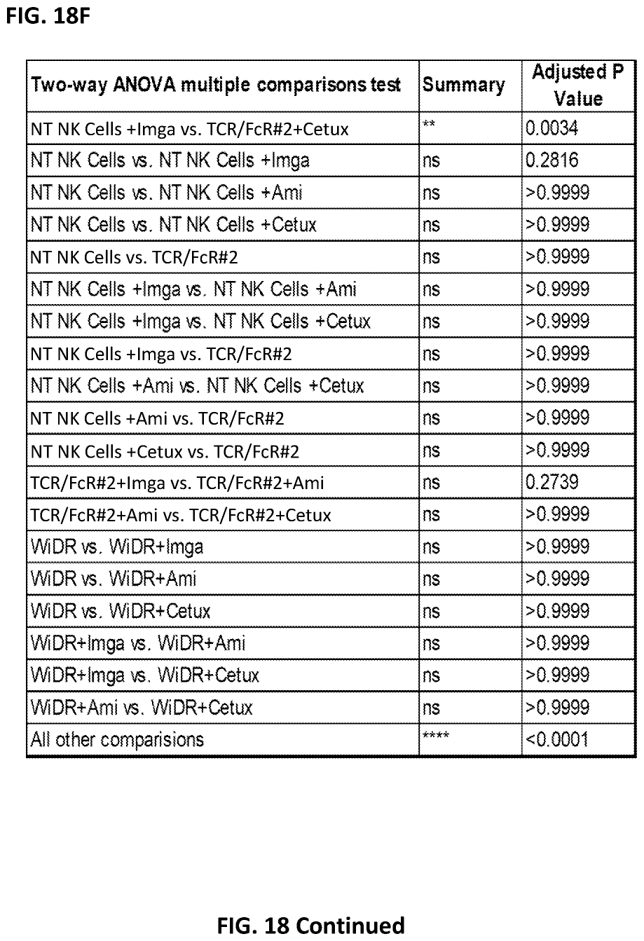

As shown in FIGS. 18A-18D, TCR/FcR #2 transduced NK cells demonstrated enhanced antitumor activity against various solid tumor cell lines when loaded with anti-EGFR antibodies Cetuximab, Imgatuzumab, or Amivantamab. Antibody loaded TCR/FcR transduced NK cells showed enhanced killing when compared to non-loaded TCR/FcR NK cells or loaded/non-loaded non-transduced (NT) NK Cells. NK cells were either non-transduced (NT) or transduced with TCR/FcR #2 and were left unloaded or loaded with Cetuximab (250 μg/ml final concentration), Amivantamab (100 μg/ml final concentration), or Imgatuzumab (100 μg/ml final concentration) for one hour at 37° C. in Click's/RPMI media followed by washing. NK cells were co-cultured with tumor cells SKOV3 (FIG. 18A), PATC-148 (FIG. 18C), or WiDR (FIG. 18E) at 2:1 E:T ratios. Compared to antibodies alone, loaded/non-loaded non-transduced (NT)NK cells, or non-loaded TCR/FcR #2 NK cells, the antibody-loaded TCR/FcR #2 transduced NK cells showed significantly increased cytotoxic activity against EGFR+ SKOV3 (FIG. 18B), PATC-148 (FIG. 18D), or WiDR (FIG. 18G) cell lines.

### Example 7—Tcr/Fcr Nk Cells can Bind Low Fcir Affinity Fc Domains and Displayed Enhanced Cytotoxicity Against Cd19 Expressing Targets

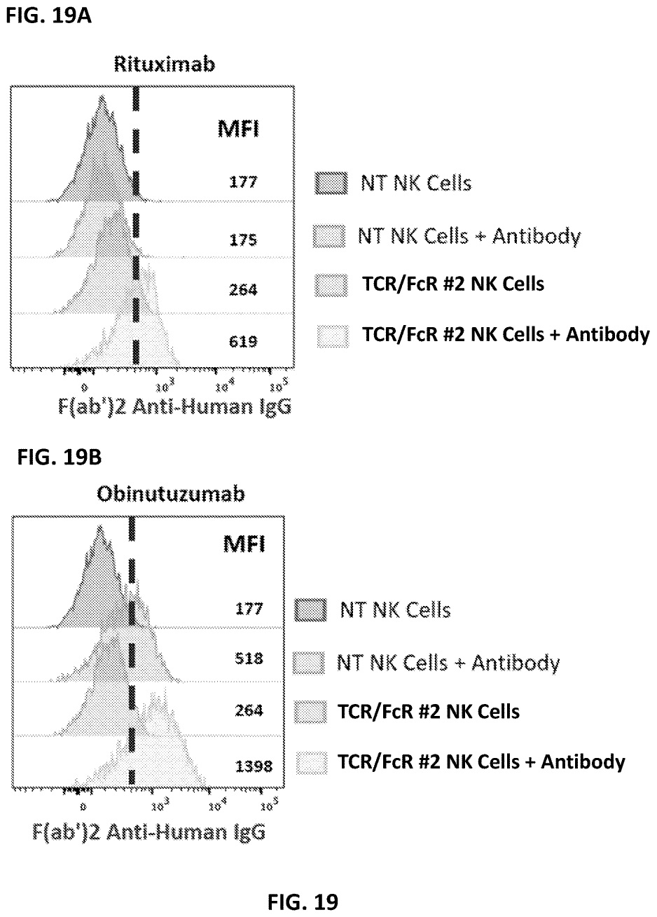

As shown in FIGS. 19A-19B, TCR/FcR #2 transduced NK Cells can be loaded with low Fcγ Receptor affinity antibody (e.g., Rituximab) or high Fcγ receptor affinity antibody (Obinutuzumab). NK cells were derived from cord blood and were expanded with irradiated (100 Gy) UAPC feeder cells (2:1 feeder cell:NK ratio) and recombinant human IL-2 (200 U/ml) in complete NK cell growth medium (Click's/RPMI). Non-transduced NK cells or NK cells transduced with TCR/FcR #2 were loaded with Rituximab (100 μg/ml final concentration) (FIG. 19A), or Obinutuzumab (100 μg/ml final concentration) (FIG. 19B) for one hour at 37° C. in Click's/RPMI media followed by washing. NK cells were stained with Alexa-Fluor647 affinity-purified F(ab′)2 fragment goat anti-human IgG (H+L) antibody and analyzed by flow cytometry. The results showed that low and high Fcγ receptor affinity antibodies were bound to TCR/FcR transduced NK cells at greater rates when compared to NT NK cells. Of note, unlike Obinutuzumab, Rituximab (which comprises an Fc domain with low binding affinity to WT CD16) was found to bind only to the CD16 ha TCR/FcR transduced NK cells.

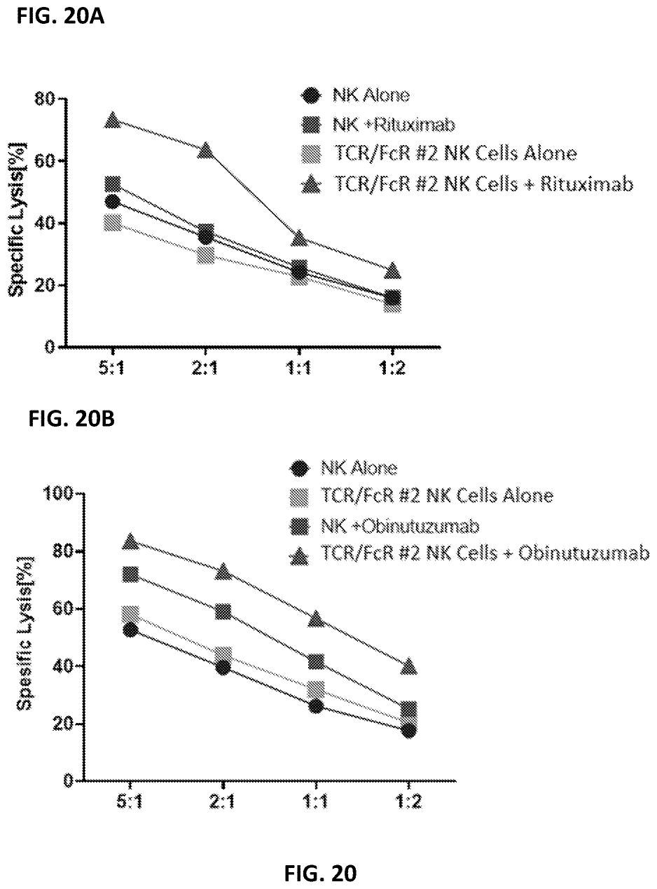

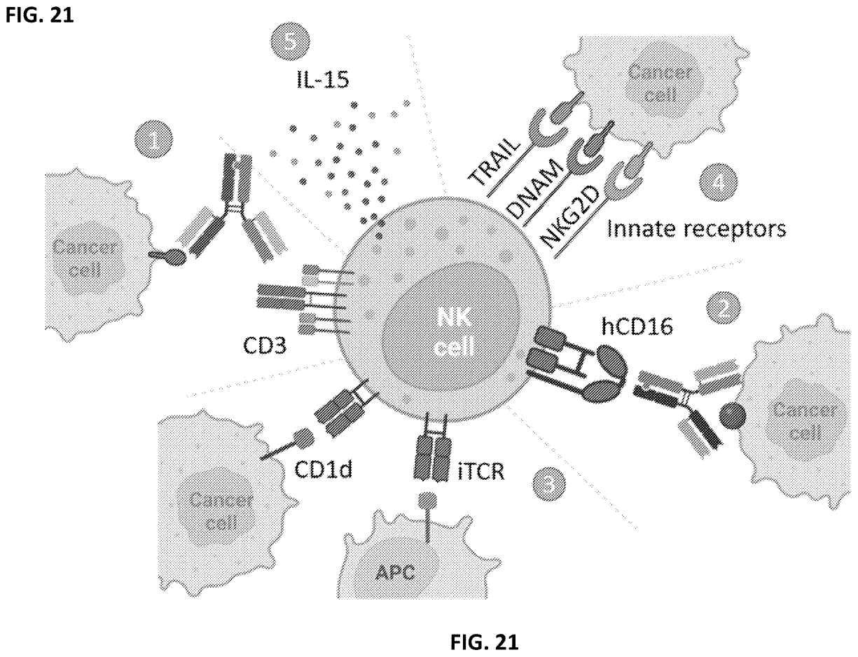

As shown in FIGS. 20A-20B, TCR/FcR #2 transduced NK cells demonstrated enhanced antitumor activity against Raji (B cell lymphoma) cells when loaded with anti-CD20 antibodies Rituximab or Obinutuzumab when compared to NT NK at various E:T ratios. NK cells were either non-transduced (NT) or transduced with TCR/FcR #2, and left unloaded or loaded with Rituximab (100 μg/ml final concentration) (FIG. 20A), or Obinutuzumab (100 μg/ml) for one hour at 37° C. in Click/RPMI media followed by washing. NK cells were co-cultured with chromium-51 labelled Raji cells at various E:T ratios (5:1, 2:1, 1:1, or 1:2), for four hours and chromium release (corresponding to the cytotoxicity of the cancer cells) was measured. Compared to non-loaded TCR/FcR NK cells or loaded NT NK cells, antibody-loaded TCR/FcR #2 transduced NK cells showed increased cytotoxicity against CD20+Raji cells.

### Example 8—Tcr/Fcr Nk Cells In Vivo

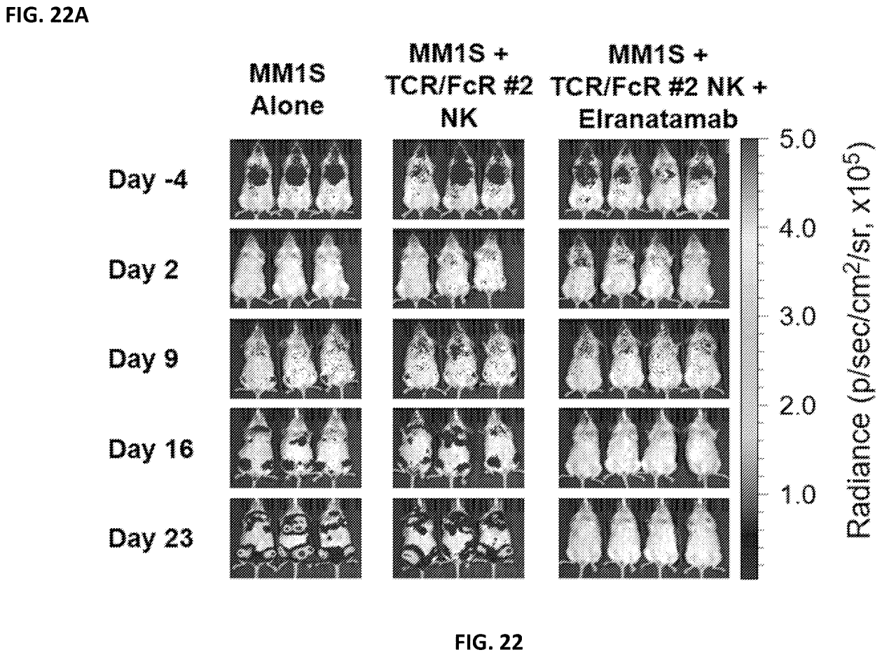

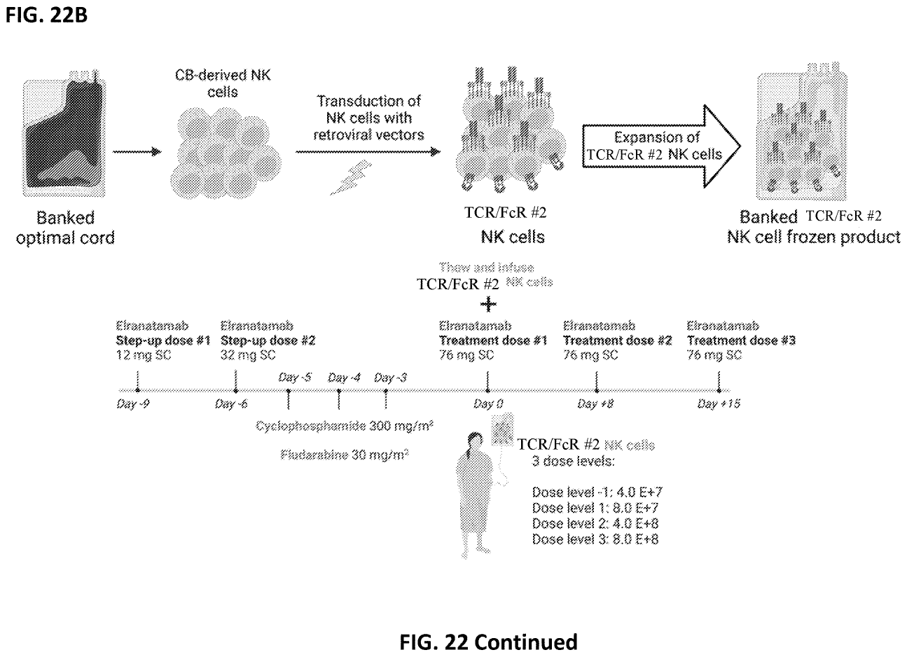

As shown in FIG. 22A, TCR/FcR and uTNK15 transduced NK cells co-injected with Elranatamab displayed enhanced control of BCMA+tumor cells (MM.1S, myeloma) in vivo. Ten-week-old female mice were irradiated on day −5 and engrafted with Ffluc-MM 1S (5×10⁵) on day −4. Cord blood derived NK cells were transduced with TCR/FcR construct #2 and uTNK15 and cryopreserved. NK cells were then thawed and 10 million transduced NK cells were infused into mice either alone or together with 10 g/ml Elranatamab via tail vein injection on day 0. Mice were subjected to weekly bioluminescence imaging (Xenogen IVIS-200 Imaging System) to monitor tumor growth. The results showed that TCR/FcR construct #2 transduced NK cells coupled with Elranatamab displayed robust tumor cell clearance relative to controls.

TCR/FcR transduced NK cells are co-injected and/or pre-loaded with antibody and display enhanced ex vivo and/or in vivo control (e.g., clearance, inhibition of proliferation, etc.) of tumor cells relative to controls. Ten-week-old mice are irradiated and engrafted with Ffluc-expressing tumor cells. Cord blood derived NK cells are obtained and engineered as described herein (e.g., transduced with TCR/FcR constructs and CD3 constructs; e.g., TCR/FcR #2 and uTNK15), and either pre-loaded with an antibody or left unloaded. Mice are injected with antibody, NK cells pre-loaded with antibody, NK cells and antibody, unloaded NK cells as monotherapy, and/or sham control on day 0. Mice are subjected to weekly bioluminescence imaging (e.g., Xenogen IVIS-200 Imaging System) to monitor tumor growth. The results show that antibody loaded engineered NK cells and/or engineered NK cells co-injected with antibody displayed robust tumor cell clearance relative to controls.

As shown in FIG. 22B, a clinical trial comprising TCR/FcR transduced NK cells with Elranatamab is performed. Cord blood derived NK cells are obtained and engineered to express TCR/FcR #2 and uTNK15. NK cells are expanded, and optionally frozen prior to use. Patients are treated with two doses of Elranatamab prior to NK cell treatment, the first step-up dose #1 occurring on day −9 and comprising 12 mg Elranatamab delivered subcutaneously, the second step-up dose #2 occurring on day −6 and comprising 32 mg of Elranatamab. Patients are treated with cyclophosphamide (300 mg/m²) and fludarabine (30 mg/m²) on days −5, −4, and −3. Patients are treated with engineered NK cells (4×10⁷, 8×10⁷, 4×10⁸, or 8×10⁸ cells) loaded Elranatamab, loading occurs ex-vivo (pre-loaded) and/or in vivo (co-injected), and patients are provided with a total known concentration of Elranatamab at about or exactly 76 mg. Patients are treated with additional Elranatamab and/or loaded engineered NK cells on day +8 (76 mg) and day +15 (76 mg).

A clinical trial comprising TCR/FcR transduced NK cells loaded with Elranatamab is performed. Cord blood derived NK cells are obtained and engineered as described herein (e.g., transduced with TCR/FcR constructs and CD3 constructs; e.g., TCR/FcR #2 and uTNK15) and optionally expanded and/or frozen. Patients are optionally treated with one or more doses of Elranatamab (e.g., subcutaneously; SC) prior to NK cell treatment (e.g., step-up dose #1, 12 mg; step-up dose #2, 32 mg), and optionally treated with cyclophosphamide (e.g., 300 mg/m²) and/or fludarabine (e.g., 30 mg/m²). Patients are treated with engineered NK cells (e.g., at 4×10⁷, 8×10⁷, 4×10⁸, or 8×10⁸ cells) loaded with Elranatamab, loading occurs ex-vivo and/or in vivo, and patients are provided with a total known concentration of Elranatamab (e.g., total concentration of about or exactly 76 mg). Patients are optionally treated with one or more additional doses comprising additional Elranatamab (e.g., total concentration of about or exactly 76 mg) and/or loaded engineered NK cells (e.g., at day +8, and day +15).

A clinical trial comprising TCR/FcR transduced NK cells loaded with one or more antibodies is performed. Cord blood derived NK cells are obtained and engineered as described herein (e.g., transduced with TCR/FcR constructs and CD3 constructs; e.g., TCR/FcR #2 and uTNK15) and optionally expanded and/or frozen. Patients are optionally treated with one or more doses of antibody prior to NK cell treatment, and optionally treated with additional agents (e.g., cyclophosphamide and/or fludarabine). Patients are treated with engineered NK cells (e.g., at 4×10⁷, 8×10⁷, 4×10⁸, or 8×10⁸ cells) loaded with antibody, loading occurs ex-vivo and/or in vivo, and patients can be provided with a total known concentration of antibody. Patients are optionally treated with one or more additional doses comprising additional antibody and/or loaded engineered NK cells.

### Example 9—Tcr/Fcr Nk Cells Targeting Her2 Displayed Enhanced Cytotoxicity

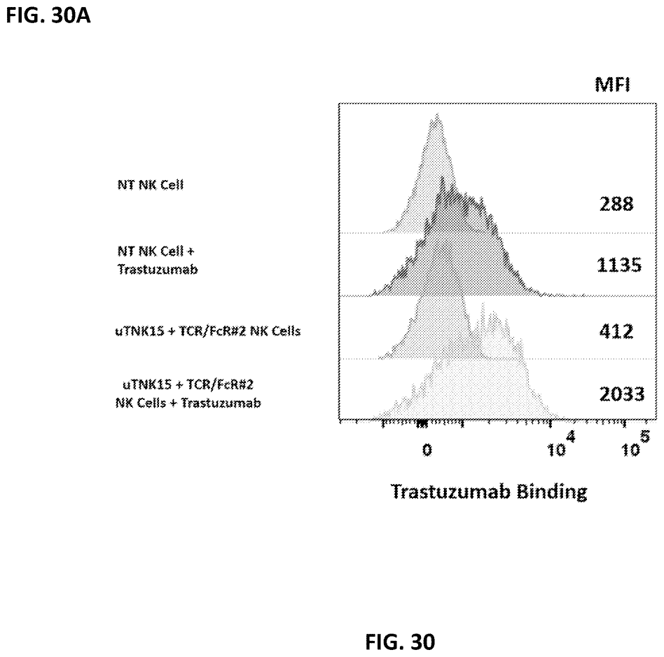

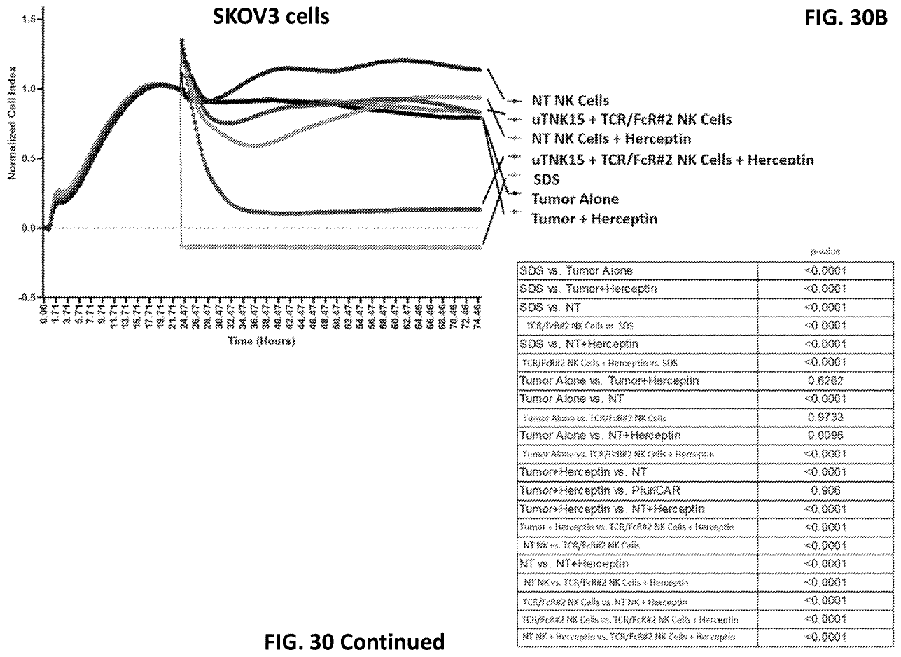

As shown in FIGS. 30A-30B, Trastuzumab (anti-HER2) bound uTNK15 and TCR/FcR #2 transduced NK cells with higher affinity than non-transduced (NT) NK cells, and Trastuzumab loaded engineered NK cells displayed superior antitumor activity against HER2+SKOV3 ovarian cancer cells in a long term xCELLigence killing assay. FIG. 30A, shows Trastuzumab binding to engineered NK cells. NK cells were extracted from cord blood and cultured in complete medium supplemented with irradiated (100 Gy) uAPC feeder cells at a ratio of 2:1 (feeder cell:NK). Additionally, recombinant human TL-2 was added at a concentration of 200 U/ml to facilitate expansion of the NK cells. Five days after expansion, natural killer (NK) cells were either transduced with the uTNK15 and TCR/FcR #2 constructs, or left non-transduced (NT). These cells were then loaded with Trastuzumab (200 g/ml for one hour at 37° C. in a complete medium). After incubation, the cells were washed and subjected to flow cytometric analysis to validate the binding. Flow cytometric analysis revealed that Trastuzumab could bind engineered NK cells with higher affinity than levels of binding observed with NT NK cells. FIG. 30B, Trastuzumab loaded uTNK15 and TCR/FcR #2 transduced NK cells showed enhanced killing compared to unloaded uTNK15 and TCR/FcR #2 transduced NK cells or loaded/unloaded NT NK Cells. NK cells were either NT (“NT” or “NT NK” shorthand for stats) or transduced with uTNK15 and TCR/FcR #2 (“TCR/FcR #2” shorthand for stats). NK cells were either left unloaded or loaded with Trastuzumab (200 g/ml for 1 hour at 37° C. in click/RPMI media & washed prior to co-culture) before co-culturing with tumor cells at 1:1 effector to target (E:T) ratio. Compared to loaded or unloaded NT NK Cells, unloaded engineered NK cells, or Trastuzumab alone, Trastuzumab-loaded engineered NK cells showed significantly increased cytotoxic activity against HER2+SKOV3 ovarian cancer cells.

### Example 10—Tcr/Fcr Nk Cells Targeting Gprc5D Displayed Enhanced Cytotoxicity

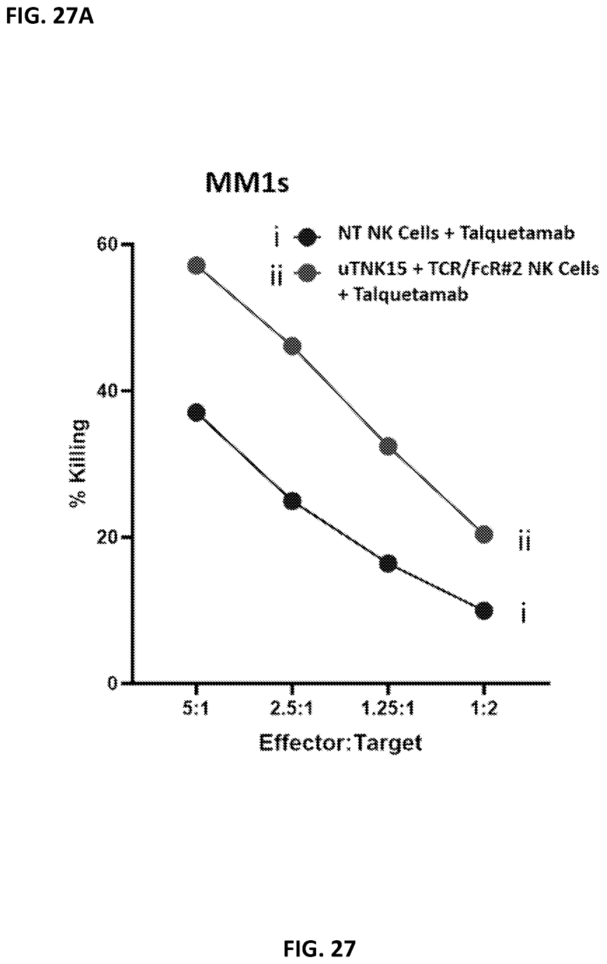

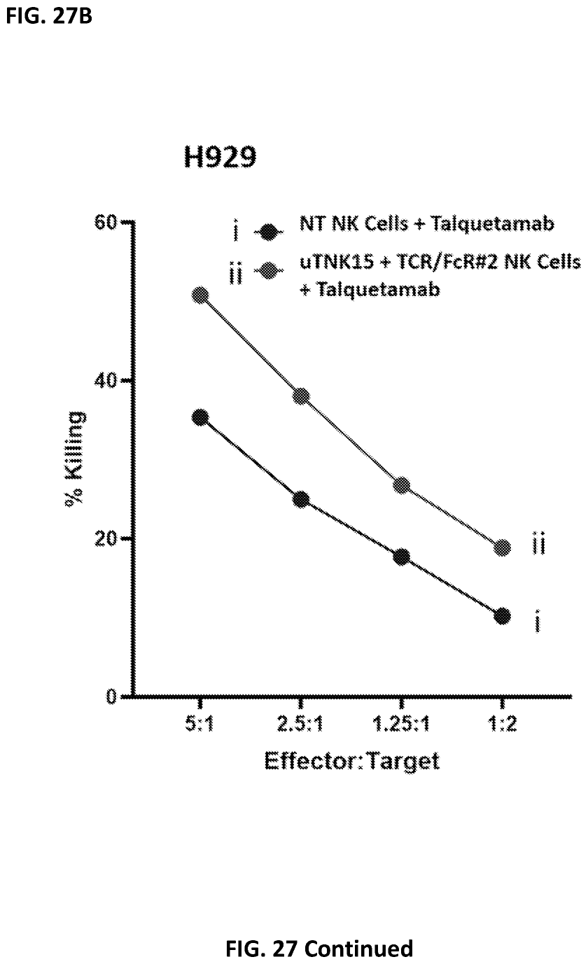

As shown in FIGS. 27A-27B, uTNK15 and TCR/FcR #2 transduced NK cells loaded with bispecific anti-GPRC5D/CD3 antibody Talquetamab (20 g/ml for 1 hour at 37° C. in click/RPMI media & washed prior to co-culture) demonstrated enhanced antitumor activity against GPRC5D+ tumor cells (e.g., MM1s cells, H929 cells) at various E:T ratios in a short term Cr51 assays when compared to Talquetamab loaded non-transduced (NT) NK cells. FIG. 27A shows the results against MM1s cells. FIG. 27B shows the results against H929 cells. Compared to Talquetamab-loaded NT NK cells, Talquetamab-loaded engineered NK cells showed enhanced cytotoxicity against GPRC5D+ tumor cells.

### Example 11—Tcr/Fcr Nk Cells Targeting Cd30 Displayed Enhanced Cytotoxicity

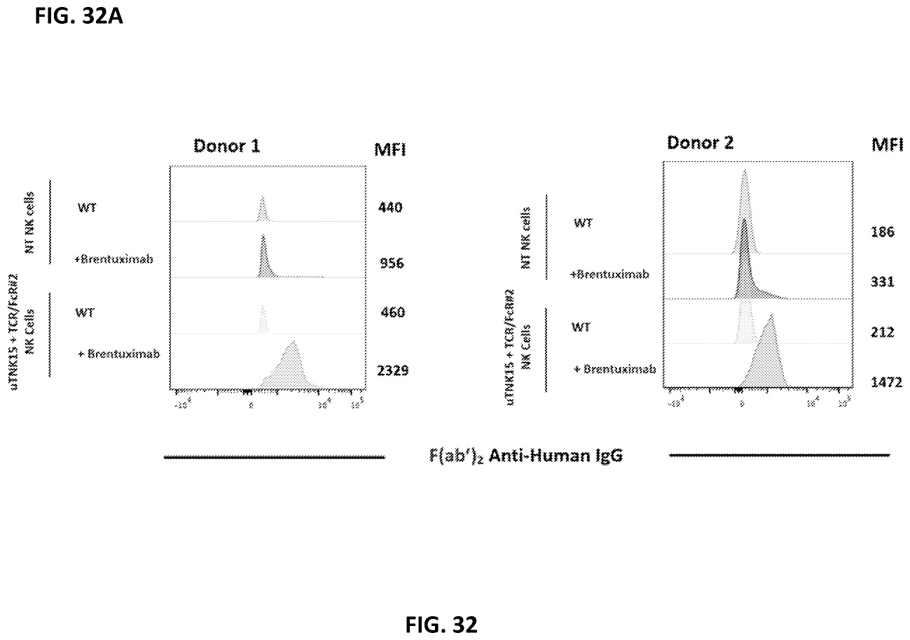

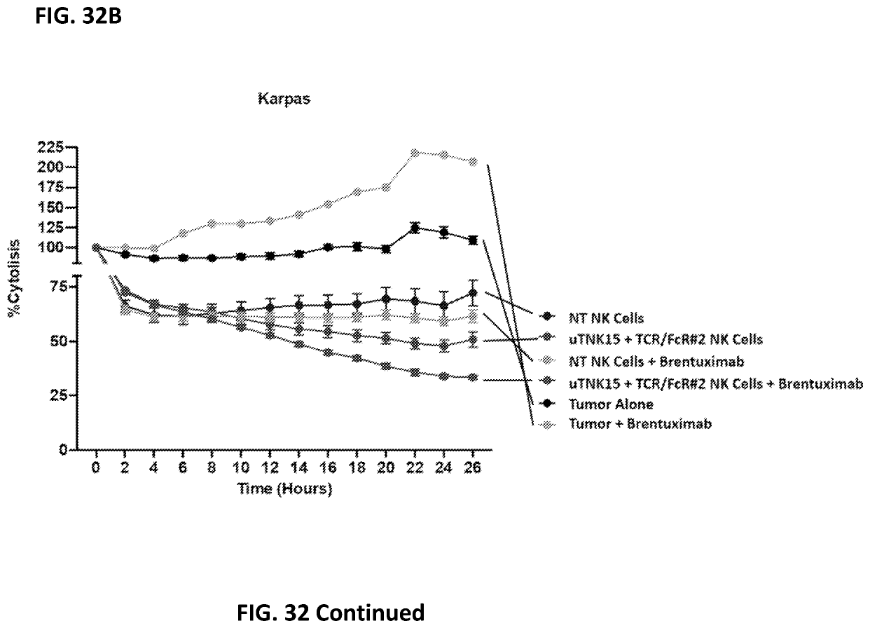

As shown in FIGS. 32A-32B, Brentuximab (anti-CD30 antibody-drug conjugate) bound to uTNK15 and TCR/FcR #2 transduced NK cells with higher affinity than non-transduced (NT) NK cells, and Brentuximab loaded engineered NK cells displayed superior antitumor activity against CD30+Karpas tumor cells in IncuCyte cytotoxicity assays. FIG. 32A, NK cells were isolated from cord blood and expand in complete media in the presence of irradiated (100 Gy) uAPC feeder cells (2:1 feeder cell:NK ratio) and recombinant human IL-2 (200 U/ml). Seven days following expansion, NK cells were either left non-transduced, or transduced with the uTNK15 and TCR/FcR #2 constructs. These cells were loaded with Brentuximab (100 g/ml for one hour at 37° C. in complete medium) and washed prior to flow cytometric validation of the Brentuximab binding. Brentuximab bound to engineered NK cells with higher affinity compared to NT NK cells, as determined by staining with F(ab′)2 Anti-Human antibody and flow cytometric analysis of NK cells. FIG. 32B, Brentuximab-loaded uTNK15 and TCR/FcR #2 transduced NK cells showed enhanced killing of CD30+(Karpas) tumor cells compared to non-loaded engineered NK cells or brentuximab-loaded NT NK Cells in IncuCyte cytotoxicity assays. NK cells were derived from cord blood expanded as described in 31A, and either not transduced or transduced with uTNK15 and TCR/FcR #2 constructs. NT NK cells or engineered NK cells were loaded with Brentuximab (100 g/ml for one hour at 37° C. in complete medium and washed prior to co-culturing) and co-cultured with CD30+Karpas tumor cell line at 1:1 effector to target ratios. Real-time cytotoxicity of effector cells against Karpas cells was measured every 2 hour over 24-hour period. Compared to non-loaded engineered NK cells or NT NK cells loaded with Brentuximab, engineered NK cells loaded with Brentuximab showed increased cytotoxicity of CD30+ Karpas cells.

All of the methods disclosed and claimed herein can be made and executed without undue experimentation in light of the present disclosure. While the compositions and methods of this invention have been described in terms of particular embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A polynucleotide comprising a sequence at least about 80%, 85%, 90%, 95%, 98%, or 100% identical to transcriptional reading frames comprising SEQ ID NO: 171-175.

2. The polynucleotide of claim 1, wherein the polynucleotide is comprised in a vector comprising at least about 80%, 85%, 90%, 95%, 98%, or 100% sequence identity to SEQ ID NOs: 177-181.

3. A polynucleotide comprising a sequence encoding a T cell receptor (TCR) alpha and TCR beta polypeptide, and/or TCR gamma and TCR delta polypeptide, and a polypeptide comprising a CD16 derived Fc binding domain.

4. The polynucleotide of claim 3, wherein the TCR polypeptides are invariant TCR (iTCR) polypeptides.

5. The polynucleotide of claim 4, wherein the iTCRα and iTCRβ polypeptides and/or polynucleotides encoding the same comprise a sequence at least about 80%, 85%, 90%, 95%, 98%, or 100% identical to any one or more of SEQ ID NOs: 51-149.

6. The polynucleotide of claim 4, wherein the iTCRβ polypeptide comprises a polynucleotide encoding a VP-DJ region that is at least about 80%, 85%, 90%, 95%, 98%, or 100% identical to any one or more of SEQ ID NOs: 75-149.

7. The polynucleotide of claim 4, wherein the iTCRβ polypeptide and/or polynucleotide encoding the same comprises a sequence at least about 80%, 85%, 90%, 95%, 98%, or 100% identical to any one or more of SEQ ID NO: NOs: 59-74.

8. The polynucleotide of claim 4, wherein the encoded iTCRβ polypeptide is at least about 80%, 85%, 90%, 95%, 98%, or 100% identical to SEQ ID NO: 60.

9. The polynucleotide of claim 8, wherein the polynucleotide encoding the iTCRβ polypeptide is at least about 80%, 85%, 90%, 95%, 98%, or 100% identical to SEQ ID NO: 59.

10. The polynucleotide of claim 4, wherein the iTCRα polypeptide and/or polynucleotide encoding the same comprises a sequence at least about 80%, 85%, 90%, 95%, 98%, or 100% identical to SEQ ID NOs: 51-52.

11. The polynucleotide of claim 3, wherein the polypeptide comprising a CD16 derived Fc binding domain comprises a human CD16A Fc binding domain.

12. The polynucleotide of claim 3, further comprising the Fc binding domain being fused in N to C terminus order to an optional hinge domain, a transmembrane domain (TMD), and one or more optional intracellular signaling domains (ICD).

13. The polynucleotide of claim 12, wherein the TMD is derived from CD16, or CD3ζ.

14. The polynucleotide of claim 12, wherein the TMD comprises or consists of a sequence with at least about 90% identity SEQ ID NOs: 163 or 167.

15. The polynucleotide of claim 12, wherein the hinge domain is derived from CD32.

16. The polynucleotide of claim 12, comprising a hinge domain that comprises or consists of a sequence with at least about 90% identity to SEQ ID NO: 161.

17. The polynucleotide of claim 12, comprising an ICD derived from CD16 and/or CD3ζ.

18. The polynucleotide of claim 3, wherein the polypeptide comprising a CD16 derived Fc binding domain does not comprise a mutation that renders the CD16 derived Fc binding domain resistant to cleavage.

19. The polynucleotide of claim 3, comprising a coding sequence that is least about 80%, 85%, 90%, 95%, 98%, or 100% identical to any one or more of SEQ ID NOs: 150-154.

20. The polynucleotide of claim 3, wherein the polynucleotide further encodes one or more cytokine sequences.

21. The polynucleotide of claim 20, wherein the cytokine comprises IL-15 and/or IL-21.

22. An engineered NK cell comprising the polynucleotide of any one of claims 1-21.

23. A method of treating a disease in an individual, the method comprising administration of the engineered NK cells of claim 22 to an individual in need thereof.

24. An engineered immune cell comprising, one or more transgenic polynucleotides encoding:
a) a CD3 protein complex comprising part or all of a single chain or any combination of CD3δ, CD3ε, CD3γ, or CD3ζ,
b) optionally at least one cytokine,
c) at least one TCRα and TCRβ chain and/or a TCRγ and TCRδ chain, and
d) a polypeptide comprising a CD16 Fc binding domain.

25. The engineered immune cell of claim 24, wherein the one or more transgenic polynucleotides comprise multicistronic transcriptional open reading frames.

26. The engineered immune cell of claim 24, wherein the cells are modified to express part or all of CD3δ, two of CD3ε, CD3γ, and/or CD3ζ.

27. The engineered immune cell of claim 24, wherein any one or more of CD3δ, CD3ε, CD3γ, and/or CD3ζ are linked to one or more heterologous intracellular signaling domains.

28. The engineered immune cell of claim 27, wherein the heterologous intracellular signaling domain is selected from the group consisting of CD16, NKG2D, DAP10, DAP12, 2B4, 4-1BB, CD2, CD28, and a combination thereof.

29. The engineered immune cell of claim 27, wherein the heterologous intracellular signaling domain comprises a DAP10 intracellular signaling domain.

30. The engineered immune cell of claim 29, wherein the heterologous intracellular signaling domain comprises an amino acid sequence at least about 85% identical to SEQ ID NO: 42.

31. The engineered immune cell of claim 27, wherein the heterologous intracellular signaling domain comprises a CD28 intracellular signaling domain.

32. The engineered immune cell of claim 31, wherein the heterologous intracellular signaling domain comprises an amino acid sequence at least about 85% identical to SEQ ID NO: 43.

33. The engineered immune cell of claim 27, wherein the heterologous intracellular signaling domain comprises a DAP10 and CD28 intracellular signaling domain.

34. The engineered immune cell of claim 33, wherein the heterologous intracellular signaling domain comprises an amino acid sequence at least about 85% identical to SEQ ID NO: 44.

35. The engineered immune cell of claim 24, wherein coding sequences for a CD3 protein complex and at least one cytokine are comprised in a first multicistronic construct, and wherein coding sequences for at least one TCRα and TCRβ chain and/or a TCRγ and TCRδ chain, and the polypeptide comprising a CD16 Fc binding domain are encoded by a second multicistronic construct.

36. The engineered immune cell of claim 24, comprising a coding sequence for a cytokine, wherein the cytokine comprises IL-2, IL-7, IL-12, IL-15, IL-18, IL-21, IL-23, and/or GMCSF.

37. The engineered immune cell of claim 36, where the cell is modified to express a polynucleotide sequence at least 85% identical to UT-NK15-DAP10 (SEQ ID NO: 45), UT-NK15-28 (SEQ ID NO: 47), or UTNK15-28-DAP10 (SEQ ID NO: 49).

38. The engineered immune cell of claim 37, wherein the cytokine comprises IL-15 and/or IL-21.

39. The engineered immune cell of claim 38, wherein the cytokine comprises IL-15 and comprises a polypeptide sequence and/or polynucleotide sequence encoding the same that is at least 85% identical to any one or more of SEQ ID NOs: 182-183.

40. The engineered immune cell of claim 38, wherein the cytokine comprises IL-21 and comprises a polypeptide sequence and/or polynucleotide sequence encoding the same that is at least 85% identical to any one or more of SEQ ID NOs: 184-187.

41. The engineered immune cell of claim 24, wherein the TCR polypeptides are invariant TCR (iTCR) polypeptides, and wherein the iTCRα and iTCRβ polypeptides and/or polynucleotides encoding the same comprise a sequence at least about 80%, 85%, 90%, 95%, 98%, or 100% identical to any one or more of SEQ ID NOs: 51-149.

42. The engineered immune cell of any one of claims 24 to 41, wherein the TCR polypeptides are invariant TCR (iTCR) polypeptides.

43. The engineered immune cell of claim 42, wherein the iTCRα and iTCRβ polypeptides and/or polynucleotides encoding the same comprise a sequence at least about 80%, 85%, 90%, 95%, 98%, or 100% identical to any one or more of SEQ ID NOs: 51-149.

44. The engineered immune cell of claim 42, wherein the iTCRβ polypeptide comprises a polynucleotide encoding a VP-DJ region that is at least about 80%, 85%, 90%, 95%, 98%, or 100% identical to any one or more of SEQ ID NOs: 75-149.

45. The engineered immune cell of claim 42, wherein the iTCRβ polypeptide and/or polynucleotide encoding the same comprises a sequence at least about 80%, 85%, 90%, 95%, 98%, or 100% identical to any one or more of SEQ ID NO: NOs: 59-74.

46. The engineered immune cell of claim 42, wherein the encoded iTCRβ polypeptide is at least about 80%, 85%, 90%, 95%, 98%, or 100% identical to SEQ ID NO: 60.

47. The engineered immune cell of claim 46, wherein the polynucleotide encoding the iTCRβ polypeptide is at least about 80%, 85%, 90%, 95%, 98%, or 100% identical to SEQ ID NO: 59.

48. The engineered immune cell of claim 42, wherein the iTCRα polypeptide and/or polynucleotide encoding the same comprises a sequence at least about 80%, 85%, 90%, 95%, 98%, or 100% identical to SEQ ID NO: 51-52.

49. The engineered immune cell of claim 24, wherein the CD16 Fc binding domain comprising polypeptide comprises a human CD16 derived Fc binding domain.

50. The engineered immune cell of claim 24, wherein the polypeptide comprising a CD16 derived Fc binding domain comprises a human CD16A Fc binding domain.

51. The engineered immune cell of claim 24, wherein the Fc binding domain is fused in N to C terminus order to an optional hinge domain, a transmembrane domain (TMD), and one or more optional intracellular signaling domains (ICD).

52. The engineered immune cell of claim 51, wherein the TMD is derived from CD16, or CD3ζ.

53. The engineered immune cell of claim 51, wherein the TMD comprises or consists of a sequence with at least about 90% identity to SEQ ID NOs: 163 or 167.

54. The engineered immune cell of claim 51, wherein the hinge domain is derived from CD32.

55. The engineered immune cell of claim 51, wherein the hinge domain comprises or consists of a sequence with at least about 90% identity to SEQ ID NO: 161.

56. The engineered immune cell of claim 51, comprising an ICD derived from CD16 and/or CD3ζ.

57. The engineered immune cell of claim 24, comprising a coding sequence that is least about 80%, 85%, 90%, 95%, 98%, or 100% identical to any one or more of SEQ ID NOs: 171-175.

58. The engineered immune cell of any one of claims 24 to 41, wherein the cell is a Natural Killer (NK) cell.

59. The engineered NK cell of claim 58, wherein the NK cells are derived from cord blood (CB), peripheral blood (PB), bone marrow, stem cells, or a combination thereof.

60. The engineered NK cell of claim 58, wherein the NK cells are primary NK cells, and are not derived from stem cells and/or induced pluripotent stem cells (iPSCs).

61. The engineered NK cell of claim 58, wherein the NK cells are loaded with (complexed with) one or more antibodies.

62. The engineered NK cell of claim 61, wherein the one or more antibodies are one or more monospecific, bispecific, or multi-specific antibodies.

63. The engineered NK cell of claim 61, wherein at least one or more antibodies comprises a glycoengineered Fc domain that has a high affinity to wild type CD16.

64. The engineered NK cell of claim 61, wherein the at least one or more antibodies comprise a non-glycoengineered Fc domain that has a low affinity to wild type CD16 Fc binding domains.

65. The engineered NK cell of claim 64, wherein the non-glycoengineered Fc domain is loaded on (complexed to) the transgenic polypeptide comprising a CD16 Fc binding domain.

66. The engineered NK cell of claim 61, wherein the one or more antibodies comprise an IgG1 and/or IgG4 Fc domain.

67. The engineered NK cell of claim 61, wherein the one or more antibodies target antigens CD3, CD16, CD28, EGFR, c-MET, CD30, PSMA, MUC17, CD33, FLT3, STEAP1, BCMA, CLDN18.2, CD123, CD19, CD20, EpCAM, CEA, GPC3, CD38, CD33, CD22, HER2, GPA33, GD2, MUC16, GPRC5D, DLL-3, CLEC12A, FcRH5, and/or SSTR.

68. The engineered NK cell of claim 61, wherein the one or more antibodies target CD3, CD16, CD28, CD19, CD20, CD30, HER2, GPRC5D, EGFR, c-MET, and/or BCMA.

69. The engineered NK cell of claim 61, wherein the one or more antibodies comprise [fam]-trastuzumab deruxtecan, Abciximab, Adalimumab, Ado-trastuzumab emtansine, Aducanumab, Alemtuzumab, Alirocumab, Amivantamab, Anifrolumab, Ansuvimab, Atezolizumab, Atoltivimab with Maftivimab and Odesivimab-ebgn (aka Inmazeb), Avelumab, Basiliximab, Belantamab mafodotin, Belimumab, Benralizumab, Bevacizumab, Bezlotoxumab, Bimekizumab, Blinatumomab, Brentuximab vedotin, Brodalumab, Brolucizumab, Burosumab, Canakinumab, Caplacizumab, Casirivimab+imdevimab, Catumaxomab, Cemiplimab, Certolizumab pegol, Cetuximab, Cevostamab, Crizanlizumab, Daclizumab, Daratumumab, Denosumab, Dinutuximab, Donanemab, Dostarlimab, Dupilumab, Durvalumab, Eculizumab, Edrecolomab, Efalizumab, Elotuzumab, Emapalumab, Emicizumab, Enfortumab vedotin, Eptinezumab, Erenumab, Evinacumab, Evolocumab, Faricimab, Fremanezumab, Galcanezumab, Gemtuzumab, Gemtuzumab-Ozogamicin, Golimumab, Guselkumab, Ibalizumab, Ibritumomab tiuxetan, Idarucizumab, Imgatuzumab, Inebilizumab, Infliximab, Inolimomab, Inotuzumab, Inotuzumab-Ozogamicin, IPH61, Ipilimumab, Isatuximab, Ixekizumab, Lanadelumab, Lecanemab, Loncastuximab tesirine, Margetuximab, Mepolizumab, Mirvetuximab soravtansine, Mogamulizumab, Mosunetuzumab, Moxetumomab pasudotox, Muromonab-CD3, Narsoplimab, Natalizumab, Naxitamab, Nebacumab, Necitumumab, Nirsevimab, Nivolumab, Obiltoxaximab, Obinutuzumab, Ocrelizumab, Ofatumumab, Olaratumab, Omalizumab, Omburtamab, Oportuzumab monatox, Palivizumab, Panitumumab, Pembrolizumab, Penpulimab, Pertuzumab, Polatuzumab vedotin, Ramucirumab, Ranibizumab, Ravulizumab, Raxibacumab, Regdanvimab, Relatlimab, Reslizumab, Retifanlimab, Risankizumab, Rituximab, Romosozumab, Sacituzumab govitecan, Sarilumab, Satralizumab, Secukinumab, Siltuximab, Sintilimab, Sotrovimab, Spesolimab, Sutimlimab, Tafasitamab, Tebentafusp, Teclistamab, Teplizumab, Teprotumumab, Tezepelumab, Tildrakizumab, Tislelizumab, Tisotumab vedotin, Tixagevimab, cilgavimab, Tocilizumab, Toripalimab, Tositumomab-I131, Tralokinumab, Trastuzumab, Tremelimumab, Ublituximab, Ustekinumab, Vedolizumab, AMG 160/Acapatamab, AMG 199/TNB 585, AMG 330, AMG 427/EMIRODATAMAB, AMG 509, AMG 701, AMG 910, APVO414/ES414/MOR209, APV0436, Catumaxomab/Removab, CC-1, CC-93269/EM801, Cibisatamab/RG7802/RO6958688, CLN-049, Elranatamab/PF-06863135, EMB-06, Epcoritamab/GEN3013, ERY974, Flotetuzumab/MGD006, Glofitamab/RG6026/RO7082859, ISB 1342/GBR 1342, JNJ-63709178, JNJ-63898081, JNJ-67571244, JNJ-75348780, Linvoseltamab/REGN 5458, M701, M802, MGD007, Mosunetuzumab/RG7828, Nivatrotamab/Hu3F8-BsAb, Odronextamab/REGN1979, REGN4018, REGN5459, REGN7075, REGN5678, Talquetamab/JNJ-64407564, Tarlatamab/AMG 757, Tepoditamab/MCLA-117, TNB-383B, TNB-486, TNB-585, XmAb13676/Plamotamab, XmAb14045/Vibecotamab, XmAb18087/Tidutamab, and/or AFM13.

70. The engineered NK cell of claim 61, wherein the one or more antibodies comprise Elranatamab, Imgatuzumab, Margetuximab, Amivantamab, Blinatumomab, Obinutuzumab, IPH61 (also known as IPH6101 or SAR443579), Teclistamab, Cetuximab, Talquetamab, Pertuzumab, Trastuzumab, Tafasitamab, Brentuximab, and/or Rituximab.

71. The engineered NK cell of claim 70, wherein the one or more antibodies comprises or consists of Elranatamab.

72. The engineered NK cell of claim 70, wherein the one or more antibodies comprises or consists of Imgatuzumab.

73. The engineered NK cell of claim 70, wherein the one or more antibodies comprises or consists of Margetuximab.

74. The engineered NK cell of claim 70, wherein the one or more antibodies comprises or consists of Amivantamab.

75. The engineered NK cell of claim 70, wherein the one or more antibodies comprises or consists of Blinatumomab.

76. The engineered NK cell of claim 70, wherein the one or more antibodies comprises or consists of Obinutuzumab.

77. The engineered NK cell of claim 70, wherein the one or more antibodies comprises or consists of IPH61.

78. The engineered NK cell of claim 70, wherein the one or more antibodies comprises or consists of Teclistamab.

79. The engineered NK cell of claim 70, wherein the one or more antibodies comprises or consists of Cetuximab.

80. The engineered NK cell of claim 70, wherein the one or more antibodies comprises or consists of Rituximab.

81. The engineered NK cell of claim 70, wherein the one or more antibodies comprises or consists of Talquetamab.

82. The engineered NK cell of claim 70, wherein the one or more antibodies comprises or consists of Pertuzumab.

83. The engineered NK cell of claim 70, wherein the one or more antibodies comprises or consists of Trastuzumab.

84. The engineered NK cell of claim 70, wherein the one or more antibodies comprises or consists of Tafasitamab.

85. The engineered NK cell of claim 70, wherein the one or more antibodies comprises or consists of Brentuximab.

86. The engineered NK cell of claim 61, wherein the NK cell expresses the one or more antibodies.

87. The engineered NK cell of claim 58, wherein the NK cell is further modified to express one or more additional heterologous proteins selected from the group consisting of an antigen receptor, a cytokine, a homing receptor, a chemokine receptor, and a combination thereof.

88. The engineered NK cell of claim 58, wherein the NK cells are pre-activated with one or more cytokines.

89. The engineered NK cell of claim 88, wherein the cytokines are IL-2, IL-7, IL-12, IL-15, IL-18, IL-21, or a combination thereof.

90. The engineered NK cell claim 58, wherein the NK cell further comprises one or more engineered mutations in an endogenous gene.

91. The engineered NK cell of claim 90, wherein the endogenous gene is GR, TGFBR2, CISH, and/or CD38.

92. A composition comprising the engineered NK cell of any one of claims 58 to 91.

93. The composition claim 92, further comprising a pharmaceutically acceptable excipient.

94. The composition claim 92 or 93, wherein the composition is comprised in a delivery device.

95. A method of treating a disease in an individual, the method comprising the step of administering to the individual a therapeutically effective amount of any one of the cells of claim 24 to 41.

96. The method of claim 95, wherein the disease is an autoimmune disease, infection, and/or cancer.

97. The method of claim 96, wherein the disease is an autoimmune disease.

98. The method of claim 97, wherein the autoimmune disease comprises a B cell related autoimmunity, systemic lupus erythematosus (SLE), Systemic scleroderma (SSc), multiple sclerosis (MS), Grave's disease, rheumatoid arthritis (RA), myositis, dermatomyositis. myasthenia gravis, Sjogren's syndrome, diffuse scleroderma, inflammatory myopathy, inflammatory myopathy, ANCA-associated systemic vasculitis, antiphospholipid syndrome, immune nephritis, ITP, refractory POEMS syndrome, amyloidosis, autoimmune hemolytic anemia, and/or vasculitis.

99. The method of claim 95, wherein the disease is cancer.

100. The method of claim 99, wherein the cancer expresses CD19, CD20, CD30, HER2, GPRC5D, EGFR, c-MET, and/or BCMA.

101. The method of claim 99, wherein the cancer is pancreatic cancer, colorectal cancer, ovarian cancer, kidney cancer, glioblastoma, breast cancer, renal cancer, myeloma, and/or leukemia.

102. The method of claim 98 or 101, further comprising administering to the individual at the same time or at different time, one or more monospecific, bispecific, and/or multispecific antibodies.

103. The method of claim 102, wherein the one or more antibodies comprise Elranatamab, Imgatuzumab, Margetuximab, Amivantamab, Blinatumomab, Obinutuzumab, IPH61 (also known as IPH6101 or SAR443579), Teclistamab, Cetuximab, Talquetamab, Pertuzumab, Trastuzumab, Tafasitamab, Brentuximab, and/or Rituximab.

104. The method of claim 102, wherein the one or more antibodies are administered at the same time, and/or the one or more antibodies and the engineered NK cells are complexed prior to administration to the individual.

105. The method of claim 102, wherein the one or more antibodies are administered more than once, including at least once at a time point after administration of the engineered NK cells.

106. The method of claim 102, wherein the one or more antibodies are administered more than once, including at least once at a time point before administration of the engineered NK cells.

107. The method of claim 102, wherein the one or more antibodies are administered more than once, including at least once at a time point before administration of the engineered NK cells and at least once at a time point after administration of the engineered NK cells.
